(19) United States
(12) Patent Application Publication
(10) Pub. No.: US 2015/0246025 A1
(43) Pub. Date: Sep. 3, 2015

(54) 2-PHENYL-5-HETEROCYCLYL-TETRAHYDRO-2H-PYRAN-3-AMINE COMPOUNDS FOR USE IN THE TREATMENT OF DIABETES AND ITS ASSOCIATED DISORDERS

(71) Applicant: CADILA HEALTHCARE LIMITED, Gujarat (IN)

(72) Inventors: Ranjit C. Desai, Ahmedabad (IN); Rajesh Bahekar, Ahmedabad (IN); Pradip Jadav, Ahmedabad (IN); Amitgiri Goswami, Ahmedabad (IN); Pankaj Patel, Ahmedabad (IN)

(21) Appl. No.: 14/436,504

(22) PCT Filed: Oct. 17, 2013
(86) PCT No.: PCT/IN2013/000627

§ 371 Date: Apr. 17, 2015

(30) Foreign Application Priority Data
Oct. 17, 2012 (IN) 3030/MUM/2012

(51) Int. Cl. A61K 31/437; A61K 31/407; A61K 45/06; C07D 487/10; A61K 31/403; C07D 471/04; C07D 471/10; A61K 31/435; C07D 405/04; C07D 487/04; C07D 491/048

(52) U.S. Cl. CPC A61K 31/437; C07D 487/04; A61K 31/407; A61K 45/06; C07D 487/10; C07D 491/048; C07D 471/04; C07D 471/10; A61K 31/435; C07D 405/04; A61K 31/403

(57) ABSTRACT

The present invention relates to novel compounds of the general formula (I) their tautomeric forms, their enantiomers, their diastereoisomers, their pharmaceutically accepted salts, or pro-drugs thereof, which are useful for the treatment or prevention of diabetes mellitus (DM), obesity and other metabolic disorders. The invention also relates to process for the manufacture of said compounds, and pharmaceutical compositions containing them and their use.

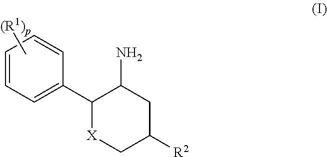

### FIELD OF INVENTION

The present invention relates to novel compounds of the general formula (I) their tautomeric forms, their enantiomers, their diastereoisomers, their pharmaceutically accepted salts, or pro-drugs thereof, which are useful for the treatment or prevention of diabetes and its associated disorders, obesity and other metabolic disorders. The invention also relates to process for the manufacture of said compounds, and pharmaceutical compositions containing them and their use.

### BACKGROUND OF THE INVENTION

The metabolic syndrome (or syndrome X) is a collection of associated disorders, affected by lifestyle, genetic disposition and environment (Lancet, 365, 1415, 2005; Diabetes, 41, 715, 1992). Obesity and diabetes are emerging as the global epidemic of the 21^{st} century and becoming major health problems worldwide (Diabetic Medicine, 14, S7-S85, 1997; Nature Med., 12, 62-66, 2006; Diabetes Care, 27, 1047-1053, 2004). Diabetes mellitus (DM) refers to a disease derived from multiple causative factors and characterized by elevated levels of plasma glucose (hyperglycemia), in fasting state or after administration of glucose during an oral glucose tolerance test (Diabetes Care, 26, 3160-3167, 2003; Diabetes Care, 33, S62, S69, 2010).

There are two generally reorganized forms of diabetes. In type 1 or Insulin-dependent diabetes mellitus (IDDM), patients produce little or no insulin (insulin deficiency), due to autoimmunological destruction of the insulin-producing pancreatic β-cells. Type 1 diabetes most commonly occurs in children. In type 2 diabetes mellitus (T2DM) or non-insulin dependent diabetes mellitus (NIDDM), patients often have plasma insulin levels that are the same or elevated compared to non-diabetic subjects (Diabetes Care, 20, 1183-1197, 1997; Diabet Med., 15, 539-553, 1998). Majority of diabetic people are diagnosed with T2DM and of these, 90% are obese or overweight (Diabetologia, 42, 499-518, 1999; Nature, 414, 782-787, 2001).

T2DM is a common chronic and progressive disease arising from a complex pathophysiology involving the dual endocrine effects of insulin resistance and impaired insulin secretion. Abnormal glucose homeostasis is associated both directly and indirectly with alterations of the lipid, lipoprotein and apolipoprotein metabolism and other metabolic and hemodynamic disease. Therefore patients with T2DM are at increased risk of macrovascular and microvascular complications, including coronary heart disease, stroke, peripheral vascular disease, hypertension, nephropathy, neuropathy, and retinopathy (Diabetes Metab., 23(5), 454-455 1997; Diabet Med., 15(7), 539-53, 1998). Thus, therapeutical control of glucose homeostasis, lipid metabolism and hypertension are critically important in the clinical management and treatment of T2DM (Med. J. Aust, 179(7), 379-383, 2003).

The treatment of T2DM typically begins with diet and exercise, followed by oral antidiabetic monotherapy (N. Engl. J. Med., 344, 1343-1350, 2001; Diabetes Care, 20, 537-544, 1997). The current antidiabetic therapeutics include compounds that increase the amount of insulin secreted by the pancreas, compounds that decrease the rate at which glucose is absorbed from the gastrointestinal tract and compounds that increase the sensitivity of target organs to insulin (Ann. Intern. Med., 147, 386-399, 2007; Clin. Ther., 29, 1236-1253, 2007). Conventional monotherapy may initially control blood glucose in some patients; however it is associated with a high secondary failure rate.

The limitations of single-agent therapy for maintaining glycemic control may be overcome, by combining multiple antidiabetic drugs (Cardiovasc. Diabetol., 10, 12-62, 2013). Current treatments for diabetic patients include various oral antihyperglycemic agents; however, over a period of time nearly half of T2DM patients lose their response to these agents and thereby require insulin therapy. Also, adverse events (such as weight gain and hypoglycemia with insulin; lactic acidosis, nausea & diarrhea with biguanides; liver toxicity and CVS risk with glitazones) associated with the existing antihyperglycemic agents raise safety concerns (Drugs, 68(15), 2131-2162, 2008; Drugs, 65(3), 385-411, 2005; Diabetes Obes Metab., 9,799-812, 2007).

Thus, along with healthy lifestyle, majority of T2DM patients need pharmacological intervention, which mainly consists of combination of oral antidiabetic drugs with subcutaneous insulin injections (Clin Ther., 29, 1236-1253, 2007). Despite large efforts to discover new antidiabetic drugs, only three classes of oral hypoglycemic agents (sulfonylureas, biguanides, and insulin sensitizers) are available for the treatment of T2DM. Except incretin therapies, most of the available anti-hyperglycemic agents including insulin promote weight gain, which further aggravates obesity-associated cardiovascular risk and insulin resistance (Diabetes Care, 27, 1535-1540, 2004; Ann. Intern. Med., 147, 386-399, 2007). Thus, there is an urgent need to develop novel agents for glycemic control that can complement with existing therapies and prevent the progression of secondary complications associated with diabetes.

Despite such epidemic proportion of the disease, only 4 out of 10 patients treated for diabetes meet the treatment targets, forcing clinicians to move from initial treatment with one agent to more aggressive intervention with multiple oral therapies, as well as insulin. Hence, new therapeutic agents which would treat diabetes along with its comorbidities are constantly needed in current regimen.

Dipeptidyl peptidase-IV (DPP-IV) is a serine protease, which selectively cleaves the N-terminal dipeptide from the penultimate position of Glucose-dependent Insulinotropic Polypeptide (GIP) and Glucagon-Like Peptide (GLP-1) thus makes them inactive (Diabetes Obes Metab., 10, 376-387, 2008; Diabetes Care, 30, 1979-1987, 2007). GLP-1 is an incretin hormone secreted by intestinal L-Cells in response to food intake. The active GLP-1 stimulates insulin secretion, inhibits glucagon release and slows gastric emptying, which together contributes for effective glucose homeostasis in patients with T2DM. Inhibition of DPPIV activity extends the duration of action of endogenous GLP-1, thereby exhibiting all the favorable attributes of GLP-1 (Lancet, 368, 1696-1705, 2006; Horm Metab Res., 36 (11-12), 867-76, 2004).

DPP-IV inhibitors offer a number of potential advantages over existing diabetes therapies, including a lowered risk of hypoglycemia, weight gain and the potential for regeneration and differentiation of pancreatic β-cells (Handbook Exp Pharmacol., 203, 53-74, 2011; Curr Med Res Opin., 23(4), 919-31, 2007). Because of these multiple benefits of GLP-1 mediated glucose homeostasis, orally bioavailable DPP-IV inhibitors has been developed as promising therapeutic agents for the treatment of T2DM (Am. J. Ther., 15(5), 484-91, 2008).

The therapeutic potential of DPP-IV inhibitors for the treatment of T2DM have been discussed and reviewed extensively (Exp. Opin. Invest. Drugs, 12, 87-100, 2003; Exp. Opin. Ther. Patents, 13, 499-510, 2003; Exp. Opin. Investig. Drugs, 13, 1091-1102, 2004; Curr. Opin. Drug Discovery Development, 11, 512-532, 2008 and Trends in Molecular Medicine, 14, 161-168, 2008). Various DPPIV inhibitors such as Vildagliptin (Galvus), Saxagliptin (Onglyza), Alogliptin (Nesina), Linagliptin (Tradjenta) and Sitagliptin (Januvia) are in clinic for the treatment of T2DM.

Patent applications WO 97/40832; WO 98/19998; WO 01/68603; WO 02/38541; WO 02/076450; WO 03/000180; WO 03/000181; WO 03/024942; WO 03/033524; WO 03/035057; WO 03/035067; WO 03/037327; WO 03/074500; WO 03/082817; WO 04/007468; WO 04/018467; WO 04/026822; WO 04/032836; WO 04/037181; WO 04/041795; WO 04/043940; WO 04/046106; WO 04/050022; WO 04/058266; WO 04/064778; WO 04/069162; WO 04/071454; WO 06/039325; WO 07/024993; WO 08/060488; WO 09/139362; WO 10/056708; WO 11/028455; WO 11/037793; WO 11/146358; WO 12/118945; WO 13/003249; WO 13/003250; U.S. Pat. Nos. 5,939,560; 6,011,155; 6,107,317; 6,110,949; 6,166,063; 6,124,305; 6,303,661; 6,432,969; 6,617,340; 0,232,676; 0,220,766; 8,415,297; 0,157,940, 6,699,871; Bioorg. Med. Chem. 17, 1783-1802, 2009 etc. represents different structural classes of DPP-IV inhibitors.

Structurally, DPP-1V enzyme resembles with several other proteases, so while designing new class of DPP-IV inhibitors, it is essential to consider selectivity of DPP-IV inhibitors over other serine protease, especially DPP-2, DPP-8 and DPP-9 (Diabetes, 54, 2988-2994, 2005; Bioorganic Med. Chem. Lett., 17, 3716-3721, 2007). Though several DPP-IV inhibitors are in the market, attempts are still underway to develop potent and selective DPP-IV inhibitors, which are better or are of comparable efficacy with the present DPP-IV inhibitors, have lesser side effects, require a lower dosage regime or frequency of administration and have advantage of treating other metabolic disorders.

### PRIOR ART

Earlier, a series of invention relating to substituted aminocyclohexanes (WO 06/127530; WO 07/87231), substituted aminopiperidines (WO 06/039325; U.S. Ser. No. 05/034,775), substituted aminotetrahydrothiopyrans (WO 11/103256; U.S. Ser. No. 11/025,182), substituted aminopiperidines (WO 11/037793; U.S. Ser. No. 10/048,871) and substituted aminotetrahydropyrans (WO 11/028455; U.S. Ser. No. 10/046,270; WO 10/056708; U.S. Ser. No. 09/063,976; WO 13/003250; U.S. Ser. No. 12/043,924; WO 13/003249; U.S. Ser. No. 12/043,922; U.S. Ser. No. 13/841,5297; U.S. Ser. No. 13/015,7940; WO 07/097931; WO 08/060488; U.S. Ser. No. 07/023,2676; WO 07/136603; WO 07/126745; WO 06/009886; U.S. Ser. No. 05/021,556; EP1761532), with a general formula of (A), wherein ‘V’ represent selected bicyclic hetero-aromatic ring systems, have been reported as DPP-IV inhibitors for the effective treatment of T2DM, by Merck Sharp & Dohme (MSD) Corporation Limited.

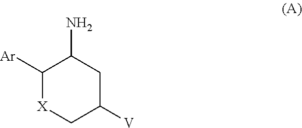

### Wherein: X=—CH₂; —NR; O; S

We herein disclose novel compounds of general formula (I) which are DPP-IV inhibitors and are useful for the prevention and treatment of diseases states mediated by DPP-IV enzyme.

### SUMMARY OF THE INVENTION

The present invention discloses novel compounds of the general formula (I) that are DPP-IV inhibitors and are useful for the prevention and treatment of disease states mediated by DPP-IV enzyme. The compounds of the present invention are useful in the treatment of human or animal body, by inhibition of DPP-IV. The compounds of this invention are therefore suitable for the prevention and treatment of disease states mediated by DPP-IV enzyme. Surprisingly it was found that some of these compounds were found to have longer half-life and an extended pharmacokinetic profile. Such properties may allow for an extended dosing interval of more than one day.

### EMBODIMENT(S) OF THE INVENTION

An embodiment of the present invention provides novel compounds of the general formula (I), their tautomeric forms, their enantiomers, their diastereoisomers, their stereoisomers, their pharmaceutically acceptable salts, and pharmaceutical compositions containing them or their suitable mixtures.

In a further embodiment of the present invention is provided pharmaceutical compositions containing compounds of the general formula (I), their tautomeric forms, their enantiomers, their diastereoisomers, their stereoisomers, their pharmaceutically acceptable salts, or their mixtures in combination with suitable carriers, solvents, diluents and other media normally employed in preparing such compositions.

In a still further embodiment is provided the use of novel compounds of the present invention as DPP-IV inhibitors, by administering a therapeutically effective and nontoxic amount of compounds of general formula (I) or their pharmaceutically acceptable compositions to the mammals for the treatment of diabetes and associated disorders.

In yet another embodiment is provided a composition comprising the compounds of formula (I) along with atleast a second suitable medicament for the treatment of diabetes and associated disorders.

In another embodiment is provided processes for preparing the compounds of the present invention.

### DESCRIPTION OF THE INVENTION

Accordingly, the present invention relates to compounds of the general formula (I) represented below & includes their solvates, hydrates as well as their pharmaceutically acceptable salts and includes their suitable pharmaceutically acceptable formulations

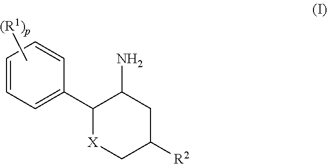

Wherein:
R¹ at each occurrence is independently selected from hydrogen, halo, cyano, nitro, hydroxyl, optionally substituted groups selected from amino, C₁₋₆ alkyl, C₂₋₆ alkenyl, C₂₋₆ alkynyl, C₁₋₆ alkoxy, C₂₋₆ alkenoxy, C₂₋₆ alkynyloxy, cycloalkoxy, aryl, cycloalkyl, carbocycle, heterocyclyl, heteroaryl, heterocycloalkyl, cycloalkyl(C₁₋₆)alkyl, heterocycloalkyl(C₁₋₆)alkyl, aralkyl, heteroarylalkyl, aryloxy, heteroaryloxy, heterocyclyloxy, wherein each of these groups, whenever applicable, is further substituted with one to three substituent(s) independently selected from hydroxy, (C₁₋₄)alkoxy, halo, cyano, amino, (C₁₋₆)alkylamino, nitro, COO(C₁₋₄)alkyl, S(O)ₙ, S(O)ₙNH₂, S(O)ₙNH(C₁₋₆)alkyl, C(O); C(O)NH(C₁₋₆)alkyl groups;
R² is selected from the following bicyclic non aromatic ring systems

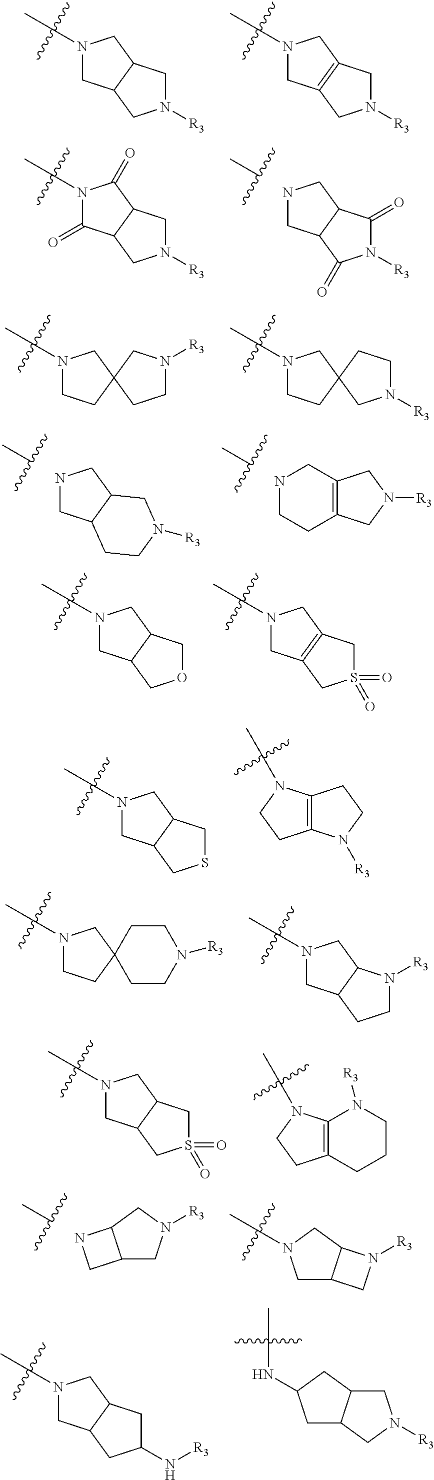

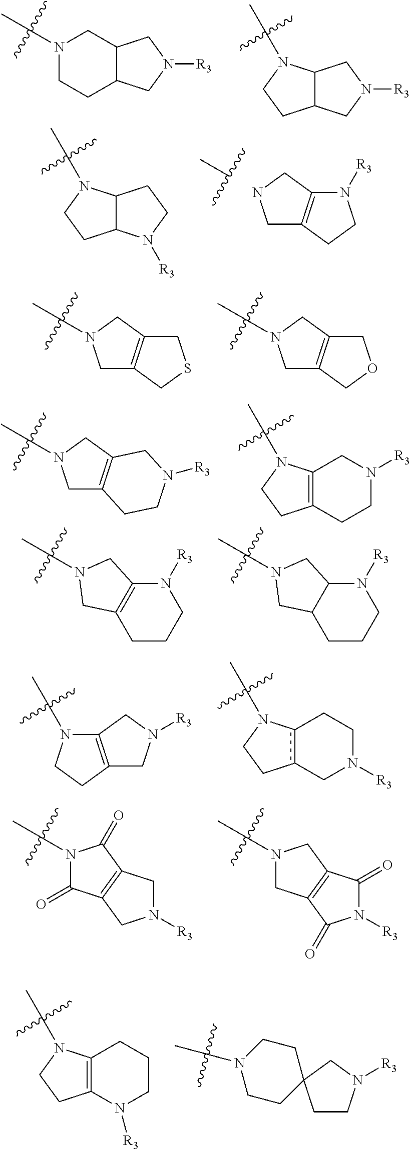

Wherein R₃ at each occurrence is independently selected from hydrogen, halo, haloalkyl, cyano, optionally substituted groups selected from amino, C₁₋₆ alkyl, C₂₋₆ alkenyl, C₂₋₆ alkynyl, aryl, cycloalkyl, carbocycle, heterocycloalkyl, cycloalkyl(C₁₋₆)alkyl, heterocycloalkyl(C₁₋₆)alkyl, S(O)ₙ, S(O)ₙ(C₁₋₆)alkyl, S(O)ₙ(C₁₋₆)aryl, S(O)ₙNH₂, S(O)ₙNH(C₁₋₆)alkyl, S(O)ₙNHcycloalkyl, S(O)ₙNHaryl, S(O)ₙNHheteroaryl, (C₁₋₆)alkylamino, nitro, COO(C₁₋₄)alkyl, S((O)═NH)-alkyl, S((O)═NH)-aryl, S((O)═NH)-cycloalkyl, S((O)═NH)-heteroaryl, S((O)═N-alkyl)-alkyl, S((O)═N-alkyl)-aryl, S((O)═N-alkyl)-cycloalkyl, S((O)═N-alkyl)-heteroaryl, S((O)═N-aryl)-alkyl, S((O)═N-aryl)-aryl, S((O)═N-aryl)-cycloalkyl, S((O)═N-aryl)-heteroaryl, S((O)═N—(SO₂-alkyl))-alkyl, S((O)═N—(SO₂-alkyl))-aryl, S((O)═N—(SO₂-alkyl))-cycloalkyl, S(O)═N—(SO₂-alkyl))-heteroaryl, S((O)═N—(SO₂-aryl))-alkyl, S((O)═N—(SO₂-aryl))-aryl, S(O)═N—(SO₂-aryl))cycloalkyl, S((O)═N—(SO₂-aryl))heteroaryl, C(O), C(O)NH(C₁₋₆)alkyl groups.

When R₃ is substituted, the preferred substituents on R₃ wherever applicable are selected from hydrogen, halo, haloalkyl, amino, cyano, methyl, ethyl, propyl, butyl, cyclopropyl, cyclobutyl, cyclopentyl, cyclohexyl, phenyl, —CH₂—COOH, —C(═O)—O-methyl, —C(═O)—O-trifluromethyl, —C(═O)—O-ethyl, —C(═O)—O-phenyl, —C(═O)—NH-methyl, —C(═O)—NH-ethyl, —C(═O)—NH-propyl, —C(═O)—NH-cyclopropyl, —C(═O)—NH-phenyl, —C(═O)—NH-trifluromethyl, —C(═O)-methyl, —C(═O)-ethyl, —C(═O)CH₂-methyl, —C(═O)CH₂-phenyl, S(O)₂-phenyl, S(O)₂-methyl, S(O)₂-ethyl, S(O)₂-propyl, S(O)₂-butyl, S(O)₂-cyclopropyl, S(O)₂-cyclobutyl, S(O)₂-cyclopentyl, S(O)₂-cyclohexyl, S(O)₂-phenyl, S(O)₂-flurophenyl, S(O)₂-cynophenyl, S(O)₂NH₂, S(O)₂NH-methyl, S(O)₂NH-ethyl, S(O)₂NH-propyl, S(O)₂NH-butyl, S(O)₂NH-pentyl, S(O)₂NH-cyclopropyl, S(O)₂NH-cyclobutyl, S(O)₂NH-cyclopentyl, S(O)₂NH-cyclohexyl, S(O)₂NH-phenyl, S((O)═NH)-methyl, S((O)═NH)-ethyl, S((O)═NH)-phenyl, S((O)═NH)-cyclopentyl, S((O)═NH)-pyridine, S((O)═N-methyl)-methyl, S((O)═N-methyl)-phenyl, S((O)═N-ethyl)-cyclopropyl, S((O)═N-methyl)-pyridine, S((O)═N-phenyl)-methyl, S((O)═N-phenyl)-phenyl, S((O)═N-phenyl)-cyclopentyl, S((O)═N-phenyl)-pyridine, S((O)═N—(SO₂-methyl))-methyl, S((O)═N—(SO₂-methyl))-phenyl, S((O)═N—(SO₂-ethyl))cyclohexyl, S((O)═N—(SO₂-methyl))pyridine, S((O)═N—(SO₂-phenyl))-methyl, S((O)═N—(SO₂-phenyl))-phenyl, S((O)═N—(SO₂-phenyl))cyclopentyl, S((O)═N—(SO₂-phenyl))-pyridine.

### Wherein

n=0-7;
p=1-5;
X=—CH₂, —NR⁴, O, S;
R⁴ is independently selected from hydrogen, halo, amino, cyano, nitro, (C₁₋₄)alkyl, (C₁₋₆)alkylcarbonyl, (C₂₋₆)alkenyl, (C₂₋₆)alkynyl, —(CH₂)—COO(C₁₋₄)alkyl, —(CH₂)—COOH, —C(═O)CH₂alkyl, —C(═O)CH₂aryl, —C(═O)CH₂heteroaryl, (CH₂)ₙaryl, (CH₂)ₙheteroaryl, (CH₂)ₙ—N-heteroaryl, (CH₂)ₙ—N-heterocyclyl, S(O)ₙ, S(O)ₙaryl, S(O)ₙalkyl, S(O)ₙ(C₁₋₆)alkyl, S(O)ₙ(C₁₋₆)aryl, S(O)ₙNH₂, S(O)ₙNH(C₁₋₆)alkyl groups.

In an alternate embodiment, when any of the groups defined above is further substituted, the substituents, if present, may be selected from those defined above.

In a preferred embodiment of the present invention,
R¹ at each occurrence is independently selected from hydrogen, halo, cyano, optionally substituted groups selected from amino, C₁₋₄ alkyl, C₂₋₆ alkenyl, C₂₋₆ alkynyl, aryl, cycloalkyl, carbocycle, heterocycloalkyl, cycloalkyl(C₁₋₆)alkyl, heterocycloalkyl(C₁₋₆)alkyl groups wherein any amino, alkyl, alkenyl, alkynyl, cycloalkyl heterocycloalkyl group is further substituted on available carbon atom with one to three subsistent(s) independently selected from hydroxy, (C₁₋₄)alkoxy, halo, cyano, amino, (C₁₋₆)alkylamino, nitro, COO(C₁₋₄)alkyl, S(O)ₙ, S(O)ₙNH₂, S(O)ₙNH(C₁₋₆)alkyl, C(O); C(O)NH(C₁₋₆)alkyl groups;
R⁴ is selected from hydrogen, halo, amino, cyano, nitro, methyl, ethyl, propyl, butyl, cyclopropyl, cyclobutyl, cyclopentyl, cyclohexyl, —CH₂COOH, —C(═O)CH₂-methyl, —C(═O)CH₂-phenyl, S(O)₂-phenyl, S(O)₂-methyl, S(O)₂NH₂, S(O)₂NH-methyl groups.

Wherein ‘n’ and ‘p’ are defined as earlier and the substituents on any of the substitutions defined above, if present, may be selected from those defined above.

In a preferred embodiment, the groups, radicals described above may be selected from:

“Alkyl”, as well as other groups having the prefix “alk”, such as alkoxy and alkanoyl, means carbon chain which may be substituted with an oxygen atom as is well understood by a skilled artisan, which may further be either linear or branched, and combinations thereof, unless the carbon chain is defined otherwise. Examples of alkyl group include but not are limited to methyl, ethyl, propyl, isopropyl, butyl, sec-butyl, tert.-butyl, pentyl, hexyl etc. Where the specified number of carbon atoms permits e.g. from C₃₋₁₀, the term alkyl also includes cycloalkyl groups, and combinations of linear or branched alkyl chains combined with cycloalkyl structures. When no number of carbon atoms is specified, C₁₋₆ is intended.

“Alkenyl” means carbon chains which contain at least one carbon-carbon double bond, and which may be linear or branched or combinations thereof, unless the carbon chain is defined otherwise. Examples of alkenyl include but not limited to vinyl, allyl, isopropenyl, hexenyl, pentenyl, heptenyl, 1-propenyl, 2-butenyl, 2-methyl-2-butenyl etc. Where the specified number of carbon atoms permits, e.g., from C₅₋₁₀, the term alkenyl also includes cycloalkenyl groups and combinations of linear, branched and cyclic structures. When no number of carbon atoms is specified, C₍₂₋₆₎ is intended.

“Alkynyl” means carbon chains which contain at least one carbon-carbon triple bond, and which may be linear or branched or combinations thereof. Examples of alkynyl include ethynyl, propargyl, 3-methyl-1-pentynyl etc. When no number of carbon atoms is specified, C₍₂₋₆₎ is intended.

As used herein, “carbocycle” or “carbocyclic residue” is intended to mean any stable monocyclic or bicyclic or tricyclic ring, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, or tetrahydronaphthyl(tetralin). In a broader perspective, the term carbocycle is intended to include, wherever applicable, the groups representing cycloalkyl, phenyl and other saturated, partially saturated or aromatic residues;
“Cycloalkyl” is the subset of alkyl and means saturated carbocyclic ring having a specified number of carbon atoms, preferably 3-6 carbon atoms. Examples of cycloalkyl include cyclopropyl, cyclobutyl, cyclopentyl, cyclohexyl, cycloheptyl etc. A cycloalkyl group generally is monocyclic unless otherwise stated. Cycloalkyl groups are saturated unless and otherwise stated.

The “alkoxy” refers to the straight or branched chain alkoxides of the number of carbon atoms specified.

The term “alkylamino” refers to straight or branched alkylamines of the number of carbon atoms specified.

“Aryl” means a mono- or polycyclic aromatic ring system containing carbon ring atoms. The preferred aryls are monocyclic or bicyclic 6-10 membered aromatic ring systems. Phenyl and naphthyl are preferred aryls.

“Heterocycle” and “heterocyclyl” refer to saturated or unsaturated non-aromatic rings or ring systems containing at least one heteroatom selected from O, S, N further optionally including the oxidized forms of sulfur, namely SO & SO₂. Examples of heterocycles include tetrahydrofuran (THF), dihydrofuran, 1,4-dioxane, morpholine, 1,4-dithiane, piperazine, piperidine, 1,3-dioxolane, imidazoline, imidazolidine, pyrrolidine, pyrroline, tetrahydropyran, dihydropyran, oxathiolane, dithiolane, 1,3-dioxane, 1,3-dithiane, oxathiane, thiomorpholine etc.

“Heteroaryl” means an aromatic or partially aromatic heterocycle that contains at least one ring heteroatom selected from O, S and N. Heteroaryls thus include heteroaryls fused to the other kinds of rings, such as aryls, cycloalkyls, and heterocycles that are not aromatic. Examples of heteroaryl groups include; pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furyl, triazinyl, thienyl, pyrimidyl, benzisoxazolyl, benzoxazolyl, benzthiazolyl, benzothiadiazolyl, dihydrobenzofuranyl, pyridazinyl, indazolyl, isoindolyl, dihydrobenzothienyl, indolinyl, pyridazinyl, indazolyl, isoindolyl, dihydrobenzothienyl, indolizinyl, cinnolinyl, phthalazinyl, quinazolinyl, napthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl etc. For heterocyclyl and heteroaryl groups, rings and ring systems containing from 3-15 carbon atoms are included, forming 1-3 rings.

“Halo/Halogen” refers to fluorine, chlorine, bromine, iodine. Chlorine and fluorine are generally preferred.

Suitable groups and substituents on the groups may be selected from those described anywhere in the specification.

The term “substituted,” as used herein, means that any one or more hydrogens on the designated atom is replaced with a selection from the indicated group, provided that the designated atom's normal valency is not exceeded, and that the substitution results in a stable compound. The term “substituted,” as used herein, means that any one or more hydrogens on the designated atom is replaced with a selection from the indicated group, provided that the designated atom's normal valency is not exceeded, and that the substitution results in a stable compound.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. Such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (Bundgard, H., Design of Prodrugs (1985), pp. 7-9, 21-24 (Elsevier, Amsterdam)). The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound.

The term ‘optional’ or ‘optionally’ means that the subsequent described event or circumstance may or may not occur, and the description includes instances where the event or circumstance occur and instances in which it does not. For example, ‘optionally substituted alkyl’ means either ‘alkyl’ or ‘substituted alkyl’. Further an optionally substituted group means unsubstituted.

Unless otherwise stated in the specification, structures depicted herein are also meant to include compounds which differ only in the presence of one or more isotopically enriched atoms.

Particularly useful compounds may be selected from but not limited to;

TABLE 1
List of compounds as DPP-IV inhibitors

| Compounds | Structures | IUPAC Names |
|---|---|---|
| 1 | 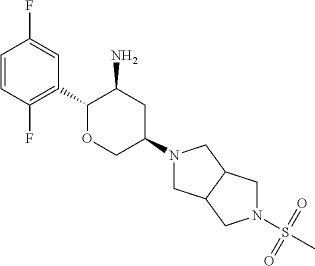 [SMILES: CS(=O)(=O)N1CC2CN([C@H]3CO[C@H](c4cc(F)ccc4F)[C@@H](N)C3)CC2C1] | (2R,3S,5R)-2-(2,5-difluorophenyl)-5-(5- (methylsulfonyl)hexahydropyrrolo[3,4- c]pyrrol-2(1H)-yl)tetrahydro-2H-pyran-3- amine |
| 2 | 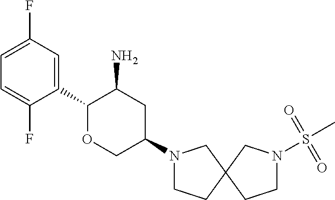 [SMILES: CS(=O)(=O)N1CCC2(CCN([C@H]3CO[C@H](c4cc(F)ccc4F)[C@@H](N)C3)C2)C1] | (2R,3S,5R)-2-(2,5-difluorophenyl)-5-(7- (methylsulfonyl)-2,7-diazaspiro[4.4]nonan-2- yl)tetrahydro-2H-pyran-3-amine |
| 3 | 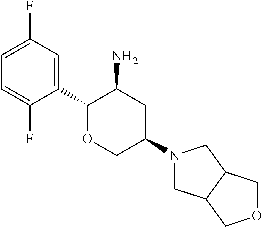 [SMILES: N[C@H]1C[C@@H](N2CC3COCC3C2)CO[C@@H]1c1cc(F)ccc1F] | (2R,3S,5R)-2-(2,5-difluorophenyl)-5- (tetrahydro-1H-furo[3,4-c]pyrrol-5(3H)- yl)tetrahydro-2H-pyran-3-amine |
| 4 | 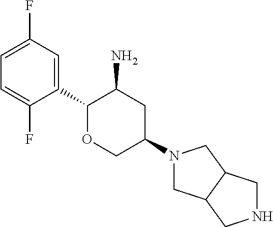 [SMILES: N[C@H]1C[C@@H](N2CC3CNCC3C2)CO[C@@H]1c1cc(F)ccc1F] | (2R,3S,5R)-2-(2,5-difluorophenyl)-5- (hexahydropyrrolo[3,4-c]pyrrol-2(1H)- yl)tetrahydro-2H-pyran-3-amine |
| 5 | 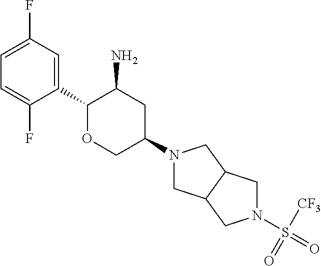 [SMILES: N[C@H]1C[C@@H](N2CC3CN(S(=O)(=O)C(F)(F)F)CC3C2)CO[C@@H]1c1cc(F)ccc1F] | (2R,3S,5R)-2-(2,5-difluorophenyl)-5-(5- ((trifluoromethyl)sulfonyl)hexahydropyrrolo[3, 4-c]pyrrol-2(1H)-yl)tetrahydro-2H-pyran-3- amine |
| 6 | 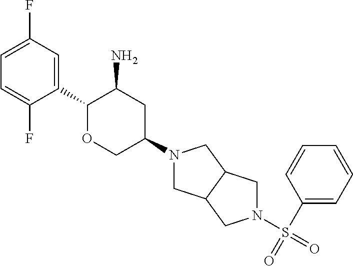 [SMILES: N[C@H]1C[C@@H](N2CC3CN(S(=O)(=O)c4ccccc4)CC3C2)CO[C@@H]1c1cc(F)ccc1F] | (2R,3S,5R)-2-(2,5-difluorophenyl)-5-(5- (phenylsulfonyl)hexahydropyrrolo[3,4- c]pyrrol-2(1H)-yl)tetrahydro-2H-pyran-3- amine |
| 7 | 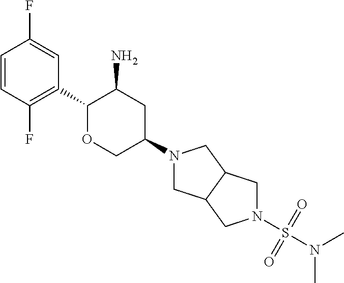 [SMILES: CN(C)S(=O)(=O)N1CC2CN([C@H]3CO[C@H](c4cc(F)ccc4F)[C@@H](N)C3)CC2C1] | 5-((3R,5S,6R)-5-amino-6-(2,5- difluorophenyl)tetrahydro-2H-pyran-3-yl)-N,N- dimethylhexahydropyrrolo[3,4-c]pyrrole- 2(1H)-sulfonamide |
| 8 | 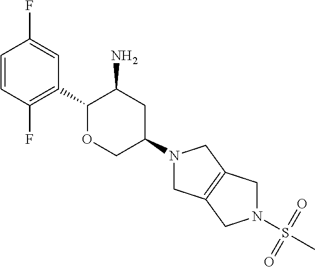 [SMILES: CS(=O)(=O)N1CC2=C(CN([C@H]3CO[C@H](c4cc(F)ccc4F)[C@@H](N)C3)C2)C1] | (2R,3S,5R)-2-(2,5-difluorophenyl)-5-(5- (methylsulfonyl)-5,6-dihydropyrrolo[3,4- c]pyrrol-2(1H,3H,4H)-yl)tetrahydro-2H-pyran- 3-amine |
| 9 | 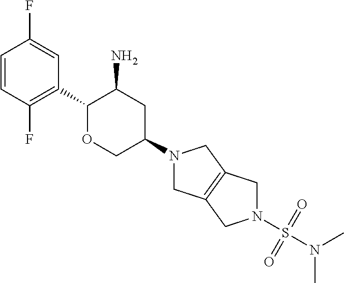 [SMILES: CN(C)S(=O)(=O)N1CC2=C(CN([C@H]3CO[C@H](c4cc(F)ccc4F)[C@@H](N)C3)C2)C1] | 5-((3R,5S,6R)-5-amino-6-(2,5- difluorophenyl)tetrahydro-2H-pyran-3-yl)-N,N- dimethyl-3,4,5,6-tetrahydropyrrolo[3,4- c]pyrrole-2(1H)-sulfonamide |
| 10 | 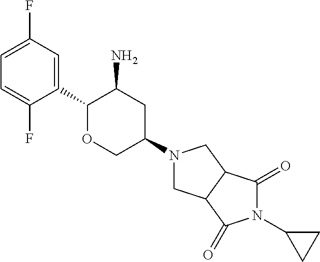 [SMILES: N[C@H]1C[C@@H](N2CC3C(=O)N(C4CC4)C(=O)C3C2)CO[C@@H]1c1cc(F)ccc1F] | 5-((3R,5S,6R)-5-amino-6-(2,5- difluorophenyl)tetrahydro-2H-pyran-3-yl)-2- cyclopropyltetrahydropyrrolo[3,4-c]pyrrole- 1,3(2H,3aH)-dione |
| 11 | 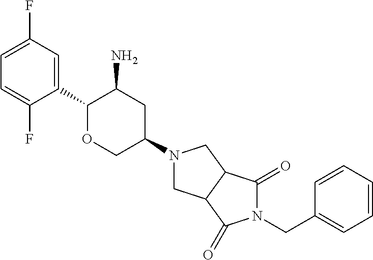 [SMILES: N[C@H]1C[C@@H](N2CC3C(=O)N(Cc4ccccc4)C(=O)C3C2)CO[C@@H]1c1cc(F)ccc1F] | 5-((3R,5S,6R)-5-amino-6-(2,5- difluorophenyl)tetrahydro-2H-pyran-3-yl)-2- benzyltetrahydropyrrolo[3,4-c]pyrrole- 1,3(2H,3aH)-dione |
| 12 | 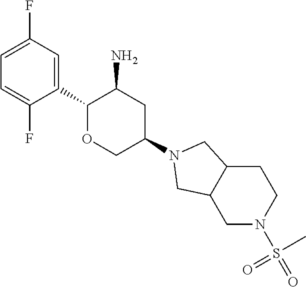 [SMILES: CS(=O)(=O)N1CCC2CN([C@H]3CO[C@H](c4cc(F)ccc4F)[C@@H](N)C3)CC2C1] | (2R,3S,5R)-2-(2,5-difluorophenyl)-5-(5- (methylsulfonyl)hexahydro-1H-pyrrolo[3,4- c]pyridin-2(3H)-yl)tetrahydro-2H-pyran-3- amine |
| 13 | 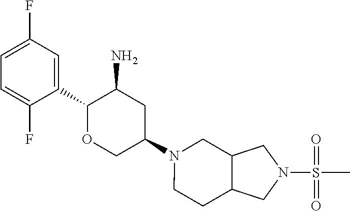 [SMILES: CS(=O)(=O)N1CC2CCN([C@H]3CO[C@H](c4cc(F)ccc4F)[C@@H](N)C3)CC2C1] | (2R,3S,5R)-2-(2,5-difluorophenyl)-5-(2- (methylsulfonyl)hexahydro-1H-pyrrolo[3,4- c]pyridin-5(6H)-yl)tetrahydro-2H-pyran-3- amine |
| 14 | 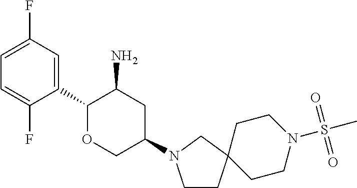 [SMILES: CS(=O)(=O)N1CCC2(CCN([C@H]3CO[C@H](c4cc(F)ccc4F)[C@@H](N)C3)C2)CC1] | (2R,3S,5R)-2-(2,5-difluorophenyl)-5-(8- (methylsulfonyl)-2,8-diazaspiro[4.5]decan-2- yl)tetrahydro-2H-pyran-3-amine |
| 15 | 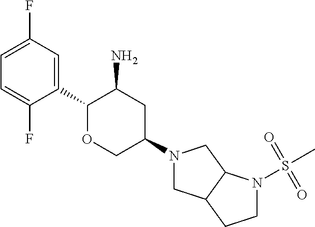 [SMILES: CS(=O)(=O)N1CCC2CN([C@H]3CO[C@H](c4cc(F)ccc4F)[C@@H](N)C3)CC21] | (2R,3S,5R)-2-(2,5-difluorophenyl)-5-(1- (methylsulfonyl)hexahydropyrrolo[3,4- b]pyrrol-5(1H)-yl)tetrahydro-2H-pyran-3- amine |
| 16 | 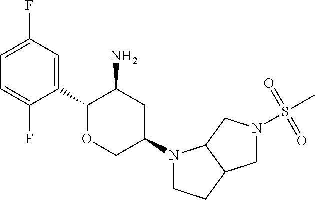 [SMILES: CS(=O)(=O)N1CC2CCN([C@H]3CO[C@H](c4cc(F)ccc4F)[C@@H](N)C3)C2C1] | (2R,3S,5R)-2-(2,5-difluorophenyl)-5-(5- (methylsulfonyl)hexahydropyrrolo[3,4- b]pyrrol-1(2H)-yl)tetrahydro-2H-pyran-3- amine |
| 17 | 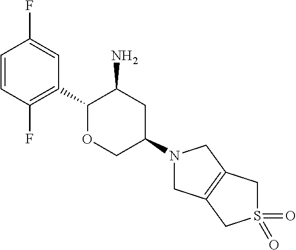 [SMILES: N[C@H]1C[C@@H](N2CC3=C(C2)CS(=O)(=O)C3)CO[C@@H]1c1cc(F)ccc1F] | 5-((3R,5S,6R)-5-amino-6-(2,5- difluorophenyl)tetrahydro-2H-pyran-3-yl)- 3,4,5,6-tetrahydro-1H-thieno[3,4-c]pyrrole 2,2- dioxide |
| 18 | 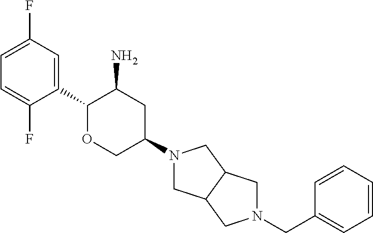 [SMILES: N[C@H]1C[C@@H](N2CC3CN(Cc4ccccc4)CC3C2)CO[C@@H]1c1cc(F)ccc1F] | (2R,3S,5R)-5-(5-benzylhexahydropyrrolo[3,4- c]pyrrol-2(1H)-yl)-2-(2,5- difluorophenyl)tetrahydro-2H-pyran-3-amine |
| 19 | 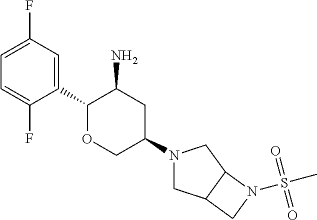 [SMILES: CS(=O)(=O)N1CC2CN([C@H]3CO[C@H](c4cc(F)ccc4F)[C@@H](N)C3)CC21] | (2R,3S,5R)-2-(2,5-difluorophenyl)-5-((1R,5S)- 6-(methylsulfonyl)-3,6- diazabicyclo[3.2.0]heptan-3-yl)tetrahydro-2H- pyran-3-amine |
| 20 | 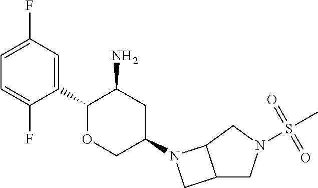 [SMILES: CS(=O)(=O)N1CC2CN([C@H]3CO[C@H](c4cc(F)ccc4F)[C@@H](N)C3)C2C1] | (2R,3S,5R)-2-(2,5-difluorophenyl)-5- ((1R,5R)-3-(methylsulfonyl)-3,6- diazabicyclo[3.2.0]heptan-6-yl)tetrahydro-2H- pyran-3-amine |
| 21 | 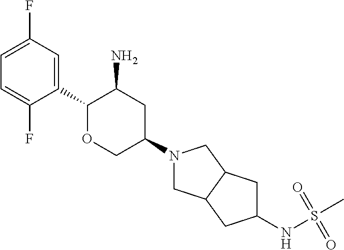 [SMILES: CS(=O)(=O)NC1CC2CN([C@H]3CO[C@H](c4cc(F)ccc4F)[C@@H](N)C3)CC2C1] | N-(2-((3R,5S,6R)-5-amino-6-(2,5- difluorophenyl)tetrahydro-2H-pyran-3- yl)octahydrocyclopenta[c]pyrro1-5- yl)methanesulfonamide |
| 22 | 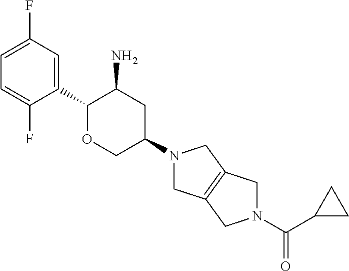 [SMILES: N[C@H]1C[C@@H](N2CC3=C(CN(C(=O)C4CC4)C3)C2)CO[C@@H]1c1cc(F)ccc1F] | (5-((3R,5S,6R)-5-amino-6-(2,5- difluorophenyl)tetrahydro-2H-pyran-3-yl)-5,6- dihydropyrrolo[3,4-c]pyrrol-2(1H,3H,4H)- yl)(cyclopropyl)methanone |
| 23 | 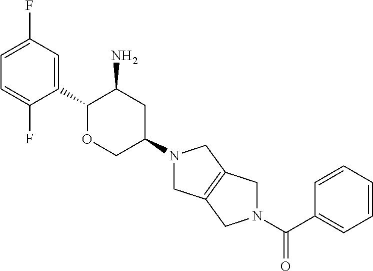 [SMILES: N[C@H]1C[C@@H](N2CC3=C(CN(C(=O)c4ccccc4)C3)C2)CO[C@@H]1c1cc(F)ccc1F] | (5-((3R,5 S,6R)-5-amino-6-(2,5- difluorophenyl)tetrahydro-2H-pyran-3-yl)-5,6- dihydropyrrolo[3,4-c]pyrrol-2(1H,3H,4H)- yl)(phenyl)methanone |
| 24 | 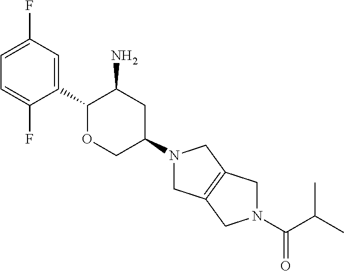 [SMILES: CC(C)C(=O)N1CC2=C(C1)CN([C@H]1CO[C@H](c3cc(F)ccc3F)[C@@H](N)C1)C2] | 1-(5-((3R,5S,6R)-5-amino-6-(2,5- difluorophenyl)tetrahydro-2H-pyran-3-yl)-5,6- dihydropyrrolo[3,4-c]pyrrol-2(1H,3H,4H)-yl)- 2-methylpropan-1-one |
| 25 | 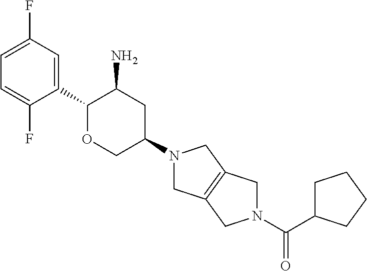 [SMILES: N[C@H]1C[C@@H](N2CC3=C(CN(C(=O)C4CCCC4)C3)C2)CO[C@@H]1c1cc(F)ccc1F] | (5-((3R,5S,6R)-5-amino-6-(2,5- difluorophenyl)tetrahydro-2H-pyran-3-yl)-5,6- dihydropyrrolo[3,4-c]pyrrol-2(1H,3H,4H)- yl)(cyclopentyl)methanone |
| 26 | 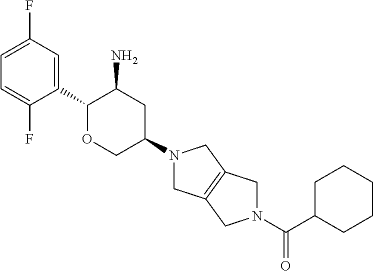 [SMILES: N[C@H]1C[C@@H](N2CC3=C(CN(C(=O)C4CCCCC4)C3)C2)CO[C@@H]1c1cc(F)ccc1F] | (5-((3R,5S,6R)-5-amino-6-(2,5- difluorophenyl)tetrahydro-2H-pyran-3-yl)-5,6- dihydropyrrolo[3,4-c]pyrrol-2(1H,3H,4H)- yl)(cyclohexyl)rnethanone |
| 27 | 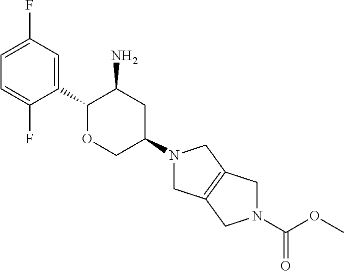 [SMILES: COC(=O)N1CC2=C(C1)CN([C@H]1CO[C@H](c3cc(F)ccc3F)[C@@H](N)C1)C2] | methyl 5-((3R,5S,6R)-5-amino-6-(2,5- difluorophenyl)tetrahydro-2H-pyran-3-yl)- 3,4,5,6-tetrahydropyrrolo[3,4-c]pyrrole-2(1H)- carboxylate |
| 28 | 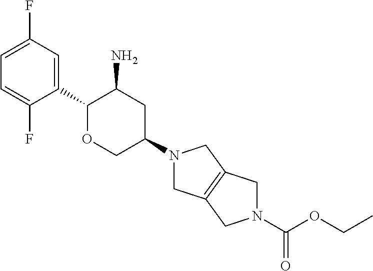 [SMILES: CCOC(=O)N1CC2=C(C1)CN([C@H]1CO[C@H](c3cc(F)ccc3F)[C@@H](N)C1)C2] | ethyl 5-((3R,5S,6R)-5-amino-6-(2,5- difluorophenyl)tetrahydro-2H-pyran-3-yl)- 3,4,5,6-tetrahydropyrrolo[3,4-c]pyrrole-2(1H)- carboxylate |
| 29 | 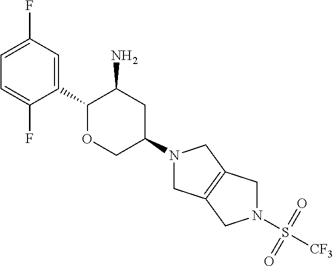 [SMILES: N[C@H]1C[C@@H](N2CC3=C(C2)CN(S(=O)(=O)C(F)(F)F)C3)CO[C@@H]1c1cc(F)ccc1F] | (2R,3S,5R)-2-(2,5-difluorophenyl)-5-(5- ((trifluoromethyl)sulfonyl)-5,6- dihydropyrrolo[3,4-c]pyrrol-2(1H,3H,4H)- yl)tetrahydro-2H-pyran-3-amine |
| 30 | 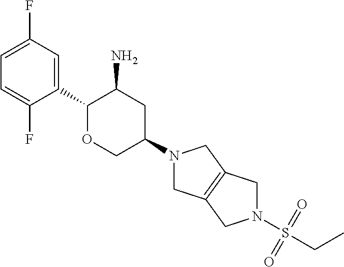 [SMILES: CCS(=O)(=O)N1CC2=C(CN([C@H]3CO[C@H](c4cc(F)ccc4F)[C@@H](N)C3)C2)C1] | (2R,3S,5R)-2-(2,5-difluorophenyl)-5-(5- (ethylsulfonyl)-5,6-dihydropyrrolo[3,4- c]pyrrol-2(1H,3H,4H)-yl)tetrahydro-2H-pyran- 3-amine |
| 31 | 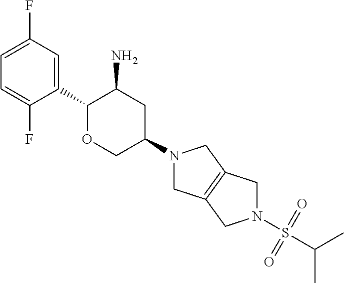 [SMILES: CC(C)S(=O)(=O)N1CC2=C(CN([C@H]3CO[C@H](c4cc(F)ccc4F)[C@@H](N)C3)C2)C1] | (2R,3S,5R)-2-(2,5-difluorophenyl)-5-(5- (isopropylsulfonyl)-5,6-dihydropyrrolo[3,4- c]pyrrol-2(1H,3H,4H)-yl)tetrahydro-2H-pyran- 3-amine |
| 32 | 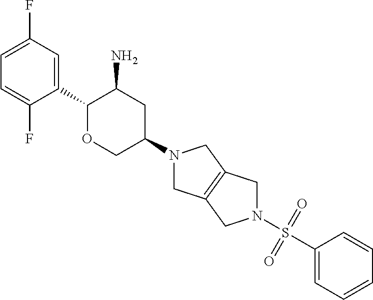 [SMILES: N[C@H]1C[C@@H](N2CC3=C(C2)CN(S(=O)(=O)c2ccccc2)C3)CO[C@@H]1c1cc(F)ccc1F] | (2R,3S,5R)-2-(2,5-difluorophenyl)-5-(5- (phenylsulfonyl)-5,6-dihydropyrrolo[3,4- c]pyrrol-2(1H,3H,4H)-yl)tetrahydro-2H-pyran- 3-amine |
| 33 | 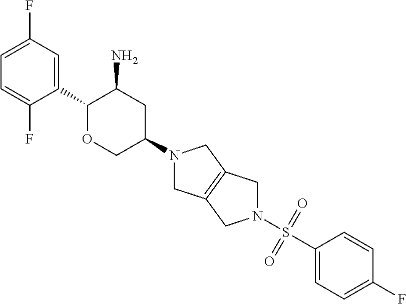 [SMILES: N[C@H]1C[C@@H](N2CC3=C(C2)CN(S(=O)(=O)c2ccc(F)cc2)C3)CO[C@@H]1c1cc(F)ccc1F] | (2R,3S,5R)-2-(2,5-difluorophenyl)-5-(5-((4- fluorophenyl)sulfonyl)-5,6-dihydropyrrolo[3,4- c]pyrrol-2(1H,3H,4H)-yl)tetrahydro-2H-pyran- 3-amine |
| 34 | 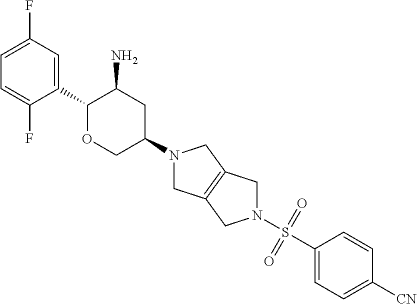 [SMILES: N#Cc1ccc(S(=O)(=O)N2CC3=C(CN([C@H]4CO[C@H](c5cc(F)ccc5F)[C@@H](N)C4)C3)C2)cc1] | 4-((5-((3R,5S,6R)-5-amino-6-(2,5- difluorophenyl)tetrahydro-2H-pyran-3-yl)-5,6- dihydropyrrolo[3,4-c]pyrrol-2(1H,3H,4H)- yl)sulfonyl)benzonitrile |
| 35 | 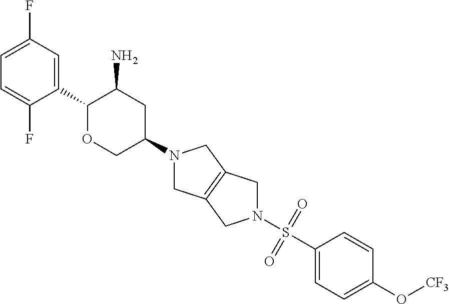 [SMILES: N[C@H]1C[C@@H](N2CC3=C(C2)CN(S(=O)(=O)c2ccc(OC(F)(F)F)cc2)C3)CO[C@@H]1c1cc(F)ccc1F] | (2R,3S,5R)-2-(2,5-difluorophenyl)-5-(5-((4- (trifluoromethoxy)phenyl)sulfonyl)-5,6- dihydropyrrolo[3,4-c]pyrrol-2(1H,3H,4H)- yl)tetrahydro-2H-pyran-3-amine |
| 36 | 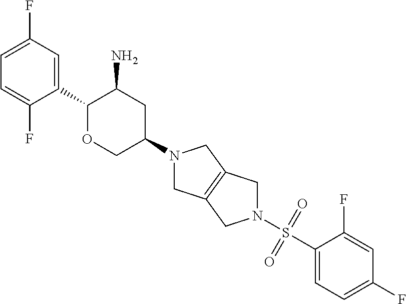 [SMILES: N[C@H]1C[C@@H](N2CC3=C(C2)CN(S(=O)(=O)c2ccc(F)cc2F)C3)CO[C@@H]1c1cc(F)ccc1F] | (2R,3S,5R)-2-(2,5-difluorophenyl)-5-(5-((2,4- difluorophenyl)sulfonyl)-5,6- dihydropyrrolo[3,4-c]pyrrol-2(1H,3H,4H)- yl)tetrahydro-2H-pyran-3-amine |
| 37 | 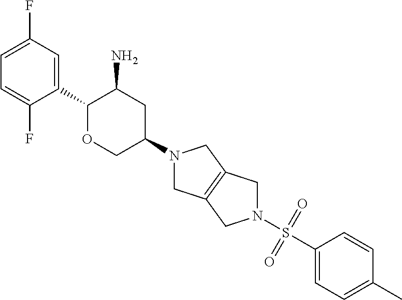 [SMILES: Cc1ccc(S(=O)(=O)N2CC3=C(CN([C@H]4CO[C@H](c5cc(F)ccc5F)[C@@H](N)C4)C3)C2)cc1] | (2R,3S,5R)-2-(2,5-difluorophenyl)-5-(5-tosyl- 5,6-dihydropyrrolo[3,4-c]pyrrol-2(1H,3H,4H)- yl)tetrahydro-2H-pyran-3-amine |
| 38 | 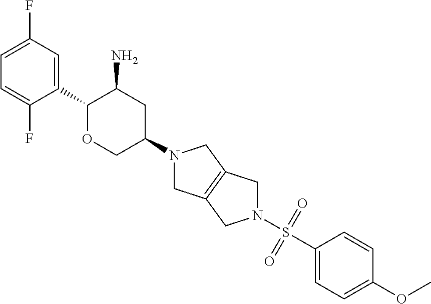 [SMILES: COc1ccc(S(=O)(=O)N2CC3=C(CN([C@H]4CO[C@H](c5cc(F)ccc5F)[C@@H](N)C4)C3)C2)cc1] | (2R,3S,5R)-2-(2,5-difluorophenyl)-5-(5-((4- methoxyphenyl)sulfonyl)-5,6- dihydropyrrolo[3,4-c]pyrrol-2(1H,3H,4H)- yl)tetrahydro-2H-pyran-3-amine |
| 39 | 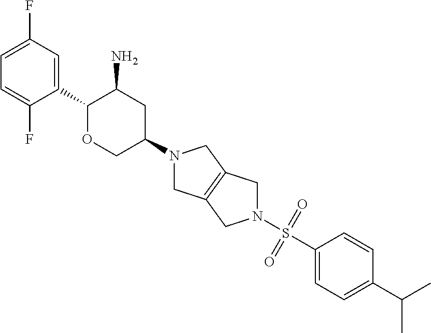 [SMILES: CC(C)c1ccc(S(=O)(=O)N2CC3=C(CN([C@H]4CO[C@H](c5cc(F)ccc5F)[C@@H](N)C4)C3)C2)cc1] | (2R,3S,5R)-2-(2,5-difluorophenyl)-5-(5-((4- isopropylphenyl)sulfonyl)-5,6- dihydropyrrolo[3,4-c]pyrrol-2(1H,3H,4H)- yl)tetrahydro-2H-pyran-3-amine |
| 40 | 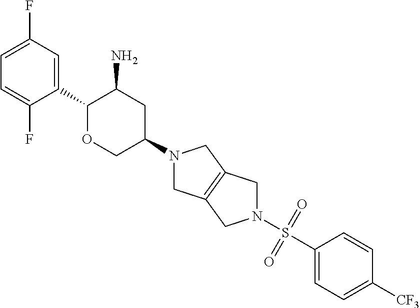 [SMILES: N[C@H]1C[C@@H](N2CC3=C(C2)CN(S(=O)(=O)c2ccc(C(F)(F)F)cc2)C3)CO[C@@H]1c1cc(F)ccc1F] | (2R,3S,5R)-2-(2,5-difluorophenyl)-5-(5-((4- (trifluoromethyl)phenyl)sulfonyl)-5,6- dihydropyrrolo[3,4-c]pyrrol-2(1H,3H,4H)- yl)tetrahydro-2H-pyran-3-amine |
| 41 | 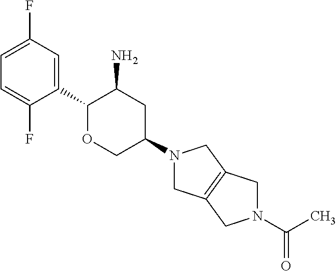 [SMILES: CC(=O)N1CC2=C(C1)CN([C@H]1CO[C@H](c3cc(F)ccc3F)[C@@H](N)C1)C2] | 1-(5-((3R,5S,6R)-5-amino-6-(2,5- difluorophenyl)tetrahydro-2H-pyran-3-yl)-5,6- dihydropyrrolo[3,4-c]pyrrol-2(1H,3H,4H)- yl)ethanone |
| 42 | 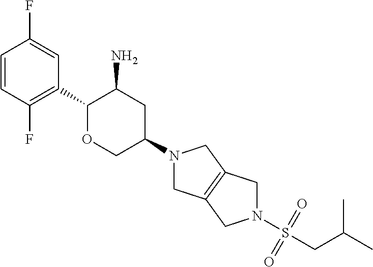 [SMILES: CC(C)CS(=O)(=O)N1CC2=C(CN([C@H]3CO[C@H](c4cc(F)ccc4F)[C@@H](N)C3)C2)C1] | (2R,3S,5R)-2-(2,5-difluorophenyl)-5-(5- (isobutylsulfonyl)-5,6-dihydropyrrolo[3,4- c]pyrrol-2(1H,3H,4H)-yl)tetrahydro-2H-pyran- 3-amine |
| 43 | 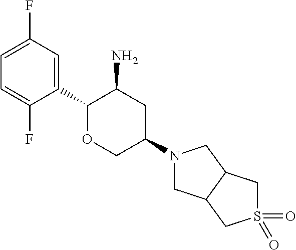 [SMILES: N[C@H]1C[C@@H](N2CC3CS(=O)(=O)CC3C2)CO[C@@H]1c1cc(F)ccc1F] | 5-((3R,5S,6R)-5-amino-6-(2,5-difluorophenyl) tetrahydro-2H-pyran-3-yl) hexahydro-1H- thieno[3,4-c]pyrrole 2,2-dioxide |
| 44 | 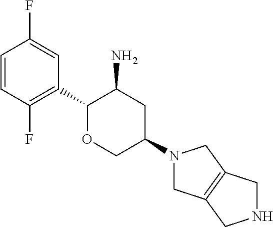 [SMILES: N[C@H]1C[C@@H](N2CC3=C(CNC3)C2)CO[C@@H]1c1cc(F)ccc1F] | (2R,3S,5R)-2-(2,5-difluorophenyl)-5-(5,6- dihydropyrrolo[3,4-c]pyrrol-2(1H,3H,4H)- yl)tetrahydro-2H-pyran-3-amine |
| 45 | 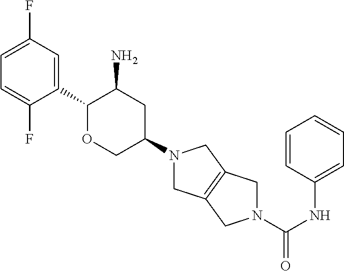 [SMILES: N[C@H]1C[C@@H](N2CC3=C(CN(C(=O)Nc4ccccc4)C3)C2)CO[C@@H]1c1cc(F)ccc1F] | 5-((3R,5S,6R)-5-amino-6-(2,5- difluorophenyl)tetrahydro-2H-pyran-3-yl)-N- phenyl-3,4,5,6-tetrahydropyrrolo[3,4-c]pyrrole- 2(1H)-carboxamide |
| 46 | 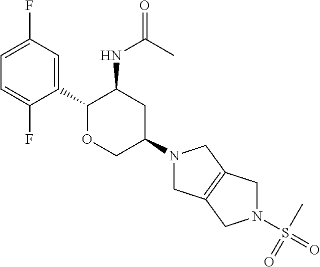 [SMILES: CC(=O)N[C@H]1C[C@@H](N2CC3=C(C2)CN(S(C)(=O)=O)C3)CO[C@@H]1c1cc(F)ccc1F] | N-((2R,3S,5R)-2-(2,5-difluorophenyl)-5-(5- (methylsulfonyl)-5,6-dihydropyrrolo[3,4- c]pyrrol-2(1H,3H,4H)-yl)tetrahydro-2H-pyran- 3-yl)acetamide |
| 47 | 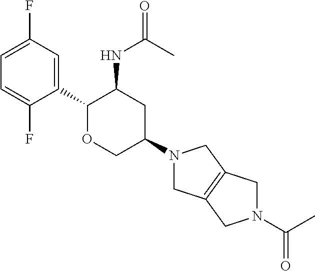 [SMILES: CC(=O)N[C@H]1C[C@@H](N2CC3=C(CN(C(C)=O)C3)C2)CO[C@@H]1c1cc(F)ccc1F] | N-((2R,3S,5R)-5-(5-acetyl-5,6- dihydropyrrolo[3,4-c]pyrrol-2(1H,3H,4H)-yl)- 2-(2,5-difluorophenyl)tetrahydro-2H-pyran-3- yl)acetamide |
| 48 | 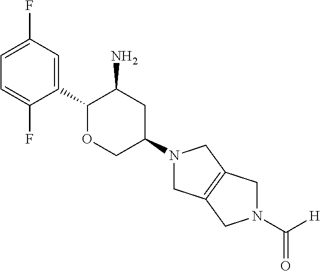 [SMILES: N[C@H]1C[C@@H](N2CC3=C(CN(C=O)C3)C2)CO[C@@H]1c1cc(F)ccc1F] | 5-((3R,5S,6R)-5-amino-6-(2,5- difluorophenyl)tetrahydro-2H-pyran-3-yl)- 3,4,5,6-tetrahydropyrrolo[3,4-c]pyrrole-2(1H)- carbaldehyde |
| 49 | 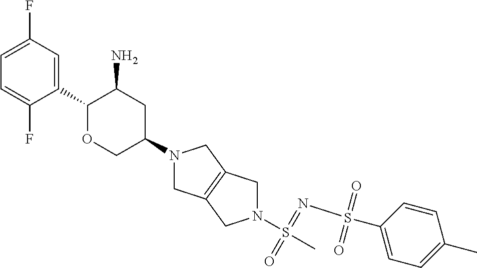 [SMILES: Cc1ccc(S(=O)(=O)N=S(C)(=O)N2CC3=C(CN([C@H]4CO[C@H](c5cc(F)ccc5F)[C@@H](N)C4)C3)C2)cc1] | (2R,3S,5R)-2-(2,5-difluorophenyl)-5-(5-(N-(4- methylbenzenesulfonyl)-S- methylsulfonimidoyl-5,6-dihydropyrrolo[3,4- c]pyrrol-2(1H,3H,4H)-yl)tetrahydro-2H-pyran- 3-amine |
| 50 | 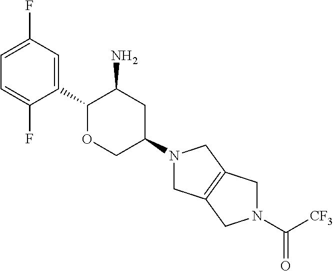 [SMILES: N[C@H]1C[C@@H](N2CC3=C(CN(C(=O)C(F)(F)F)C3)C2)CO[C@@H]1c1cc(F)ccc1F] | 1-(5-((3R,5S,6R)-5-amino-6-(2,5- difluorophenyl)tetrahydro-2H-pyran-3-yl)-5,6- dihydropyrrolo[3,4-c]pyrrol-2(1H,3H,4H)-yl)- 2,2,2-trifluoroethanone |

Or pharmaceutically acceptable salts of any of the compounds above.

Following is a list of abbreviations used in the description of the preparation of the compounds of the present invention:
ACN: Acetonitrile
AIBN: 2-2′-azobisisobutyronitrile
BOC: tert-Butyloxy carbonyl
Cs₂CO₃: Cesium carbonate
DBU: 1,8-Diazabicyclo[5.4.0]undac-7-ene
DCM: Dichloro methane
de: diastereomeric excess
DIEA: Diisopropyl ethyl amine
DIPE: Diisopropyl ether
DMA: N,N-Dimethyl acetamide
EtOH: Ethanol
h: hours
HBr: Hydrobromic acid
HCl: Hydrochloric acid
HPLC: High performance liquid chromatography
IPA: Isopropyl alcohol
MeOH: Methanol
Na₂CO₃: Sodium carbonate
Na₂S₂O₃: Sodium thiosulfate
Na₂SO₄: Sodium sulfate
NaBH₄: Sodium borohydride
NaHCO₃: Sodium bicarbonate/sodium hydrogen carbonate
NaHSO₃: Sodium hydrogen sulfite
NaOH: Sodium hydroxide
PCC: Pyridinium chlorochromate
PDC: Pyridinum dichromate
PTSA: p-Toluene sulphonic acid
TFA: Trifluoro acetic acid
THF: Tetrahydrofuran
TLC: Thin layer chromatography

The novel compounds of the present invention were prepared using the reactions and techniques described below, together with conventional techniques known to those skilled in the art of organic synthesis, or variations thereon as appreciated by those skilled in the art.

The reactions can be performed in solvents appropriate to the reagents and materials employed and are suitable for the transformations being effected. Preferred methods include, but not limited to those described below, where all symbols are as defined earlier unless and otherwise defined below.

The compounds of the formula (I) can be prepared as described in schemes below along with suitable modifications/variations which are well within the scope of a person skilled in the art.

Substituted benzaldehyde (1) can be treated with nitromethane in the presence of appropriate base to give compound (2) or can be prepared by the method reported in literature (for e.g. in WO 10/056708, WO 11/028455, WO 13/003250, U.S. Ser. No. 13/841,5297, WO 13/122920 & BMCL., 23(19), 5361-5366, 2013) along with their suitable modifications as may be necessary. Compound (2) can be oxidized to compound (3) using suitable oxidizing agents such as Desmartine periodinane, Jone's reagent, Swern oxidation, Pyridinium dicromate (PDC), Pyridinium chlorocromate (PCC) etc. Compound (3) can be treated with 3-Iodo-2-(iodomethyl)-prop-1-ene using appropriate base to give nitro pyrane (4), which upon subsequent reduction of endocyclic double bond and treatment with appropriate base followed by crystallization provided trans-pyrane (5). Nitro pyrane (5) can conveniently be reduced by variety of methods familiar to those skilled in the art. Chiral resolution of resulting amino pyrane (6) followed by its Boc protection provide compound (7), which upon oxidation in suitable system facilitated the formation of intermediate-1.

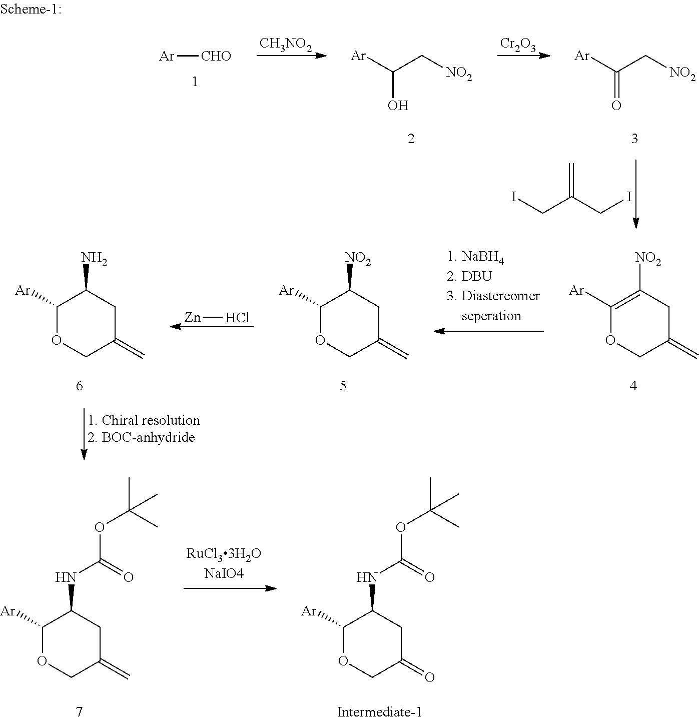

Intermediate-1 and the substituents representing R² present in the compounds of general formula (I) are separately known in the literature or can be conveniently prepared by variety of methods familiar to those skilled in art or by methods described in the literature (for e.g. in Bioorg. Med. Chem. Lett., 19, 1682-1685, 2009; Heterocycles 41, 1291-1298, 1995; JOC 46, 2757-2764, 1981), CN 101619064 (2010), WO 101654 (2012), WO 153554 (2009) including their suitable variations).

Novel compounds of general formula (I) of the present invention can be prepared by treating intermediate-1 with the appropriate substituent R². Further, R² can also be prepared using the methods available in the literature or can be prepared by various methods known to those skilled in art (WO 2010/056708, WO 2011/028455, WO 2013/003250, US 2013/8415297, WO 2013/122920 & BMCL., 23(19), 5361-5366, 2013etc.). A synthetic route to compound of present invention is given in Scheme-2.

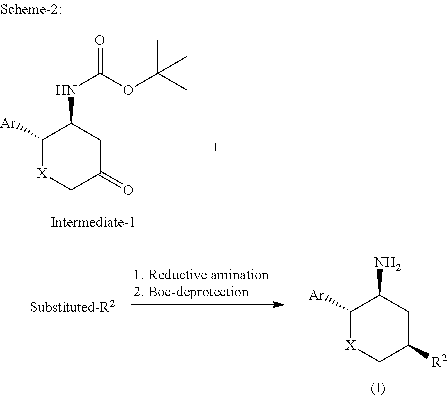

As illustrated in Scheme-2, the compounds of the present invention with structural formula (I) can be prepared by reductive amination of Intermediate-1 (obtained from the Scheme-1), with substituent-R², using appropriate reagent such as decaborane, sodiumtriacetoxy borohydride or sodium cyanoborohydride in solvents such as methanol, ethanol, tetrahydrofuran, dichloromethane, N,N-dimethyl acetamide or N,N-dimethyl formamide. Upon removal of Boc group either by treatment with trifluoroacetic acid, 4N HCl in dioxane or by passing HCl gas in to the reaction solution provides the compounds of the general formula (I). Compounds of the present invention can be isolated either as free amine form or as a salt corresponding to the acid used such as trifluoroacetic acid, hydrochloric acid, hydrobromic acid, oxalic acid, maleic acid, fumeric acid, succinic acid, p-toluene sulfonic acid or benzene sulfonic acid. The compounds can be purified where ever required, by recrystallization, trituration, precipitation, preparative thin layer chromatography, flash chromatography or by preparative HPLC method.

The compounds of the present invention can be used either alone or in combination with one or more therapeutic agents selected from insulin, insulin derivatives and mimetics, insulin secretagogues, insulin sensitizers, biguanide agents, alpha-glucosidase inhibitors, insulinotropic sulfonylurea receptor ligands, meglitinides, GLP-1, GLP-1 analogs, DPP-IV inhibitors, GPR-119 activators, sodium-dependent glucose co-transporter (SGLT2) inhibitors, PPAR modulators, non-glitazone type PPAR delta agonist, HMG-CoA reductase inhibitors, cholesterol-lowering drugs, rennin inhibitors, anti-thrombotic and anti-platelet agents and anti-obesity agents or pharmaceutically acceptable salts thereof. Such use will depend on the condition of the patient being treated and is well within the scope of a skilled practitioner.

The invention is further illustrated by the following non-limiting examples which describe the preferred way of carrying out the present invention. These are provided without limiting the scope of the present invention in any way.

¹H NMR spectral data given in the examples (vide infra) are recorded using a 400 MHz spectrometer (Bruker AVANCE-400) and reported in δ scale. Until and otherwise mentioned the solvent used for NMR is CDCl₃ using TMS as the internal standard.

### Synthesis of Intermediate-1: tert-butyl((2R,3S)-2-(2,5-difluorophenyl)-5-oxotetrahydro-2H-pyran-3-yl)carbamate

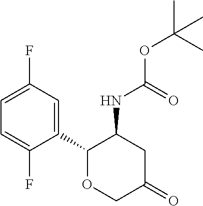 [SMILES: CC(C)(C)OC(=O)N[C@H]1CC(=O)CO[C@@H]1c1cc(F)ccc1F]

### Step-1: 1-(2,5-difluorophenyl)-2-nitroethanol (2)

To a solution of NaOH (25.3 g) in Water and MeOH at 0° C. was added a solution of 2,5-difluorobenzaldehyde (1, 57.3 ml) and nitromethane (34.2 ml) in MeOH drop wise, over a period of 30 min. After completion of reaction, reaction mixture was neutralized with glacial CH₃COOH. Ethyl acetate was added and the layers separated. The organic layer was washed successively with aqueous sat. Na₂CO₃ solution, and saturated brine solution. The organic layer was dried over anhydrous Na₂SO₄, filtered and concentrated to afford 2 (112 g, 97% yield) that was used without further purification in next step.

¹H NMR: (CDCl₃, 400 MHz): δ 7.31-7.33 (m, 1H), 7.08-7.01 (m, 2H), 5.73 (dd, 1H, J₁=9.2 Hz, J₂=2.4 Hz), 4.65 (dd, 1H, J₁=13.6 Hz, J₂=2.4 Hz), 4.53 (dd, 1H, J₁=9.2 Hz, J₂=13.6 Hz), 2.96 (bs, 1H); ESI-MS: (+ve mode) 204.1 (M+H)⁺ (100%); HPLC: 99.2%.

### Step-2: 1-(2,5-difluorophenyl)-2-nitroethanone (3)

1-(2,5-difluorophenyl)-2-nitroethanol (2, 1:00 g) was dissolved in Acetone and cooled to 0-5° C. Jones reagent was added drop wise to it in such a way that reaction temperature should not rise above 10° C. After completion of reaction, reaction mixture was cool to 0° C. and IPA was added drop wise to quench excess of Jones reagent. Solid residue precipitated was filtered and washed with acetone. Combined filtrate was evaporated to dryness to give light green oil, cooled it in ice bath and added 1.0 L of cold water, white solid precipitated. The solid obtained was filtered, washed with water and dried to get 3 (67 g, 67.7% yield).

¹H NMR: (DMSO-d₆, 400 MHz): δ 7.75-7.64 (m, 2H), 7.55-7.49 (m, 1H), 6.30 (d, 2H, J=2.8 Hz); ESI-MS: (+ve mode) 201.1 (M+H)⁺ (70%); HPLC: 98.3%.

### Step-3: 6-((2,5-difluorophenyl)-3-methylene-5-nitro-3,4-dihydro-2H-pyran (4)

1-(2,5-difluorophenyl)-2-nitroethanone (3, 56.3 g) and 3-iodo-2-(iodomethyl)prop-1-ene (90.5 g) were dissolved in DMA at 25° C. To it added Cs₂CO₃ (210 g) in a single portion and stirred for 4 h at 25-30° C. After completion of reaction, reaction mixture was filtered through by-flow, washed with DIPE. Filtrate was dumped in cold 1N HCl solution (1.75 L), extracted with DIPE (2×850 ml), combined extracts were washed with brine, separated and evaporated to dryness. Oily residue obtained was stirred in cold IPA, solid precipitated was filtered, washed and dried to get 4 (37.3 g, 53% yield) as light yellow solid.

¹H NMR: (CDCl₃, 400 MHz): δ 7.14-7.03 (m, 3H), 5.37 (s, 1H), 5.28 (s, 1H), 4.61 (s, 1H), 3.60 (t, 2H, J=1.6 Hz); ESI-MS: (+ve mode) 254.1 (M+H)⁺ (50%), 271.0 (M+Na)⁺ (90%); HPLC: 99.3%.

### Step-4: trans-2-(2,5-difluorophenyl)-5-methylene-3-nitrotetrahydro-2H-pyran (5)

6-(2,5-difluorophenyl)-3-methylene-5-nitro-3,4-dihydro-2H-pyran (4, 35 g) was dissolved in MeOH (525 ml) to it added NaBH₄ (15.7 g) portion wise maintaining temperature 0-5° C. over a period of 30 min. Stirred the reaction mixture for 30 min at 0-5° C., quenched with drop wise addition of 6N aqueous HCl solution. To the reaction mixture, cold water (1.05 L) was added, with stirring at 0° C. to get white solid. Solid was filtered, washed with water and dried to get 2-(2,5-difluorophenyl)-5-methylene-3-nitrotetrahydro-2H-pyran (30.7 g) as a mixture of diastereomers (trans:cis: 65:35).

Product thus obtained was dissolved in IPA (92 ml) by heating it to 90° C., from which trans-2-(2,5-difluorophenyl)-5-methylene-3-nitrotetrahydro-2H-pyran was crystallized upon gradual cooling. Crystalline product was filtered, washed with IPA and dried to get trans-2-(2,5-difluorophenyl)-5-methylene-3-nitrotetrahydro-2H-pyran (16.9 g). Filtrate was evaporated to dryness, residue obtained was dissolved in THF, DBU was added, stirred for 15 h at 25° C. Reaction mixture was evaporated to dryness and extracted with ethyl acetate. Combined organic layer was washed with 1N HCl solution, water and brine solution. Organic layer was evaporated to dryness to get diasteriomeric mixture of 2-(2,5-difluorophenyl)-5-methylene-3-nitrotetrahydro-2H-pyran (13.4 g), which was further treated with IPA as above to get trans-2-(2,5-difluorophenyl)-5-methylene-3-nitrotetrahydro-2H-pyran (7.4 g, 29 mmol).

trans-2-(2,5-difluorophenyl)-5-methylene-3-nitrotetrahydro-2H-pyran (24.3 g) obtained was further dissolved in IPA by heating it to 90° C. This was subsequently allowed to cool gradually to room temperature and the crystalline product was filtered, washed with cold IPA and dried to get trans-2-(2,5-difluorophenyl)-5-methylene-3-nitrotetrahydro-2H-pyran as a white crystals (5, 20.8 g, 59% yield).

¹H NMR: (CDCl₃, 400 MHz): δ 7.14-7.10 (m, 1H), 7.06-6.99 (m, 2H), 5.11 (s, 1H), 5.09 (s, 1H), 5.06 (d, 2H, J=9.2 Hz), 4.76 (ddd, 1H, Jⱼ=5.6 Hz, J₂=9.6 Hz, J₃=14.0 Hz), 4.38 (d, 1H, J=12.4 Hz), 4.24 (d, 1H, J=12.4 Hz), 3.09 (d, 2H, J=8.0 Hz); ESI-MS: (+ve mode) 256.1 (M+H)⁺ (100%); HPLC: 99.7%.

### Step-5: trans-2-(2,5-difluorophenyl)-5-methylenetetrahydro-2H-pyran-3-amine (6)

To a vigorously stirred suspension of trans-2-(2,5-difluorophenyl)-5-methylene-3-nitrotetrahydro-2H-pyran (5, 20.5 g) and zinc (61.9 g) in EtOH was added 6 N HCl solution drop wise and stirred for 1 h at 0° C. After completion of reaction, reaction mixture was treated with DCM and ammonia solution. The resulting solid was filtered and washed with DCM. In the filtrate, organic layer was separated and washed with water, saturated brine, dried over anhydrous Na₂SO₄ and evaporated to yield trans-2-(2,5-difluorophenyl)-5-methylenetetrahydro-2H-pyran-3-amine as an off white solid (6, 17.4 g, 97% yield).

¹H NMR: (CDCl₃, 400 MHz): δ 7.26-7.14 (m, 1H), 7.05-6.93 (m, 2H), 4.92 (dd, 2H, J₁=1.6 Hz, J₂=5.2 Hz), 4.36 (d, 1H, J=9.2 Hz), 4.30 (dd, 1H, J₁=1.6 Hz, J₂=12.8 Hz), 4.27 (d, 1H, J=12.8 Hz), 2.85-2.73 (m, 2H) 2.22-2.16 (m, 1H); ESI-MS: (+ve mode) 226.3 (M+H)⁺ (100%); HPLC: 94.9%.

### Step-6: tert-butyl((2R,3S)-2-(2,5-difluorophenyl)-5-methylenetetrahydro-2H-pyran-3-yl) carbamate (7)

D(−)Tartaric acid (12.5 g) was dissolved in methanol to get a clear solution, to it was added a solution of trans-2-(2,5-difluorophenyl)-5-methylenetetrahydro-2H-pyran-3-amine (6, 17 g) dissolved in MeOH (59.5 ml) at 25° C. and the reaction mixture was stirred for 15 h at 25° C. The solid was filtered, washed with methanol and dried. Solid thus obtained was suspended in MeOH (119 ml) and refluxed for 1 h, & cooled gradually to 25° C. and stirred for 15 h. The obtained solid was filtered, washed with MeOH and dried to get (2R,3S)-2-(2,5-difluorophenyl)-5-methylenetetrahydro-2H-pyran-3-amine as a tartrate salt (14.2 g).

The tartrate salt was dissolved in ACN and water, to it added Na₂CO₃ (10 g) portion wise at 25-30° C. Reaction mixture was cooled to 0-5° C. and Boc-anhydride (9.9 g) was added. Reaction mixture was stirred for 2 h, concentrated to remove ACN, to the residue obtained was added ice cold water (150 ml) and stirred for 30 min. The solid precipitated was filtered, washed with water and dried to get tert-butyl((2R,3S)-2-(2,5-difluorophenyl)-5-methylenetetrahydro-2H-pyran-3-yl) carbamate as a white solid (7, 12.06 g, 49% yield).

¹H NMR: (CDCl₃, 400 MHz): δ 7.20-7.30 (m, 1H), 6.93-6.99 (m, 21-1), 4.95 (d, 2H, J=10.4 Hz), 4.47 (d, 2H, J=9.2 Hz), 4.30 (dd, 1H, J₁=12.8 Hz, J₂=1.60 Hz), 4.06 (d, 1H, J=12.8 Hz), 3.70 (d, 1H, J=8.4 Hz), 2.83 (dd, 1H, J₁=12.8 Hz, J₂=4.0 Hz), 2.27 (t, 1H, J=12.4 Hz), 1.26 (s, 9H); ESI-MS: (+ve mode) 326.5 (M+H)⁺ (100%); HPLC: 96.4%.

### Step-7: tert-butyl((2R,3S)-2-(2,5-difluorophenyl)-5-oxotetrahydro-2H-pyran-3-yl)carbamate (Intermediate-1)

Tert-butyl((2R,3S)-2-(2,5-difluorophenyl)-5-methylenetetrahydro-2H-pyran-3-yl) carbamate (7, 10 g) was dissolved in DCM and ACN, to it added solution of NaIO₄ (19.75 g) dissolved in water (150 ml) followed by RuCl₃3H₂O (160 mg) at 25° C. Reaction mixture was stirred for 3 h. After completion of reaction, diluted it with DCM and added water (150 ml), layers were separated and aqueous layer was extracted with DCM. Combined organic layer was washed with 10% aqueous Na₂S₂O₃ solution, water and brine. Organic layer was evaporated to dryness to get tert-butyl((2R,3S)-2-(2,5-difluorophenyl)-5-oxotetrahydro-2H-pyran-3-yl)carbamate as a white crystalline powder (8.5 g, 84% yield).

¹H NMR: (CDCl₃, 400 MHz): δ 7.20-7.30 (m, 1H), 6.96-7.04 (m, 2H), 4.83 (d, 1H, J=8.0 Hz), 4.61 (m, 1H), 4.29 (dd, 1H, J₁=16.4 Hz, J₂=1.60 Hz), 4.11 (d, 1H, J=16.4 Hz), 3.02-3.07 (m, 1H), 2.60-2.80 (m, 1H), 1.30 (s, 9H); ESI-MS: (+ve mode) 328.4 (M+H)⁺ (40%); HPLC: 98.9%.

### Synthesis of substituent R²[hexahydro-1H-furo[3,4-c]pyrrole; (2a)]

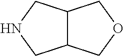

Synthesis of substituent R²(hexahydro-1H-furo[3,4-c]pyrrole; 2a) was carried out as shown in Scheme-3 and the stepwise procedure is depicted below:

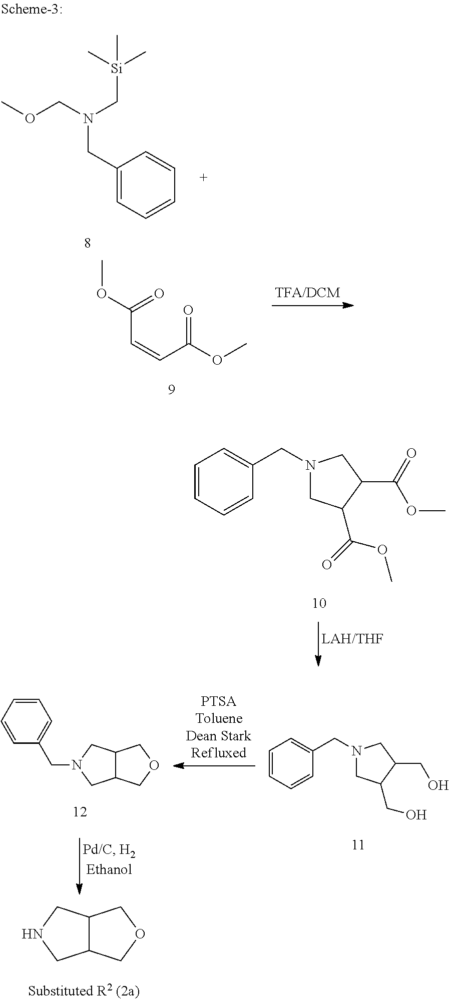

### Step-1: 1-Benzyl-pyrrolidine-3,4-dicarboxylic acid dimethyl ester (10)

N-benzyl-1-methoxy-N-((trimethylsilyl)methyl)methanamine (8, 21.4 g) and dimethyl maleate (9, 10 g) were dissolved in DCM (200 ml). To the reaction mixture TFA (0.54 ml, 6.94 mmol) was added and stirred for 3 h. After completion of reaction, reaction mixture was neutralized with saturated NaHCO₃ solution (100 ml). Organic layer was washed with water, brine solution, dried over anhydrous Na₂SO₄ and evaporated under reduced pressure to get 1-Benzyl-pyrrolidine-3,4-dicarboxylic acid dimethyl ester (10) as a light yellow color oil (16.7 g, 87% yield).

¹H NMR: (CDCl₃, 400 MHz): δ 7.25-7.13 (m, 5H), 3.72 (s, 2H), 3.58 (s, 6H), 3.26-3.20 (m, 2H), 3.08-3.04 (m, 2H), 3.04-2.63 (m, 2H); ESI-MS: (+ve mode) 277.9 (M+H)⁺ (60%), 299.9 (M+Na) (80%; HPLC: 90%.

### Step-2: (1-Benzylpyrrolidine-3,4-diyl)dimethanol (11)

1-Benzyl-pyrrolidine-3,4-dicarboxylic acid dimethyl ester (10, 15 g), dissolved in THF (30 ml) was added to a suspension of LiAlH₄ (4.3 g) and stirred for 2 h at 25° C. Reaction mixture was quenched with water (2 ml) and 2N NaOH solution (2 ml). The reaction mixture was filtered, dried over anhydrous Na₂SO₄ and evaporated under reduced pressure to get (1-Benzylpyrrolidine-3,4-diyl)dimethanol (11) as a yellow color oil (11.6 g, 97% yield).

¹H NMR: (CDCl₃, 400 MHz): δ 7.25-7.13 (m, 5H), 3.67 (s, 2H), 3.64-3.47 (m, 4H), 2.70-2.65 (m, 2H), 2.44-2.39 (m, 2H), 2.15-2.11 (m, 2H); ESI-MS: (+ve mode) 222.1 (M+H)⁺ (85%); HPLC: 94%.

### Step-3: 5-Benzyl-hexahydro-furo[3,4-c]pyrrole (12)

A mixture of 1-Benzylpyrrolidine-3,4-diyl)dimethanol (11, 10 g) and PTSA (1.94 g) in dry toluene (100 ml) was refluxed at 140° C. for 16 h. The reaction mixture was cooled and basified with 1N NaOH solution (100 ml), organic layer was separated off, washed with water, brine solution and dried to yield 5-Benzyl-hexahydro-furo[3,4-c]pyrrole (12) as an oil (5.9 g, 64% yield).

¹H NMR: (CDCl₃, 400 MHz): δ 7.05-7.23 (m, 5H), 3.77-3.67 (s, 4H), 3.49 (s, 2H), 2.27-2.25 (m, 4H) 2.26-2.25 (m, 2H); ESI-MS: (+ve mode) 204.2 (M+H)⁺ (89%); HPLC: 84%.

### Step-4: hexahydro-1H-furo[3,4-c]pyrrole (2a)

5-Benzyl-hexahydro-furo[3,4-c]pyrrole (12, 5 g) was dissolved in EtOH (50 ml) and hydrogenated in presence of 10% Pd/C (0.5 g) at 60 psi. Filtered the reaction mixture was filtered, evaporated to dryness to get hexahydro-1H-furo[3,4-c]pyrrole (2a) as a colorless oil (2.56 g, 92% yield).

¹H NMR: (CDCl₃, 400 MHz): δ 3.67-3.58 (m, 4H) 3.43-3.33 (m, 2H), 2.97-2.88 (m, 4H); ESI-MS: (+ve mode) 113.8 (M+H)⁺ (55%); GC: 92%.

### Synthesis of substituent R²: [(3,4,5,6-tetrahydro-1H-thieno[3,4-c]pyrrole 2,2-dioxide hydrobromide; (2b)]

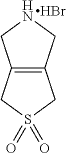

Synthesis of substituent R² (3,4,5,6-tetrahydro-1H-thieno[3,4-c]pyrrole 2,2-dioxide hydrobromide; (2b) was carried out as shown in Scheme-4 and the stepwise procedure is depicted below:

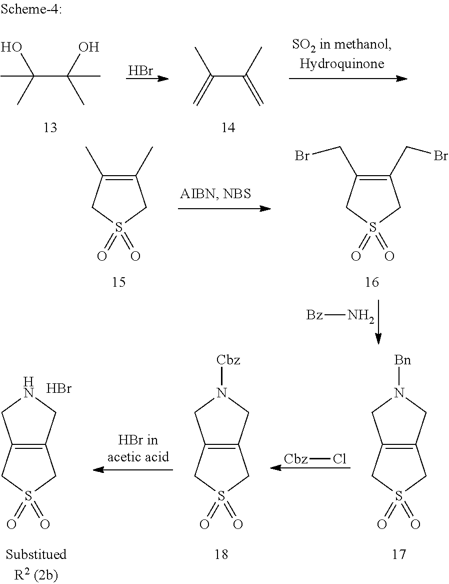

### Step-1: 2,3-dimethylbuta-1,3-diene (14)

To 2,3-dimethylbutane-2,3-diol (13, 85 g), 48% aqueous HBr was added to get the colorless solution. Mixture was fractionally distilled, washed twice with water and dried over anhydrous CaCl₂. Mixture was redistilled and the fraction of 69-70° C. was collected to get 2,3-dimethylbuta-1,3-diene (14, 38 g. 64% yield).

¹H NMR: (CDCl₃, 400 MHz): δ 5.06 (2H, s), 4.97 (2H, s), 1.92 (61-1, s); ESI-MS: (+ve mode) 83.3 (M+H)⁺ (70%).

### Step-2: 3,4-dimethyl-2,5-dihydrothiophene 1,1-dioxide (15)

A mixture of hydroquinone (492 mg) and 2,3-dimethylbuta-1,3-diene (14, 31.96 ml) was placed in sealed tube and a solution of sulfur dioxide in MeOH (140 ml) was added. Reaction mixture was heated at 85° C. for 4 h and cooled to room temperature. Crystals obtained was filtered, washed with cold methanol and dried to get 3,4-dimethyl-2,5-dihydrothiophene 1,1-dioxide (15) as white crystalline solid (30 gm, 72% yield).

¹H NMR: (CDCl₃, 400 MHz): δ 3.73 (4H, d, J=1.2 Hz), 1.78 (6H, t, J=1.2 Hz); ESI-MS: (+ve mode) 147.2 (M+H)⁺ (70%), 169.1 (M+Na)⁺ (40%).

### Step-3: 3,4-bis(bromomethyl)-2,5-dihydrothiophene 1,1-dioxide (16)

A mixture of 3,4-dimethyl-2,5-dihydrothiophene 1,1-dioxide (15, 20 g), 1-bromopyrrolidine-2,5-dione (53.5 g), and AIBN (400 mg) in CHCl₃ was heated for 15 hr. After completion of reaction, filtrate was evaporated under reduced pressure. The residue obtained was recrystallize from methanol to get 3,4-bis(bromomethyl)-2,5-dihydrothiophene 1,1-dioxide as a white crystals (16, 19 g, 45% yield).

¹H NMR: (CDCl₃, 400 MHz): δ 4.06 (4H, s), 4.01 (4H, s); ESI-MS: (+ve mode) 303.8 (M+H)⁺ (90%), 305.7 (M+2H)⁺ (70%).

### Step-4: 5-benzyl-3,4,5,6-tetrahydro-1H-thieno[3,4-c]pyrrole 2,2-dioxide (17)

Mixture of 3,4-bis(bromomethyl)-2,5-dihydrothiophene 1,1-dioxide (16, 12 g) and phenylmethanamine (10.84 ml) in acetonitrile was stirred at 25° C. for 2 hr. After completion of reaction, solvent was removed under reduced pressure, ethyl acetate and 1N NaOH were added, organic layer was separated and aq layer was extracted with ethyl acetate. The combined organic layer was washed with brine, dried over anhydrous Na₂SO₄ and concentrated under reduced pressure to give 5-benzyl-3,4,5,6-tetrahydro-1H-thieno[3,4-c]pyrrole 2,2-dioxide (17) as a solid compound (3.7 g, 38% yield).

¹H NMR: (CDCl₃, 400 MHz): δ 7.34-7.29 (5H, m), 3.88 (2H, s), 3.77 (4H, s), 3.61 (4H, s); ESI-MS: (+ve mode) 250.3 (M+H)⁺ (100%).

### Step-5: benzyl 4,6-dihydro-1H-thieno[3,4-c]pyrrole-5(3H)-carboxylate 2,2-dioxide (18)

A mixture of 5-benzyl-3,4,5,6-tetrahydro-1H-thieno[3,4-c]pyrrole 2,2-dioxide (17, 3.6 g) and CBZ-Cl (13.5 ml) in toluene was stirred for 3 hr. After completion of reaction, diethyl ether was added till solid precipitated out. Solid was filtered and dried under reduced pressure to get benzyl 4,6-dihydro-1H-thieno[3,4-c]pyrrole-5(3H)-carboxylate 2,2-dioxide (18, 2.7 g, 64% yield).

¹H NMR: (CDCl₃, 400 MHz): δ 7.38-7.35 (5H, m), 5.19 (2H, s), 4.31 (4H, s), 3.88 (4H, d, J=13.6 Hz); ESI-MS: (+ve mode) 294.4 (M+H)⁺ (80%).

### Step-6: 3,4,5,6-tetrahydro-1H-thieno[3,4-c]pyrrole 2,2-dioxide hydrobromide (2b)

To a solution of benzyl 4,6-dihydro-1H-thieno[3,4-c]pyrrole-5(3H)-carboxylate 2,2-dioxide (18, 3.7 g) in glacial acetic acid, HBr in glacial acetic acid was added and the reaction mixture was stirred at 25° C. for 3 h. After completion of reaction, diethyl ether was added to afford sticky solid, solvent was decanted and added minimum amount of methanol to get the crystalline solid as 3,4,5,6-tetrahydro-1H-thieno[3,4-c]pyrrole 2,2-dioxide hydrobromide as a hydrobromide salt (2b, 1.5 g, 50% yield).

¹H NMR: (CDCl₃, 400 MHz): δ 9.43 (2H, bs), 4.08 (4H, s), 4.02 (4H, s); ESI-MS: (+ve mode) 160.4 (M+H)⁺ (88%).

The other groups representing R² as described elsewhere in the specification were sourced commercially or were prepared either by similar processes as described above with suitable modifications as are necessary which are within the scope of a skilled person or prepared following literature processes. Such literature processes including suitable variations thereof are incorporated herein as references.

### Synthesis of Compound 1: (2R,3S,5R)-2-(2,5-difluorophenyl)-5-(5-(methylsulfonyl)-hexahydro-pyrrolo[3,4-c]pyrrol-2(1H)-yl)tetrahydro-2H-pyran-3-amine

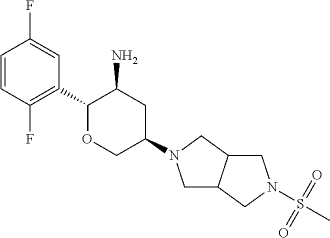 [SMILES: CS(=O)(=O)N1CC2CN([C@H]3CO[C@H](c4cc(F)ccc4F)[C@@H](N)C3)CC2C1]

### Step-1: Synthesis of tert-butyl((2R,3S,5R)-2-(2,5-difluorophenyl)-5-(5-(methylsulfonyl)-hexahydropyrrolo[3,4-c]pyrrol-2(1H)-yl)tetrahydro-2H-pyran-3-yl)carbamate

Under nitrogen atmosphere ((2R,3S)-2-(2,5-difluorophenyl)-5-oxotetrahydro-2H-pyran-3-yl)carbamate (Intermediate-1; 250 mg) and 5-(methylsulfonyl)octahydropyrrolo[3,4-c]pyrrol-2-ium 4-methylbenzenesulfonate (substituent-R²; 172 mg) was dissolved in anhydrous DMA to get the pale yellow clear solution. Reaction mixture was cool to 0-5° C. and sodiumtriacetoxyborohydride (211 mg) was added. The reaction mixture was stirred at 0-5° C. for 2 h, poured in ice cold water, solid precipitated was filtered, washed with water and dried to get the title compound as a white solid (234 mg, 61% yield).

### Step-2: Synthesis of (2R,3S,5R)-2-(2,5-difluorophenyl)-5-(5-(methylsulfonyl)-hexahydro-pyrrolo[3,4-c]pyrrol-2(1H)-yl)tetrahydro-2H-pyran-3-amine

Compound of step-1 (tert-butyl((2R,3S,5R)-2-(2,5-difluorophenyl)-5-(5-(methylsulfonyl)hexahydropyrrolo[3,4-c]pyrrol-2(1H)-yl)tetrahydro-2H-pyran-3-yl)carbamate; 210 mg) was treated with HCl in dioxane solution at 15-25° C. for 2 h. Solvent was removed under reduced pressure and water was added to get clear solution, which was extracted with DCM. Aqueous layer was basified with saturated aqueous NaHCO₃ solution and extracted with DCM. Combined organic layer was washed with water (50 ml), evaporated to get (2R,3S,5R)-2-(2,5-difluorophenyl)-5-(5-(methylsulfonyl)hexahydropyrrolo[3,4-c]pyrrol-2(1H)-yl)tetrahydro-2H-pyran-3-amine as a white solid (160 mg, 95% yield).

¹H NMR: (CD₃OD, 400 MHz): 7.31-7.27 (m, 1H), 7.24-7.20 (m, 2H), 4.68 (d, 1H, J=10 Hz), 4.46-4.42 (m, 1H), 3.98-3.96 (m, 1H), 3.87-3.83 (m, 1H), 3.77 (t, 1H, J=10.8 Hz), 3.71-3.67 (m, 1H), 3.62-3.56 (m, 1H), 3.41-3.33 (m, 4H), 3.30-3.23 (m, 4H), 2.95 (s, 3H), 2.78-2.69 (m, 1H), 2.15 (q, 1H, J=11.6 Hz); ESI-MS: (+ve mode) 402.0 (M+H)⁺ (100%), 423.8 (M+Na)⁺ (50%); HPLC: 98.2%.

### Synthesis of Compound 2: (2R,3S,5R)-2-(2,5-difluorophenyl)-5-(7-(methylsulfonyl)-2,7-diazaspiro[4.4]-nonan-2-yl)tetrahydro-2H-pyran-3-amine

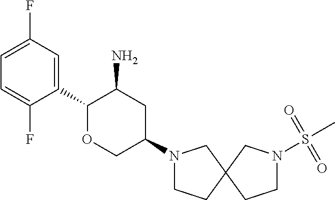 [SMILES: CS(=O)(=O)N1CCC2(CCN([C@H]3CO[C@H](c4cc(F)ccc4F)[C@@H](N)C3)C2)C1]

### Step-1: Synthesis of tert-butyl((2R,3S,5R)-2-(2,5-difluorophenyl)-5-(7-(methylsulfonyl)-2,7-diazaspiro[4.4]nonan-2-yl)tetrahydro-2H-pyran-3-yl)carbamate

Under inert atmosphere ((2R,3S)-2-(2,5-difluorophenyl)-5-oxotetrahydro-2H-pyran-3-yl)carbamate (Intermediate-1; 250 mg) and 2-(methylsulfonyl)-2,7-diazaspiro[4.4]nonane (substituent-R²; 172 mg) were dissolved in anhydrous MeOH, Decaborane (28 mg) was added to this reaction mixture at 25-30° C. and stirred for 15 h. MeOH was removed from the reaction mixture and residue obtained was purified by column chromatography using 0 to 2% MeOH in DCM as an eluent system to get the title compound as a white solid (264 mg, 67% yield).

### Step-2: Synthesis of (2R,3S,5R)-2-(2,5-difluorophenyl)-5-(7-(methylsulfonyl)-2,7-diazaspiro[4.4]nonan-2-yl)tetrahydro-2H-pyran-3-amine

Compound of step-1 (tert-butyl((2R,3S,5R)-2-(2,5-difluorophenyl)-5-(7-(methylsulfonyl)-2,7-diazaspiro[4.4]nonan-2-yl)tetrahydro-2H-pyran-3-yl)carbamate; 250 mg) was dissolved in DCM, to it TFA was added and stirred at 25° C. for 2 h. After completion of reaction, mixture was evaporated to dryness and residue obtained was neutralized with 2.5% ammonium hydroxide, solvents were removed under reduced pressure and residue was triturated with diethyl ether to get the title compound as a white powder (189 mg, 94% yield).

¹H NMR: (CD₃OD, 400 MHz): 7.33-7.25 (m, 3H), 4.85-4.82 (d, 1H, J=10.4 Hz), 4.51-4.49 (d, 2H, J=6.8 Hz), 3.84-3.82 (m, 2H), 3.78-3.67 (m, 4H), 3.51 (t, 2H, J=6.8 Hz), 3.43-3.35 (m, 2H), 3.07 (s, 3H), 2.89-2.86 (m, 1H), 2.25-2.19 (m, 2H), 2.17-2.08 (m, 3H); ESI-MS: (+ve mode) 416.1 (M+H)⁺ (100%); HPLC: 98.2%.

### Synthesis of Compound 3: (2R,3S,5R)-2-(2,5-difluorophenyl)-5-(tetrahydro-1H-furon[3,4-c]pyrrol-5(3H)-yl)tetrahydro-2H-pyran-3-amine

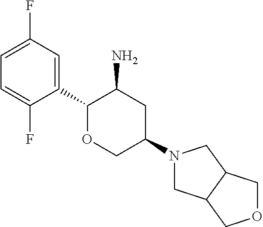 [SMILES: N[C@H]1C[C@@H](N2CC3COCC3C2)CO[C@@H]1c1cc(F)ccc1F]

### Step-1: Synthesis of tert-butyl((2R,3S,5R)-2-(2,5-difluorophenyl)-5-(tetrahydro-1H-furo[3,4-c]pyrrol-5(3H)-yl)tetrahydro-2H-pyran-3-yl)carbamate

Hexahydro-1H-furo[3,4-c]pyrrol-5-ium 4-methylbenzenesulfonate (substituent-R²; 445 mg) was dissolved in DMA, Intermediate-1 (150 mg) and DIEA (556 mg) were added to it and the solution was stirred for 30 min. Glacial CH₃COOH (413 mg) was added to this mixture and stirred at 25° C. for 15 min. Sodium cyanoborohydride was added and stirred for 3 h. Reaction mixture was cooled and added to a mixture of ethyl acetate) and saturated aqueous NaHCO₃ solution. Organic layer was washed with water, brine, dried over anhydrous Na₂SO₄, filtered and evaporated to dryness to give diastereomeric mixture of the title compound, which was purified by flash column chromatography using 0-3% methanol in DCM as an eluent system to get tert-butyl((2R,3S,5R)-2-(2,5-difluorophenyl)-5-(tetrahydro-1H-furo[3,4-c]pyrrol-5(3H)-yl)tetrahydro-2H-pyran-3-yl)carbamate as a white solid (132 mg, 67% yield).

### Step-2: Synthesis of (2R,3S,5R)-2-(2,5-difluorophenyl)-5-(tetrahydro-1H-furo[3,4-c]pyrrol-5(3H)-yl)tetrahydro-2H-pyran-3-amine

Compound of the step-1 (tert-butyl((2R,3S,5R)-2-(2,5-difluorophenyl)-5-(tetrahydro-1H-furo[3,4-c]pyrrol-5(3H)-yl)tetrahydro-2H-pyran-3-yl)carbamate; 132 mg) was dissolved in anhydrous MeOH to get the clear solution. HCl gas was bubbled through this solution for 2 h. Solvent was removed under reduced pressure and residue was dissolved in water, basified with saturated aqueous NaHCO₃ solution and extracted with DCM. Combined organic layer was washed with water and saturated brine solution, evaporated to dryness to get the 2R,3S,5R-2-(2,5-difluorophenyl)-5-(tetrahydro-1H-furo[3,4-c]pyrrol-5(3H)-yl)tetrahydro-2H-pyran-3-amine as a white solid (98 mg, 97% yield).

¹H NMR: (CD₃OD, 400 MHz): 7.18-7.19 (m, 1H), 7.13-7.11 (m, 2H), 4.55-4.54 (d, 1H, J=10.4 Hz), 4.3 (m, 1H), 3.77-3.74 (m, 2H), 3.63-3.62 (m, 2H), 3.60-3.56 (m, 5H), 3.04-3.03 (m, 4H), 2.6-2.7 (m, 2H), 1.97-1.94 (m, 1H); ESI-MS: (+ve mode) 324.9 (M+H)⁺ (100%), 347 (M+Na)⁺ (25%); HPLC: 96.6%.

Using either of the above procedures, following additional compounds were prepared by suitable reductive amination of intermediate-1 with appropriate substituent R² followed by removal of amine protecting group.

Compound 4: (2R,3S,5R)-2-(2,5-difluorophenyl)-5-(hexahydropyrrolo[3,4-c]pyrrol-2(1H)-yl)tetrahydro-2H-pyran-3-amine

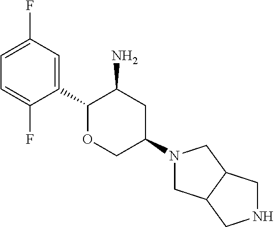 [SMILES: N[C@H]1C[C@@H](N2CC3CNCC3C2)CO[C@@H]1c1cc(F)ccc1F]

¹H NMR: (CD₃OD, 400 MHz): 7.29-7.27 (m, 1H), 7.23-7.20 (m, 2H), 4.64 (d, 1H, J=10.4 Hz), 4.38-4.35 (dd, 1H, Jⱼ=2.4 Hz, J₂=10.4 Hz), 3.69 (t, 1H, J=11 Hz), 3.57-3.53 (m, 4H), 3.34-3.30 (m, 8H), 2.68-2.65 (m, 1H), 2.04 (q, 1H, J=11.6 Hz); ESI-MS: (+ve mode) 323.9 (M+H)⁺ (100%), 345.9 (M+Na)⁺ (20%); HPLC: 98.6%.

Compound 5: (2R,3S,5R)-2-(2,5-difluorophenyl)-5-(5-((trifluoromethyl)sulfonyl) hexahydropyrrolo[3,4-c]pyrrol-2(1H)-yl)tetrahydro-2H-pyran-3-amine

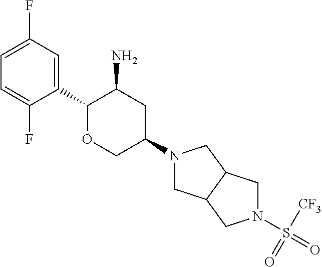 [SMILES: N[C@H]1C[C@@H](N2CC3CN(S(=O)(=O)C(F)(F)F)CC3C2)CO[C@@H]1c1cc(F)ccc1F]

¹H NMR: (CD₃OD, 400 MHz): δ 7.45-7.43 (m, 1H), 7.24-7.19 (m, 2H), 4.80-4.72 (m, 1H), 4.47-4.30 (m, 1H), 3.93-3.82 (m, 2H), 3.60-3.81 (m, 6H), 3.28-3.18 (m, 2H), 3.08-2.93 (m, 2H), 2.71-2.52 (m, 2H), 2.23-2.08 (m, 1H); ESI-MS: (+ve mode) 456.0 (M+H)⁺ (100%); HPLC: 95.0%.

Compound 6: (2R,3S,5R)-2-(2,5-difluorophenyl)-5-(5-(phenylsulfonyl)hexahydropyrrolo[3,4-c]pyrrol-2(1H)-yl)tetrahydro-2H-pyran-3-amine

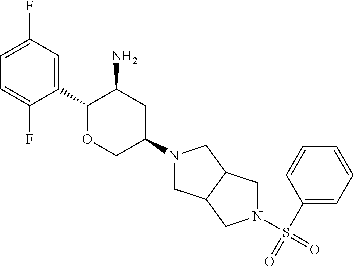 [SMILES: N[C@H]1C[C@@H](N2CC3CN(S(=O)(=O)c4ccccc4)CC3C2)CO[C@@H]1c1cc(F)ccc1F]

¹H NMR: (CD₃OD, 400 MHz): δ 7.85-7.82 (m, 2H), 7.73-7.64 (m, 3H), 7.31-7.28 (m, 1H), 7.24-7.21 (m, 2H), 4.66-4.64 (m, 1H), 4.42-4.39 (m, 1H), 3.81-3.72 (m, 3H), 3.69-3.66 (m, 2H), 3.39-3.36 (m, 2H), 3.06-3.00 (m, 4H), 2.95-2.83 (m, 2H), 2.73-2.70 (m, 1H), 2.05-2.02 (m, 1H); ESI-MS: (+ve mode) 464.0 (M+H)⁺ (100%); HPLC: 95.68%.

### Compound 7: 5-((3R,5S,6R)-5-amino-6-(2,5-difluorophenyl)tetrahydro-2H-pyran-3-yl)-N,N-dimethylhexahydropyrrolo[3,4-c]pyrrole-2(1H)-sulfonamide

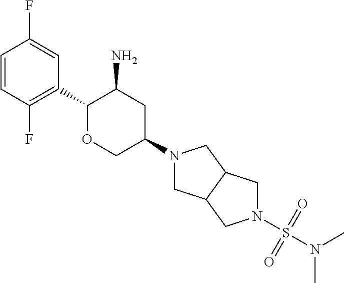 [SMILES: CN(C)S(=O)(=O)N1CC2CN([C@H]3CO[C@H](c4cc(F)ccc4F)[C@@H](N)C3)CC2C1]

¹H NMR: (CD₃OD, 400 MHz): 7.29-7.26 (m, 1H), 7.24-7.21 (m, 2H), 4.67-4.65 (m, 1H 4.45-4.43 (m, 2H), 3.93-3.32 (m, 2H), 3.77-3.72 (m, 1H), 3.69-3.66 (m, 1H), 3.61-3.55 (m, 2H), 3.36 (s, 3H), 3.30-3.29 (s, 3H), 2.88 (s, 6H), 2.77-2.74 (m, 1H), 2.14-2.07 (m, 1H); ESI-MS: (+ve mode) 431.1 (M+H)⁺ (100%), 453 (M+Na)⁺; HPLC: 97.50%.

### Compound 8: (2R,3S,5R)-2-(2,5-difluorophenyl)-5-(5-(methylsulfonyl)-5,6-dihydropyrrolo[3,4-c]pyrrol-2(1H,3H,4H)-yl)tetrahydro-2H-pyran-3-amine

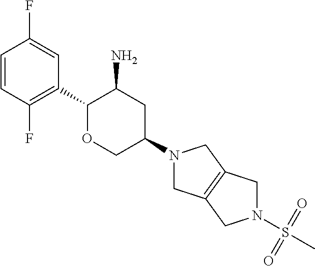 [SMILES: CS(=O)(=O)N1CC2=C(CN([C@H]3CO[C@H](c4cc(F)ccc4F)[C@@H](N)C3)C2)C1]

¹H NMR: (CD₃OD 400 MHz): 7.32-7.28 (m, 1H), 7.26-7.23 (m, 2H), 4.77 (d, 1H, J=10 Hz), 4.32 (dd, 1H, J₁=2.0 Hz, J₂=10.8 Hz), 4.19 (s, 4H), 3.89-3.83 (m, 4H), 3.70-3.65 (m, 1H), 3.61 (t, 1H, J=11.6 Hz), 3.53-3.46 (m, 1H), 3.04 (s, 3H), 2.65-2.62 (dd, 1H, J₁=1.2 Hz, J₂=12 Hz), 1.84 (q, 1H, J=12 Hz); ESI-MS: (+ve mode) 400.0 (M+H)⁺ (100%); HPLC: 99.4%.

### Compound 9: 5-((3R,5S,6R)-5-amino-6-(2,5-difluorophenyl)tetrahydro-2H-pyran-3-yl)-N,N-dimethyl-3,4,5,6-tetrahydropyrrolo pyrrole-2(1H)-sulfonamide

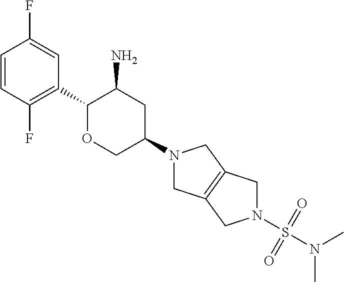 [SMILES: CN(C)S(=O)(=O)N1CC2=C(CN([C@H]3CO[C@H](c4cc(F)ccc4F)[C@@H](N)C3)C2)C1]

¹H NMR: (CD₃OD, 400 MHz): −7.25-7.22 (m, 1H), 7.18-7.13 (m, 2H), 4.41 (d, J=9.6 Hz, 1H), 4.22-4.19 (m, 1H), 4.11 (s, 4H), 3.59 (s, 4H), 3.37 (t, J=10.8 Hz, 1H), 3.22-3.14 (m, 1H), 3.05-2.95 (m, 1H), 2.82 (s, 6H), 2.50-2.41 (m, 1H), 1.55 (q, J=12.0 Hz, 1H). ESI-MS: (+ve mode) 429.15 (100%) (M+H)⁺; HPLC: 95.18%.

### Compound 10: 5-((3R,5S,6R)-5-amino-6-(2,5-difluorophenyl)tetrahydro-2H-pyran-3-yl)-2-cyclopropyltetrahydropyrrolo[3,4-c]pyrrole-1,3(2H,3aH)-dione

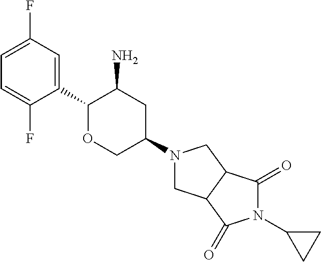 [SMILES: N[C@H]1C[C@@H](N2CC3C(=O)N(C4CC4)C(=O)C3C2)CO[C@@H]1c1cc(F)ccc1F]

¹H NMR: (CD₃OD, 400 MHz): −7.30-7.26 (m, 1H), 7.23-7.18 (m, 2H), 4.53 (d, J=10.0 Hz, 1H), 4.27-4.23 (m, 1H), 3.48-3.41 (m, 2H), 3.38-3.31 (m, 2H), 3.29-3.21 (m, 2H), 2.77-2.69 (m 1H), 2.65-2.61 (m, 2H), 2.60-2.54 (m, 1H), 2.53-2.49 (m, 1H), 1.65 (q, J=12.0 Hz, 1H), 1.92-0.87 (m, 4H). ESI-MS: (+ve mode) 391.9 (100) (M+H)⁺; HPLC: 98.30%.

### Compound 11: 5-((3R,5S,6R)-5-amino-6-(2,5-difluorophenyl)tetrahydro-2H-pyran-3-yl)-2-benzyltetrahydropyrrolo[3,4-c]pyrrole-1,3(2H,3aH)-dione

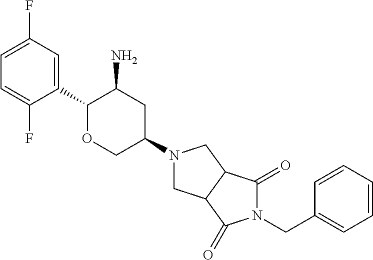 [SMILES: N[C@H]1C[C@@H](N2CC3C(=O)N(Cc4ccccc4)C(=O)C3C2)CO[C@@H]1c1cc(F)ccc1F]

¹H NMR: (CD₃OD, 400 MHz): 7.35-7.30 (m, 6H), 7.21-7.20 (m, 2H), 4.66 (s, 2H), 4.55 (d, 1H, J=10 Hz), 4.27-4.25 (m, 1H), 3.48-3.44 (m, 2H), 3.42-3.36 (m, 4H), 2.80-2.74 (m, 1H), 2.69-2.68 (m, 2H), 2.55-2.52 (m, 1H), 1.66 (q, 1H, J=11.6 Hz); ESI-MS: (+ve mode) 441.9 (M+H)⁺ (100%); HPLC: 97.2%.

### Compound 12: (2R,3S,5R)-2-(2,5-difluorophenyl)-5-(5-(methylsulfonyl)hexahydro-1H-pyrrolo[3,4-c]pyridin-2(3H)-yl)tetrahydro-2H-pyran-3-amine

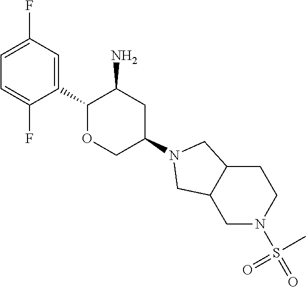 [SMILES: CS(=O)(=O)N1CCC2CN([C@H]3CO[C@H](c4cc(F)ccc4F)[C@@H](N)C3)CC2C1]

¹H NMR: (CD₃OD, 400 MHz): 7.26-7.23 (m, 3H), 4.66-4.63 (m, 1H), 3.58-3.48 (m, 7H), 3.31 (s, 3H), 3.13-3.14 (m, 2H), 2.95 (m, 1H), 2.94-2.66 (m, 3H), 2.24-2.22 (m, 1H), 2.09-2.05 (m, 3H), 1.89-1.94 (m, 1H); ESI-MS: (+ve mode) 416.07 (M+H)⁺ (100%); HPLC: 95.3%.

### Compound 13: (2R,3S,5R)-2-(2,5-difluorophenyl)-5-(2-(methylsulfonyl)hexahydro-1H-pyrrolo[3,4-]pyridin-5(6H)-yl)tetrahydro-2H-pyran-3-amine

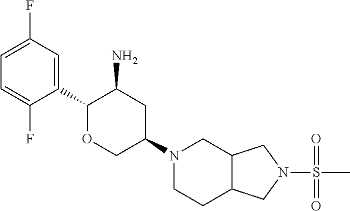 [SMILES: CS(=O)(=O)N1CC2CCN([C@H]3CO[C@H](c4cc(F)ccc4F)[C@@H](N)C3)CC2C1]

¹H NMR: (CD₃OD, 400 MHz): 7.29-7.36 (m, 3H), 4.61-4.63 (m, 1H), 3.48-3.37 (m, 7H), 3.34 (s, 3H), 3.13-3.14 (m, 2H), 2.98 (m, 1H), 2.94-2.61 (m, 3H), 2.24-2.22 (m, 1H), 2.05-2.01 (m, 3H), 1.91-1.84 (m, 1H); ESI-MS: (+ve mode) 416.07 (M+H)⁺ (100%); HPLC: 96.6%.

### Compound 14: (2R,3S,5R)-2-(2,5-difluorophenyl)-5-(8-(methylsulfonyl)-2,8-diazaspiro[4.5]decan-2-yl)tetrahydro-2H-pyran-3-amine

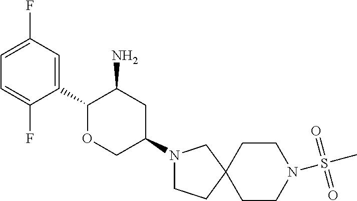 [SMILES: CS(=O)(=O)N1CCC2(CCN([C@H]3CO[C@H](c4cc(F)ccc4F)[C@@H](N)C3)C2)CC1]

¹H NMR: (CD₃OD, 400 MHz): 7.30-7.28 (m, 1H), 7.26-7.22 (m, 2H), 4.74-4.71 (m, 1H), 4.30-4.24 (m, 1H), 3.87-3.84 (m, 2H), 3.75-3.61 (m, 2H), 3.61 (s, 3H), 3.58-3.60 (m, 2H), 3.31-3.30 (m, 2H), 3.26-3.22 (m, 3H), 2.97-2.84 (m, 4H), 2.20-2.10 (m, 2H), 2.04-1.95 (m, 1H), 1.93-1.82 (m, 1H); ESI-MS: (+ve mode) 464.0 (M+H)⁺ (100%); HPLC: 95.32%.

### Compound 15: (2R,3S,5R)-2-(2,5-difluorophenyl)-5-(1-(methylsulfonyl)hexahydropyrrolo[3,4-b]pyrrol-5(1H)-yl)tetrahydro-2H-pyran-3-amine

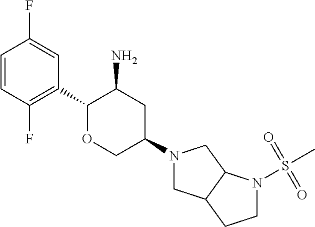 [SMILES: CS(=O)(=O)N1CCC2CN([C@H]3CO[C@H](c4cc(F)ccc4F)[C@@H](N)C3)CC21]

¹H NMR: (CD₃OD, 400 MHz): 7.30-7.26 (m, 1H), 7.22-7.20 (m, 2H), 4.67-4.65 (d, 1H, J=10 Hz), 4.44-4.38 (m, 2H,3.85-3.82 (m, 1H), 3.76-3.71 (m, 1H), 3.64-3.46 (m, 6H), 3.33-3.29 (m, 2H), 2.97 (s, 3H), 2.76-2.72 (m, 1H), 2.28-2.22 (m, 1H), 2.13 (q, 1H, J=12 Hz), 1.96-1.92 (m, 1H); ESI-MS: (+ve mode) 402.1 (M+H)⁺ (100%), 424.1 (M+Na)⁺ (10%); HPLC: 95.6%.

### Compound 16: (2R,3S,5R)-2-(2,5-difluorophenyl)-5-(5-(methylsulfonyl)hexahydropyrrolo[3,4-b]pyrrol-1(2H)-yl)tetrahydro-2H-pyran-3-amine

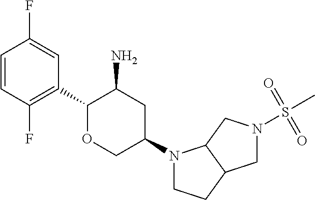 [SMILES: CS(=O)(=O)N1CC2CCN([C@H]3CO[C@H](c4cc(F)ccc4F)[C@@H](N)C3)C2C1]

¹H NMR: (CD₃OD, 400 MHz): 7.29-7.27 (m, 1H), 7.23-7.20 (m, 2H), 4.65-4.63 (m, 2H), 4.47-4.44 (m, 41), 4.14-4.10 (m, 1H), 3.66-3.48 (m, 4H), 3.48-3.43 (m, 4H), 3.31-3.25 (m, 1H), 2.69 (s, 3H), 2.65-2.62 (m, 1H), 2.42-2.32 (m, 1H), 2.01-1.98 (m, 1H), 1.89-1.78 (m, 1H); ESI-MS: (+ve mode) 402.1 (M+H)⁺ (100%), 424 (M+Na)⁺; HPLC: 97.55%.

### Compound 17: 5-((3R,5S,6R)-5-amino-6-(2,5-difluorophenyl)tetrahydro-2H-pyran-3-yl)-3,4,5,6-tetrahydro-1H-thieno[3,4-c]pyrrole 2,2-dioxide

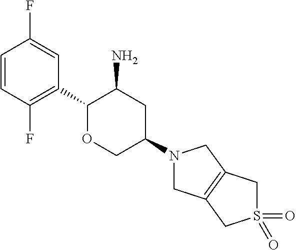 [SMILES: N[C@H]1C[C@@H](N2CC3=C(C2)CS(=O)(=O)C3)CO[C@@H]1c1cc(F)ccc1F]

¹H NMR: (CD₃OD, 400 MHz): 7.30-7.328 (m, 1H), 7.24-7.20 (m, 2H), 4.66-4.65 (d, 1H, J=10 Hz), 4.40-4.38 (t, 1H, J=6.8 Hz), 4.19-4.14 (m, 4H), 3.95-3.90 (m, 4H), 3.71-3.58 (m, 3H), 2.65-2.62 (m, 1H), 2.00 (q, 1H, J=12 Hz); ESI-MS: (+ve mode) 371.0 (M+H)⁺ (100%), 393.1 (M+Na) (55%); HPLC: 96.75%.

### Compound 18: (2R,3S,5R)-5-(5-benzylhexahydropyrrolo[3,4-c]pyrrol-2(1H)-yl)-2-(2,5-difluorophenyl)tetrahydro-2H-pyran-3-amine

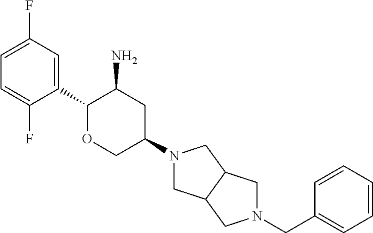 [SMILES: N[C@H]1C[C@@H](N2CC3CN(Cc4ccccc4)CC3C2)CO[C@@H]1c1cc(F)ccc1F]

¹H NMR: (CD₃OD, 400 MHz): δ 7.51-7.49 (m, 5H), 7.25-7.23 (m, 1H), 7.22-7.20 (m, 2H), 4.59 (d, 1H, J=10 Hz), 4.39 (s, 2H), 4.37-4.34 (m, 1H), 3.98-3.95 (m, 1H), 3.88-3.83 (m, 1H), 3.77 (t, 1H, J=10.8 Hz), 3.34-3.31 (m, 8H), 3.06-3.02 (m, 2H), 2.57-2.54 (m, 1H), 1.91-1.87 (q, 1H, J=11.6 Hz); ESI-MS: (+ve mode) 414.2 (M+H)⁺ (100%); HPLC: 96.32%.

### Compound 19: (2R,3S,5R)-2-(2,5-difluorophenyl)-5-(6-(methylsulfonyl)-3,6-diazabicyclo[3.2.0]heptan-3-yl)tetrahydro-2H-pyran-3-amine

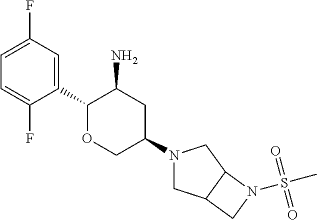 [SMILES: CS(=O)(=O)N1CC2CN([C@H]3CO[C@H](c4cc(F)ccc4F)[C@@H](N)C3)CC21]

¹H NMR: (CD₃OD, 400 MHz): δ 7.31-7.29 (m, 1H), 7.25-7.21 (m, 2H), 5.00-4.97 (m, 1H), 4.68 (d, 1H, J=10.0 Hz), 4.44-4.40 (m, 1H), 4.18 (t, 1H, J=8.4 Hz), 3.81-3.76 (m, 2H), 3.71 (d, 1H, J=11.2 Hz), 3.65-3.62 (m, 1H), 3.59-3.56 (m, 1H), 3.39-3.35 (m, 2H), 3.12-3.04 (m, 1H), 3.02 (s, 3H), 3.00-2.94 (m, 1H), 2.74-2.72 (m, 1H), 2.10 (q, 1H, J=12.0 Hz); ESI-MS: (+ve mode) 388.10 (100%) (M+H)⁺, 410.05 (M+Na)⁺ (20%); HPLC: 96.02%.

### Compound 20: (2R,3S,5R)-2-(2,5-difluorophenyl)-5-(3-(methylsulfonyl)-3,6-diazabicyclo[3.2.0]heptan-6-yl)tetrahydro-2H-pyran-3-amine

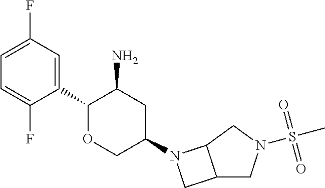 [SMILES: CS(=O)(=O)N1CC2CN([C@H]3CO[C@H](c4cc(F)ccc4F)[C@@H](N)C3)C2C1]

¹H NMR: (CD₃OD, 400 MHz): δ 7.34-7.32 (m, 1H), 7.29-7.26 (m, 2H), 5.01-4.98 (m, 1H), 4.68 (d, 1H, J=10.0 Hz), 4.44-4.40 (m, 1H), 4.28-4.21 (m, 1H), 3.98-3.83 (m, 2H), 3.74-3.70 (m, 2H), 3.65-3.59 (m, 1H), 3.55-3.48 (m, 2H), 3.33-3.29 (m, 2H), 3.07 (s, 3H), 2.61-2.58 (m, 1H), 1.88-1.79 (m, 1H); ESI-MS: (+ve mode) 388.15 (100%) (M+H)⁺, 410.10 (M+Na)⁺ (10%); HPLC: 97.49%.

### Compound 21: N-(2-((3R,5S,6R)-5-amino-6-(2,5-difluorophenyl)tetrahydro-2H-pyran-3-yl)octahydrocyclopenta[c]pyrrol-5-yl)methanesulfonamide

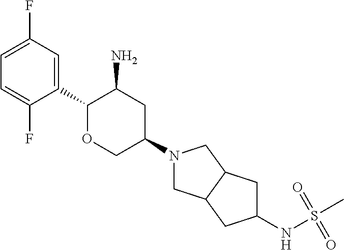 [SMILES: CS(=O)(=O)NC1CC2CN([C@H]3CO[C@H](c4cc(F)ccc4F)[C@@H](N)C3)CC2C1]

¹H NMR: (CD₃OD, 400 MHz): δ 7.20-7.17 (m, 1H), 7.14-7.11 (m, 2H), 4.56 (d, 1H, J=10.0 Hz), 4.34-4.31 (m, 1H), 3.66-3.61 (m, 3H), 3.51-3.45 (m, 4H), 2.89-2.87 (m, 4H), 2.82-2.81 (m, 2H), 2.65-2.62 (m, 1H), 2.22-2.19 (m, 2H), 2.09-1.99 (m, 1H), 1.51-1.48 (m, 2H); ESI-MS: (+ve mode) 416.05 (M+H)⁺ (100%); HPLC: 96.02%.

### Compound 22: (2R,3S,5R)-5-(5-(cyclopropanecarbonyl)-5,6-dihydropyrrolo[3,4-c]pyrrol-2(1H,3H,4H)-yl)-2-(2,5-difluorophenyl)tetrahydro-2H-pyran-3-amine

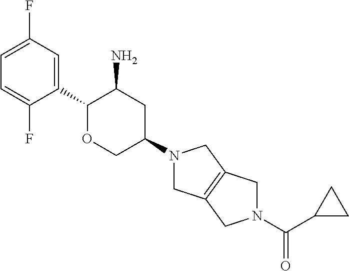 [SMILES: N[C@H]1C[C@@H](N2CC3=C(CN(C(=O)C4CC4)C3)C2)CO[C@@H]1c1cc(F)ccc1F]

¹H NMR: (CD₃OD, 400 MHz): 7.04-6.98 (m, 3H), 4.55-4.45 (m, 1H), 4.40-4.30 (m, 2H), 4.18-4.15 (m, 1H), 4.08-4.07 (m, 2H), 3.54-3.53 (m, 4H), 3.40-3.38 (m, 1H), 3.25-3.20 (m, 1H), 2.85-2.75 (m, 1H), 2.40-2.22 (m, 1H), 1.75-1.60 (m, 1H), 1.55-1.40 (m, 1H), 0.83-0.81 (m, 2H), 0.78-0.75 (m, 2H); ESI-MS: (+ve mode) 390.15 (M+H)⁺ (100%); HPLC: 95.86%.

### Compound 23: (5-((3R,5S,6R)-5-amino-6-(2,5-difluorophenyl)tetrahydro-2H-pyran-3-yl)-5,6-dihydropyrrolo[3,4-c]pyrrol-2(1H,3H,4H)-yl)(phenyl)methanone

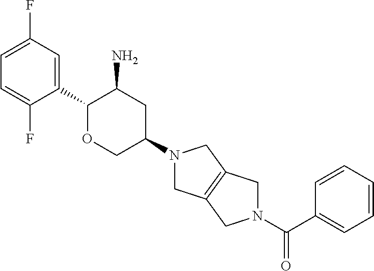 [SMILES: N[C@H]1C[C@@H](N2CC3=C(CN(C(=O)c4ccccc4)C3)C2)CO[C@@H]1c1cc(F)ccc1F]

¹H NMR: (CD₃OD, 400 MHz): δ 7.59-7.47 (m, 5H), 7.32-7.28 (m, 1H), 7.26-7.22 (m, 2H), 4.72 (d, 1H, J=10.4 Hz), 4.48-4.43 (m, 3H), 4.33-4.29 (m, 4H), 4.23-4.21 (m, 2H), 3.91-3.87 (m, 1H), 3.76 (t, 1H, J=10.8 Hz), 3.66-3.60 (m, 1H), 2.79-2.75 (m, 1H), 2.08 (q, 1H, J=11.6 Hz); ESI-MS: (+ve mode) 426.15 (M+H)⁺ (100%), 464.35 (M+K)⁺ (10%); HPLC: 95.70%.

### Compound 24: 1-(5-((3R,5S,6R)-5-amino-6-(2,5-difluorophenyl)tetrahydro-2H-pyran-3-yl)-5,6-dihydropyrrolo[3,4-c]pyrrol-2(1H,3H,4H)-yl)-2-methylpropan-1-one

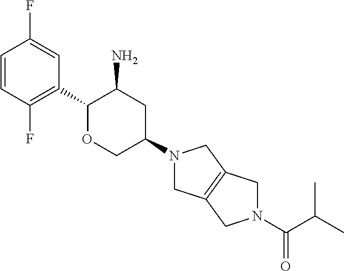 [SMILES: CC(C)C(=O)N1CC2=C(C1)CN([C@H]1CO[C@H](c3cc(F)ccc3F)[C@@H](N)C1)C2]

¹H NMR: (CD₃OD, 400 MHz): δ 7.28-7.24 (m, 1H), 7.20-7.14 (m, 2H), 4.47 (d, 1H, J=9.6 Hz), 4.36 (s, 2H), 4.25-4.22 (m, 1H), 4.16 (s, 2H), 3.63 (s, 4H), 3.41 (t, 1H, J=10.8 Hz), 3.36-3.27 (m, 1H), 3.08-3.05 (m, 1H), 2.78-2.73 (m, 1H), 2.52-2.49 (m, 1H), 1.61 (q, 1H, J=11.6 Hz), 1.12 (d, 6H, J=6.4 Hz); ESI-MS: (+ve mode) 392.20 (100%) (M+H)⁺; HPLC: 95.48%.

### Compound 25: (5-((3R,5S,6R)-5-amino-6-(2,5-difluorophenyl)tetrahydro-2H-pyran-3-yl)-5,6-dihydropyrrolo[3,4-c]pyrrol-2(1H,3H,4H)-yl(cyclonentyl)methanone

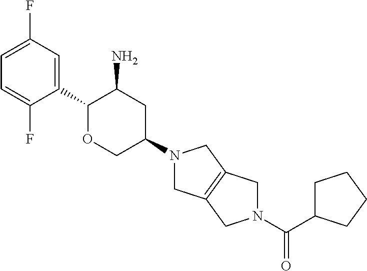 [SMILES: N[C@H]1C[C@@H](N2CC3=C(CN(C(=O)C4CCCC4)C3)C2)CO[C@@H]1c1cc(F)ccc1F]

¹H NMR: (CD₃OD, 400 MHz): 7.32-7.26 (m, 1H), 7.25-7.22 (m, 2H), 4.70 (d, 1H, J=10 Hz), 4.47-4.44 (m, 3H), 4.25-4.23 (m, 5H), 3.76-3.73 (m, 1H), 3.65-3.62 (m, 3H), 2.95-2.89 (m, 1H), 2.85-2.75 (m, 1H), 2.00 (q, 1H, J=11.6 Hz), 1.95-1.90 (m, 2H), 1.78-1.77 (m, 4H), 1.67-1.64 (m, 2H); ESI-MS: (+ve mode) 418.2 (M+H)⁺ (100%), 440.3 (M+Na)⁺; HPLC: 95.64%.

### Compound 26: (5-((3R,5S,6R)-5-amino-6-(2,5-difluorophenyl)tetrahydro-2H-pyran-3-yl)-5,6-dihydropyrrolo[3,4-c]pyrrol-2(1H,3H,4H)-yl)(cyclohexyl)methanone

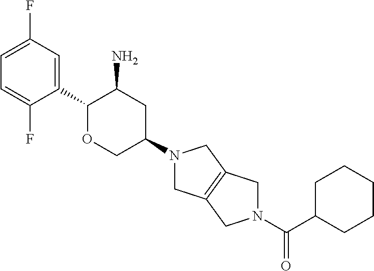 [SMILES: N[C@H]1C[C@@H](N2CC3=C(CN(C(=O)C4CCCCC4)C3)C2)CO[C@@H]1c1cc(F)ccc1F]

¹H NMR: (CD₃OD, 400 MHz): 7.33-7.30 (m, 1H), 7.25-7.19 (m, 2H), 4.51 (d, 1H, J=9.2 Hz), 4.41 (s, 2H), 4.30-4.27 (m, 1H), 4.20 (s, 2H), 3.68 (s, 4H), 3.48-3.40 (m, 1H), 3.09-3.08 (m, 1H), 2.53-2.50 (m, 1H), 1.88-1.76 (m, 5H), 1.66-1.63 (m, 1H), 1.57-1.48 (m, 3H), 1.46-1.34 (m, 4H); ESI-MS: (+ve mode) 432.2 (M+H)⁺ (100%); HPLC: 95.2%.

### Compound 27: (2R,3S,5R)-2-(2,5-difluorophenyl)-5-(5-(methoxycarbonyl)-5,6-dihydropyrrolo-[3,4-c]pyrrol-2(1H,3H,4H)-yl)tetrahydro-2H-pyran-3-amine

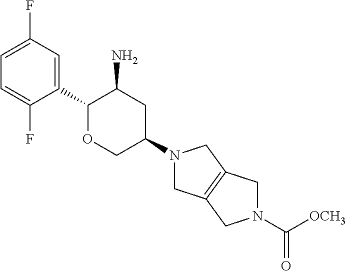 [SMILES: COC(=O)N1CC2=C(C1)CN([C@H]1CO[C@H](c3cc(F)ccc3F)[C@@H](N)C1)C2]

¹H NMR: (CD₃OD, 400 MHz): 7.31-7.25 (m, 3H), 4.71 (d, 1H, J=10.4 Hz), 4.43-4.39 (m, 1H), 4.23-4.21 (m, 4H), 4.20-4.19 (m, 4H), 3.76 (s, 3H), 3.69-3.64 (m, 2H), 3.54-3.50 (m, 1H), 2.72-2.70 (m, 1H), 2.06-2.03 (m, 1H); ESI-MS: (+ve mode) 380.10 (M)⁺ (100%); HPLC: 95.07%.

### Compound 28: (2R,3S,5R)-2-(2,5-difluorophenyl)-5-(5-(ethoxycarbonyl)-5,6-dihydropyrrolo[3,4-c]pyrrol-2(1H,3H,4H)-yl)tetrahydro-2H-pyran-3-amine

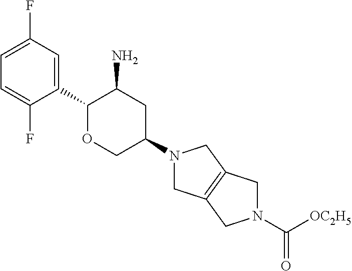 [SMILES: CCOC(=O)N1CC2=C(C1)CN([C@H]1CO[C@H](c3cc(F)ccc3F)[C@@H](N)C1)C2]

¹H NMR: (D2O, 400 MHz): 7.34-7.25 (m, 3H), 4.86 (d, 1H, J=10.4 Hz), 4.49-4.38 (m, 1H), 4.26-4.23 (m, 4H), 4.21-4.19 (m, 4H), 4.16 (q, 2H, J=7.2 Hz), 4.10-4.07 (m, 1H), 3.85-3.74 (m, 2H), 2.83-2.85 (m, 1H), 2.15-2.06 (m, 1H), 1.28 (t, 3H, J=14.4 Hz); ESI-MS: (+ve mode) 394.15 (M)⁺ (100%); HPLC: 95.72%.

### Compound 29: (2R,3S,5R)-2-(2,5-difluorophenyl)-5-(5-((trifluoromethyl)sulfonyl)-5,6-dihydropyrrolo[3,4-c]pyrrol-2(1H,3H,4H)-yl)tetrahydro-2H-pyran-3-amine

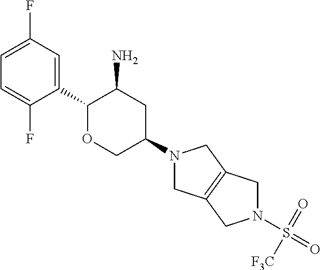 [SMILES: N[C@H]1C[C@@H](N2CC3=C(C2)CN(S(=O)(=O)C(F)(F)F)C3)CO[C@@H]1c1cc(F)ccc1F]

¹H NMR: (CD₃OD, 400 MHz): 7.30-7.27 (m, 1H), 7.25-7.21 (m, 2H), 4.49 (d, 1H, J=10 Hz), 4.40 (s, 4H), 4.28-4.26 (m, 1H), 3.72-3.67 (m, 4H), 3.46-3.44 (m, 1H), 3.31-3.30 (m, 1H), 3.11-3.06 (m, 1H), 2.53-2.50 (m, 1H), 1.67-1.58 (m, 1H); ESI-MS: (+ve mode) 454.1 (M+H)⁺ (100%); HPLC: 96.5%.

### Compound 30: (2R,3S,5R)-2-(2,5-difluorophenyl)-5-(5-(ethylsulfonyl)-5,6-dihydropyrrolo[3,4-c]pyrrol-2(1H,3H,4H)-yl)tetrahydro-2H-pyran-3-amine

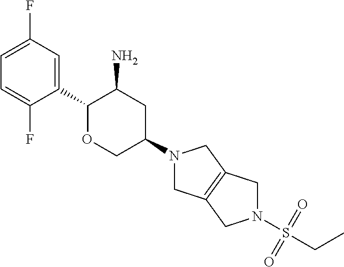 [SMILES: CCS(=O)(=O)N1CC2=C(CN([C@H]3CO[C@H](c4cc(F)ccc4F)[C@@H](N)C3)C2)C1]

¹H NMR: (CD₃OD 400 MHz): δ 7.32-7.29 (m, 1H), 7.26-7.23 (m, 2H), 4.72 (d, 1H, J=10.4 Hz), 4.46-4.44 (m, 1H), 4.30-4.22 (m, 8H), 3.91-3.86 (m, 1H), 3.76 (t, 1H, J=11.0 Hz), 3.66-3.60 (m, 1H), 3.18 (q, 2H, J=7.2 Hz), 2.78-2.75 (m, 1H), 2.09 (q, 1H, J=11.6 Hz), 1.37 (t, 3H, J=7.2 Hz); ESI-MS: (+ve mode) 414.1 (100%) (M+H)⁺; HPLC: 95.48%.

### Compound 31: (2R,3S,5R)-2-(2,5-difluorophenyl)-5-(5-(isopropylsulfonyl)-5,6-dihydropyrrolo[3,4-c]pyrrol-2(1H,3H,4H)-yl)tetrahydro-2H-pyran-3-amine

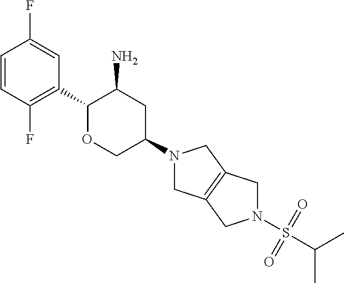 [SMILES: CC(C)S(=O)(=O)N1CC2=C(CN([C@H]3CO[C@H](c4cc(F)ccc4F)[C@@H](N)C3)C2)C1]

¹H NMR: (CD₃OD, 400 MHz): δ 7.25-7.22 (m, 1H), 7.18-7.14 (m, 2H), 4.42 (d, 1H, J=9.6 Hz), 4.23-4.20 (m, 5H), 3.60 (s, 4H), 3.49-3.35 (m, 2H), 3.24-3.18 (m, 1H), 3.06-3.00 (m, 1H), 2.46 (d, 1H, J=12.0 Hz), 1.35 (q, 1H, J=11.6 Hz), 1.35 (d, 6H, J=6.8 Hz); ESI-MS: (+ve mode) 428.20 (100%) (M+H)⁺; HPLC: 95.52%.

### Compound 32: (2R,3S,5R)-2-(2,5-difluorophenyl)-5-(5-(phenylsulfonyl)-5,6-dihydropyrrolo[3,4-c]pyrrol-2(1H,3H,4H)-yl)tetrahydro-2H-pyran-3-amine

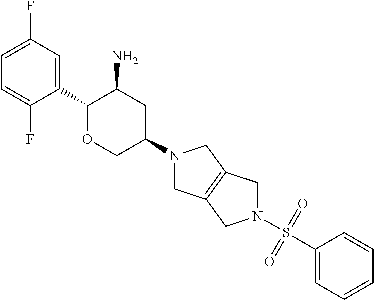 [SMILES: N[C@H]1C[C@@H](N2CC3=C(C2)CN(S(=O)(=O)c2ccccc2)C3)CO[C@@H]1c1cc(F)ccc1F]

¹H NMR: (CD₃OD, 400 MHz): 7.89-7.87 (m, 2H), 7.70-7.59 (m, 3H), 7.27-7.20 (m, 3H), 4.65-462 (m, 1H), 4.35-4.32 (m, 1H), 4.20-4.10 (m, 4H), 4.09-4.00 (m, 4H), 3.72-3.57 (m, 3H), 2.67-2.65 (m, 1H), 1.96-1.93 (m, 1H); ESI-MS: (+ve mode) 462.15 (M+H)⁺ (100%), 484.10 (M+Na)⁺ (25%); HPLC: 96.69%.

### Compound 33: (2R,3S,5R)-2-(2,5-difluorophenyl)-5-(5-((4-fluorophenyl)sulfonyl)-5,6-dihydropyrrolo[3,4-c]pyrrol-2 (1H,3H,4H)-yl)tetrahydro-2H-pyran-3-amine

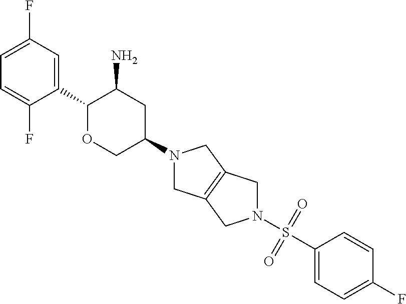 [SMILES: N[C@H]1C[C@@H](N2CC3=C(C2)CN(S(=O)(=O)c2ccc(F)cc2)C3)CO[C@@H]1c1cc(F)ccc1F]

¹H NMR: (CD₃OD, 400 MHz): 8.00-7.96 (m, 2H), 7.42-7.38 (m, 2H), 7.29-7.25 (m, 1H), 7.23-7.18 (m, 2H), 4.44 (d, 1H, J=10 Hz), 4.21-4.19 (m, 1H), 4.16 (s, 4H), 3.54-3.53 (m, 5H), 3.25-3.20 (m, 1H), 3.02-3.00 (m, 1H), 2.44-2.437 (m, 1H), 1.56-1.53 (m, 1H); ESI-MS: (+ve mode) 480.2 (M+H)⁺ (100%); HPLC: 95.5%

### Compound 34: 4454(3R,5S,6R)-5-amino-6-(2,5-difluorophenyl)tetrahydro-2H-pyran-3-yl)-5,6-dihydropyrrolo[3,4-c]pyrrol-2(1H,3H,4H)-yl)sulfonyl)benzonitrile

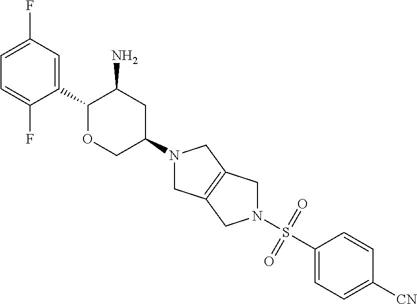 [SMILES: N#Cc1ccc(S(=O)(=O)N2CC3=C(CN([C@H]4CO[C@H](c5cc(F)ccc5F)[C@@H](N)C4)C3)C2)cc1]

¹H NMR: (CD₃OD, 400 MHz): δ 8.07 (dd, 2H, J₁=2.0 Hz, J₂=6.8 Hz), 8.01 (dd, 2H, Jⱼ=2.0 Hz, J₂=6.8 Hz), 7.30-7.22 (m, 3H), 4.69 (d, 1H, J=10.0 Hz), 4.40-4.36 (m, 1H), 4.23-4.17 (m, 8H), 3.88-3.84 (m, 1H), 3.71 (t, 1H, J=10.8 Hz), 3.63-3.57 (m, 1H), 2.74-2.71 (m, 1H), 2.07 (q, 1H, J=12.0 Hz); ESI-MS: (+ve mode) 487.15 (M+H)⁺ (100°); HPLC: 96.23%.

### Compound 35: (2R,3S,5R)-2-(2,5-difluorophenyl)-5-(5-((4-(trifluoromethoxy)phenyl)sulfonyl)-5,6-dihydropyrrolo[3,4-c]pyrrol-2(1H,3H,4H)-yl)tetrahydro-2H-pyran-3-amine

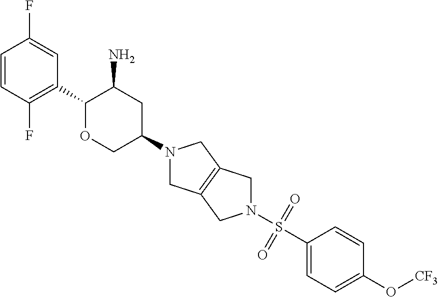 [SMILES: N[C@H]1C[C@@H](N2CC3=C(C2)CN(S(=O)(=O)c2ccc(OC(F)(F)F)cc2)C3)CO[C@@H]1c1cc(F)ccc1F]

¹H NMR: (CD₃OD, 400 MHz): δ 7.99 (d, 2H, J=8.8 Hz), 7.51 (d, 2H, J=8.4 Hz), 7.23-7.20 (m, 1H), 7.17-7.13 (m, 2H), 4.40 (d, 1H, J=10.8 Hz), 4.15-4.12 (m, 5H), 3.49 (s, 4H), 3.36-3.33 (m, 1H), 3.22-3.18 (m, 1H), 2.99-2.93 (m, 1H), 2.42-2.39 (m, 1H), 1.50 (q, 1H, J=11.2 Hz); ESI-MS: (+ve mode) 546.25 (100%) (M+H)⁺; HPLC: 96.75%.

### Compound 36: (2R,3S,5R)-2-(2,5-difluorophenyl)-5-(5((2,4-difluorophenyl)sulfonyl)-5,6-dihydropyrrolo[3,4-c]pyrrol-2(1H,3H,4H)-yl)tetrahydro-2H-pyran-3-amine

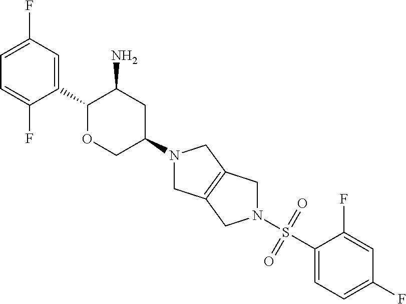 [SMILES: N[C@H]1C[C@@H](N2CC3=C(C2)CN(S(=O)(=O)c2ccc(F)cc2F)C3)CO[C@@H]1c1cc(F)ccc1F]

¹H NMR: (CDCl₃, 400 MHz): 7.96-7.90 (m, 1H), 7.15-7.11 (m, 1H), 7.06-7.69 (m, 4H), 4.20-4.12 (m, 6H), 3.59 (s, 4H), 3.31 (t, 1H, J=10.8 Hz), 2.94-2.89 (m, 1H), 2.84-2.78 (m, 1H), 2.37-2.33 (m, 1H), 1.36 (q, 1H, J=12 Hz); ESI-MS: (+ve mode) 498.15 (M+H)⁺ (100%), 520.20 (M+Na)⁺; HPLC: 96.95%.

### Compound 37: (2R,3S,5R)-2-(2,5-difluorophenyl)-5-(5-tosylhexahydrocyclopenta[c]pyrrol-2(1H)-yl)tetrahydro-2H-pyran-3-amine

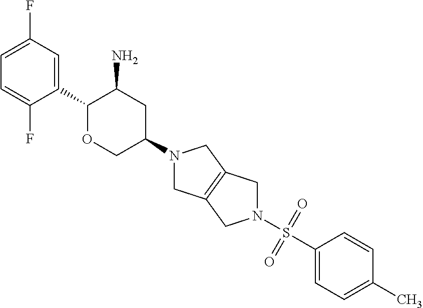 [SMILES: Cc1ccc(S(=O)(=O)N2CC3=C(CN([C@H]4CO[C@H](c5cc(F)ccc5F)[C@@H](N)C4)C3)C2)cc1]

¹H NMR: (CD₃OD, 400 MHz): 7.76-7.74 (d, 2H, J=8.0 Hz), 7.43-7.41 (d, 2H, J=8.0 Hz), 7.27-7.20 (m, 3H), 4.65-4.62 (m, 1H), 4.33-4.31 (m, 1H), 4.16-4.05 (m, 8H), 3.78-3.70 (m, 1H), 3.64-3.55 (m, 2H), 2.67-2.65 (m, 1H), 2.42 (s, 3H), 1.97-1.94 (m, 1H); ESI-MS: (+ve mode) 476.20 (M+H)⁺ (100%); HPLC: 95.16%.

### Compound 38: (2R,3S,5R)-2-(2,5-difluorophenyl)-5-(5-((4-methoxyphenyl)sulfonyl)-5,6-dihydropyrrolo[3,4-c]pyrrol-2(1H,3H,4H)-yl)tetrahydro-2H-pyran-3-amine

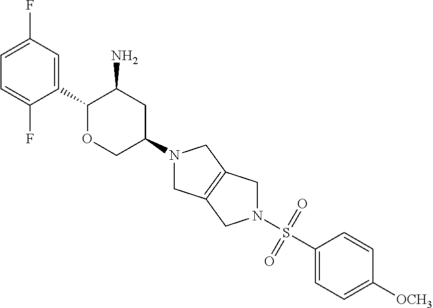 [SMILES: COc1ccc(S(=O)(=O)N2CC3=C(CN([C@H]4CO[C@H](c5cc(F)ccc5F)[C@@H](N)C4)C3)C2)cc1]

¹H NMR: (CD₃OD, 400 MHz): 7.84-7.81 (m, 2H), 7.29-7.22 (m, 3H), 7.15-7.12 (m, 2H), 4.68 (d, 1H, J=10.4 Hz), 4.37-4.33 (m, 1H), 4.17-4.1 (m, 8H), 3.88 (s, 3H), 3.80-3.78 (m, 1H), 3.68-3.61 (m, 2H), 2.72-2.68 (m, 1H), 2.06-1.99 (m, 1H); ESI-MS: (+ve mode) 492.2 (M+H)⁺ (100%); HPLC: 95.67%.

### Compound 39: (2R,3S,5R)-2-(2,5-difluorophenyl)-5-(5-((4-methoxyphenyl)sulfonyl)-5,6-dihydropyrrolo[3,4-c]pyrrol-2(1H,3H,4H)-yl)tetrahydro-2H-pyran-3-amine

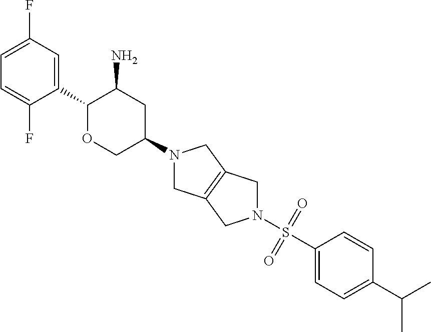 [SMILES: CC(C)c1ccc(S(=O)(=O)N2CC3=C(CN([C@H]4CO[C@H](c5cc(F)ccc5F)[C@@H](N)C4)C3)C2)cc1]

¹H NMR: (CD₃OD, 400 MHz): 7.81 (d, 2H, J=8.4 Hz), 7.50 (d, 2H, J=8.4 Hz), 7.29-7.21 (m, 3H), 4.50 (d, 1H, J=10 Hz), 4.36-4.31 (m, 1H), 4.17-4.19 (m, 4H), 4.01-3.97 (m, 4H), 3.62-3.55 (m, 3H), 3.31-3.01 (m, 1H), 2.63-2.61 (m, 1H), 1.93-1.90 (m, 1H), 1.30 (d, 6H, J=6.8 Hz); ESI-MS: (+ve mode) 504.25 (M)⁺ (100%); HPLC: 97.13%.

### Compound 40: (2R,3S,5R)-2-(2,5-difluorophenyl)-5-(5-((4-(trifluoromethyl)phenyl)sulfonyl)-5,6-dihydropyrrolo[3,4-c]pyrrol-2(1H,3H,4H)-yl)tetrahydro-2H-pyran-3-amine

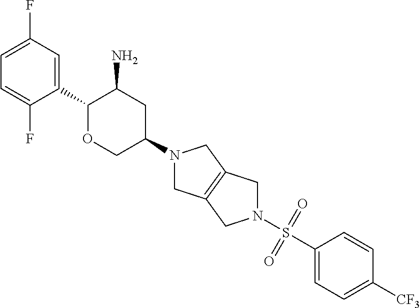 [SMILES: N[C@H]1C[C@@H](N2CC3=C(C2)CN(S(=O)(=O)c2ccc(C(F)(F)F)cc2)C3)CO[C@@H]1c1cc(F)ccc1F]

¹H NMR: (CD₃OD, 400 MHz): δ 8.11 (d, 2H, J=8.4 Hz), 7.97 (d, 2H, J=8.4 Hz), 7.29-7.21 (m, 3H), 4.67 (d, 1H, J=10.0 Hz), 4.37-4.34 (m, 1H), 4.27-4.23 (m, 4H), 4.12-4.09 (m, 4H), 3.79-3.72 (m, 1H), 3.65 (t, 1H, J=10.8 Hz), 3.58-3.57 (m, 1H), 2.68-2.65 (m, 1H), 2.00 (q, 1H, J=11.6 Hz); ESI-MS: (+ve mode) 530.25 (M+H)⁺ (100%); HPLC: 95.73%.

### Compound 41: (2R,3S,5R)-5-(5-acetyl-5,6-dihydropyrrolo[3,4-c]pyrrol-2(1H,3H,4H)-yl)-2-(2,5-difluorophenyl)tetrahydro-2H-pyran-3-amine

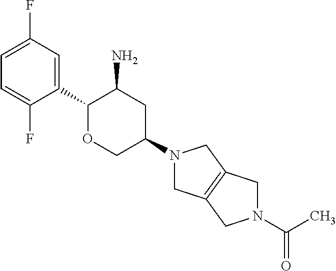 [SMILES: CC(=O)N1CC2=C(C1)CN([C@H]1CO[C@H](c3cc(F)ccc3F)[C@@H](N)C1)C2]

¹H NMR: (CD₃OD, 400 MHz): 7.20-7.09 (m, 3H), 4.58 (s, 1H), 4.30-4.28 (m, 2H), 4.20-4.10 (m, 3H), 3.63-3.61 (m, 4H), 3.40-3.35 (m, 1H), 2.97-2.94 (m, 2H), 2.42-2.38 (m, 1H), 2.13 (s, 3H) 2.10-2.08 (m, 1H); ESI-MS: (+ve mode) 364.10 (M+H)⁺ (100%); HPLC: 96.52%.

### Compound 42: (2R,3S,5R)-2-(2,5-difluorophenyl)-5-(5-(isobutylsulfonyl)-5,6-dihydropyrrolo[3,4-c]pyrrol-2(1H,3H,4H)-yl)tetrahydro-2H-pyran-3-amine

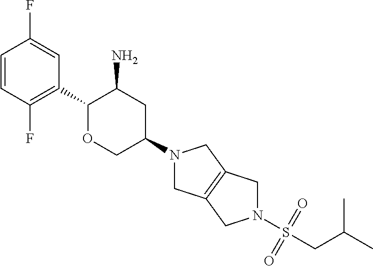 [SMILES: CC(C)CS(=O)(=O)N1CC2=C(CN([C@H]3CO[C@H](c4cc(F)ccc4F)[C@@H](N)C3)C2)C1]

¹H NMR: (CD₃OD, 400 MHz): −7.31-7.27 (m, 1H), 7.24-7.20 (m, 2H), 4.67 (d, 1H, J=10.0 Hz), 4.42-4.40 (m, 1H), 4.22 (s, 4H), 4.16-4.12 (m, 4H), 3.77-3.72 (m, 1H), 3.70 (t, 1H, J=10.8 Hz), 3.61-3.56 (m, 1H), 2.99 (d, 2H, J=6.8 Hz), 2.73-2.70 (m, 1H), 2.24 (hep, 1H, J=6.4 Hz), 2.02 (q, 1H, J=11.6 Hz), 1.11 (d, 6H, J=6.8 Hz). ESI-MS: (+ve mode) 442.15 (M+H)⁺ (100%); HPLC: 98.12%.

### Compound 43: 5-((3R,5S,6R)-5-amino-6-(2,5-difluorophenyl)tetrahydro-2H-pyran-3-yl)hexahydro-1H-thieno[3,4-c]pyrrole 2,2-dioxide

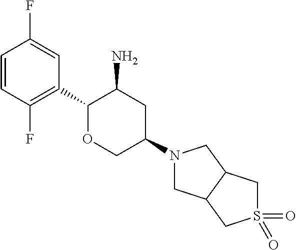 [SMILES: N[C@H]1C[C@@H](N2CC3CS(=O)(=O)CC3C2)CO[C@@H]1c1cc(F)ccc1F]

¹H NMR: (D₂O, 400 MHz): −δ 7.35-7.28 (m, 3H), 4.86 (d, 1H, J=10.4 Hz), 4.53-4.51 (m, 1H), 4.14-4.05 (m, 2H), 3.86-3.74 (m, 3H), 3.60-3.52 (m, 2H), 3.47-3.43 (m, 4H), 3.34 (d, 2H, J=14 Hz), 2.90-2.88 (m, 1H), 2.14-2.11 (m, 1H). ESI-MS: (+ve mode) 373.1 (M+H)⁺ (100%); HPLC: 95.61%.

### Compound 44: (2R,3S,5R)-2-(2,5-difluorophenyl)-5-(5,6-dihydropyrrolo[3,4-c]pyrrol-2(1H,3H,4H)-yl)tetrahydro-2H-pyran-3-amine

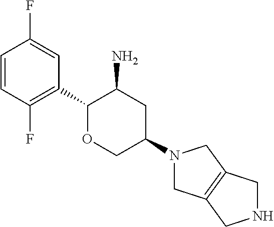 [SMILES: N[C@H]1C[C@@H](N2CC3=C(CNC3)C2)CO[C@@H]1c1cc(F)ccc1F]

¹H NMR: (D₂O, 400 MHz): −δ 7.34-7.25 (m, 3H), 4.87 (d, 1H, J 12 Hz), 4.52-4.48 (m, 1H), 4.43-4.40 (m, 4H), 4.24 (s, 4H), 4.13-4.09 (m, 1H), 3.82 (t, 1H, J=11.2 Hz), 3.78-3.74 (m, 1H), 2.88-2.85 (m, 1H), 2.13 (q, 1H, J=12 Hz). ESI-MS: (+ve mode) 322.1 (M+H)⁺ (100%); HPLC: 95.44%.

### Compound 45: 5-((3R,5S,6R)-5-amino-6-(2,5-difluorophenyl)tetrahydro-2H-pyran-3-yl)-N-phenyl-3,4,5,6-tetrahydropyrrolo[3,4-c]pyrrole-2(1H)-carboxamide

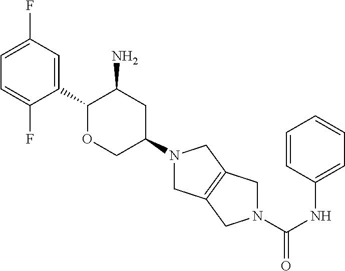 [SMILES: N[C@H]1C[C@@H](N2CC3=C(CN(C(=O)Nc4ccccc4)C3)C2)CO[C@@H]1c1cc(F)ccc1F]

¹H NMR: (CD₃OD, 400 MHz): −δ 7.30-7.21 (m, 7H), 7.04 (t, 1H, J=7.4 Hz), 4.73 (d, 1H, J=10.4 Hz), 4.45-4.43 (m, 1H), 4.29-4.26 (m, 8H), 3.93-3.90 (m, 1H), 3.76 (t, 1H, J=10.8 Hz), 3.67-3.60 (m, 1H), 2.82-2.79 (m, 1H), 2.08 (q, 1H, J=12 Hz). ESI-MS: (+ve mode) 441.1 (M+H)⁺ (100%); HPLC: 96.20%.

### Compound 46: N-((2R,3S,5R)-2-(2,5-difluorophenyl)-5-(5-(methylsulfonyl)-5,6-dihydropyrrolo[3,4-c]pyrrol-2(1H,3H,4H)-yl)tetrahydro-2H-pyran-3-yl)acetamide

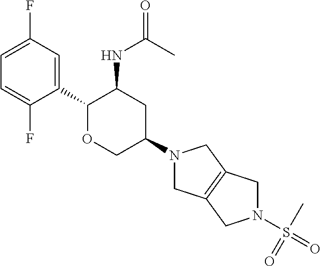 [SMILES: CC(=O)N[C@H]1C[C@@H](N2CC3=C(C2)CN(S(C)(=O)=O)C3)CO[C@@H]1c1cc(F)ccc1F]

¹H NMR: (CDCl₃, 400 MHz): −δ 7.28-7.19 (m, 1H), 7.00-6.92 (m, 2H), 5.45 (d, 1H, J=9.2 Hz), 4.38 (d, 1H, J=10 Hz), 4.22-4.18 (m, 1H), 4.14 (s, 4H), 4.12-4.03 (m, 1H), 3.55 (s, 4H), 3.36 (t, 1H, J=10.8 Hz), 3.01-2.94 (m, 1H), 2.86 (s, 3H), 2.48-2.44 (m, 1H), 1.82 (s, 3H), 1.50 (q, 1H, J=11.6 Hz). ESI-MS: (+ve mode) 442.1 (M+H)⁺ (100%); HPLC: 96.44%.

### Compound 47: N-((2R,3S,5R)-5-(5-acetyl-5,6-dihydropyrrolo[3,4-c]pyrrol-2(1H,3H,4H)-yl)-2-(2,5-difluorophenyl)tetrahydro-2H-pyran-3-yl)acetamide

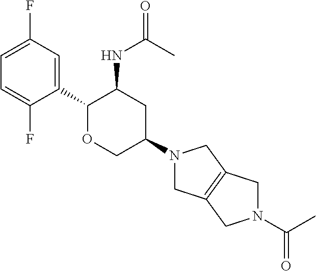 [SMILES: CC(=O)N[C@H]1C[C@@H](N2CC3=C(CN(C(C)=O)C3)C2)CO[C@@H]1c1cc(F)ccc1F]

¹H NMR: (CDCl₃, 400 MHz): −δ 7.24-7.19 (m, 1H), 7.00-6.93 (m, 2H), 5.43 (d, 1H, J=9.2 Hz), 4.39 (d, 1H, J=10 Hz), 4.20 (s, 5H), 4.09-4.07 (m, 1H), 3.57 (s, 4H), 3.37 (t, 1H, J=10.8 Hz), 3.01-2.95 (m, 1H), 2.49-2.45 (m, 1H), 2.07 (s, 3H), 1.83 (s, 3H), 1.48 (q, 1H, J=11.6 Hz). ESI-MS: (+ve mode) 406.1 (M+H)⁺ (100%); HPLC: 96.44%.

### Compound 48: 5-((3R,5S,6R)-5-amino-6-(2,5-difluorophenyl)tetrahydro-2H-pyran-3-yl)-3,4,5,6-tetrahydropyrrolo[3,4-c]pyrrole-2(1H)-carbaldehyde

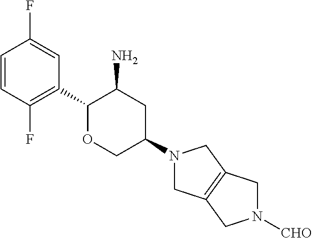 [SMILES: N[C@H]1C[C@@H](N2CC3=C(CN(C=O)C3)C2)CO[C@@H]1c1cc(F)ccc1F]

¹H NMR: (CD₃OD, 400 MHz): −δ 8.25 (s, 1H), 7.31-7.28 (m, 1H), 7.24-7.20 (m, 2H), 4.71 (d, 1H, J=10.0 Hz), 4.46-4.42 (m, 3H), 4.31-4.23 (m, 6H), 3.89-3.85 (m, 1H), 3.76 (t, 1H, J=10.8 Hz), 3.65-3.59 (m, 1H), 2.78-2.75 (m, 1H), 2.08 (q, 1H, J=11.6 Hz); ESI-MS: (+ve mode) 350.1 (M+H)⁺ (100%); HPLC: 98.78%.

### Compound 49: (2R,3S,5R)-2-(2,5-difluorophenyl)-5-(5-(N-(4-methylbenzenesulfonyl)-S-methylsulfonimidoyl)-5,6-dihydropyrrolo[3,4-c]pyrrol-2(1H,3H,4H)-yl)tetrahydro-2H-pyran-3-amine

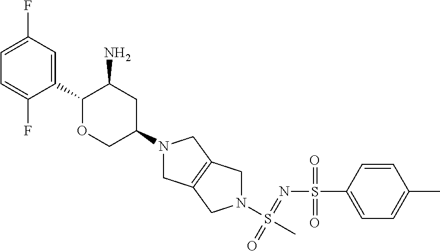 [SMILES: Cc1ccc(S(=O)(=O)N=S(C)(=O)N2CC3=C(CN([C@H]4CO[C@H](c5cc(F)ccc5F)[C@@H](N)C4)C3)C2)cc1]

¹H NMR: (CD₃OD, 400 MHz): −δ 7.80 (d, 2H, J=8.0 Hz), 7.36 (d, 2H, J 8.0 Hz), 7.31-7.29 (m, 1H), 7.24-7.21 (m, 2H), 4.70 (d, 1H, J=10.0 Hz), 4.41 (d, 1H, J=8.0 Hz), 4.34-4.31 (m, 4H), 4.15 (s, 1H), 3.74-3.70 (m, 2H), 3.64-3.58 (m, 1H), 3.24 (s, 3H), 2.74-2.71 (m, 1H), 2.42 (s, 3H), 2.05 (q, 1H, J=11.6 Hz); ESI-MS: (+ve mode) 553.2 (M+H)⁺ (100%); HPLC: 97.39%.

### Compound 50: 1-(5-((3R,5S,6R)-5-amino-6-(2,5-difluorophenyl)tetrahydro-2H-pyran-3-yl)-5,6-dihydropyrrolo[3,4-c]pyrrol-2(1H,3H,4H)-yl)-2,2,2-trifluoroethanone

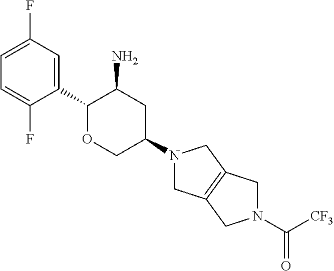 [SMILES: N[C@H]1C[C@@H](N2CC3=C(CN(C(=O)C(F)(F)F)C3)C2)CO[C@@H]1c1cc(F)ccc1F]

¹H NMR: (CD₃OD, 400 MHz): −δ 7.31-7.27 (m, 1H), 7.24-7.20 (m, 2H), 4.70 (d, 1H, J=10.0 Hz), 4.57 (s, 2H), 4.44-4.39 (m, 3H), 4.21 (s, 4H), 3.82-3.69 (m, 2H), 3.64-3.57 (m, 1H), 2.75-2.72 (m, 1H), 2.04 (q, 1H, J=11.6 Hz); ESI-MS: (+ve mode) 418.2 (M+H)⁺ (100%); HPLC: 99.18%.

Using the above procedures, following compounds (Table-2) can be prepared by accompanying reductive amination of intermediate-1 with appropriate substituent R² followed by removal of amine protecting group.

TABLE 2

| Compounds | Structures | IUPAC Names |
|---|---|---|
| 51 | 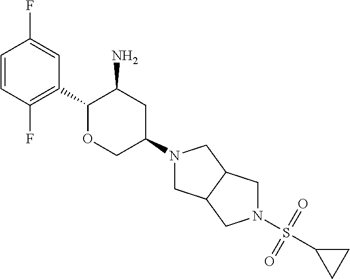 [SMILES: N[C@H]1C[C@@H](N2CC3CN(S(=O)(=O)C4CC4)CC3C2)CO[C@@H]1c1cc(F)ccc1F] | (2R,3S,5R)-5-(5- (cyclopropylsulfonyl)hexahydropyrrolo [3,4-c]pyrrol-2(1H)-yl)-2-(2,5- difluorophenyl)tetrahydro-2H-pyran-3- amine |
| 52 | 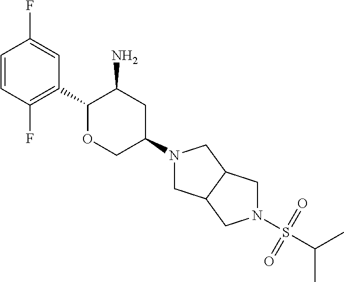 [SMILES: CC(C)S(=O)(=O)N1CC2CN([C@H]3CO[C@H](c4cc(F)ccc4F)[C@@H](N)C3)CC2C1] | (2R,3S,5R)-2-(2,5-difluorophenyl)-5-(5- (isopropylsulfonyl)hexahydropyrrolo[3,4- c]pyrrol-2(1H)-yl)tetrahydro-2H-pyran- 3-amine |
| 53 | 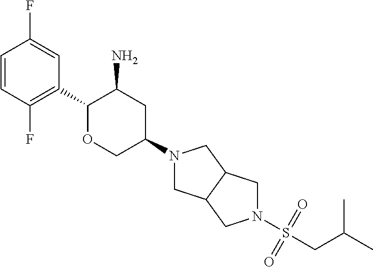 [SMILES: CC(C)CS(=O)(=O)N1CC2CN([C@H]3CO[C@H](c4cc(F)ccc4F)[C@@H](N)C3)CC2C1] | (2R,3S,5R)-2-(2,5-difluorophenyl)-5-(5- (isobutylsulfonyl)hexahydropyrrolo[3,4- c]pyrrol-2(1H)-yl)tetrahydro-2H-pyran- 3-amine |
| 54 | 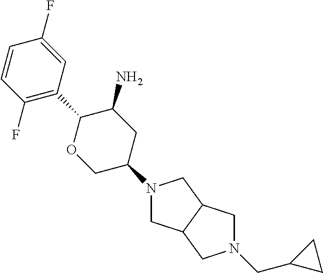 [SMILES: N[C@H]1C[C@@H](N2CC3CN(CC4CC4)CC3C2)CO[C@@H]1c1cc(F)ccc1F] | (2R,3S,5R)-5-(5- (cyclopropylmethyl)hexahydropyrrolo[3, 4-c]pyrrol-2(1H)-yl)-2-(2,5- difluorophenyl)tetrahydro-2H-pyran-3- amine |
| 55 | 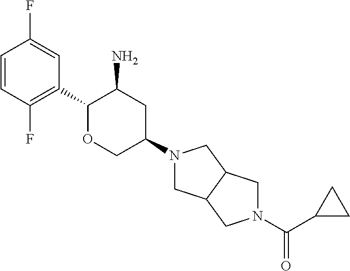 [SMILES: N[C@H]1C[C@@H](N2CC3CN(C(=O)C4CC4)CC3C2)CO[C@@H]1c1cc(F)ccc1F] | (5-((3R,5S,6R)-5-amino-6-(2,5- difluorophenyl)tetrahydro-2H-pyran-3- yl)hexahydropyrrolo[3,4-c]pyrrol-2(1H)- yl)(cyclopropyl)methanone |
| 56 | 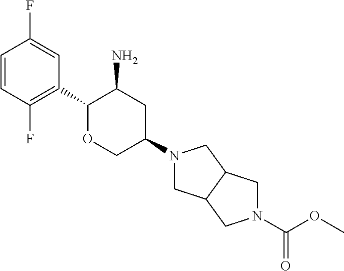 [SMILES: COC(=O)N1CC2CN([C@H]3CO[C@H](c4cc(F)ccc4F)[C@@H](N)C3)CC2C1] | methyl 5-((3R,5S,6R)-5-amino-6-(2,5- difluorophenyl)tetrahydro-2H-pyran-3- yl)hexahydropyrrolo[3,4-c]pyrrole- 2(1H)-carboxylate |
| 57 | 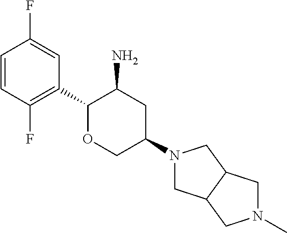 [SMILES: CN1CC2CN([C@H]3CO[C@H](c4cc(F)ccc4F)[C@@H](N)C3)CC2C1] | (2R,3S,5R)-2-(2,5-difluorophenyl)-5-(5- methylhexahydropyrrolo[3,4-c]pyrrol- 2(1H)-yl)tetrahydro-2H-pyran-3-amine |
| 58 | 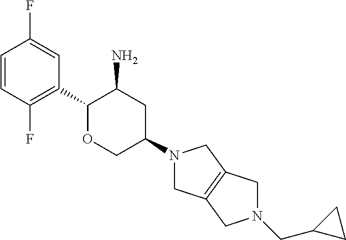 [SMILES: N[C@H]1C[C@@H](N2CC3=C(CN(CC4CC4)C3)C2)CO[C@@H]1c1cc(F)ccc1F] | (2R,3S,5R)-5-(5-(cyclopropylmethyl)- 5,6-dihydropyrrolo[3,4-c]pyrrol- 2(1H,3H,4H)-yl)-2-(2,5- difluorophenyl)tetrahydro-2H-pyran-3- amine |
| 59 | 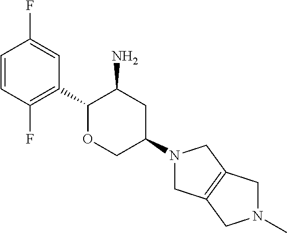 [SMILES: CN1CC2=C(C1)CN([C@H]1CO[C@H](c3cc(F)ccc3F)[C@@H](N)C1)C2] | (2R,3S,5R)-2-(2,5-difluorophenyl)-5-(5- methyl-5,6-dihydropyrrolo[3,4-c]pyrrol- 2(1H,3H,4H)-yl)tetrahydro-2H-pyran-3- amine |
| 60 | 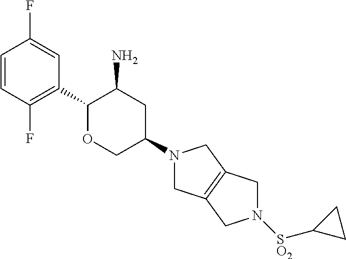 [SMILES: N[C@H]1C[C@@H](N2CC3=C(C2)CN(S(=O)(=O)C2CC2)C3)CO[C@@H]1c1cc(F)ccc1F] | (2R,3S,5R)-5-(5-(cyclopropylsulfonyl)- 5,6-dihydropyrrolo[3,4-c]pyrrol- 2(1H,3H,4H)-yl)-2-(2,5- difluorophenyl)tetrahydro-2H-pyran-3- amine |
| 61 | 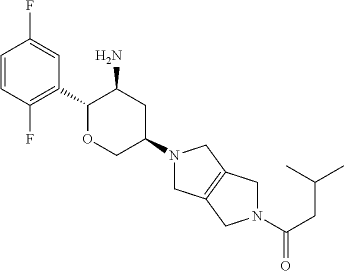 [SMILES: CC(C)CC(=O)N1CC2=C(C1)CN([C@H]1CO[C@H](c3cc(F)ccc3F)[C@@H](N)C1)C2] | 1-(5-((3R,5S,6R)-5-amino-6-(2,5- difluorophenyl)tetrahydro-2H-pyran-3- yl)-5,6-dihydropyrrolo[3,4-c]pyrrol- 2(1H,3H,4H)-yl)-3-methylbutan-1-one |
| 62 | 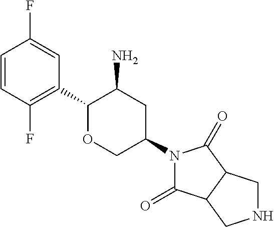 [SMILES: N[C@H]1C[C@@H](N2C(=O)C3CNCC3C2=O)CO[C@@H]1c1cc(F)ccc1F] | 2-((3R,5S,6R)-5-amino-6-(2,5- difluorophenyl)tetrahydro-2H-pyran-3- yl)tetrahydropyrrolo[3,4-c]pyrrole- 1,3(2H,3aH)-dione |
| 63 | 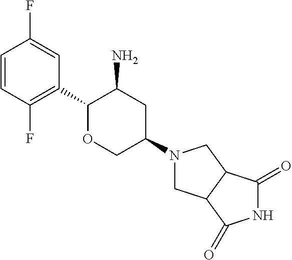 [SMILES: N[C@H]1C[C@@H](N2CC3C(=O)NC(=O)C3C2)CO[C@@H]1c1cc(F)ccc1F] | 5-((3R,5S,6R)-5-amino-6-(2,5- difluorophenyl)tetrahydro-2H-pyran-3- yl)tetrahydropyrrolo[3,4-c]pyrrole- 1,3(2H,3aH)-dione |
| 64 | 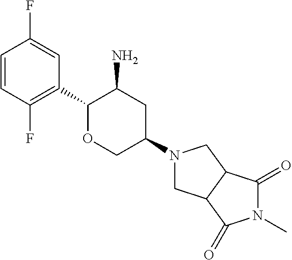 [SMILES: CN1C(=O)C2CN([C@H]3CO[C@H](c4cc(F)ccc4F)[C@@H](N)C3)CC2C1=O] | 5-((3R,5S,6R)-5-amino-6-(2,5- difluorophenyl)tetrahydro-2H-pyran-3- yl)-2-methyltetrahydropyrrolo[3,4- c]pyrrole-1,3(2H,3aH)-dione |
| 65 | 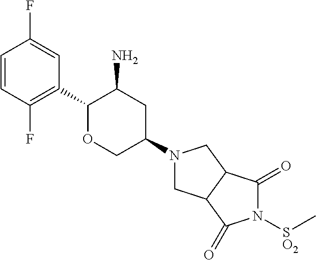 [SMILES: CS(=O)(=O)N1C(=O)C2CN([C@H]3CO[C@H](c4cc(F)ccc4F)[C@@H](N)C3)CC2C1=O] | 5-((3R,5S,6R)-5-amino-6-(2,5- difluorophenyl)tetrahydro-2H-pyran-3- yl)-2- (methylsulfonyl)tetrahydropyrrolo[3,4- c]pyrrole-1,3(2H,3aH)-dione |
| 66 | 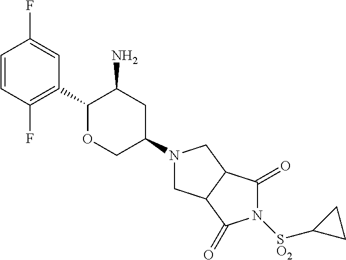 [SMILES: N[C@H]1C[C@@H](N2CC3C(=O)N(S(=O)(=O)C4CC4)C(=O)C3C2)CO[C@@H]1c1cc(F)ccc1F] | 5-((3R,5S,6R)-5-amino-6-(2,5- difluorophenyl)tetrahydro-2H-pyran-3- yl)-2- (cyclopropylsulfonyl)tetrahydropyrrolo [3,4-c]pyrrole-1,3(2H,3aH)-dione |
| 67 | 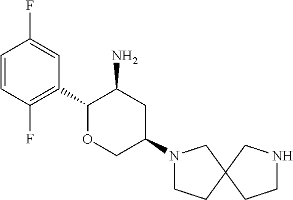 [SMILES: N[C@H]1C[C@@H](N2CCC3(CCNC3)C2)CO[C@@H]1c1cc(F)ccc1F] | (2R,3S,5R)-2-(2,5-difluorophenyl)-5- (2,7-diazaspiro[4.4]nonan-2- yl)tetrahydro-2H-pyran-3-amine |
| 68 | 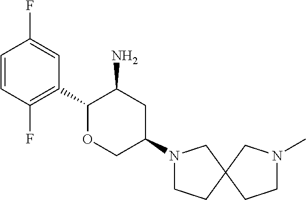 [SMILES: CN1CCC2(CCN([C@H]3CO[C@H](c4cc(F)ccc4F)[C@@H](N)C3)C2)C1] | (2R,3S,5R)-2-(2,5-difluorophenyl)-5-(7- methyl-2,7-diazaspiro[4.4]nonan-2- yl)tetrahydro-2H-pyran-3-amine |
| 69 | 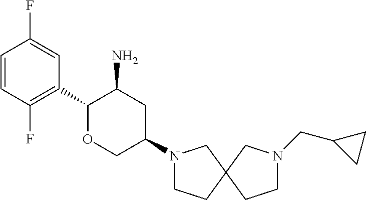 [SMILES: N[C@H]1C[C@@H](N2CCC3(CCN(CC4CC4)C3)C2)CO[C@@H]1c1cc(F)ccc1F] | (2R,3S,5R)-5-(7-(cyclopropylmethyl)- 2,7-diazaspiro[4.4]nonan-2-yl)-2-(2,5- difluorophenyl)tetrahydro-2H-pyran-3- amine |
| 70 | 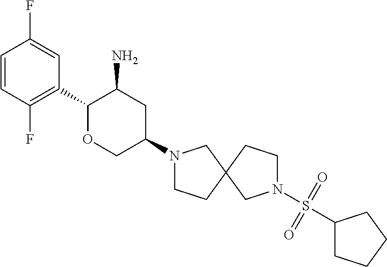 [SMILES: N[C@H]1C[C@@H](N2CCC3(CCN(S(=O)(=O)C4CCCC4)C3)C2)CO[C@@H]1c1cc(F)ccc1F] | (2R,3S,5R)-5-(7-(cyclopentylsulfonyl)- 2,7-diazaspiro[4.4]nonan-2-yl)-2-(2,5- difluorophenyl)tetrahydro-2H-pyran-3- amine |
| 71 | 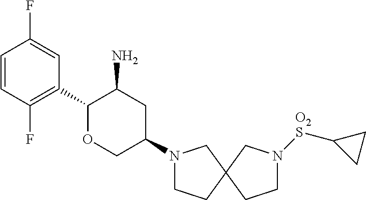 [SMILES: N[C@H]1C[C@@H](N2CCC3(CCN(S(=O)(=O)C4CC4)C3)C2)CO[C@@H]1c1cc(F)ccc1F] | (2R,3S,5R)-5-(7-(cyclopropylsulfonyl)- 2,7-diazaspiro[4.4]nonan-2-yl)-2-(2,5- difluorophenyl)tetrahydro-2H-pyran-3- amine |
| 72 | 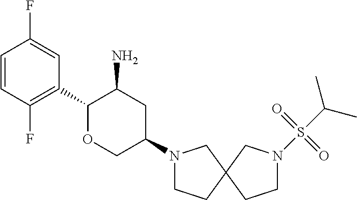 [SMILES: CC(C)S(=O)(=O)N1CCC2(CCN([C@H]3CO[C@H](c4cc(F)ccc4F)[C@@H](N)C3)C2)C1] | (2R,3S,5R)-2-(2,5-difluorophenyl)-5-(7- (isopropylsulfonyl)-2,7- diazaspiro[4.4]nonan-2-yl)tetrahydro- 2H-pyran-3-amine |
| 73 | 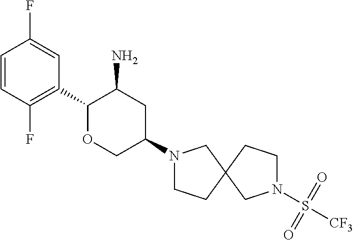 [SMILES: N[C@H]1C[C@@H](N2CCC3(CCN(S(=O)(=O)C(F)(F)F)C3)C2)CO[C@@H]1c1cc(F)ccc1F] | (2R,3S,5R)-2-(2,5-difluorophenyl)-5-(7- ((trifluoromethyl)sulfonyl)-2,7- diazaspiro[4.4]nonan-2-yl)tetrahydro- 2H-pyran-3-amine |
| 74 | 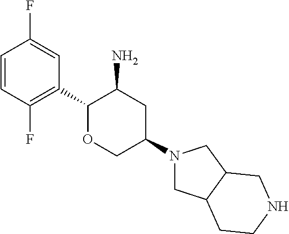 [SMILES: N[C@H]1C[C@@H](N2CC3CCNCC3C2)CO[C@@H]1c1cc(F)ccc1F] | (2R,3S,5R)-2-(2,5-difluorophenyl)-5- (hexahydro-lH-pyrrolo[3,4-c]pyridin- 2(3H)-yl)tetrahydro-2H-pyran-3-amine |
| 75 | 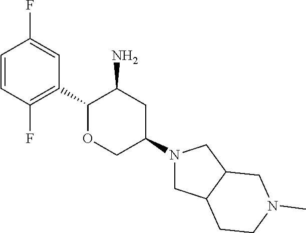 [SMILES: CN1CCC2CN([C@H]3CO[C@H](c4cc(F)ccc4F)[C@@H](N)C3)CC2C1] | (2R,3S,5R)-2-(2,5-difluorophenyl)-5-(5- methylhexahydro-1H-pyrrolo[3,4- c]pyridin-2(3H)-yl)tetrahydro-2H-pyran- 3-amine |
| 76 | 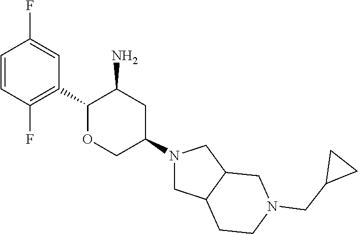 [SMILES: N[C@H]1C[C@@H](N2CC3CCN(CC4CC4)CC3C2)CO[C@@H]1c1cc(F)ccc1F] | (2R,3S,5R)-5-(5- (cyclopropylmethyl)hexahydro-1H- pyrrolo[3,4-c]pyridin-2(3H)-yl)-2-(2,5- difluorophenyl)tetrahydro-2H-pyran-3- amine |
| 77 | 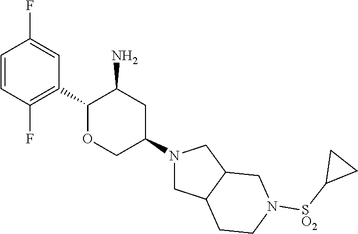 [SMILES: N[C@H]1C[C@@H](N2CC3CCN(S(=O)(=O)C4CC4)CC3C2)CO[C@@H]1c1cc(F)ccc1F] | (2R,3S,5R)-5-(5- (cyclopropylsulfonyl)hexahydro-1H- pyrrolo[3,4-c]pyridin-2(3H)-yl)-2-(2,5- difluorophenyl)tetrahydro-2H-pyran-3- amine |
| 78 | 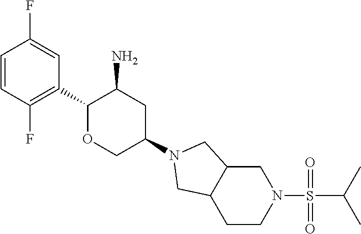 [SMILES: CC(C)S(=O)(=O)N1CCC2CN([C@H]3CO[C@H](c4cc(F)ccc4F)[C@@H](N)C3)CC2C1] | (2R,3S,5R)-2-(2,5-difluorophenyl)-5-(5- (isopropylsulfonyl)hexahydro-1H- pyrrolo[3,4-c]pyridin-2(3H)- yl)tetrahydro-2H-pyran-3-amine |
| 79 | 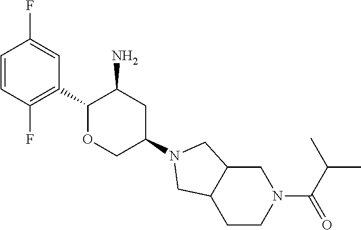 [SMILES: CC(C)C(=O)N1CCC2CN([C@H]3CO[C@H](c4cc(F)ccc4F)[C@@H](N)C3)CC2C1] | 1-(2-((3R,5S,6R)-5-amino-6-(2,5- difluorophenyl)tetrahydro-2H-pyran-3- yl)hexahydro-1H-pyrrolo[3,4-c]pyridin- 5(6H)-yl)-2-methylpropan-1-one |
| 80 | 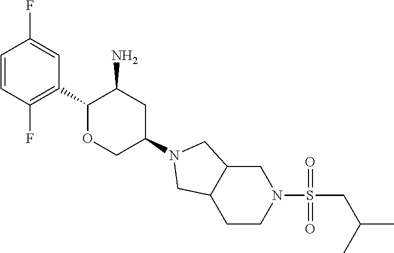 [SMILES: CC(C)CS(=O)(=O)N1CCC2CN([C@H]3CO[C@H](c4cc(F)ccc4F)[C@@H](N)C3)CC2C1] | (2R,3S,5R)-2-(2,5-difluorophenyl)-5-(5- (isobutylsulfonyl)hexahydro-1H- pyrrolo[3,4-c]pyridin-2(3H)- yl)tetrahydro-2H-pyran-3-amine |
| 81 | 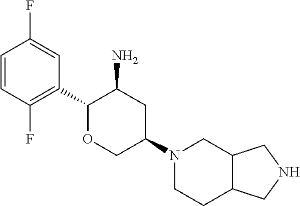 [SMILES: N[C@H]1C[C@@H](N2CCC3CNCC3C2)CO[C@@H]1c1cc(F)ccc1F] | (2R,3S,5R)-2-(2,5-difluorophenyl)-5- (hexahydro-1H-pyrrolo[3,4-c]pyridin- 5(6H)-yl)tetrahydro-2H-pyran-3-amine |
| 82 | 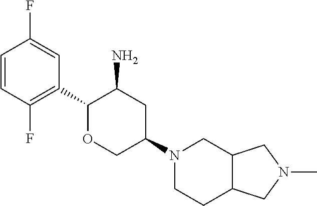 [SMILES: CN1CC2CCN([C@H]3CO[C@H](c4cc(F)ccc4F)[C@@H](N)C3)CC2C1] | (2R,3S,5R)-2-(2,5-difluorophenyl)-5-(2- methylhexahydro-1H-pyrrolo[3,4- c]pyridin-5(6H)-yl)tetrahydro-2H-pyran- 3-amine |
| 83 | 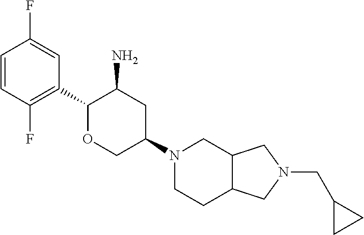 [SMILES: N[C@H]1C[C@@H](N2CCC3CN(CC4CC4)CC3C2)CO[C@@H]1c1cc(F)ccc1F] | (2R,3S,5R)-5-(2- (cyclopropylmethyl)hexahydro-1H- pyrrolo[3,4-c]pyridin-5(6H)-yl)-2-(2,5- difluorophenyl)tetrahydro-2H-pyran-3- amine |
| 84 | 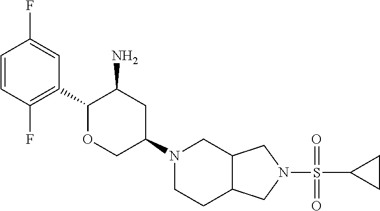 [SMILES: N[C@H]1C[C@@H](N2CCC3CN(S(=O)(=O)C4CC4)CC3C2)CO[C@@H]1c1cc(F)ccc1F] | (2R,3S,5R)-5-(2- (cyclopropylsulfonyl)hexahydro-1H- pyrrolo[3,4-c]pyridin-5(6H)-yl)-2-(2,5- difluorophenyl)tetrahydro-2H-pyran-3- amine |
| 85 | 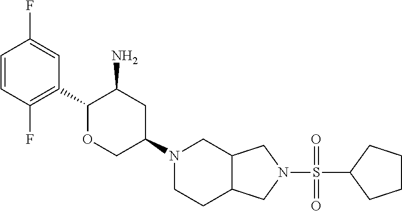 [SMILES: N[C@H]1C[C@@H](N2CCC3CN(S(=O)(=O)C4CCCC4)CC3C2)CO[C@@H]1c1cc(F)ccc1F] | (2R,3S,5R)-5-(2- (cyclopentylsulfonyl)hexahydro-1H- pyrrolo[3,4-c]pyridin-5(6H)-yl)-2-(2,5- difluorophenyl)tetrahydro-2H-pyran-3- amine |
| 86 | 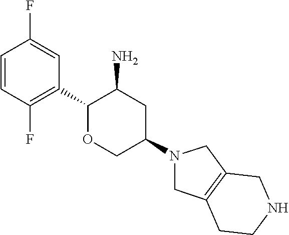 [SMILES: N[C@H]1C[C@@H](N2CC3=C(CNCC3)C2)CO[C@@H]1c1cc(F)ccc1F] | (2R,3S,5R)-2-(2,5-difluorophenyl)-5- (4,5,6,7-tetrahydro-1H-pyrrolo[3,4- c]pyridin-2(3H)-yl)tetrahydro-2H-pyran- 3-amine |
| 87 | 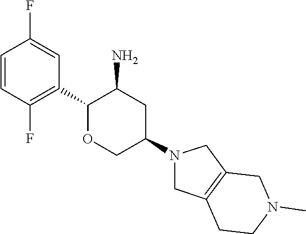 [SMILES: CN1CCC2=C(C1)CN([C@H]1CO[C@H](c3cc(F)ccc3F)[C@@H](N)C1)C2] | (2R,3S,5R)-2-(2,5-difluorophenyl)-5-(5- methyl-4,5,6,7-tetrahydro-1H- pyrrolo[3,4-c]pyridin-2(3H)- yl)tetrahydro-2H-pyran-3-amine |
| 88 | 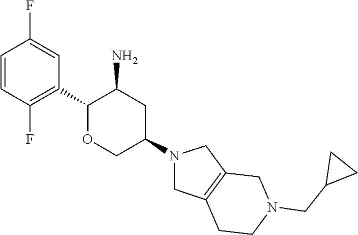 [SMILES: N[C@H]1C[C@@H](N2CC3=C(CN(CC4CC4)CC3)C2)CO[C@@H]1c1cc(F)ccc1F] | (2R,3S,5R)-5-(5-(cyclopropylmethyl)- 4,5,6,7-tetrahydro-1H-pyrrolo[3,4- c]pyridin-2(3H)-yl)-2-(2,5- difluorophenyl)tetrahydro-2H-pyran-3- amine |
| 89 | 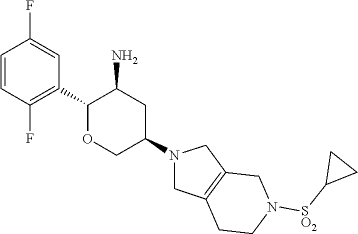 [SMILES: N[C@H]1C[C@@H](N2CC3=C(C2)CN(S(=O)(=O)C2CC2)CC3)CO[C@@H]1c1cc(F)ccc1F] | (2R,3S,5R)-5-(5-(cyclopropylsulfonyl)- 4,5,6,7-tetrahydro-1H-pyrrolo[3,4- c]pyridin-2(3H)-yl)-2-(2,5- difluorophenyl)tetrahydro-2H-pyran-3- amine |
| 90 | 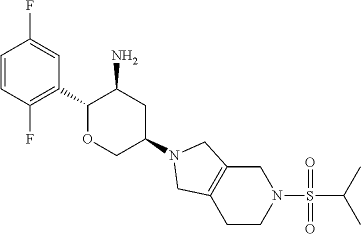 [SMILES: CC(C)S(=O)(=O)N1CCC2=C(CN([C@H]3CO[C@H](c4cc(F)ccc4F)[C@@H](N)C3)C2)C1] | (2R,3S,5R)-2-(2,5-difluorophenyl)-5-(5- (isopropylsulfonyl)-4,5,6,7-tetrahydro- 1H-pyrrolo[3,4-c]pyridin-2(3H)- yl)tetrahydro-2H-pyran-3-amine |
| 91 | 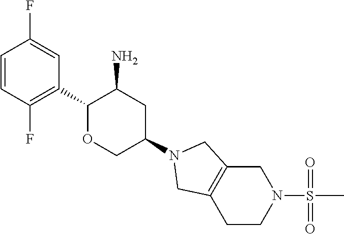 [SMILES: CS(=O)(=O)N1CCC2=C(CN([C@H]3CO[C@H](c4cc(F)ccc4F)[C@@H](N)C3)C2)C1] | (2R,3S,5R)-2-(2,5-difluorophenyl)-5-(5- (methylsulfonyl)-4,5,6,7-tetrahydro-1H- pyrrolo[3,4-c]pyridin-2(3H)- yl)tetrahydro-2H-pyran-3-amine |
| 92 | 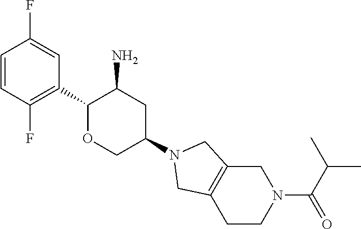 [SMILES: CC(C)C(=O)N1CCC2=C(C1)CN([C@H]1CO[C@H](c3cc(F)ccc3F)[C@@H](N)C1)C2] | 1-(2-((3R,5S,6R)-5-amino-6-(2,5- difluorophenyl)tetrahydro-2H-pyran-3- yl)-2,3,6,7-tetrahydro-1H-pyrrolo(3,4- c]pyridin-5(4H)-yl)-2-methylpropan-1- one |
| 93 | 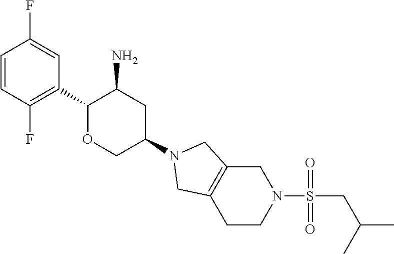 [SMILES: CC(C)CS(=O)(=O)N1CCC2=C(CN([C@H]3CO[C@H](c4cc(F)ccc4F)[C@@H](N)C3)C2)C1] | (2R,3S,5R)-2-(2,5-difluorophenyl)-5-(5- (isobutylsulfonyl)-4,5,6,7-tetrahydro-1H- pyrrolo[3,4-c]pyridin-2(3H)- yl)tetrahydro-2H-pyran-3-amine |
| 94 | 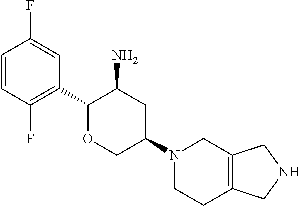 [SMILES: N[C@H]1C[C@@H](N2CCC3=C(CNC3)C2)CO[C@@H]1c1cc(F)ccc1F] | (2R,3S,5R)-2-(2,5-difluorophenyl)-5- (2,3,6,7-tetrahydro-1H-pyrrolo[3,4- c]pyridin-5(4H)-yl)tetrahydro-2H-pyran- 3-amine |
| 95 | 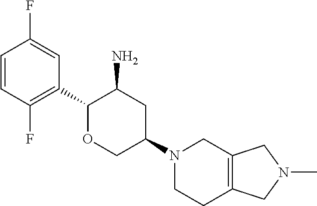 [SMILES: CN1CC2=C(C1)CN([C@H]1CO[C@H](c3cc(F)ccc3F)[C@@H](N)C1)CC2] | (2R,3S,5R)-2-(2,5-difluorophenyl)-5-(2- methyl-2,3,6,7-tetrahydro-1H- pyrrolo[3,4-c]pyridin-5(4H)- yl)tetrahydro-2H-pyran-3-amine |
| 96 | 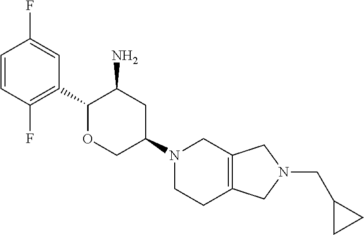 [SMILES: N[C@H]1C[C@@H](N2CCC3=C(CN(CC4CC4)C3)C2)CO[C@@H]1c1cc(F)ccc1F] | (2R,3S,5R)-5-(2-(cyclopropylmethyl)- 2,3,6,7-tetrahydro-1H-pyrrolo[3,4-c] pyridin-5(4H)-yl)-2-(2,5- difluorophenyl)tetrahydro-2H-pyran-3- amine |
| 97 | 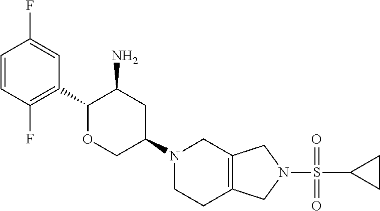 [SMILES: N[C@H]1C[C@@H](N2CCC3=C(C2)CN(S(=O)(=O)C2CC2)C3)CO[C@@H]1c1cc(F)ccc1F] | (2R,3S,5R)-5-(2-(cyclopropylsulfonyl)- 2,3,6,7-tetrahydro-1H-pyrrolo[3,4- c]pyridin-5(4H)-yl)-2-(2,5- difluorophenyl)tetrahydro-2H-pyran-3- amine |
| 98 | 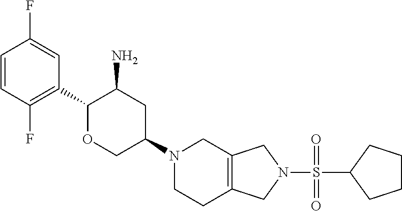 [SMILES: N[C@H]1C[C@@H](N2CCC3=C(C2)CN(S(=O)(=O)C2CCCC2)C3)CO[C@@H]1c1cc(F)ccc1F] | (2R,3S,5R)-5-(2-(cyclopentylsulfonyl)- 2,3,6,7-tetrahydro-1H-pyrrolo[3,4- c]pyridin-5(4H)-yl)-2-(2,5- difluorophenyl)tetrahydro-2H-pyran-3- amine |
| 99 | 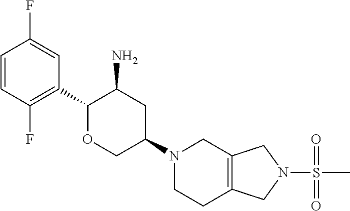 [SMILES: CS(=O)(=O)N1CC2=C(CN([C@H]3CO[C@H](c4cc(F)ccc4F)[C@@H](N)C3)CC2)C1] | (2R,3S,5R)-2-(2,5-difluorophenyl)-5-(2- (methylsulfonyl)-2,3,6,7-tetrahydro-1H- pyrrolo[3,4-c]pyridin-5(4H)- yl)tetrahydro-2H-pyran-3-amine |
| 100 | 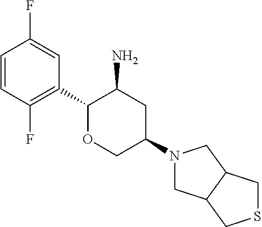 [SMILES: N[C@H]1C[C@@H](N2CC3CSCC3C2)CO[C@@H]1c1cc(F)ccc1F] | (2R,3S,5R)-2-(2,5-difluorophenyl)-5- (tetrahydro-1H-thieno[3,4-c]pyrrol- 5(3H)-yl)tetrahydro-2H-pyran-3-amine |
| 101 | 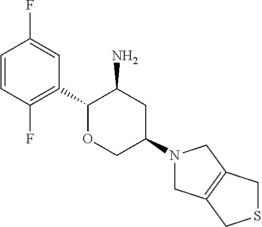 [SMILES: N[C@H]1C[C@@H](N2CC3=C(CSC3)C2)CO[C@@H]1c1cc(F)ccc1F] | (2R,3S,5R)-2-(2,5-difluorophenyl)-5- (1H-thieno[3,4-c]pyrrol-5(3H,4H,6H)- yI)tetrahydro-2H-pyran-3-amine |
| 102 | 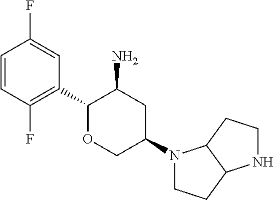 [SMILES: N[C@H]1C[C@@H](N2CCC3NCCC32)CO[C@@H]1c1cc(F)ccc1F] | (2R,3S,5R)-2-(2,5-difluorophenyl)-5- (hexahydropyrrolo[3,2-b]pyrrol-1(2H)- yl)tetrahydro-2H-pyran-3-amine |
| 103 | 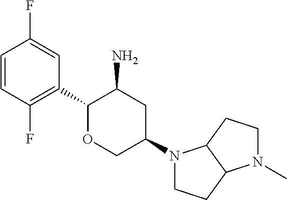 [SMILES: CN1CCC2C1CCN2[C@H]1CO[C@H](c2cc(F)ccc2F)[C@@H](N)C1] | (2R,3S,5R)-2-(2,5-difluorophenyl)-5-(4- methylhexahydropyrrolo[3,2-b]pyrrol- 1(2H)-yl)tetrahydro-2H-pyran-3-amine |
| 104 | 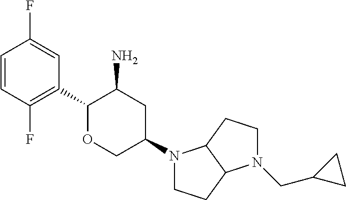 [SMILES: N[C@H]1C[C@@H](N2CCC3C2CCN3CC2CC2)CO[C@@H]1c1cc(F)ccc1F] | (2R,3S,5R)-5-(4- (cyclopropylmethyl)hexahydropyrrolo[3, 2-b)pyrrol-1(2H)-yl)-2-(2,5- difluorophenyl)tetrahydro-2H-pyran-3- amine |
| 105 | 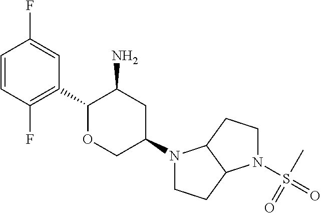 [SMILES: CS(=O)(=O)N1CCC2C1CCN2[C@H]1CO[C@H](c2cc(F)ccc2F)[C@@H](N)C1] | (2R,3S,5R)-2-(2,5-difluorophenyl)-5-(4- (methylsulfonyl)hexahydropyrrolo[3,2- b]pyrrol-1(2H)-yl)tetrahydro-2H-pyran- 3-amine |
| 106 | 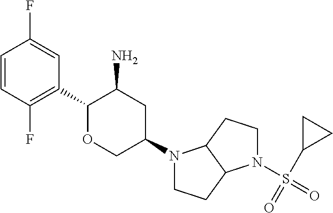 [SMILES: N[C@H]1C[C@@H](N2CCC3C2CCN3S(=O)(=O)C2CC2)CO[C@@H]1c1cc(F)ccc1F] | (2R,3S,5R)-5-(4- (cyclopropylsulfonyl)hexahydropyrrolo [3,2-b]pyrrol-1(2H)-yl)-2-(2,5- difluorophenyl)tetrahydro-2H-pyran-3- amine |
| 107 | 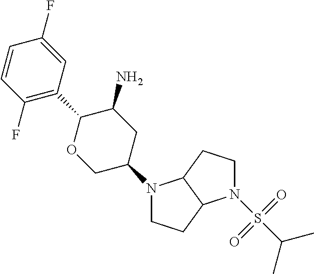 [SMILES: CC(C)S(=O)(=O)N1CCC2C1CCN2[C@H]1CO[C@H](c2cc(F)ccc2F)[C@@H](N)C1] | (2R,3S,5R)-2-(2,5-difluorophenyl)-5-(4- (isopropylsulfonyl)hexahydropyrrolo[3,2- b]pyrrol-1(2H)-yl)tetrahydro-2H-pyran- 3-amine |
| 108 | 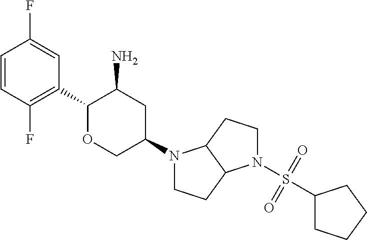 [SMILES: N[C@H]1C[C@@H](N2CCC3C2CCN3S(=O)(=O)C2CCCC2)CO[C@@H]1c1cc(F)ccc1F] | (2R,3S,5R)-5-(4- (cyclopentylsulfonyl)hexahydropyrrolo [3,2-b]pyrrol-1(2H)-yl)-2-(2,5- difluorophenyl)tetrahydro-2H-pyran-3- amine |
| 109 | 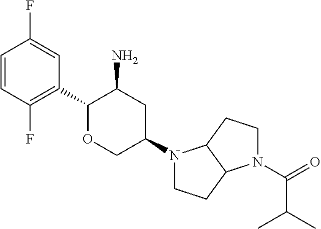 [SMILES: CC(C)C(=O)N1CCC2C1CCN2[C@H]1CO[C@H](c2cc(F)ccc2F)[C@@H](N)C1] | 1-(4-((3R,5S,6R)-5-amino-6-(2,5- difluorophenyl)tetrahydro-2H-pyran-3- yl)hexahydropyrrolo[3,2-b]pyrrol-1(2H)- yl)-2-methylpropan-1-one |
| 110 | 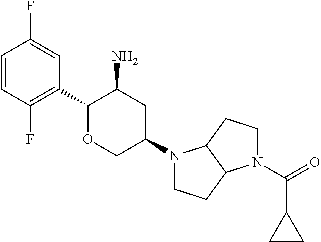 [SMILES: N[C@H]1C[C@@H](N2CCC3C2CCN3C(=O)C2CC2)CO[C@@H]1c1cc(F)ccc1F] | (4-((3R,5S,6R)-5-amino-6-(2,5- difluorophenyl)tetrahydro-2H-pyran-3- yl)hexahydropyrrolo[3,2-b]pyrrol-1(2H)- yl)(cyclopropyl)methanone |
| 111 | 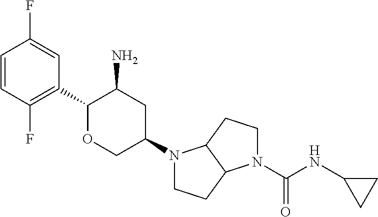 [SMILES: N[C@H]1C[C@@H](N2CCC3C2CCN3C(=O)NC2CC2)CO[C@@H]1c1cc(F)ccc1F] | 4-((3R,5S,6R)-5-amino-6-(2,5- difluorophenyl)tetrahydro-2H-pyran-3- yl)-N-cyclopropylhexahydropyrrolo[3,2- b]pyrrole-1(2H)-carboxamide |
| 112 | 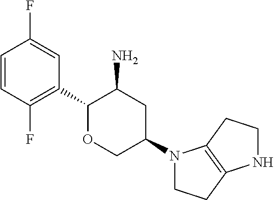 [SMILES: N[C@H]1C[C@@H](N2CCC3=C2CCN3)CO[C@@H]1c1cc(F)ccc1F] | (2R,3S,5R)-2-(2,5-difluorophenyl)-5- (2,3,5,6-tetrahydropyrrolo[3,2-b]pyrrol- 1(4H)-yl)tetrahydro-2H-pyran-3-amine |
| 113 | 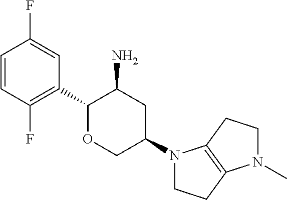 [SMILES: CN1CCC2=C1CCN2[C@H]1CO[C@H](c2cc(F)ccc2F)[C@@H](N)C1] | (2R,3S,5R)-2-(2,5-difluorophenyl)-5-(4- methyl-2,3,5,6-tetrahydropyrrolo[3,2- b]pyrrol-1(4H)-yl)tetrahydro-2H-pyran- 3-amine |
| 114 | 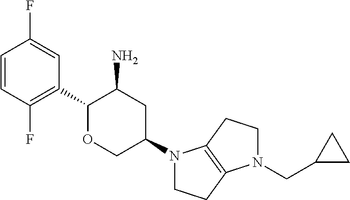 [SMILES: N[C@H]1C[C@@H](N2CCC3=C2CCN3CC2CC2)CO[C@@H]1c1cc(F)ccc1F] | (2R,3S,5R)-5-(4-(cyclopropylmethyl)- 2,3,5,6-tetrahydropyrrolo[3,2-b]pyrrol- 1(4H)-yl)-2-(2,5- difluorophenyl)tetrahydro-2H-pyran-3- amine |
| 115 | 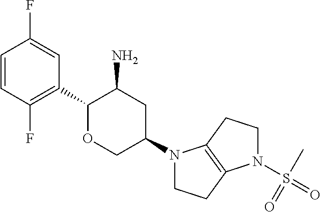 [SMILES: CS(=O)(=O)N1CCC2=C1CCN2[C@H]1CO[C@H](c2cc(F)ccc2F)[C@@H](N)C1] | (2R,3S,5R)-2-(2,5-difluorophenyl)-5-(4- (methylsulfonyl)-2,3,5,6- tetrahydropyrrolo[3,2-b]pyrrol-1(4H)- yl)tetrahydro-2H-pyran-3-amine |
| 116 | 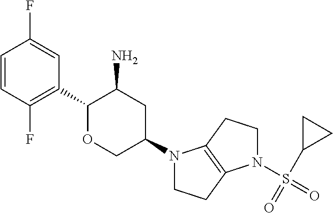 [SMILES: N[C@H]1C[C@@H](N2CCC3=C2CCN3S(=O)(=O)C2CC2)CO[C@@H]1c1cc(F)ccc1F] | (2R,3S,5R)-5-(4-(cyclopropylsulfonyl)- 2,3,5,6-tetrahydropyrrolo[3,2-b]pyrrol- 1(4H)-yl)-2-(2,5- difluorophenyl)tetrahydro-2H-pyran-3- amine |
| 117 | 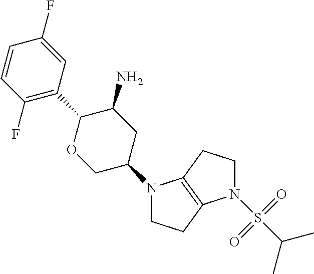 [SMILES: CC(C)S(=O)(=O)N1CCC2=C1CCN2[C@H]1CO[C@H](c2cc(F)ccc2F)[C@@H](N)C1] | (2R,3S,5R)-2-(2,5-difluorophenyl)-5-(4- (isopropylsulfonyl)-2,3,5,6- tetrahydropyrrolo[3,2-b]pyrrol-1(4H)- yl)tetrahydro-2H-pyran-3-amine |
| 118 | 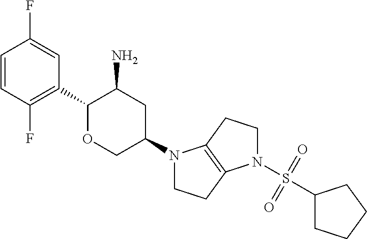 [SMILES: N[C@H]1C[C@@H](N2CCC3=C2CCN3S(=O)(=O)C2CCCC2)CO[C@@H]1c1cc(F)ccc1F] | (2R,3S,5R)-5-(4-(cyclopentylsulfonyl)- 2,3,5,6-tetrahydropyrrolo[3,2-b]pyrrol- 1(4H)-yl)-2-(2,5- difluorophenyl)tetrahydro-2H-pyran-3- amine |
| 119 | 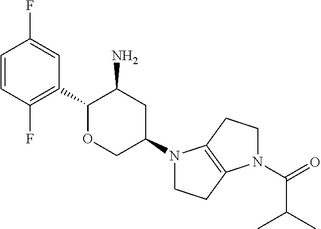 [SMILES: CC(C)C(=O)N1CCC2=C1CCN2[C@H]1CO[C@H](c2cc(F)ccc2F)[C@@H](N)C1] | 1-(4-((3R,5S,6R)-5-amino-6-(2,5- difluorophenyl)tetrahydro-2H-pyran-3- y1)-2,3,5,6-tetrahydropyrrolo[3,2-b] pyrrol-1(4H)-yl)-2-methylpropan-1- one |
| 120 | 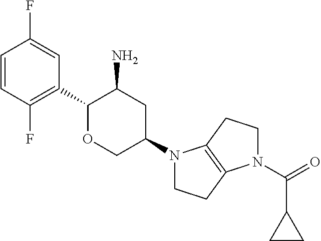 [SMILES: N[C@H]1C[C@@H](N2CCC3=C2CCN3C(=O)C2CC2)CO[C@@H]1c1cc(F)ccc1F] | (4-((3R,5S,6R)-5-amino-6-(2,5- difluorophenyl)tetrahydro-2H-pyran-3- yl)-2,3,5,6-tetrahydropyrrolo[3,2- b]pyrrol-1(4H)- yl)(cyclopropyl)methanone |
| 121 | 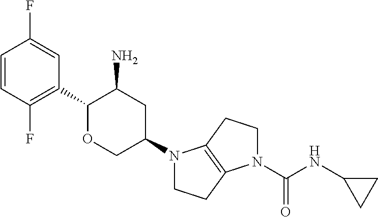 [SMILES: N[C@H]1C[C@@H](N2CCC3=C2CCN3C(=O)NC2CC2)CO[C@@H]1c1cc(F)ccc1F] | 4-((3R,5S,6R)-5-amino-6-(2,5- difluorophenyl)tetrahydro-2H-pyran-3- yl)-N-cyclopropyl-2,3,5,6- tetrahydropyrrolo[3,2-b]pyrrole-1(4H)- carboxamide |
| 122 | 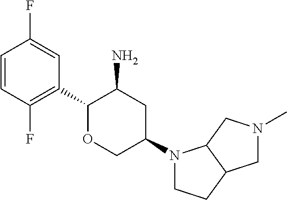 [SMILES: CN1CC2CCN([C@H]3CO[C@H](c4cc(F)ccc4F)[C@@H](N)C3)C2C1] | (2R,3S,5R)-2-(2,5-difluorophenyl)-5-(5- methylhexahydropyrrolo[3,4-b]pyrrol- 1(2H)-yl)tetrahydro-2H-pyran-3-amine |
| 123 | 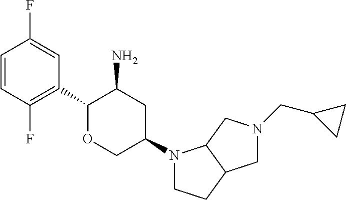 [SMILES: N[C@H]1C[C@@H](N2CCC3CN(CC4CC4)CC32)CO[C@@H]1c1cc(F)ccc1F] | (2R,3S,5R)-5-(5- (cyclopropylmethyl)hexahydropyrrolo[3, 4-b]pyrrol-1(2H)-yl)-2-(2,5- difluorophenyl)tetrahydro-2H-pyran-3- amine |
| 124 | 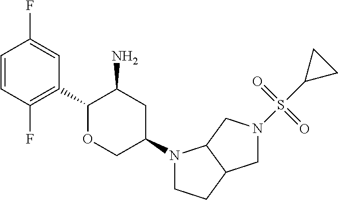 [SMILES: N[C@H]1C[C@@H](N2CCC3CN(S(=O)(=O)C4CC4)CC32)CO[C@@H]1c1cc(F)ccc1F] | (2R,3S,5R)-5-(5- (cyclopropylsulfonyl)hexahydropyrrolo [3,4-b]pyrrol-1(2H)-yl)-2-(2,5- difluorophenyl)tetrahydro-2H-pyran-3- amine |
| 125 | 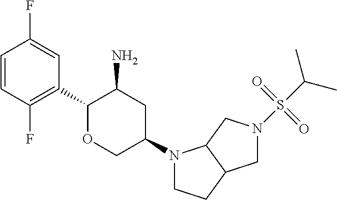 [SMILES: CC(C)S(=O)(=O)N1CC2CCN([C@H]3CO[C@H](c4cc(F)ccc4F)[C@@H](N)C3)C2C1] | (2R,3S,5R)-2-(2,5-difluorophenyl)-5-(5- (isopropylsulfonyl)hexahydropyrrolo[3,4- b]pyrrol-1(2H)-yl)tetrahydro-2H-pyran- 3-amine |
| 126 | 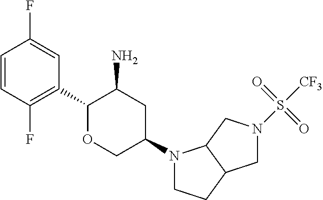 [SMILES: N[C@H]1C[C@@H](N2CCC3CN(S(=O)(=O)C(F)(F)F)CC32)CO[C@@H]1c1cc(F)ccc1F] | (2R,3S,5R)-2-(2,5-difluorophenyl)-5-(5- ((trifluoromethyl)sulfonyl)hexahydropyrrolo [3,4-b]pyrrol-1(2H)-yl)tetrahydro-2H- pyran-3-amine |
| 127 | 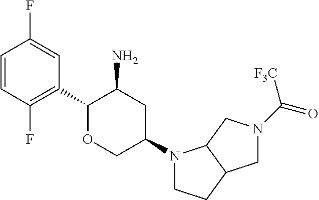 [SMILES: N[C@H]1C[C@@H](N2CCC3CN(C(=O)C(F)(F)F)CC32)CO[C@@H]1c1cc(F)ccc1F] | 1-(1-((3R,5S,6R)-5-amino-6-(2,5- difluorophenyl)tetrahydro-2H-pyran-3- yl)hexahydropyrrolo[3,4-b]pyrrol-5(1H)- yl)-2,2,2-trifluoroethanone |
| 128 | 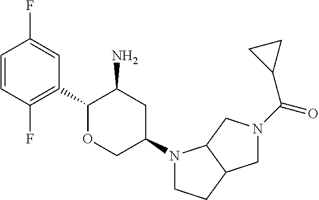 [SMILES: N[C@H]1C[C@@H](N2CCC3CN(C(=O)C4CC4)CC32)CO[C@@H]1c1cc(F)ccc1F] | (1-((3R,5S,6R)-5-amino-6-(2,5- difluorophenyl)tetrahydro-2H-pyran-3- yl)hexahydropyrrolo[3,4-b]pyrrol-5(1H)- yl)(cyclopropyl)methanone |
| 129 | 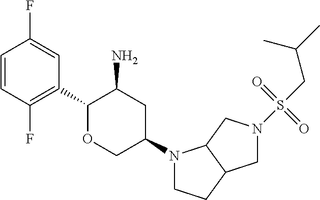 [SMILES: CC(C)CS(=O)(=O)N1CC2CCN([C@H]3CO[C@H](c4cc(F)ccc4F)[C@@H](N)C3)C2C1] | (2R,3S,5R)-2-(2,5-difluorophenyl)-5-(5- (isobutylsulfonyl)hexahydropyrrolo[3,4- b]pyrrol-1(2H)-yl)tetrahydro-2H-pyran- 3-amine |
| 130 | 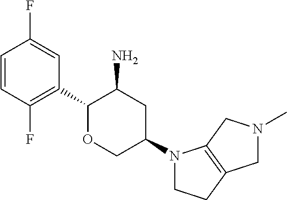 [SMILES: CN1CC2=C(C1)N([C@H]1CO[C@H](c3cc(F)ccc3F)[C@@H](N)C1)CC2] | (2R,3S,5R)-2-(2,5-difluorophenyl)-5-(5- methyl-2,3,5,6-tetrahydropyrrolo[3,4- b]pyrrol-1(4H)-yl)tetrahydro-2H-pyran- 3-amine |
| 131 | 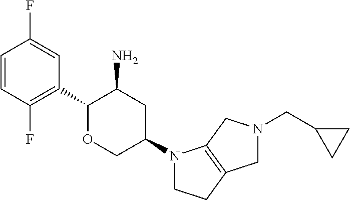 [SMILES: N[C@H]1C[C@@H](N2CCC3=C2CN(CC2CC2)C3)CO[C@@H]1c1cc(F)ccc1F] | (2R,3S,5R)-5-(5-(cyclopropylmethyl)- 2,3,5,6-tetrahydropyrrolo[3,4-b]pyrrol- 1(4H)-yl)-2-(2,5- difluorophenyl)tetrahydro-2H-pyran-3- amine |
| 132 | 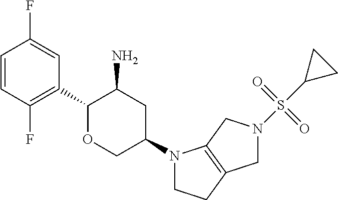 [SMILES: N[C@H]1C[C@@H](N2CCC3=C2CN(S(=O)(=O)C2CC2)C3)CO[C@@H]1c1cc(F)ccc1F] | (2R,3S,5R)-5-(5-(cyclopropylsulfonyl)- 2,3,5,6-tetrahydropyrrolo[3,4-b]pyrrol- 1(4H)-yl)-2-(2,5- difluorophenyl)tetrahydro-2H-pyran-3- amine |
| 133 | 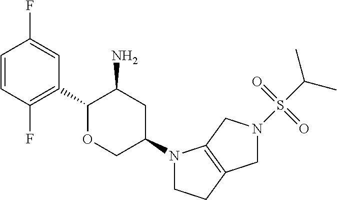 [SMILES: CC(C)S(=O)(=O)N1CC2=C(C1)N([C@H]1CO[C@H](c3cc(F)ccc3F)[C@@H](N)C1)CC2] | (2R,3S,5R)-2-(2,5-difluorophenyl)-5-(5- (isopropylsulfonyl)-2,3,5,6- tetrahydropyrrolo[3,4-b]pyrrol-1(4H)- yl)tetrahydro-2H-pyran-3-amine |
| 134 | 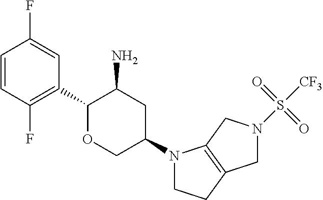 [SMILES: N[C@H]1C[C@@H](N2CCC3=C2CN(S(=O)(=O)C(F)(F)F)C3)CO[C@@H]1c1cc(F)ccc1F] | (2R,3S,5R)-2-(2,5-difluorophenyl)-5-(5- ((trifluoromethyl)sulfonyl)-2,3,5,6- tetrahydropyrrolo[3,4-b]pyrrol-1(4H)- yl)tetrahydro-2H-pyran-3-amine |
| 135 | 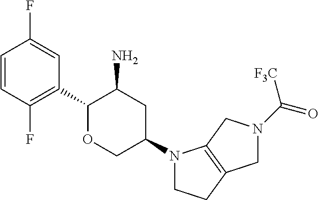 [SMILES: N[C@H]1C[C@@H](N2CCC3=C2CN(C(=O)C(F)(F)F)C3)CO[C@@H]1c1cc(F)ccc1F] | 1-(1-((3R,5S,6R)-5-amino-6-(2,5- difluorophenyl)tetrahydro-2H-pyran-3- yl)-2,3-dihydropyrrolo[3,4-b]pyrrol- 5(1H,4H,6H)-yl)-2,2,2-trifluoroethanone |
| 136 | 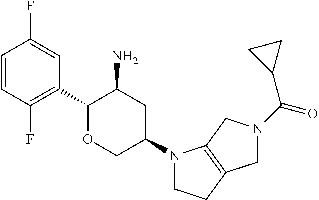 [SMILES: N[C@H]1C[C@@H](N2CCC3=C2CN(C(=O)C2CC2)C3)CO[C@@H]1c1cc(F)ccc1F] | (1-((3R,5S,6R)-5-amino-6-(2,5- difluorophenyl)tetrahydro-2H-pyran-3- yl)-2,3-dihydropyrrolo[3,4-b]pyrrol- 5(1H,4H,6H)-yl)(cyclopropyl)methanone |
| 137 | 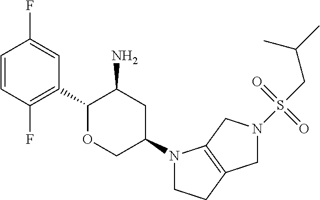 [SMILES: CC(C)CS(=O)(=O)N1CC2=C(C1)N([C@H]1CO[C@H](c3cc(F)ccc3F)[C@@H](N)C1)CC2] | (2R,3S,5R)-2-(2,5-difluorophenyl)-5-(5- (isobutylsulfonyl)-2,3,5,6- tetrahydropyrrolo[3,4-b]pyrrol-1(4H)- yl)tetrahydro-2H-pyran-3-amine |
| 138 | 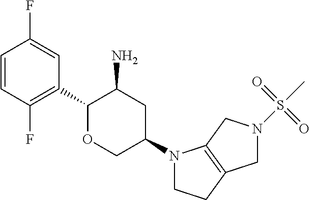 [SMILES: CS(=O)(=O)N1CC2=C(C1)N([C@H]1CO[C@H](c3cc(F)ccc3F)[C@@H](N)C1)CC2] | (2R,3S,5R)-2-(2,5-difluorophenyl)-5-(5- (methylsulfonyl)-2,3,5,6- tetrahydropyrrolo[3,4-b]pyrrol-1(4H)- yl)tetrahydro-2H-pyran-3-amine |
| 139 | 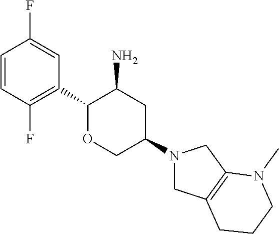 [SMILES: CN1CCCC2=C1CN([C@H]1CO[C@H](c3cc(F)ccc3F)[C@@H](N)C1)C2] | (2R,3S,5R)-2-(2,5-difluorophenyl)-5-(1- methyl-3,4-dihydro-1H-pyrrolo[3,4- b]pyridin-6(2H,5H,7H)-yl)tetrahydro- 2H-pyran-3-amine |
| 140 | 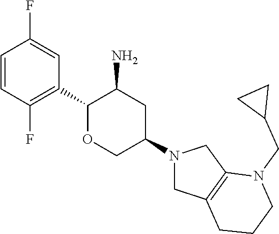 [SMILES: N[C@H]1C[C@@H](N2CC3=C(C2)N(CC2CC2)CCC3)CO[C@@H]1c1cc(F)ccc1F] | (2R,3S,5R)-5-(1-(cyclopropylmethyl)- 3,4-dihydro-1H-pyrrolo[3,4-b]pyridin- 6(2H,5H,7H)-yl)-2-(2,5- difluorophenyl)tetrahydro-2H-pyran-3- amine |
| 141 | 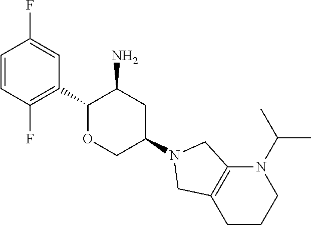 [SMILES: CC(C)N1CCCC2=C1CN([C@H]1CO[C@H](c3cc(F)ccc3F)[C@@H](N)C1)C2] | (2R,3S,5R)-2-(2,5-difluorophenyl)-5-(1- isopropyl-3,4-dihydro-1H-pyrrolo[3,4- b]pyridin-6(2H,5H,7H)-yl)tetrahydro- 2H-pyran-3-amine |
| 142 | 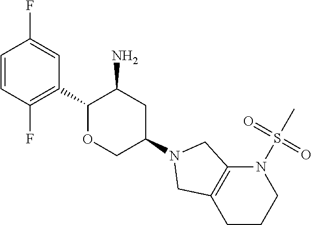 [SMILES: CS(=O)(=O)N1CCCC2=C1CN([C@H]1CO[C@H](c3cc(F)ccc3F)[C@@H](N)C1)C2] | (2R,3S,5R)-2-(2,5-difluorophenyl)-5-(1- (methylsulfonyl)-3,4-dihydro-1H- pyrrolo[3,4-b]pyridin-6(2H,5H,7H)- yl)tetrahydro-2H-pyran-3-amine |
| 143 | 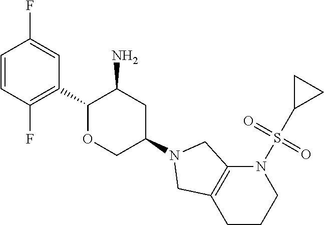 [SMILES: N[C@H]1C[C@@H](N2CC3=C(C2)N(S(=O)(=O)C2CC2)CCC3)CO[C@@H]1c1cc(F)ccc1F] | (2R,3S,5R)-5-(1-(cyclopropylsulfonyl)- 3,4-dihydro-1H-pyrrolo[3,4-b]pyridin- 6(2H,5H,7H)-yl)-2-(2,5- difluorophenyl)tetrahydro-2H-pyran-3- amine |
| 144 | 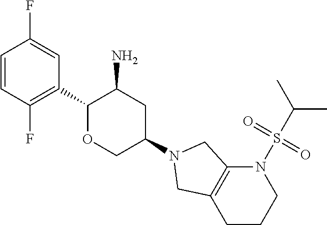 [SMILES: CC(C)S(=O)(=O)N1CCCC2=C1CN([C@H]1CO[C@H](c3cc(F)ccc3F)[C@@H](N)C1)C2] | (2R,3S,5R)-2-(2,5-difluorophenyl)-5-(1- (isopropylsulfonyl)-3,4-dihydro-1H- pyrrolo[3,4-b]pyridin-6(2H,5H,7H)- yl)tetrahydro-2H-pyran-3-amine |
| 145 | 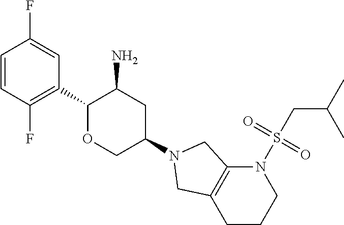 [SMILES: CC(C)CS(=O)(=O)N1CCCC2=C1CN([C@H]1CO[C@H](c3cc(F)ccc3F)[C@@H](N)C1)C2] | (2R,3S,5R)-2-(2,5-difluorophenyl)-5-(1- (isobutylsulfonyl)-3,4-dihydro-1H- pyrrolo[3,4-b]pyridin-6(2H,5H,7H)- yl)tetrahydro-2H-pyran-3-amine |
| 146 | 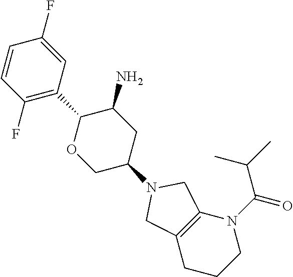 [SMILES: CC(C)C(=O)N1CCCC2=C1CN([C@H]1CO[C@H](c3cc(F)ccc3F)[C@@H](N)C1)C2] | 1-(6-((3R,5S,6R)-5-amino-6-(2,5- difluorophenyl)tetrahydro-2H-pyran-3- yl)-2,3,4,5,6,7-hexahydro-1H- pyrrolo[3,4-b]pyridin-1-yl)-2- methylpropan-1-one |
| 147 | 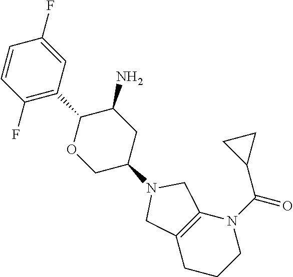 [SMILES: N[C@H]1C[C@@H](N2CC3=C(C2)N(C(=O)C2CC2)CCC3)CO[C@@H]1c1cc(F)ccc1F] | (6-((3R,5S,6R)-5-amino-6-(2,5- difluorophenyl)tetrahydro-2H-pyran-3- yl)-2,3,4,5,6,7-hexahydro-1H- pyrrolo[3,4-b]pyridin-1- yl)(cyclopropyl)methanone |
| 148 | 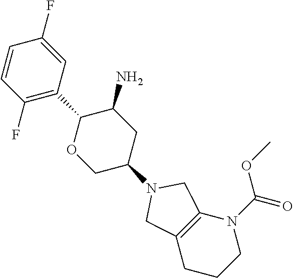 [SMILES: COC(=O)N1CCCC2=C1CN([C@H]1CO[C@H](c3cc(F)ccc3F)[C@@H](N)C1)C2] | methyl 6-((3R,5S,6R)-5-amino-6-(2,5- difluorophenyl)tetrahydro-2H-pyran-3- yl)-2,3,4,5,6,7-hexahydro-1H- pyrrolo[3,4-b]pyridine-1-carboxylate |
| 149 | 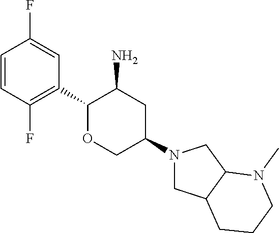 [SMILES: CN1CCCC2CN([C@H]3CO[C@H](c4cc(F)ccc4F)[C@@H](N)C3)CC21] | (2R,3S,5R)-2-(2,5-difluorophenyl)-5-(1- methylhexahydro-1H-pyrrolo[3,4- b]pyridin-6(2H)-yl)tetrahydro-2H-pyran- 3-amine |
| 150 | 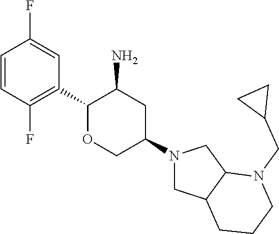 [SMILES: N[C@H]1C[C@@H](N2CC3CCCN(CC4CC4)C3C2)CO[C@@H]1c1cc(F)ccc1F] | (2R,3S,5R)-5-(1- (cyclopropylmethyl)hexahydro-1H- pyrrolo[3,4-b]pyridin-6(2H)-yl)-2-(2,5- difluorophenyl)tetrahydro-2H-pyran-3- amine |
| 151 | 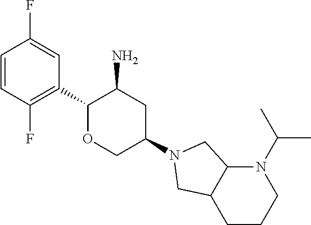 [SMILES: CC(C)N1CCCC2CN([C@H]3CO[C@H](c4cc(F)ccc4F)[C@@H](N)C3)CC21] | (2R,3S,5R)-2-(2,5-difluorophenyl)-5-(1- isopropylhexahydro-1H-pyrrolo[3,4- b]pyridin-6(2H)-yl)tetrahydro-2H-pyran- 3-amine |
| 152 | 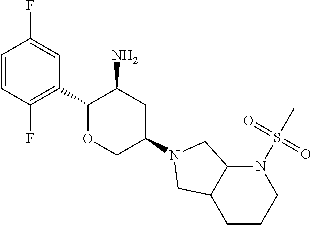 [SMILES: CS(=O)(=O)N1CCCC2CN([C@H]3CO[C@H](c4cc(F)ccc4F)[C@@H](N)C3)CC21] | (2R,3S,5R)-2-(2,5-difluorophenyl)-5-(1- (methylsulfonyl)hexahydro-1H- pyrrolo[3,4-b]pyridin-6(2H)- yl)tetrahydro-2H-pyran-3-amine |
| 153 | 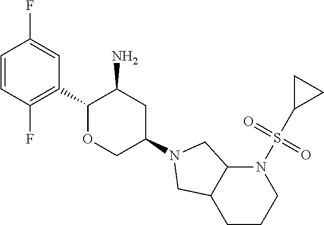 [SMILES: N[C@H]1C[C@@H](N2CC3CCCN(S(=O)(=O)C4CC4)C3C2)CO[C@@H]1c1cc(F)ccc1F] | (2R,3S,5R)-5-(1- (cyclopropylsulfonyl)hexahydro-1H- pyrrolo[3,4-b]pyridin-6(2H)-yl)-2-(2,5- difluorophenyl)tetrahydro-2H-pyran-3- amine |
| 154 | 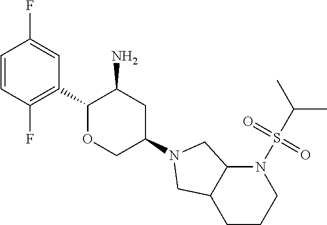 [SMILES: CC(C)S(=O)(=O)N1CCCC2CN([C@H]3CO[C@H](c4cc(F)ccc4F)[C@@H](N)C3)CC21] | (2R,3S,5R)-2-(2,5-difluorophenyl)-5-(1- (isopropylsulfonyl)hexahydro-1H- pyrrolo[3,4-b]pyridin-6(2H)- yl)tetrahydro-2H-pyran-3-amine |
| 155 | 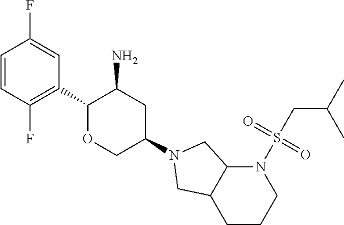 [SMILES: CC(C)CS(=O)(=O)N1CCCC2CN([C@H]3CO[C@H](c4cc(F)ccc4F)[C@@H](N)C3)CC21] | (2R,3S,5R)-2-(2,5-difluorophenyl)-5-(1- (isobutylsulfonyl)hexahydro-1H- pyrrolo[3,4-b]pyridin-6(2H)- yl)tetrahydro-2H-pyran-3-amine |
| 156 | 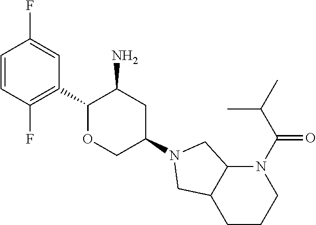 [SMILES: CC(C)C(=O)N1CCCC2CN([C@H]3CO[C@H](c4cc(F)ccc4F)[C@@H](N)C3)CC21] | 1-(6-((3R,5S,6R)-5-amino-6-(2,5- difluorophenyl)tetrahydro-2H-pyran-3- yl)octahydro-1H-pyrrolo[3,4-b]pyridin- 1-yl)-2-methylpropan-1-one |
| 157 | 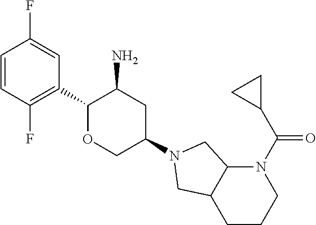 [SMILES: N[C@H]1C[C@@H](N2CC3CCCN(C(=O)C4CC4)C3C2)CO[C@@H]1c1cc(F)ccc1F] | (6-((3R,5S,6R)-5-amino-6-(2,5- difluorophenyl)tetrahydro-2H-pyran-3- yl)octahydro-1H-pyrrolo[3,4-b]pyridin- 1-yl)(cyclopropyl)methanone |
| 158 | 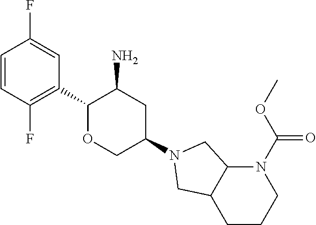 [SMILES: COC(=O)N1CCCC2CN([C@H]3CO[C@H](c4cc(F)ccc4F)[C@@H](N)C3)CC21] | methyl 6-((3R,5S,6R)-5-amino-6-(2,5- difluorophenyl)tetrahydro-2H-pyran-3- yl)octahydro-1H-pyrrolo[3,4-b]pyridine- 1-carboxylate |
| 159 | 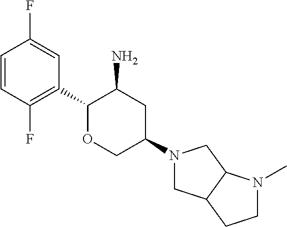 [SMILES: CN1CCC2CN([C@H]3CO[C@H](c4cc(F)ccc4F)[C@@H](N)C3)CC21] | (2R,3S,5R)-2-(2,5-difluorophenyl)-5-(1- methylhexahydropyrrolo[3,4-b]pyrrol- 5(1H)-yl)tetrahydro-2H-pyran-3-amine |
| 160 | 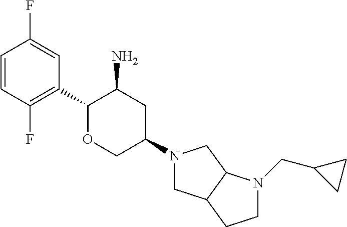 [SMILES: N[C@H]1C[C@@H](N2CC3CCN(CC4CC4)C3C2)CO[C@@H]1c1cc(F)ccc1F] | (2R,3S,5R)-5-(1- (cyclopropylmethyl)hexahydropyrrolo [3,4-b]pyrrol-5(1H)-yl)-2-(2,5- difluorophenyl)tetrahydro-2H-pyran-3- amine |
| 161 | 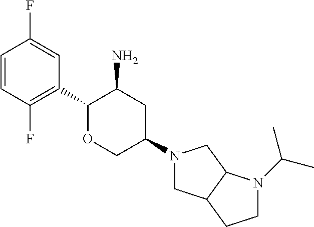 [SMILES: CC(C)N1CCC2CN([C@H]3CO[C@H](c4cc(F)ccc4F)[C@@H](N)C3)CC21] | (2R,3S,5R)-2-(2,5-difluorophenyl)-5-(1- isopropylhexahydropyrrolo[3,4-b]pyrrol- 5(1H)-yl)tetrahydro-2H-pyran-3-amine |
| 162 | 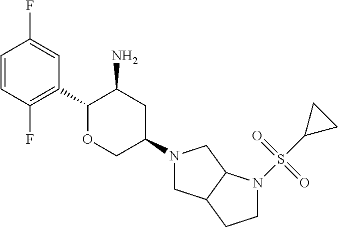 [SMILES: N[C@H]1C[C@@H](N2CC3CCN(S(=O)(=O)C4CC4)C3C2)CO[C@@H]1c1cc(F)ccc1F] | (2R,3S,5R)-5-(1- (cyclopropylsulfonyl)hexahydropyrrolo [3,4-b]pyrrol-5(1H)-yl)-2-(2,5- difluorophenyl)tetrahydro-2H-pyran-3- amine |
| 163 | 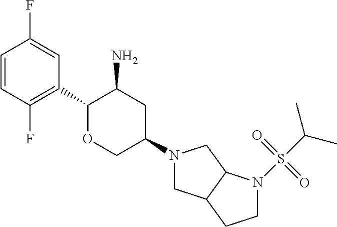 [SMILES: CC(C)S(=O)(=O)N1CCC2CN([C@H]3CO[C@H](c4cc(F)ccc4F)[C@@H](N)C3)CC21] | (2R,3S,5R)-2-(2,5-difluorophenyl)-5-(1- (isopropylsulfonyl)hexahydropyrrolo[3,4-b] pyrrol-5(1H)-yl)tetrahydro-2H-pyran- 3-amine |
| 164 | 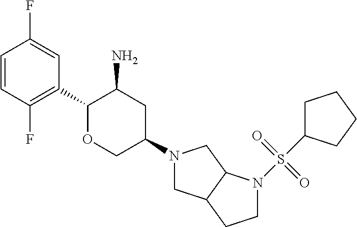 [SMILES: N[C@H]1C[C@@H](N2CC3CCN(S(=O)(=O)C4CCCC4)C3C2)CO[C@@H]1c1cc(F)ccc1F] | (2R,3S,5R)-5-(1- (cyclopentylsulfonyl)hexahydropyrrolo [3,4-b]pyrrol-5(1H)-yl)-2-(2,5- difluorophenyl)tetrahydro-2H-pyran-3- amine |
| 165 | 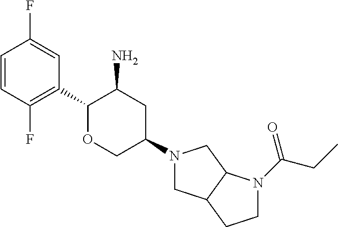 [SMILES: CCC(=O)N1CCC2CN([C@H]3CO[C@H](c4cc(F)ccc4F)[C@@H](N)C3)CC21] | 1-(5-((3R,5S,6R)-5-amino-6-(2,5- difluorophenyl)tetrahydro-2H-pyran-3- yl)hexahydropyrrolo[3,4-b]pyrrol-1(2H)- yl)propan-1-one |
| 166 | 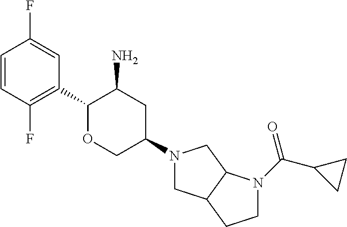 [SMILES: N[C@H]1C[C@@H](N2CC3CCN(C(=O)C4CC4)C3C2)CO[C@@H]1c1cc(F)ccc1F] | (5-((3R,5S,6R)-5-amino-6-(2,5- difluorophenyl)tetrahydro-2H-pyran-3- yl)hexahydropyrrolo[3,4-b]pyrrol-1(2H)- yl)(cyclopropyl)methanone |
| 167 | 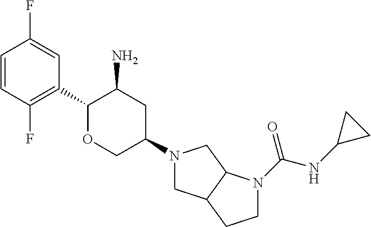 [SMILES: N[C@H]1C[C@@H](N2CC3CCN(C(=O)NC4CC4)C3C2)CO[C@@H]1c1cc(F)ccc1F] | 5-((3R,5S,6R)-5-amino-6-(2,5- difluorophenyl)tetrahydro-2H-pyran-3- yl)-N-cyclopropylhexahydropyrrolo[3,4- b]pyrrole-1(2H)-carboxamide |
| 168 | 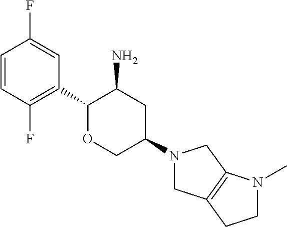 [SMILES: CN1CCC2=C1CN([C@H]1CO[C@H](c3cc(F)ccc3F)[C@@H](N)C1)C2] | (2R,3S,5R)-2-(2,5-difluorophenyl)-5-(1- methyl-2,3-dihydropyrrolo[3,4-b]pyrrol- 5(1H,4H,6H)-yl)tetrahydro-2H-pyran-3- amine |
| 169 | 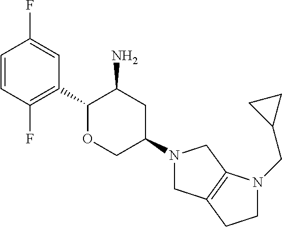 [SMILES: N[C@H]1C[C@@H](N2CC3=C(C2)N(CC2CC2)CC3)CO[C@@H]1c1cc(F)ccc1F] | (2R,3S,5R)-5-(1-(cyclopropylmethyl)- 2,3-dihydropyrrolo[3,4-b]pyrrol- 5(1H,4H,6H)-yl)-2-(2,5- difluorophenyl)tetrahydro-2H-pyran-3- amine |
| 170 | 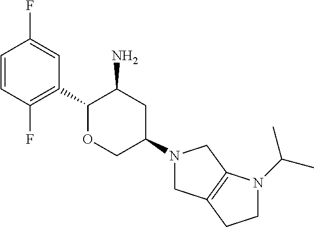 [SMILES: CC(C)N1CCC2=C1CN([C@H]1CO[C@H](c3cc(F)ccc3F)[C@@H](N)C1)C2] | (2R,3S,5R)-2-(2,5-difluorophenyl)-5-(1- isopropyl-2,3-dihydropyrrolo[3,4- b]pyrrol-5(1H,4H,6H)-yl)tetrahydro-2H- pyran-3-amine |
| 171 | 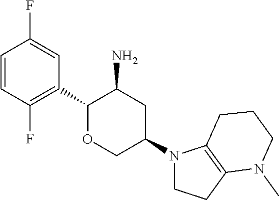 [SMILES: CN1CCCC2=C1CCN2[C@H]1CO[C@H](c2cc(F)ccc2F)[C@@H](N)C1] | (2R,3S,5R)-2-(2,5-difluorophenyl)-5-(4- methyl-2,3,4,5,6,7-hexahydro-1H- pyrrolo[3,2-b]pyridin-1-yl)tetrahydro- 2H-pyran-3-amine |
| 172 | 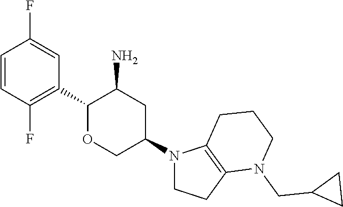 [SMILES: N[C@H]1C[C@@H](N2CCC3=C2CCCN3CC2CC2)CO[C@@H]1c1cc(F)ccc1F] | (2R,3S,5R)-5-(4-(cyclopropylmethyl)- 2,3,4,5,6,7-hexahydro-1H-pyrrolo[3,2- b]pyridin-1-yl)-2-(2,5- difluorophenyl)tetrahydro-2H-pyran-3- amine |
| 173 | 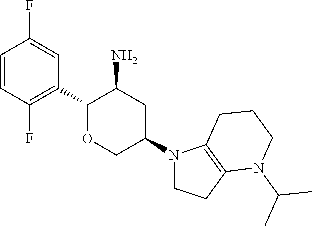 [SMILES: CC(C)N1CCCC2=C1CCN2[C@H]1CO[C@H](c2cc(F)ccc2F)[C@@H](N)C1] | (2R,3S,5R)-2-(2,5-difluorophenyl)-5-(4- isopropyl-2,3,4,5,6,7-hexahydro-1H- pyrrolo[3,2-b]pyridin-1-yl)tetrahydro- 2H-pyran-3-amine |
| 174 | 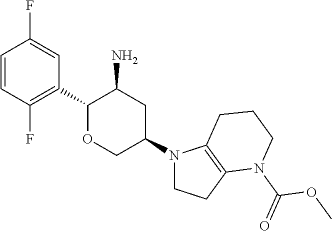 [SMILES: COC(=O)N1CCCC2=C1CCN2[C@H]1CO[C@H](c2cc(F)ccc2F)[C@@H](N)C1] | methyl 1-((3R,5S,6R)-5-amino-6-(2,5- difluorophenyl)tetrahydro-2H-pyran-3- yl)-2,3,6,7-tetrahydro-1H-pyrrolo[3,2- b]pyridine-4(5H)-carboxylate |
| 175 | 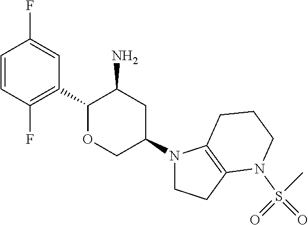 [SMILES: CS(=O)(=O)N1CCCC2=C1CCN2[C@H]1CO[C@H](c2cc(F)ccc2F)[C@@H](N)C1] | (2R,3S,5R)-2-(2,5-difluorophenyl)-5-(4- (methylsulfonyl)-2,3,4,5,6,7-hexahydro- 1H-pyrrolo[3,2-b]pyridin-1- yl)tetrahydro-2H-pyran-3-amine |
| 176 | 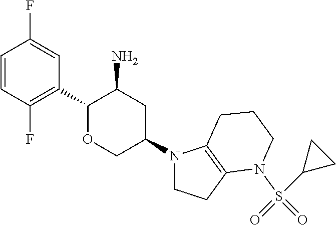 [SMILES: N[C@H]1C[C@@H](N2CCC3=C2CCCN3S(=O)(=O)C2CC2)CO[C@@H]1c1cc(F)ccc1F] | (2R,3S,5R)-5-(4-(cyclopropylsulfonyl)- 2,3,4,5,6,7-hexahydro-1H-pyrrolo[3,2- b]pyridin-1-yl)-2-(2,5- difluorophenyl)tetrahydro-2H-pyran-3- amine |
| 177 | 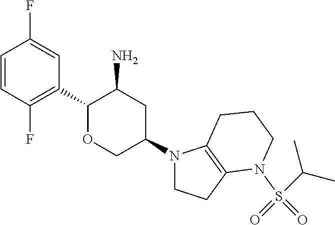 [SMILES: CC(C)S(=O)(=O)N1CCCC2=C1CCN2[C@H]1CO[C@H](c2cc(F)ccc2F)[C@@H](N)C1] | (2R,3S,5R)-2-(2,5-difluorophenyl)-5-(4- (isopropylsulfonyl)-2,3,4,5,6,7- hexahydro-1H-pyrrolo[3,2-b]pyridin-1- yl)tetrahydro-2H-pyran-3-amine |
| 178 | 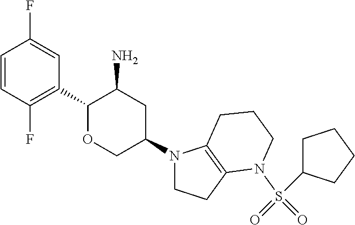 [SMILES: N[C@H]1C[C@@H](N2CCC3=C2CCCN3S(=O)(=O)C2CCCC2)CO[C@@H]1c1cc(F)ccc1F] | (2R,3S,5R)-5-(4-(cyclopentylsulfonyl)- 2,3,4,5,6,7-hexahydro-1H-pyrrolo[3,2- b]pyridin-1-yl)-2-(2,5- difluorophenyl)tetrahydro-2H-pyran-3- amine |
| 179 | 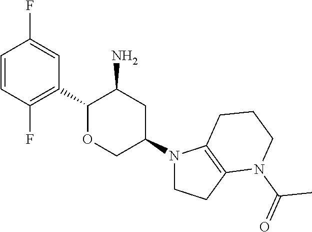 [SMILES: CC(=O)N1CCCC2=C1CCN2[C@H]1CO[C@H](c2cc(F)ccc2F)[C@@H](N)C1] | 1-(1-((3R,5S,6R)-5-amino-6-(2,5- difluorophenyl)tetrahydro-2H-pyran-3- yl)-2,3,6,7-tetrahydro-1H-pyrrolo[3,2- b]pyridin-4(5H)-yl)ethanone |
| 180 | 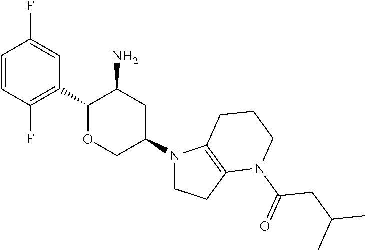 [SMILES: CC(C)CC(=O)N1CCCC2=C1CCN2[C@H]1CO[C@H](c2cc(F)ccc2F)[C@@H](N)C1] | 1-(1-((3R,5S,6R)-5-amino-6-(2,5- difluorophenyl)tetrahydro-2H-pyran-3- yl)-2,3,6,7-tetrahydro-1H-pyrrolo[3,2- b]pyridin-4(5H)-yl)-3-methylbutan-1-one |
| 181 | 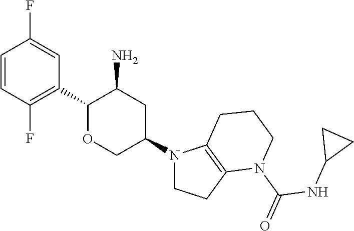 [SMILES: N[C@H]1C[C@@H](N2CCC3=C2CCCN3C(=O)NC2CC2)CO[C@@H]1c1cc(F)ccc1F] | 1-((3R,5S,6R)-5-amino-6-(2,5- difluorophenyl)tetrahydro-2H-pyran-3- yl)-N-cyclopropyl-2,3,6,7-tetrahydro-1H- pyrrolo[3,2-b]pyridine-4(5H)- carboxamide |
| 182 | 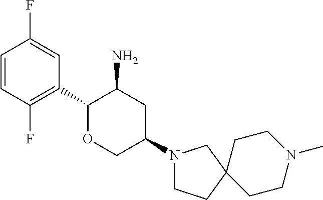 [SMILES: CN1CCC2(CC1)CCN([C@H]1CO[C@H](c3cc(F)ccc3F)[C@@H](N)C1)C2] | (2R,3S,5R)-2-(2,5-difluorophenyl)-5-(8- methyl-2,8-diazaspiro[4.5]decan-2- yl)tetrahydro-2H-pyran-3-amine |
| 183 | 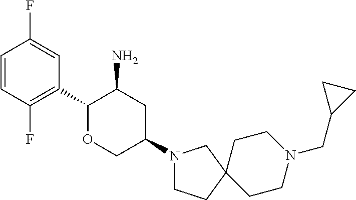 [SMILES: N[C@H]1C[C@@H](N2CCC3(CCN(CC4CC4)CC3)C2)CO[C@@H]1c1cc(F)ccc1F] | (2R,3S,5R)-5-(8-(cyclopropylmethyl)- 2,8-diazaspiro[4.5]decan-2-yl)-2-(2,5- difluorophenyl)tetrahydro-2H-pyran-3- amine |
| 184 | 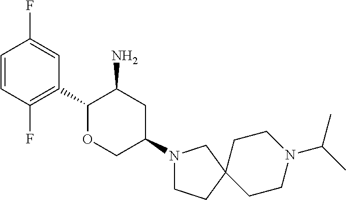 [SMILES: CC(C)N1CCC2(CC1)CCN([C@H]1CO[C@H](c3cc(F)ccc3F)[C@@H](N)C1)C2] | (2R,3S,5R)-2-(2,5-difluorophenyl)-5-(8- isopropyl-2,8-diazaspiro[4.5]decan-2- yl)tetrahydro-2H-pyran-3-amine |
| 185 | 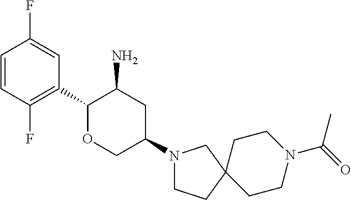 [SMILES: CC(=O)N1CCC2(CC1)CCN([C@H]1CO[C@H](c3cc(F)ccc3F)[C@@H](N)C1)C2] | 1-(2-((3R,5S,6R)-5-amino-6-(2,5- difluorophenyl)tetrahydro-2H-pyran-3- yl)-2,8-diazaspiro[4.5]decan-8- yl)ethanone |
| 186 | 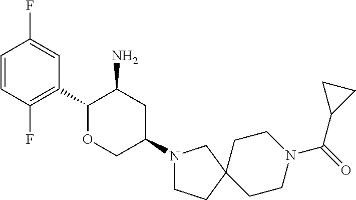 [SMILES: N[C@H]1C[C@@H](N2CCC3(CCN(C(=O)C4CC4)CC3)C2)CO[C@@H]1c1cc(F)ccc1F] | (2-((3R,5S,6R)-5-amino-6-(2,5- difluorophenyl)tetrahydro-2H-pyran-3- yl)-2,8-diazaspiro[4.5]decan-8- yl)(cyclopropyl)methanone |
| 187 | 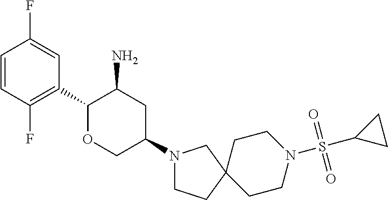 [SMILES: N[C@H]1C[C@@H](N2CCC3(CCN(S(=O)(=O)C4CC4)CC3)C2)CO[C@@H]1c1cc(F)ccc1F] | (2R,3S,5R)-5-(8-(cyclopropylsulfonyl)- 2,8-diazaspiro[4.5]decan-2-yl)-2-(2,5- difluorophenyl)tetrahydro-2H-pyran-3- amine |
| 188 | 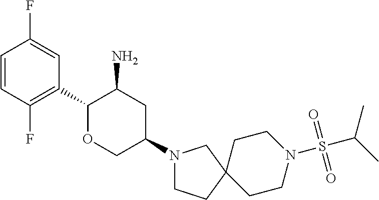 [SMILES: CC(C)S(=O)(=O)N1CCC2(CCN([C@H]3CO[C@H](c4cc(F)ccc4F)[C@@H](N)C3)C2)CC1] | (2R,3S,5R)-2-(2,5-difluorophenyl)-5-(8- (isopropylsulfonyl)-2,8- diazaspiro[4.5]decan-2-yl)tetrahydro-2H- pyran-3-amine |
| 189 | 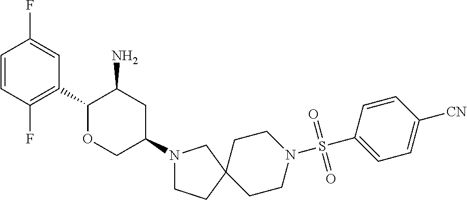 [SMILES: N#Cc1ccc(S(=O)(=O)N2CCC3(CCN([C@H]4CO[C@H](c5cc(F)ccc5F)[C@@H](N)C4)C3)CC2)cc1] | 4-((2-((3R,5S,6R)-5-amino-6-(2,5- difluorophenyl)tetrahydro-2H-pyran-3- yl)-2,8-diazaspiro[4.5]decan-8- yl)sulfonyl)benzonitrile |
| 190 | 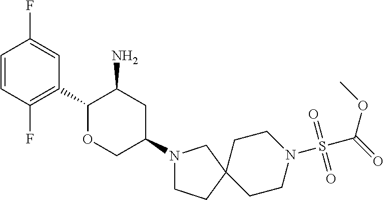 [SMILES: COC(=O)S(=O)(=O)N1CCC2(CCN([C@H]3CO[C@H](c4cc(F)ccc4F)[C@@H](N)C3)C2)CC1] | methyl 2-((3R,5S,6R)-5-amino-6-(2,5- difluorophenyl)tetrahydro-2H-pyran-3- yl)-2,8-diazaspiro[4.5]decane-8- carboxylate |
| 191 | 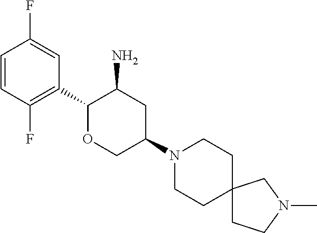 [SMILES: CN1CCC2(CCN([C@H]3CO[C@H](c4cc(F)ccc4F)[C@@H](N)C3)CC2)C1] | (2R,3S,5R)-2-(2,5-difluorophenyl)-5-(2- methyl-2,8-diazaspiro[4.5]decan-8- yI)tetrahydro-2H-pyran-3-amine |
| 192 | 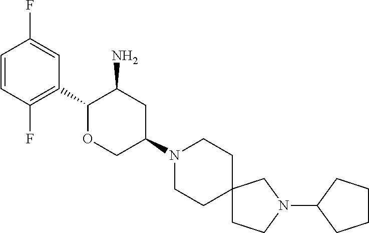 [SMILES: N[C@H]1C[C@@H](N2CCC3(CCN(C4CCCC4)C3)CC2)CO[C@@H]1c1cc(F)ccc1F] | (2R,3S,5R)-5-(2-cyclopentyl-2,8- diazaspiro[4.5]decan-8-yl)-2-(2,5- difluorophenyl)tetrahydro-2H-pyran-3- amine |
| 193 | 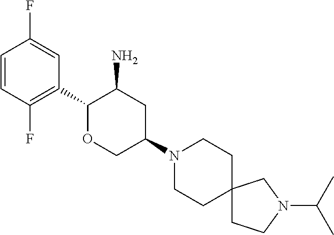 [SMILES: CC(C)N1CCC2(CCN([C@H]3CO[C@H](c4cc(F)ccc4F)[C@@H](N)C3)CC2)C1] | (2R,3S,5R)-2-(2,5-difluorophenyl)-5-(2- isopropyl-2,8-diazaspiro[4.5]decan-8- yl)tetrahydro-2H-pyran-3-amine |
| 194 | 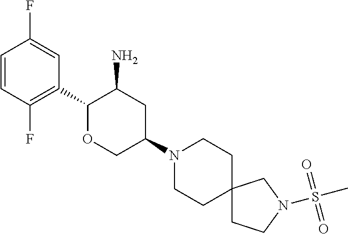 [SMILES: CS(=O)(=O)N1CCC2(CCN([C@H]3CO[C@H](c4cc(F)ccc4F)[C@@H](N)C3)CC2)C1] | (2R,3S,5R)-2-(2,5-difluorophenyl)-5-(2- (methylsulfonyl)-2,8- diazaspiro[4.5]decan-8-yl)tetrahydro-2H- pyran-3-amine |
| 195 | 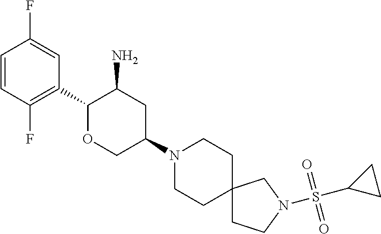 [SMILES: N[C@H]1C[C@@H](N2CCC3(CC2)CCN(S(=O)(=O)C2CC2)C3)CO[C@@H]1c1cc(F)ccc1F] | (2R,3S,5R)-5-(2-(cyclopropylsulfonyl)- 2,8-diazaspiro[4.5]decan-8-yl)-2-(2,5- difluorophenyl)tetrahydro-2H-pyran-3- amine |
| 196 | 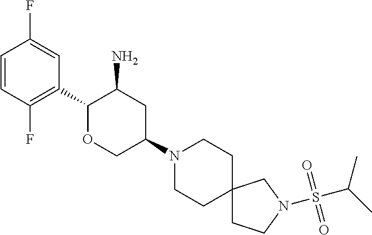 [SMILES: CC(C)S(=O)(=O)N1CCC2(CCN([C@H]3CO[C@H](c4cc(F)ccc4F)[C@@H](N)C3)CC2)C1] | (2R,3S,5R)-2-(2,5-difluorophenyl)-5-(2- (isopropylsulfonyl)-2,8- diazaspiro[4.5]decan-8-yl)tetrahydro-2H- pyran-3-amine |
| 197 | 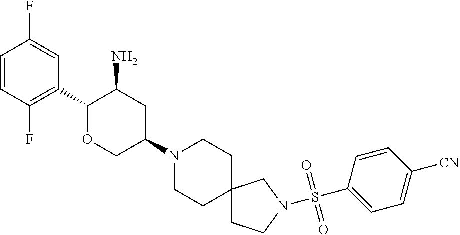 [SMILES: N#Cc1ccc(S(=O)(=O)N2CCC3(CCN([C@H]4CO[C@H](c5cc(F)ccc5F)[C@@H](N)C4)CC3)C2)cc1] | 4-((8-((3R,5S,6R)-5-amino-6-(2,5- difluorophenyl)tetrahydro-2H-pyran-3- yl)-2,8-diazaspiro[4.5]decan-2- yl)sulfonyl)benzonitrile |
| 198 | 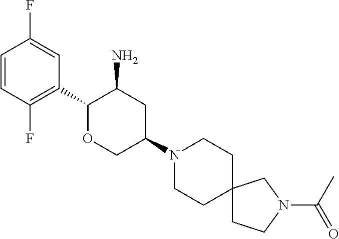 [SMILES: CC(=O)N1CCC2(CCN([C@H]3CO[C@H](c4cc(F)ccc4F)[C@@H](N)C3)CC2)C1] | 1-(8-((3R,5S,6R)-5-amino-6-(2,5- difluorophenyl)tetrahydro-2H-pyran-3- yl)-2,8-diazaspiro[4.5]decan-2- yI)ethanone |
| 199 | 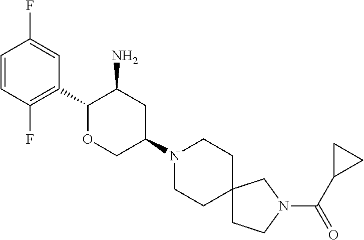 [SMILES: N[C@H]1C[C@@H](N2CCC3(CCN(C(=O)C4CC4)C3)CC2)CO[C@@H]1c1cc(F)ccc1F] | (8-((3R,5S,6R)-5-amino-6-(2,5- difluorophenyl)tetrahydro-2H-pyran-3- yl)-2,8-diazaspiro[4.5]decan-2- yl)(cyclopropyl)methanone |
| 200 | 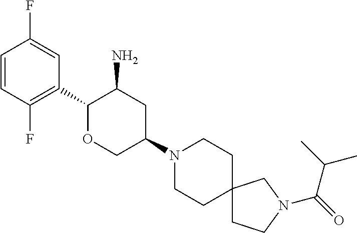 [SMILES: CC(C)C(=O)N1CCC2(CCN([C@H]3CO[C@H](c4cc(F)ccc4F)[C@@H](N)C3)CC2)C1] | 1-(8-((3R,5S,6R)-5-amino-6-(2,5- difluorophenyl)tetrahydro-2H-pyran-3- yl)-2,8-diazaspiro[4.5]decan-2-yl)-2- methylpropan-1-one |
| 201 | 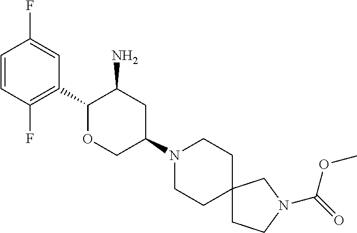 [SMILES: COC(=O)N1CCC2(CCN([C@H]3CO[C@H](c4cc(F)ccc4F)[C@@H](N)C3)CC2)C1] | methyl 8-((3R,5S,6R)-5-amino-6-(2,5- difluorophenyl)tetrahydro-2H-pyran-3- yl)-2,8-diazaspiro[4.5]decane-2- carboxylate |
| 202 | 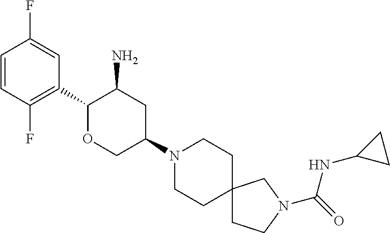 [SMILES: N[C@H]1C[C@@H](N2CCC3(CCN(C(=O)NC4CC4)C3)CC2)CO[C@@H]1c1cc(F)ccc1F] | 8-((3R,5S,6R)-5-amino-6-(2,5- difluorophenyl)tetrahydro-2H-pyran-3- yl)-N-cyclopropyl-2,8- diazaspiro[4.5]decane-2-carboxamide |
| 203 | 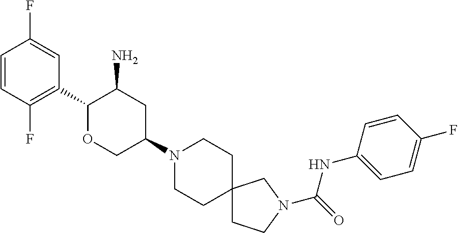 [SMILES: N[C@H]1C[C@@H](N2CCC3(CCN(C(=O)Nc4ccc(F)cc4)C3)CC2)CO[C@@H]1c1cc(F)ccc1F] | 8-((3R,5S,6R)-5-amino-6-(2,5- difluorophenyl)tetrahydro-2H-pyran-3- yl)-N-(4-fluorophenyl)-2,8- diazaspiro[4.5]decane-2-carboxamide |
| 204 | 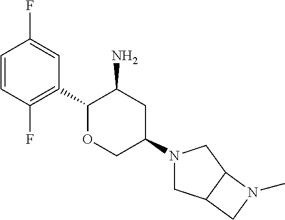 [SMILES: CN1CC2CN([C@H]3CO[C@H](c4cc(F)ccc4F)[C@@H](N)C3)CC21] | (2R,3S,5R)-2-(2,5-difluorophenyl)-5-(6- methyl-3,6-diazabicyclo[3.2.0]heptan-3- yl)tetrahydro-2H-pyran-3-amine |
| 205 | 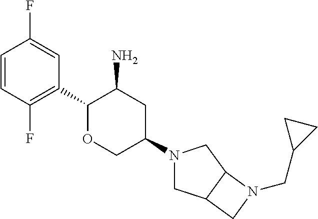 [SMILES: N[C@H]1C[C@@H](N2CC3CN(CC4CC4)C3C2)CO[C@@H]1c1cc(F)ccc1F] | (2R,3S,5R)-5-(6-(cyclopropylmethyl)- 3,6-diazabicyclo[3.2.0]heptan-3-yl)-2- (2,5-difluorophenyl)tetrahydro-2H- pyran-3-amine |
| 206 | 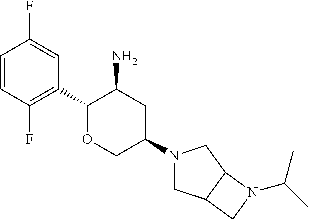 [SMILES: CC(C)N1CC2CN([C@H]3CO[C@H](c4cc(F)ccc4F)[C@@H](N)C3)CC21] | (2R,3S,5R)-2-(2,5-difluorophenyl)-5-(6- isopropyl-3,6-diazabicylo[3.2.0]heptan- 3-yl)tetrahydro-2H-pyran-3-amine |
| 207 | 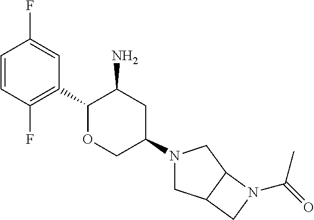 [SMILES: CC(=O)N1CC2CN([C@H]3CO[C@H](c4cc(F)ccc4F)[C@@H](N)C3)CC21] | 1-(3-((3R,5S,6R)-5-amino-6-(2,5- difluorophenyl)tetrahydro-2H-pyran-3- yl)-3,6-diazabicyclo[3.2.0]heptan-6- yl)ethanone |
| 208 | 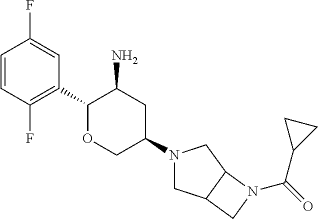 [SMILES: N[C@H]1C[C@@H](N2CC3CN(C(=O)C4CC4)C3C2)CO[C@@H]1c1cc(F)ccc1F] | (3-((3R,5S,6R)-5-amino-6-(2,5- difluorophenyl)tetrahydro-2H-pyran-3 yl)-3,6-diazabicyclo[3.2.0]heptan-6- yl)(cyclopropyl)methanone |
| 209 | 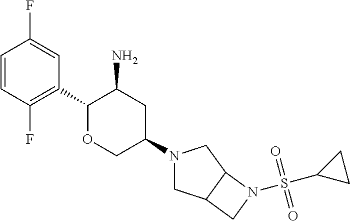 [SMILES: N[C@H]1C[C@@H](N2CC3CN(S(=O)(=O)C4CC4)C3C2)CO[C@@H]1c1cc(F)ccc1F] | (2R,3S,5R)-5-(6-(cyclopropylsulfonyl)- 3,6-diazabicyclo[3.2.0]heptan-3-yl)-2- (2,5-difluorophenyl)tetrahydro-2H- pyran-3-amine |
| 210 | 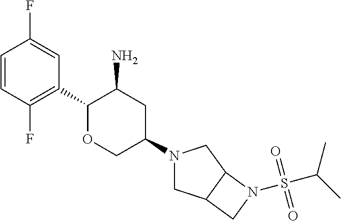 [SMILES: CC(C)S(=O)(=O)N1CC2CN([C@H]3CO[C@H](c4cc(F)ccc4F)[C@@H](N)C3)CC21] | (2R,3S,5R)-2-(2,5-difluorophenyl)-5-(6- (isopropylsulfonyl)-3,6- diazabicyclo[3.2.0]heptan-3- yl)tetrahydro-2H-pyran-3-amine |
| 211 | 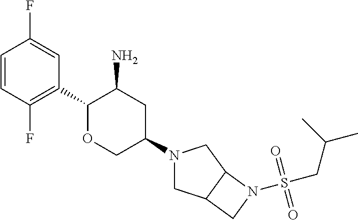 [SMILES: CC(C)CS(=O)(=O)N1CC2CN([C@H]3CO[C@H](c4cc(F)ccc4F)[C@@H](N)C3)CC21] | (2R,3S,5R)-2-(2,5-difluorophenyl)-5-(6- (isobutylsulfonyl)-3,6- diazabicyclo[3.2.0]heptan-3- yl)tetrahydro-2H-pyran-3-amine |
| 212 | 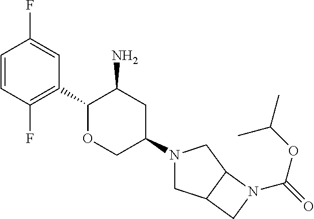 [SMILES: CC(C)OC(=O)N1CC2CN([C@H]3CO[C@H](c4cc(F)ccc4F)[C@@H](N)C3)CC21] | isopropyl 3-((3R,5S,6R)-5-amino-6-(2,5- difluorophenyl)tetrahydro-2H-pyran-3- yl)-3,6-diazabicyclo[3.2.0]heptane-6- carboxylate |
| 213 | 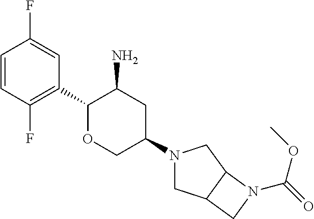 [SMILES: COC(=O)N1CC2CN([C@H]3CO[C@H](c4cc(F)ccc4F)[C@@H](N)C3)CC21] | methyl 3-((3R,5S,6R)-5-amino-6-(2,5- difluorophenyl)tetrahydro-2H-pyran-3- yl)-3,6-diazabicyclo[3.2.0]heptane-6- carboxylate |
| 214 | 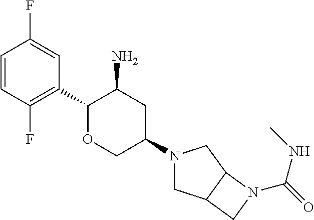 [SMILES: CNC(=O)N1CC2CN([C@H]3CO[C@H](c4cc(F)ccc4F)[C@@H](N)C3)CC21] | 3-((3R,5S,6R)-5-amino-6-(2,5- difluorophenyl)tetrahydro-2H-pyran-3- yl)-N-methyl-3,6- diazabicyclo[3.2.0]heptane-6- carboxamide |
| 215 | 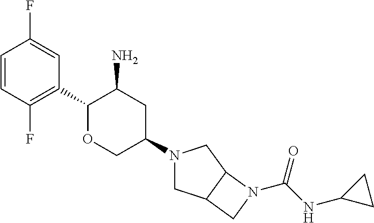 [SMILES: N[C@H]1C[C@@H](N2CC3CN(C(=O)NC4CC4)C3C2)CO[C@@H]1c1cc(F)ccc1F] | 3-((3R,5S,6R)-5-amino-6-(2,5- difluorophenyl)tetrahydro-2H-pyran-3- yl)-N-cyclopropyl-3,6- diazabicyclo[3.2.0]heptane-6- carboxamide |
| 216 | 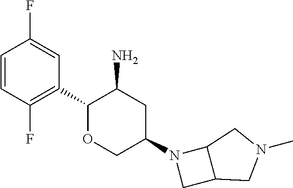 [SMILES: CN1CC2CN([C@H]3CO[C@H](c4cc(F)ccc4F)[C@@H](N)C3)C2C1] | (2R,3S,5R)-2-(2,5-difluorophenyl)-5-(3- methyl-3,6-diazabicyclo[3.2.0]heptan-6- yl)tetrahydro-2H-pyran-3-amine |
| 217 | 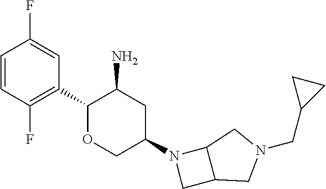 [SMILES: N[C@H]1C[C@@H](N2CC3CN(CC4CC4)CC32)CO[C@@H]1c1cc(F)ccc1F] | (2R,3S,5R)-5-(3-(cyclopropylmethyl)- 3,6-diazabicyclo[3.2.0]heptan-6-yl)-2- (2,5-difluorophenyl)tetrahydro-2H- pyran-3-amine |
| 218 | 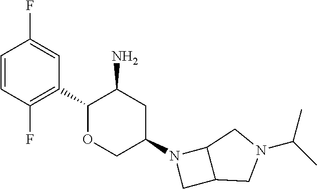 [SMILES: CC(C)N1CC2CN([C@H]3CO[C@H](c4cc(F)ccc4F)[C@@H](N)C3)C2C1] | (2R,3S,5R)-2-(2,5-difluorophenyl)-5-(3- isopropyl-3,6-diazabicyclo[3.2.0]heptan- 6-yl)tetrahydro-2H-pyran-3-amine |
| 219 | 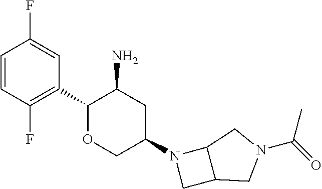 [SMILES: CC(=O)N1CC2CN([C@H]3CO[C@H](c4cc(F)ccc4F)[C@@H](N)C3)C2C1] | 1-(6-((3R,5S,6R)-5-amino-6-(2,5- difluorophenyl)tetrahydro-2H-pyran-3- yl)-3,6-diazabicyclo[3.2.0]heptan-3- yl)ethanone |
| 220 | 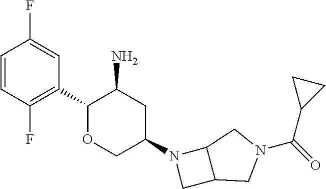 [SMILES: N[C@H]1C[C@@H](N2CC3CN(C(=O)C4CC4)CC32)CO[C@@H]1c1cc(F)ccc1F] | (6-((3R,5S,6R)-5-amino-6-(2,5- difluorophenyl)tetrahydro-2H-pyran-3- yl)-3,6-diazabicyclo[3.2.0]heptan-3- yl)(cyclopropyl)methanone |
| 221 | 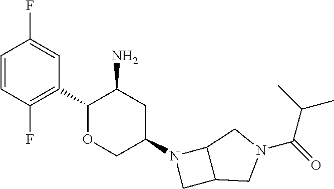 [SMILES: CC(C)C(=O)N1CC2CN([C@H]3CO[C@H](c4cc(F)ccc4F)[C@@H](N)C3)C2C1] | 1-(6-((3R,5S,6R)-5-amino-6-(2,5- difluorophenyl)tetrahydro-2H-pyran-3- yl)-3,6-diazabicyclo[3.2.0]heptan-3-yl)- 2- methylpropan-1-one |
| 222 | 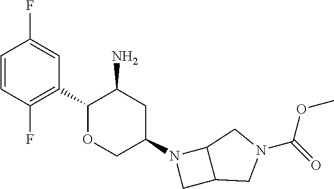 [SMILES: COC(=O)N1CC2CN([C@H]3CO[C@H](c4cc(F)ccc4F)[C@@H](N)C3)C2C1] | methyl 6-((3R,5S,6R)-5-amino-6-(2,5- difluorophenyl)tetrahydro-2H-pyran-3- yl)-3,6-diazabicyclo[3.2.0]heptane-3- carboxylate |
| 223 | 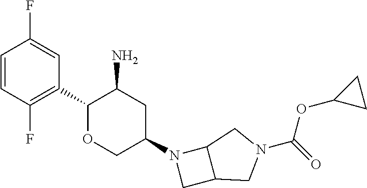 [SMILES: N[C@H]1C[C@@H](N2CC3CN(C(=O)OC4CC4)CC32)CO[C@@H]1c1cc(F)ccc1F] | cyclopropyl 6-((3R,5S,6R)-5-amino-6- (2,5-difluorophenyl)tetrahydro-2H- pyran-3-yl)-3,6- diazabicyclo[3.2.0]heptane-3-carboxylate |
| 224 | 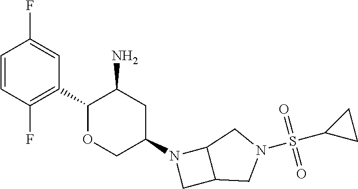 [SMILES: N[C@H]1C[C@@H](N2CC3CN(S(=O)(=O)C4CC4)CC32)CO[C@@H]1c1cc(F)ccc1F] | (2R,3S,5R)-5-(3-(cyclopropylsulfonyl)- 3,6-diazabicyclo[3.2.0]heptan-6-yl)-2- (2,5-difluorophenyl)tetrahydro-2H- pyran-3-amine |
| 225 | 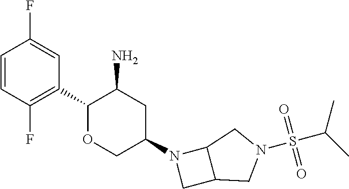 [SMILES: CC(C)S(=O)(=O)N1CC2CN([C@H]3CO[C@H](c4cc(F)ccc4F)[C@@H](N)C3)C2C1] | (2R,3S,5R)-2-(2,5-difluorophenyl)-5-(3- (isopropylsulfonyl)-3,6- diazabicyclo[3.2.0]heptan-6- yl)tetrahydro-2H-pyran-3-amine |
| 226 | 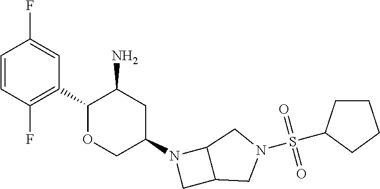 [SMILES: N[C@H]1C[C@@H](N2CC3CN(S(=O)(=O)C4CCCC4)CC32)CO[C@@H]1c1cc(F)ccc1F] | (2R,3S,5R)-5-(3-(cyclopentylsulfonyl)- 3,6-diazabicyclo[3.2.0]heptan-6-yl)-2- (2,5-difluorophenyl)tetrahydro-2H- pyran-3-amine |
| 227 | 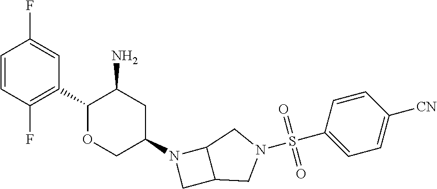 [SMILES: N#Cc1ccc(S(=O)(=O)N2CC3CN([C@H]4CO[C@H](c5cc(F)ccc5F)[C@@H](N)C4)C3C2)cc1] | 4-((6-((3R,5S,6R)-5-amino-6-(2,5- difluorophenyl)tetrahydro-2H-pyran-3- yl)-3,6-diazabicyclo[3.2.0]heptan-3- yl)sulfonyl)benzonitrile |
| 228 | 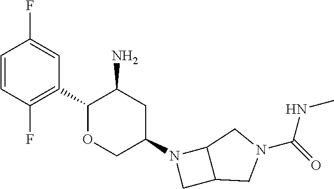 [SMILES: CNC(=O)N1CC2CN([C@H]3CO[C@H](c4cc(F)ccc4F)[C@@H](N)C3)C2C1] | 6-((3R,5S,6R)-5-amino-6-(2,5- difluorophenyl)tetrahydro-2H-pyran-3- yl)-N-methyl-3,6- diazabicyclo[3.2.0]heptane-3- carboxamide |
| 229 | 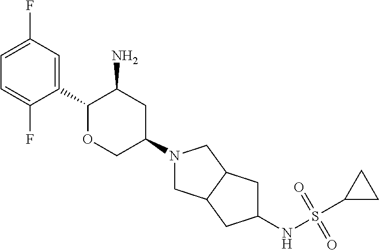 [SMILES: N[C@H]1C[C@@H](N2CC3CC(NS(=O)(=O)C4CC4)CC3C2)CO[C@@H]1c1cc(F)ccc1F] | N-(2-((3R,5S,6R)-5-amino-6-(2,5- difluorophenyl)tetrahydro-2H-pyran-3- yl)octahydrocyclopenta[c]pyrrol-5- yl)cyclopropanesulfonamide |
| 230 | 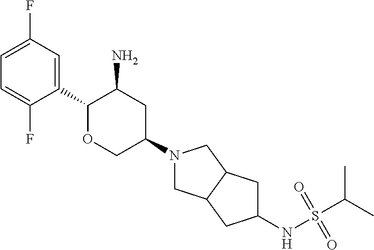 [SMILES: CC(C)S(=O)(=O)NC1CC2CN([C@H]3CO[C@H](c4cc(F)ccc4F)[C@@H](N)C3)CC2C1] | N-(2-((3R,5S,6R)-5-amino-6-(2,5- difluorophenyl)tetrahydro-2H-pyran-3- yl)octahydrocyclopenta[c]pyrrol-5- yl)propane-2-sulfonamide |
| 231 | 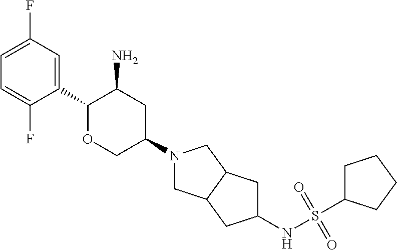 [SMILES: N[C@H]1C[C@@H](N2CC3CC(NS(=O)(=O)C4CCCC4)CC3C2)CO[C@@H]1c1cc(F)ccc1F] | N-(2-((3R,5S,6R)-5-amino-6-(2,5- difluorophenyl)tetrahydro-2H-pyran-3- yl)octahydrocyclopenta[c]pyrrol-5- yl)cyclopentanesulfonamide |
| 232 | 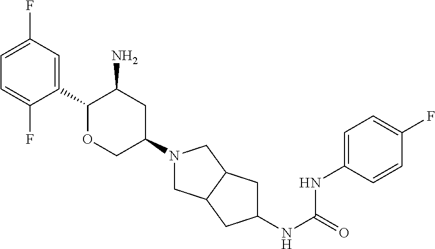 [SMILES: N[C@H]1C[C@@H](N2CC3CC(NC(=O)Nc4ccc(F)cc4)CC3C2)CO[C@@H]1c1cc(F)ccc1F] | 1-(2-((3R,5S,6R)-5-amino-6-(2,5- difluorophenyl) tetrahydro-2H-pyran-3- yl)octahydrocyclopenta[c] pyrrol-5-yl)-3- (4-fluorophenyl)urea |
| 233 | 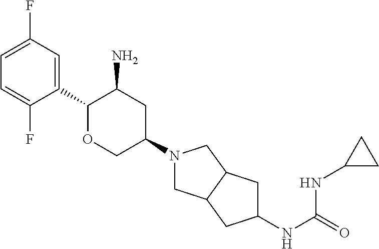 [SMILES: N[C@H]1C[C@@H](N2CC3CC(NC(=O)NC4CC4)CC3C2)CO[C@@H]1c1cc(F)ccc1F] | 1-(2-((3R,5S,6R)-5-amino-6-(2,5- difluorophenyl)tetrahydro-2H-pyran-3- yl)octahydrocyclopenta[c]pyrrol-5-yl)-3- cyclopropylurea |
| 234 | 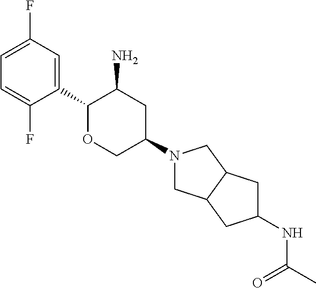 [SMILES: CC(=O)NC1CC2CN([C@H]3CO[C@H](c4cc(F)ccc4F)[C@@H](N)C3)CC2C1] | N-(2-((3R,5S,6R)-5-amino-6-(2,5- difluorophenyl)tetrahydro-2H-pyran-3- yl)octahydrocyclopenta[c]pyrrol-5- yl)acetamide |
| 235 | 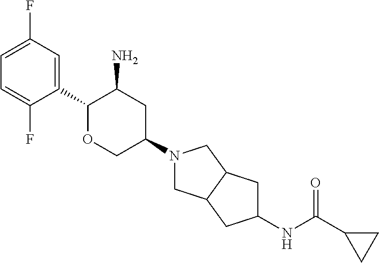 [SMILES: N[C@H]1C[C@@H](N2CC3CC(NC(=O)C4CC4)CC3C2)CO[C@@H]1c1cc(F)ccc1F] | N-(2-((3R,5S,6R)-5-amino-6-(2,5- difluorophenyl) tetrahydro-2H-pyran-3- yl)octahydrocyclopenta[c] pyrrol-5- yl)cyclopropanecarboxamide |
| 236 | 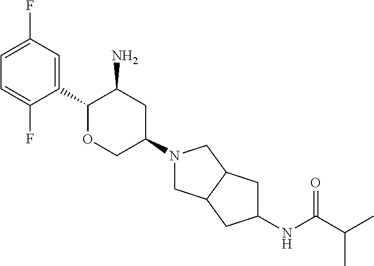 [SMILES: CC(C)C(=O)NC1CC2CN([C@H]3CO[C@H](c4cc(F)ccc4F)[C@@H](N)C3)CC2C1] | N-(2-((3R,5S,6R)-5-amino-6-(2,5- difluorophenyl)tetrahydro-2H-pyran-3- yl)octahydrocyclopenta[c]pyrrol-5- yl)isobutyramide |
| 237 | 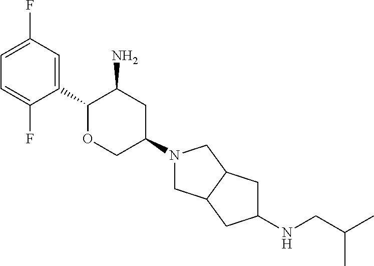 [SMILES: CC(C)CNC1CC2CN([C@H]3CO[C@H](c4cc(F)ccc4F)[C@@H](N)C3)CC2C1] | 2-((3R,5S,6R)-5-amino-6-(2,5- difluorophenyl)tetrahydro-2H-pyran-3- yl)-N- isobutyloctahydrocyclopenta[c]pyrrol-5- amine |
| 238 | 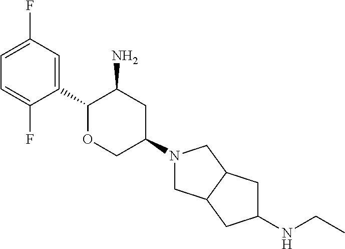 [SMILES: CCNC1CC2CN([C@H]3CO[C@H](c4cc(F)ccc4F)[C@@H](N)C3)CC2C1] | 2-((3R,5S,6R)-5-amino-6-(2,5- difluorophenyl)tetrahydro-2H-pyran-3- yl)-N-ethyloctahydrocyclopenta[c]pyrrol- 5-amine |
| 239 | 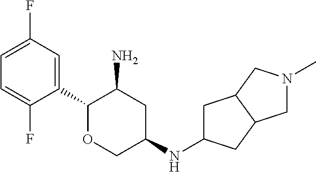 [SMILES: CN1CC2CC(N[C@H]3CO[C@H](c4cc(F)ccc4F)[C@@H](N)C3)CC2C1] | (2R,3S,5R)-2-(2,5-difluorophenyl)-N5- (2-methyloctahydrocyclopenta[c]pyrrol- 5-yl)tetrahydro-2H-pyran-3,5-diamine |
| 240 | 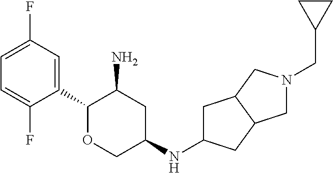 [SMILES: N[C@H]1C[C@@H](NC2CC3CN(CC4CC4)CC3C2)CO[C@@H]1c1cc(F)ccc1F] | (2R,3S,5R)-N5-(2- (cyclopropylmethyl)octahydrocyclopenta[c] pyrrol-5-yl)-2-(2,5- difluorophenyl)tetrahydro-2H-pyran-3,5- diamine |
| 241 | 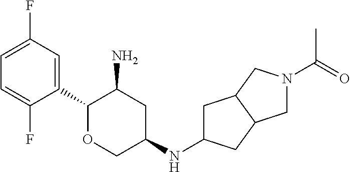 [SMILES: CC(=O)N1CC2CC(N[C@H]3CO[C@H](c4cc(F)ccc4F)[C@@H](N)C3)CC2C1] | 1-(5-(((3R,5S,6R)-5-amino-6-(2,5- difluorophenyl)tetrahydro-2H-pyran-3- yl)amino)hexahydrocyclopenta[c]pyrrol- 2(1H)-yl)ethanone |
| 242 | 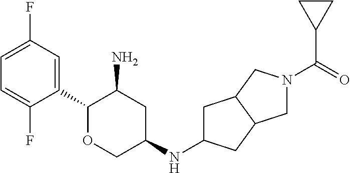 [SMILES: N[C@H]1C[C@@H](NC2CC3CN(C(=O)C4CC4)CC3C2)CO[C@@H]1c1cc(F)ccc1F] | (5-(((3R,5S,6R)-5-amino-6-(2,5- difluorophenyl)tetrahydro-2H-pyran-3- yl)amino)hexahydrocyclopenta[c]pyrrol- 2(1H)-yl)(cyclopropyl)methanone |
| 243 | 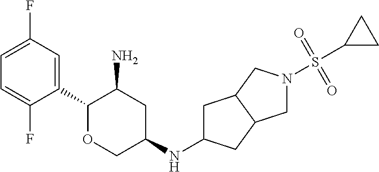 [SMILES: N[C@H]1C[C@@H](NC2CC3CN(S(=O)(=O)C4CC4)CC3C2)CO[C@@H]1c1cc(F)ccc1F] | (2R,3S,5R)-N5-(2-(cyclopropylsulfonyl) octahydrocyclopenta[c] pyrrol-5-yl)-2-(2,5- difluorophenyl)tetrahydro-2H-pyran-3,5- diamine |
| 244 | 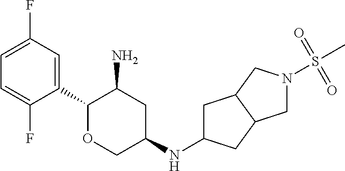 [SMILES: CS(=O)(=O)N1CC2CC(N[C@H]3CO[C@H](c4cc(F)ccc4F)[C@@H](N)C3)CC2C1] | (2R,3S,5R)-2-(2,5-difluorophenyl)-N5- (2- (methylsulfonyl)octahydrocyclopenta[c] pyrrol-5-yl)tetrahydro-2H-pyran-3,5- diamine |
| 245 | 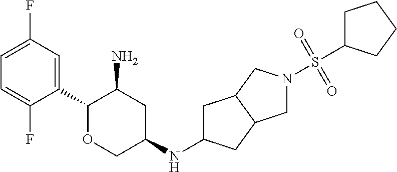 [SMILES: N[C@H]1C[C@@H](NC2CC3CN(S(=O)(=O)C4CCCC4)CC3C2)CO[C@@H]1c1cc(F)ccc1F] | (2R,3S,5R)-N5-(2-(cyclopentylsulfonyl) octahydrocyclopenta[c] pyrrol-5-yl)-2-(2,5- difluorophenyl)tetrahydro-2H-pyran-3,5- diamine |
| 246 | 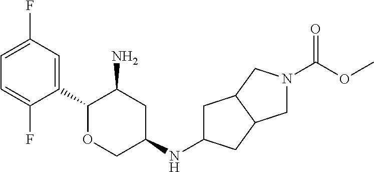 [SMILES: COC(=O)N1CC2CC(N[C@H]3CO[C@H](c4cc(F)ccc4F)[C@@H](N)C3)CC2C1] | methyl 5-(((3R,5S,6R)-5-amino-6-(2,5- difluorophenyl)tetrahydro-2H-pyran-3- yl)amino)hexahydrocyclopenta[c]pyrrole- 2(1H)-carboxylate |
| 247 | 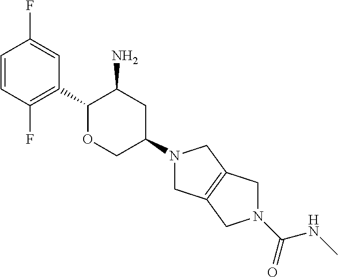 [SMILES: CNC(=O)N1CC2=C(C1)CN([C@H]1CO[C@H](c3cc(F)ccc3F)[C@@H](N)C1)C2] | 5-((3R,5S,6R)-5-amino-6-(2,5- difluorophenyl)tetrahydro-2H-pyran-3- yl)-N-methyl-3,4,5,6- tetrahydropyrrolo[3,4-c]pyrrole-2(1H)- carboxamide |
| 248 | 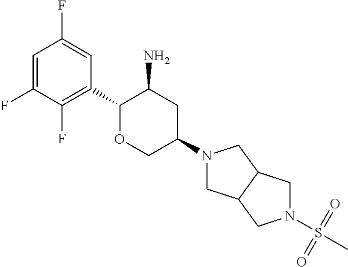 [SMILES: CS(=O)(=O)N1CC2CN([C@H]3CO[C@H](c4cc(F)cc(F)c4F)[C@@H](N)C3)CC2C1] | (2R,3S,5R)-5-(5- (methylsulfonyl)hexahydropyrrolo[3,4-c] pyrrol-2(1H)-yl)-2-(2,3,5- trifluorophenyl)tetrahydro-2H-pyran-3- amine |
| 249 | 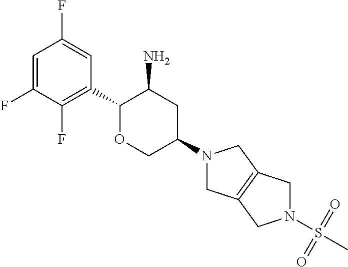 [SMILES: CS(=O)(=O)N1CC2=C(CN([C@H]3CO[C@H](c4cc(F)cc(F)c4F)[C@@H](N)C3)C2)C1] | (2R,3S,5R)-5-(5-(methylsulfonyl)-5,6- dihydropyrrolo[3,4-c]pyrrol- 2(1H,3H,4H)-yl)-2-(2,3,5- trifluorophenyl)tetrahydro-2H-pyran-3- amine |
| 250 | 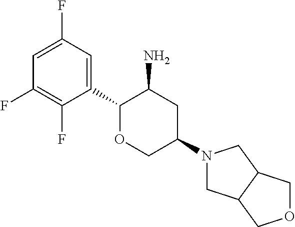 [SMILES: N[C@H]1C[C@@H](N2CC3COCC3C2)CO[C@@H]1c1cc(F)cc(F)c1F] | (2R,3S,5R)-5-(tetrahydro-1H-furo[3,4-c] pyrrol-5(3H)-yl)-2-(2,3,5- trifluorophenyl)tetrahydro-2H-pyran-3- amine |
| 251 | 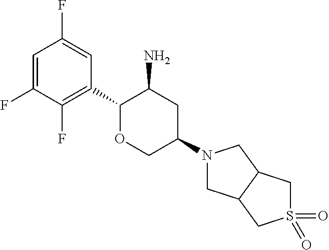 [SMILES: N[C@H]1C[C@@H](N2CC3CS(=O)(=O)CC3C2)CO[C@@H]1c1cc(F)cc(F)c1F] | 5-((3R,5S,6R)-5-amino-6-(2,3,5- trifluorophenyl)tetrahydro-2H-pyran-3- yl)hexahydro-1H-thieno[3,4-c]pyrrole 2,2-dioxide |
| 252 | 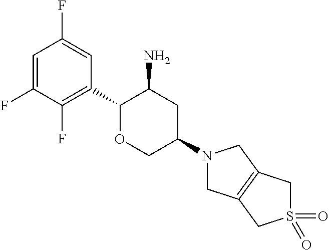 [SMILES: N[C@H]1C[C@@H](N2CC3=C(C2)CS(=O)(=O)C3)CO[C@@H]1c1cc(F)cc(F)c1F] | 5-((3R,5S,6R)-5-amino-6-(2,3,5- trifluorophenyl)tetrahydro-2H-pyran-3- yl)-3,4,5,6-tetrahydro-1H-thieno[3,4- c]pyrrole 2,2-dioxide |
| 253 | 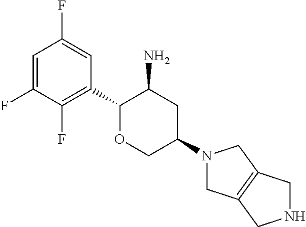 [SMILES: N[C@H]1C[C@@H](N2CC3=C(CNC3)C2)CO[C@@H]1c1cc(F)cc(F)c1F] | (2R,3S,5R)-5-(5,6-dihydropyrrolo[3,4-c] pyrrol-2(1H,3H,4H)-yl)-2-(2,3,5- trifluorophenyl)tetrahydro-2H-pyran-3- amine |
| 254 | 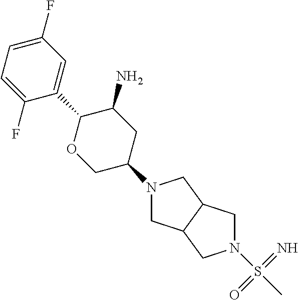 [SMILES: CS(=N)(=O)N1CC2CN([C@H]3CO[C@H](c4cc(F)ccc4F)[C@@H](N)C3)CC2C1] | (2R,3S,5R)-2-(2,5-difluorophenyl)-5-(5- (S-methylsulfonimidoyl) hexahydropyrrolo[3,4-c] pyrrol-2(1H)-yl)tetrahydro-2H- pyran-3-amine |
| 255 | 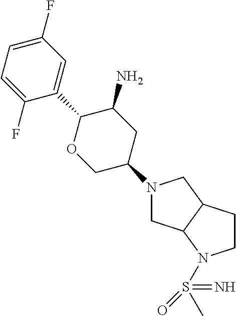 [SMILES: CS(=N)(=O)N1CCC2CN([C@H]3CO[C@H](c4cc(F)ccc4F)[C@@H](N)C3)CC21] | (2R,3S,5R)-2-(2,5-difluorophenyl)-5-(1- (S-methylsulfonimidoyl) hexahydropyrrolo[3,4-b] pyrrol-5(1H)-yl)tetrahydro-2H- pyran-3-amine |
| 256 | 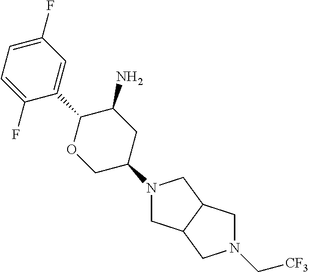 [SMILES: N[C@H]1C[C@@H](N2CC3CN(CC(F)(F)F)CC3C2)CO[C@@H]1c1cc(F)ccc1F] | (2R,3S,5R)-2-(2,5-difluorophenyl)-5-(5- (2,2,2- trifluoroethyl)hexahydropyrrolo[3,4-c] pyrrol-2(1H)-yl)tetrahydro-2H-pyran- 3-amine |
| 257 | 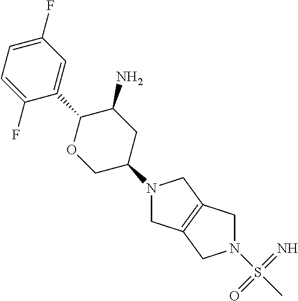 [SMILES: CS(=N)(=O)N1CC2=C(CN([C@H]3CO[C@H](c4cc(F)ccc4F)[C@@H](N)C3)C2)C1] | (2R,3S,5R)-2-(2,5-difluorophenyl)-5-(5- (S-methylsulfonimidoyl)-5,6- dihydropyrrolo[3,4-c]pyrrol- 2(1H,3H,4H)-yl)tetrahydro-2H-pyran-3- amine |

### Testing of Compounds of the Invention

In Vitro DPP-IV Inhibitory Activity Using Enzymatic Assay:

In vitro enzyme (DPP-IV) inhibitory activity was determined using fluorescence-based assay (Anal. Biochem., 200, 352, 1992). The Gly-Pro-AMC was used as a substrate (which is cleaved by the enzymes to release the fluorescent AMC) and soluble human proteins (DPP-IV enzyme) produced in a baculovirus expression system (Life Technologies) was used as the enzyme source. The H-Gly-Pro-AMC (200 μM) was incubated with DPP-IV enzyme in the presence of various concentrations (30 & 100 nM) of test compounds. Reaction was carried out at pH 7.8 (HEPES buffer 25 mM containing 1.0% BSA, 140 mM NaCl, 16 mM MgCl2, 2.8% DMSO) in a total volume of 100 μl at 25° C. for 30 min., in the dark. Reaction was terminated with acetic acid (25 μl of 25% solution). Activity (fluorescence) was measured using Spectra Max fluorometer (Molecular Devices, Sunnyvale Calif.) by exciting at 380 nm and emission at 460 nm. In-vitro DPP-IV inhibitory activity of some of the representative compounds are listed in Table-3.

TABLE 3
In vitro DPP-IV inhibitory activity of test compounds

| | % In-vitro DPPIV Inhibition |
|---|---|
| Compounds | IC₅₀ |
| 1 | ++ |
| 2 | +++ |
| 3 | + |
| 4 | + |
| 5 | + |
| 6 | + |
| 7 | +++ |
| 8 | +++ |
| 9 | +++ |
| 10 | + |
| 11 | + |
| 12 | + |
| 13 | +++ |
| 14 | + |
| 15 | + |
| 16 | + |
| 17 | +++ |
| 18 | + |
| 19 | + |
| 20 | + |
| 21 | + |
| 22 | ++ |
| 23 | ++ |
| 24 | ++ |
| 25 | ++ |
| 26 | ++ |
| 27 | +++ |
| 28 | +++ |
| 29 | ++ |
| 30 | +++ |
| 31 | +++ |
| 32 | +++ |
| 33 | ++ |
| 34 | +++ |
| 35 | ++ |
| 36 | +++ |
| 37 | ++ |
| 38 | +++ |
| 39 | +++ |
| 40 | ++ |
| 41 | + |
| 42 | ++ |
| 43 | ++ |
| 44 | ++ |
| 45 | +++ |
| 46 | + |
| 47 | + |
| 48 | +++ |
| 49 | +++ |
| 50 | +++ |

+ indicates IC₅₀ <100 nM;
++ indicates IC₃₀ <30 nM and
+++ indicates IC₅₀ <10 nM;
DPP-IV inhibitory activity determined by fluorescence-based assay;
fluorescence measured using Spectra Max fluorometer (Molecular Devices, CA) by exciting at 380 nm and emission at 460 nm.

### In Vivo Efficacy Studies:

a) Demonstration of In Vivo Efficacy (Antihyperglycaemic/Antidiabetic Activity) of Test Compounds in C57BL/6J Mice, Oral Routes of Administration.

### Animals

Acute single dose 120-min time-course experiments were carried out in male C57BL/6J mice, age 8-12 weeks, bred in-house. Animals were housed in groups of 6 animals per cage, for a week, in order to habituate them to vivarium conditions (25±4° C., 60-65% relative humidity, 12: 12 h light: dark cycle, with lights on at 7.30 am). All the animal experiments were carried out according to the internationally valid guidelines following approval by the ‘Zydus Research Center animal ethical committee’.

### Procedure

The in-vivo glucose lowering properties of the test compounds were evaluated in C57BL/6J (mild hyperglycemic) animal models as described below. Two days prior to the study, the animals were randomized and divided into groups (n=6), based upon their fed glucose levels. On the day of experiment, food was withdrawn from all the cages, water was given ad-libitum and were kept for overnight fasting. Vehicle (normal saline)/test compounds were administered orally, on a body weight basis. Soon after the 0 min. blood collection from each animal, the subsequent blood collections were done at 30, 60 and 120 or upto 240 min., via retro-orbital route, under light ether anesthesia (Diabetes Obesity Metabolism, 7, 307, 2005; Diabetes, 52, 751, 2003).

Blood samples were centrifuged and the separated serum was immediately subjected for the glucose estimation. Serum for insulin estimation was stored at −70° C. until used for the insulin estimation. The glucose estimation was carried out with DPEC-GOD/POD method (Ranbaxy Fine Chemicals Limited, Diagnostic division, India), using Spectramax-190, in 96-microwell plate reader (Molecular devices Corporation, Sunnyvale, Calif.). Mean values of duplicate samples were calculated using Microsoft excel and the Graph Pad Prism software (Ver 4.0) was used to plot a 0 min base line corrected line graph, area under the curve (0-120 min AUC) and base line corrected area under the curve (0 min BCAUC). The AUC and BCAUC obtained from graphs were analyzed for one way ANOVA, followed by Dunnett's post test, using Graph Pad prism software. Changes in the blood glucose levels, with selected compounds are shown in Table-4.

TABLE 4
in vivo anti-diabetic activity of test compounds, in mice

| | In vivo OGTT C57; |
|---|---|
| Compounds | % Glucose change |
| 1 | −22.50 ± 4.5 |
| 2 | −25.70 ± 1.8 |
| 3 | −27.60 ± 1.6 |
| 4 | −16.50 ± 3.8 |
| 5 | −7.60 ± 1.3 |
| 6 | −7.30 ± 1.6 |
| 7 | −7.90 ± 1.2 |
| 8 | −49.10 ± 3.6 |
| 9 | −34.70 ± 4.4 |
| 10 | −12.50 ± 1.3 |
| 11 | −25.60 ± 3.2 |
| 12 | −9.80 ± 1.5 |
| 13 | −26.40 ± 1.6 |
| 14 | −14.20 ± 3.4 |
| 15 | −42.03 ± 1.8 |
| 16 | −29.40 ± 6.8 |
| 17 | −27.30 ± 3.3 |
| 18 | −12.60 ± 4.0 |
| 19 | −20.60 ± 3.0 |
| 20 | −8.40 ± 1.7 |
| 21 | −15.02 ± 4.0 |
| 22 | −25.10 ± 2.0 |
| 23 | −22.01 ± 1.2 |
| 24 | −18.90 ± 5.7 |
| 25 | −12.20 ± 2.5 |
| 26 | −20.10 ± 1.4 |
| 27 | −13.70 ± 1.2 |
| 28 | −35.02 ± 6.1 |
| 29 | −27.70 ± 3.1 |
| 30 | −35.01 ± 1.4 |
| 31 | −28.10 ± 3.2 |
| 32 | −25.30 ± 3.9 |
| 33 | −16.80 ± 1.6 |
| 34 | −30.10 ± 3.4 |
| 35 | −20.50 ± 3.8 |
| 36 | −22.20 ± 2.5 |
| 37 | −25.05 ± 3.2 |
| 38 | −22.04 ± 2.6 |
| 39 | −25.08 ± 3.6 |
| 40 | −14.01 ± 1.5 |
| 41 | −6.04 ± 2.6 |
| 42 | −25.05 ± 2.8 |
| 43 | −15.50 ± 3.6 |
| 44 | −12.50 ± 1.5 |
| 45 | −34.9 ± 4.4 |
| 46 | −9.3 ± 3.8 |
| 47 | 8.6 ± 5.9 |
| 48 | −38.5 ± 1.5 |
| 50 | −30.02 ± 1.4 |

Acute single dose 120−min time−course experiments, in male C57BL/6J mice (in vivo glucose reduction), with test compounds;
n = 6, all values are Mean ± SEM;
Test compounds administered via oral route of administration, dose 0,3 mg/kg, po.

Pharmacokinetic Study in Wistar Rats

The pharmacokinetic parameters of test compounds were determined in male wistar rats (n=6). Briefly, test compounds were administered orally/iv on a body weight basis to overnight fasted rats. Serial blood samples were collected in microcentrifuge tubes containing EDTA at pre-dose and post-dose after compounds administration, over a period of 168 hrs. Blood was collected at various time points and centrifuged at 4° C. The obtained plasma was frozen, stored at −70° C. and the concentrations of compounds in plasma were determined by the LC-MS/MS (Shimadzu LC 1 OAD, USA), using YMC hydrosphere C₁₈ (2.0×50 mm, 3 μm) column (YMC Inc., USA). The pharmacokinetic parameters, such as Tmax, t_{1/2}, AUC and % F were calculated using a non-compartmental model of WinNonlin software version 5.2.1. PK parameters of representative test compounds are shown in Table-5.

TABLE 5
Pharmacokinetic (PK) parameters of test compounds in rats

| Compounds | Cmax (ng/ml) | t_{1/2} (h) | AUC (h · ng/ml) |
|---|---|---|---|
| 1 | 148.25 ± 34.17 | 33.30 ± 2.07 | 888.29 ± 129.47 |
| 3 | 300.78 ± 44.27 | 48.20 ± 11.05 | 2967.69 ± 1070.68 |
| 8 | 459.04 ± 52.17 | 59.48 ± 6.44 | 4751.59 ± 646.66 |
| 17 | 418.83 ± 45.50 | 32.46 ± 5.91 | 1554.33 ± 114.41 |

* Vehicle: Tween80:PEG400:0.5% Na-CMC in purified water :: 5:5:90, v/v/v; n = 6; Mean ± SD; Dose: 2 mg/kg, orally

The novel compounds of the present invention can be formulated into suitable pharmaceutically acceptable compositions by combining with suitable excipients by techniques and processes and concentrations as are well known.

The compounds of Formula (I) or pharmaceutical compositions containing them are useful as antidiabetic compounds suitable for humans and other warm blooded animals, and may be administered either by oral, topical or parenteral administration.

The novel compounds of the present invention can be formulated into suitable pharmaceutically acceptable compositions by combining with suitable excipients by techniques and processes and concentrations as are well known. Thus, a pharmaceutical composition comprising the compounds of the present invention may comprise a suitable binder, suitable bulking agent &/or diluent and any other suitable agents as may be necessary. Optionally, the pharmaceutical composition may be suitably coated with suitable coating agents.

The compounds of the present invention (I) are DPP-IV inhibitors and are useful in the treatment of disease states mediated by DPP-IV enzyme, preferably diabetes and related disorders.

The quantity of active component, that is, the compounds of Formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

While the present invention has been described in terms of its specific embodiments, certain modifications and equivalents will be apparent to those skilled in the art and are intended to be included within the scope of the present invention.

1. Compound having the structure of general formula (I) 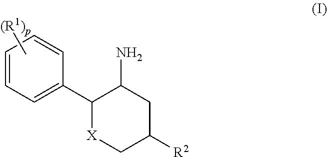
Wherein:
R¹ at each occurrence is independently selected from hydrogen, halo, cyano, nitro, hydroxyl, optionally substituted groups selected from amino, C₁₋₆ alkyl, C₂₋₆ alkenyl, C₂₋₆ alkynyl, C₁₋₆ alkoxy, C₂₋₆ alkenoxy, C₂₋₆ alkynyloxy, cycloalkoxy, aryl, cycloalkyl, carbocycle, heterocyclyl, heteroaryl, heterocycloalkyl, cycloalkyl(C₁₋₆)alkyl, heterocycloalkyl(C₁₋₆)alkyl, aralkyl, heteroarylalkyl, aryloxy, heteroaryloxy, heterocyclyloxy groups; R² is selected from the following bicyclic non-aromatic ring systems: 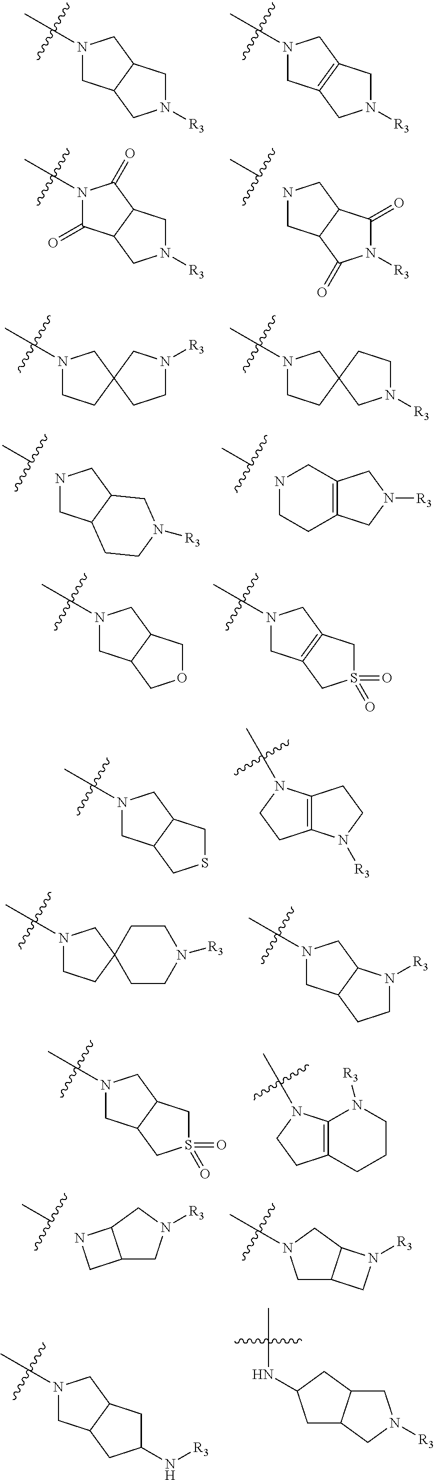 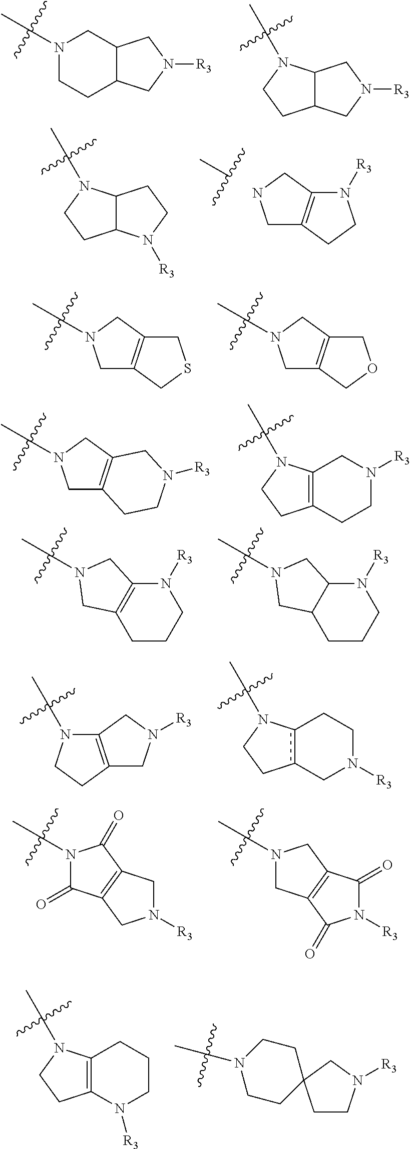
Wherein R₃ at each occurrence is independently selected from hydrogen, halo, haloalkyl, cyano, optionally substituted groups selected from amino, C₁₋₆ alkyl, C₂₋₆ alkenyl, C₂₋₆ alkynyl, aryl, cycloalkyl, carbocycle, heterocycloalkyl, cycloalkyl(C₁₋₆)alkyl, heterocycloalkyl(C₁₋₆)alkyl, S(O)ₙ, S(O)ₙ(C₁₋₆)alkyl, S(O)ₙ(C₁₋₆)aryl, S(O)ₙNH₂, S(O)ₙNH(C₁₋₆)alkyl, S(O)ₙNHcycloalkyl, S(O)ₙNHaryl, S(O)ₙNHheteroaryl, (C₁₋₆)alkylamino, nitro, COO(C₁₋₄)alkyl, S((O)═NH)-alkyl, S((O)═NH)-aryl, S((O)═NH)-cycloalkyl, S((O)═NH)-heteroaryl, S((O)═N-alkyl)-alkyl, S((O)═N-alkyl)-aryl, S((O)═N-alkyl)cycloalkyl, S((O)═N-alkyl)-heteroaryl, S((O)═N-aryl)-alkyl, S((O)═N-aryl)-aryl, S((O)═N-aryl)-cycloalkyl, S((O)═N-aryl)-heteroaryl, S((O)═N—(SO₂-alkyl))-alkyl, S((O)═N—(SO₂-alkyl))-aryl, S((O)═N—(SO₂-alkyl))-cycloalkyl, S((O)═N—(SO₂-alkyl))-heteroaryl, S((O)═N—(SO₂-aryl))-alkyl, S((O)═N—(SO₂-aryl))-aryl, S((O)═N—(SO₂-aryl))-cycloalkyl, S((O)═N—(SO₂-aryl))-heteroaryl, C(O), C(O)NH(C₁₋₆)alkyl groups;
n=0, 1, 2, 3, 4, 5, 6, 7; p=1-5; X=—CH₂, —NR⁴, O, S;
R⁴ is independently selected from hydrogen, halo, amino, cyano, nitro, (Ci-4)alkyl, (C₁₋₆)alkylcarbonyl, (C₂₋₆)alkenyl, (C₂₋₆)alkynyl, —(CH₂)ₙCOO(C₁₋₄)alkyl, —(CH₂)ₙCOOH, —C(═O)CH₂alkyl, —C(═O)CH₂aryl, —C(═O)CH₂heteroaryl, (CH₂)ₙaryl, (CH₂)ₙheteroaryl, (CH₂)ₙ—N-heteroaryl, (CH₂)ₙ—N-heterocyclyl, S(O)ₙ, S(O)ₙaryl, S(O)ₙalkyl, S(O)ₙ(C₁₋₆)alkyl, S(O)ₙ(C₁₋₆)aryl, S(O)ₙNH₂, S(O)ₙNH(C₁₋₆)alkyl groups.

2. The compound as claimed in claim 1 wherein R¹ at each occurrence is independently selected from hydrogen, halo, cyano, optionally substituted groups selected from amino, C₁₋₄ alkyl, C₂₋₆ alkenyl, C₂₋₆ alkynyl, aryl, cycloalkyl, carbocycle, heterocycloalkyl, cycloalkyl(C₁₋₆)alkyl, heterocycloalkyl(C₁₋₆)alkyl groups.

3. The compound as claimed in claim 1 wherein the substituents on R¹ are independently selected from hydroxy, (C₁₋₄)alkoxy, halo, cyano, amino, (C₁₋₆)alkylamino, nitro, COO(C₁₋₄)alkyl, S(O)ₙ, S(O)ₙNH₂, S(O)ₙNH(C₁₋₆)alkyl, C(O); C(O)NH(C₁₋₆)alkyl groups.

4. The compounds claimed in claim 1, wherein R⁴ is independently selected from hydrogen, halo, amino, cyano, nitro, methyl, ethyl, propyl, butyl, cyclopropyl, cyclobutyl, cyclopentyl, cyclohexyl, —CH₂—COOH, —C(═O)CH₂-methyl, —C(═O)CH₂-phenyl, S(O)₂-phenyl, S(O)₂-methyl, S(O)₂NH₂, S(O)₂NH-methyl groups.

5. The compound as claimed in wherein when R₃ is substituted, the substituents on R₃ are selected from hydrogen, halo haloalkyl, amino, cyano, methyl, ethyl, propyl, butyl, cyclopropyl, cyclobutyl, cyclopentyl, cyclohexyl, phenyl, —CH₂—COOH, —C(═O)—O-methyl, —C(═O)—O-trifluromethyl, —C(═O)—O-ethyl, —C(═O)—O-phenyl, —C(═O)—NH-methyl, —C(═O)—NH— ethyl, —C(═O)—NH-propyl, —C(═O)—NH-cyclopropyl, —C(═O)—NH-phenyl, —C(═O)—NH-trifluromethyl, —C(═O)-methyl, —C(═O)-ethyl, —C(═O)CH₂-methyl, —C(═O)CH₂-phenyl, S(O)₂-phenyl, S(O)₂-methyl, S(O)₂-ethyl, S(O)₂-propyl, S(O)₂-butyl, S(O)₂-cyclopropyl, S(O)₂-cyclobutyl, S(O)₂-cyclopentyl, S(O)₂-cyclohexyl, S(O)₂-phenyl, S(O)₂-flurophenyl, S(O)₂-cynophenyl, S(O)₂NH₂, S(O)₂NH-methyl, S(O)₂NH-ethyl, S(O)₂NH-propyl, S(O)₂NH-butyl, S(O)₂NH-pentyl, S(O)₂NH-cyclopropyl, S(O)₂NH-cyclobutyl, S(O)₂NH-cyclopentyl, S(O)₂NH-cyclohexyl, S(O)₂NH-phenyl, S((O)═NH)-methyl, S((O)═NH)-ethyl, S((O)═NH)-phenyl, S((O)═NH)-cyclopentyl, S((O)═NH)-pyridine, S((O)═N-methyl)-methyl, S((O)═N-methyl)-phenyl, S((O)═N-ethyl)-cyclopropyl, S((O)═N-methyl)-pyridine, S((O)═N-phenyl)-methyl, S((O)═N-phenyl)-phenyl, S((O)═N-phenyl)-cyclopentyl, S((O)═N-phenyl)-pyridine, S((O)═N—(SO₂-methyl))-methyl, S((O)═N—(SO₂-methyl))-phenyl, S((O)═N—(SO₂-ethyl))-cyclohexyl, S((O)═N—(SO₂-methyl))-pyridine, S((O)═N—(SO₂-phenyl))-methyl, S((O)═N—(SO₂-phenyl))-phenyl, S((O)═N—(SO₂-phenyl))-cyclopentyl, S((O)═N—(SO₂-phenyl))-pyridine.

6. A compound as claimed in claim 1 selected from the group comprising of:

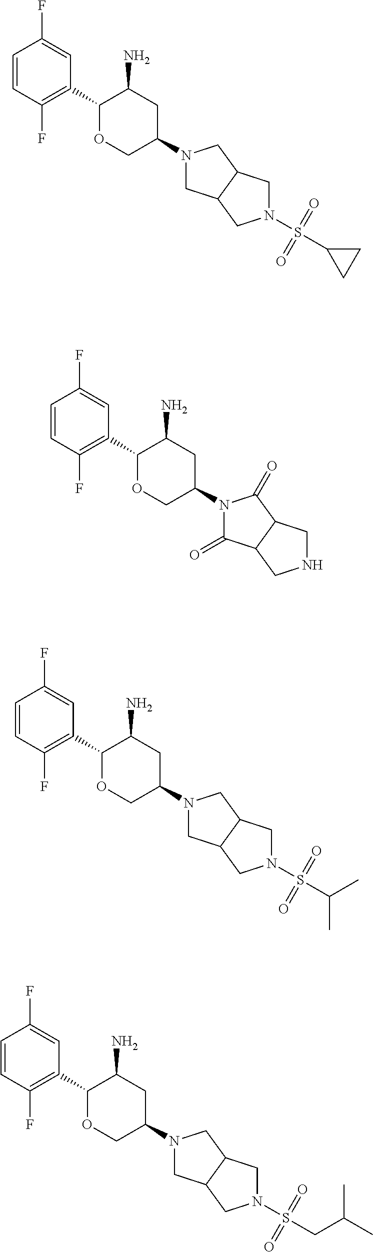 [SMILES: N[C@H]1C[C@@H](N2CC3CN(S(=O)(=O)C4CC4)CC3C2)CO[C@@H]1c1cc(F)ccc1F ; N[C@H]1C[C@@H](N2C(=O)C3CNCC3C2=O)CO[C@@H]1c1cc(F)ccc1F ; CC(C)S(=O)(=O)N1CC2CN([C@H]3CO[C@H](c4cc(F)ccc4F)[C@@H](N)C3)CC2C1 ; CC(C)CS(=O)(=O)N1CC2CN([C@H]3CO[C@H](c4cc(F)ccc4F)[C@@H](N)C3)CC2C1]

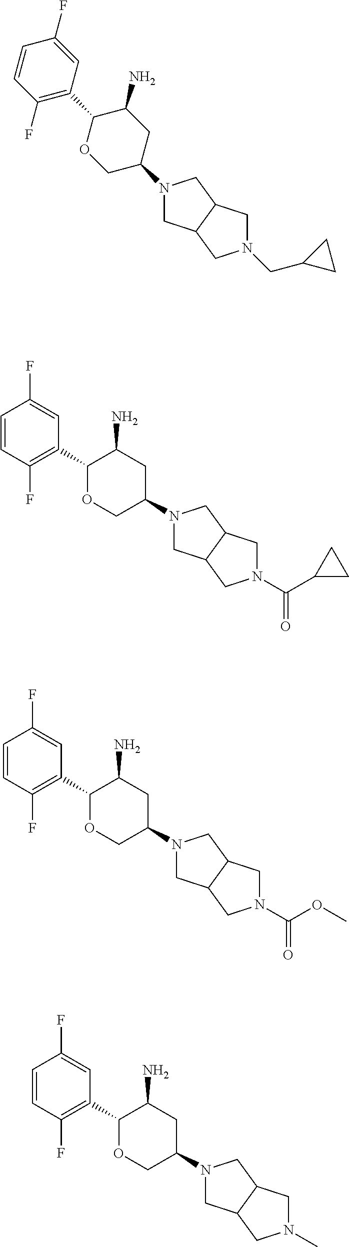 [SMILES: N[C@H]1C[C@@H](N2CC3CN(CC4CC4)CC3C2)CO[C@@H]1c1cc(F)ccc1F ; N[C@H]1C[C@@H](N2CC3CN(C(=O)C4CC4)CC3C2)CO[C@@H]1c1cc(F)ccc1F ; COC(=O)N1CC2CN([C@H]3CO[C@H](c4cc(F)ccc4F)[C@@H](N)C3)CC2C1 ; CN1CC2CN([C@H]3CO[C@H](c4cc(F)ccc4F)[C@@H](N)C3)CC2C1]

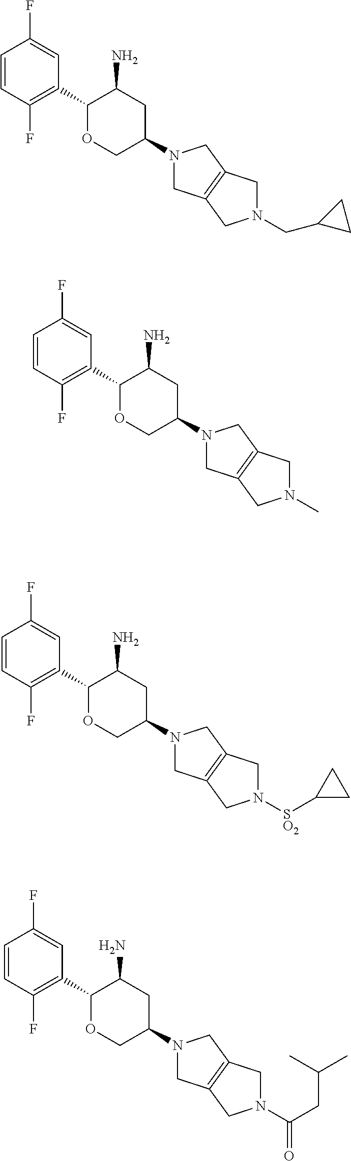 [SMILES: N[C@H]1C[C@@H](N2CC3=C(CN(CC4CC4)C3)C2)CO[C@@H]1c1cc(F)ccc1F ; CN1CC2=C(C1)CN([C@H]1CO[C@H](c3cc(F)ccc3F)[C@@H](N)C1)C2 ; N[C@H]1C[C@@H](N2CC3=C(C2)CN(S(=O)(=O)C2CC2)C3)CO[C@@H]1c1cc(F)ccc1F ; CC(C)CC(=O)N1CC2=C(C1)CN([C@H]1CO[C@H](c3cc(F)ccc3F)[C@@H](N)C1)C2]

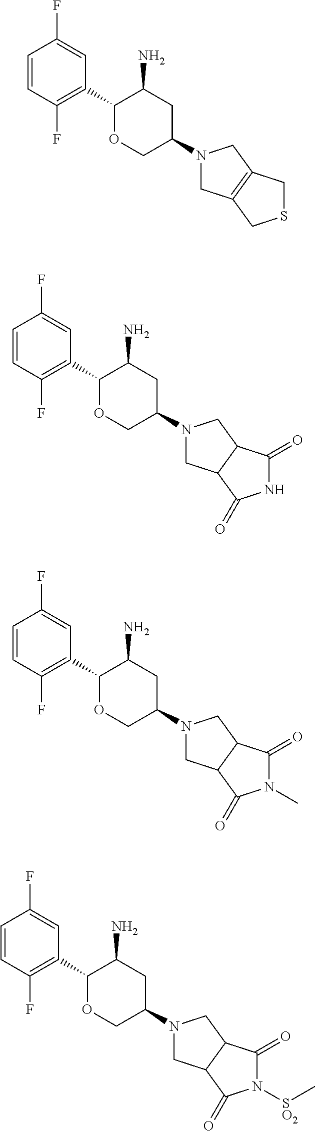 [SMILES: N[C@H]1C[C@@H](N2CC3=C(CSC3)C2)CO[C@@H]1c1cc(F)ccc1F ; N[C@H]1C[C@@H](N2CC3C(=O)NC(=O)C3C2)CO[C@@H]1c1cc(F)ccc1F ; CN1C(=O)C2CN([C@H]3CO[C@H](c4cc(F)ccc4F)[C@@H](N)C3)CC2C1=O ; CS(=O)(=O)N1C(=O)C2CN([C@H]3CO[C@H](c4cc(F)ccc4F)[C@@H](N)C3)CC2C1=O]

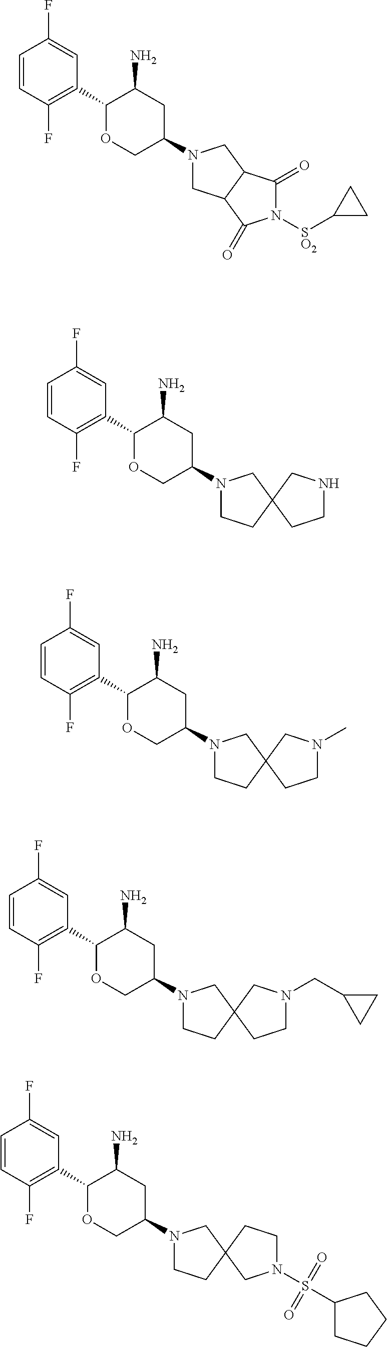 [SMILES: N[C@H]1C[C@@H](N2CC3C(=O)N(S(=O)(=O)C4CC4)C(=O)C3C2)CO[C@@H]1c1cc(F)ccc1F ; N[C@H]1C[C@@H](N2CCC3(CCNC3)C2)CO[C@@H]1c1cc(F)ccc1F ; CN1CCC2(CCN([C@H]3CO[C@H](c4cc(F)ccc4F)[C@@H](N)C3)C2)C1 ; N[C@H]1C[C@@H](N2CCC3(CCN(CC4CC4)C3)C2)CO[C@@H]1c1cc(F)ccc1F ; N[C@H]1C[C@@H](N2CCC3(CCN(S(=O)(=O)C4CCCC4)C3)C2)CO[C@@H]1c1cc(F)ccc1F]

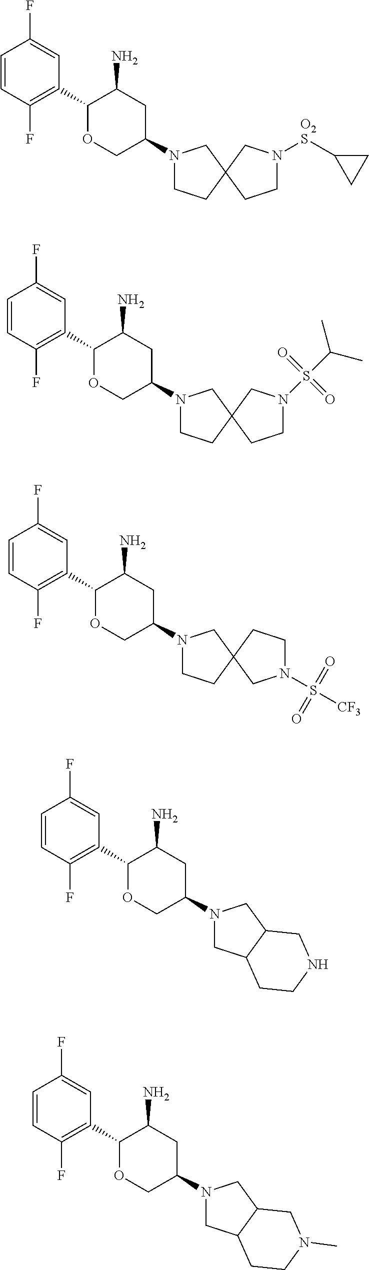 [SMILES: N[C@H]1C[C@@H](N2CCC3(CCN(S(=O)(=O)C4CC4)C3)C2)CO[C@@H]1c1cc(F)ccc1F ; CC(C)S(=O)(=O)N1CCC2(CCN([C@H]3CO[C@H](c4cc(F)ccc4F)[C@@H](N)C3)C2)C1 ; N[C@H]1C[C@@H](N2CCC3(CCN(S(=O)(=O)C(F)(F)F)C3)C2)CO[C@@H]1c1cc(F)ccc1F ; N[C@H]1C[C@@H](N2CC3CCNCC3C2)CO[C@@H]1c1cc(F)ccc1F ; CN1CCC2CN([C@H]3CO[C@H](c4cc(F)ccc4F)[C@@H](N)C3)CC2C1]

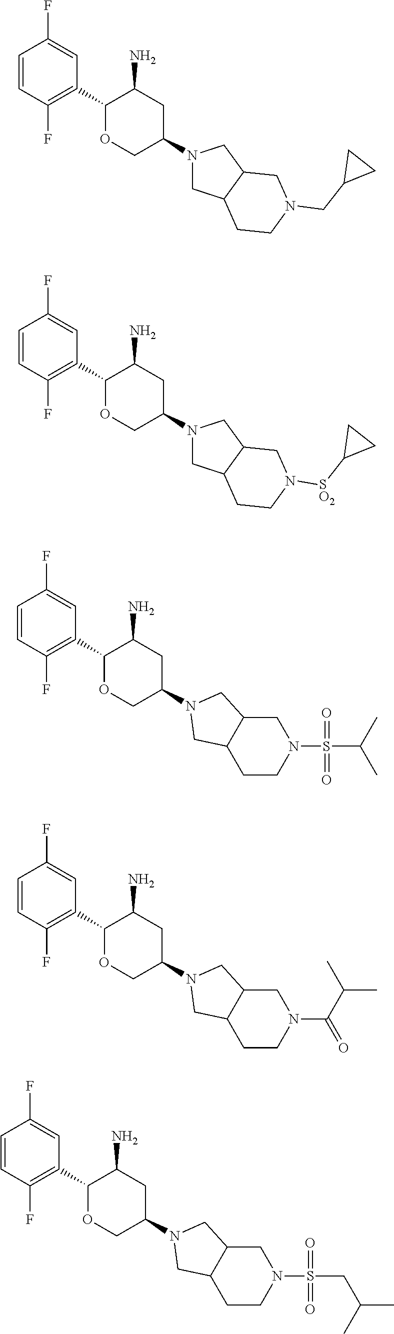 [SMILES: N[C@H]1C[C@@H](N2CC3CCN(CC4CC4)CC3C2)CO[C@@H]1c1cc(F)ccc1F ; N[C@H]1C[C@@H](N2CC3CCN(S(=O)(=O)C4CC4)CC3C2)CO[C@@H]1c1cc(F)ccc1F ; CC(C)S(=O)(=O)N1CCC2CN([C@H]3CO[C@H](c4cc(F)ccc4F)[C@@H](N)C3)CC2C1 ; CC(C)C(=O)N1CCC2CN([C@H]3CO[C@H](c4cc(F)ccc4F)[C@@H](N)C3)CC2C1 ; CC(C)CS(=O)(=O)N1CCC2CN([C@H]3CO[C@H](c4cc(F)ccc4F)[C@@H](N)C3)CC2C1]

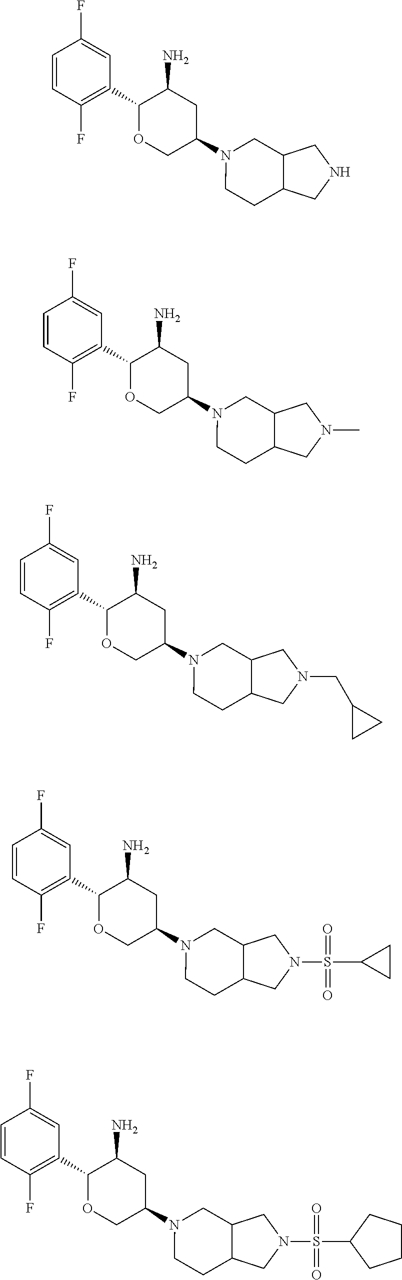 [SMILES: N[C@H]1C[C@@H](N2CCC3CNCC3C2)CO[C@@H]1c1cc(F)ccc1F ; CN1CC2CCN([C@H]3CO[C@H](c4cc(F)ccc4F)[C@@H](N)C3)CC2C1 ; N[C@H]1C[C@@H](N2CCC3CN(CC4CC4)CC3C2)CO[C@@H]1c1cc(F)ccc1F ; N[C@H]1C[C@@H](N2CCC3CN(S(=O)(=O)C4CC4)CC3C2)CO[C@@H]1c1cc(F)ccc1F ; N[C@H]1C[C@@H](N2CCC3CN(S(=O)(=O)C4CCCC4)CC3C2)CO[C@@H]1c1cc(F)ccc1F]

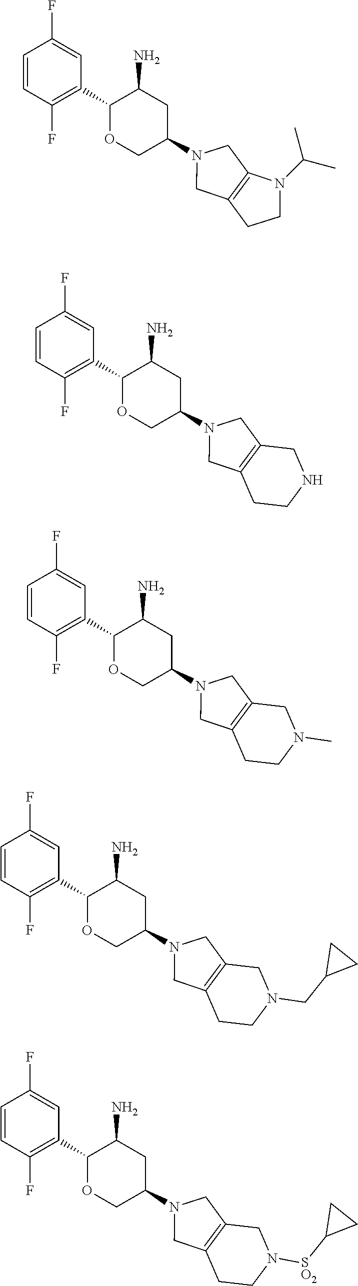 [SMILES: CC(C)N1CCC2=C1CN([C@H]1CO[C@H](c3cc(F)ccc3F)[C@@H](N)C1)C2 ; N[C@H]1C[C@@H](N2CC3=C(CNCC3)C2)CO[C@@H]1c1cc(F)ccc1F ; CN1CCC2=C(C1)CN([C@H]1CO[C@H](c3cc(F)ccc3F)[C@@H](N)C1)C2 ; N[C@H]1C[C@@H](N2CC3=C(CN(CC4CC4)CC3)C2)CO[C@@H]1c1cc(F)ccc1F ; N[C@H]1C[C@@H](N2CC3=C(C2)CN(S(=O)(=O)C2CC2)CC3)CO[C@@H]1c1cc(F)ccc1F]

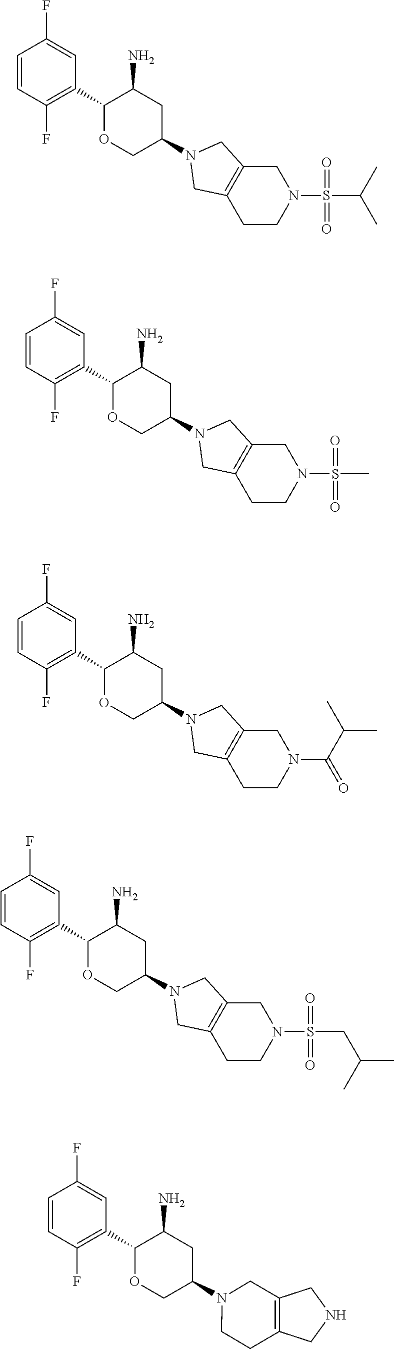 [SMILES: CC(C)S(=O)(=O)N1CCC2=C(CN([C@H]3CO[C@H](c4cc(F)ccc4F)[C@@H](N)C3)C2)C1 ; CS(=O)(=O)N1CCC2=C(CN([C@H]3CO[C@H](c4cc(F)ccc4F)[C@@H](N)C3)C2)C1 ; CC(C)C(=O)N1CCC2=C(C1)CN([C@H]1CO[C@H](c3cc(F)ccc3F)[C@@H](N)C1)C2 ; CC(C)CS(=O)(=O)N1CCC2=C(CN([C@H]3CO[C@H](c4cc(F)ccc4F)[C@@H](N)C3)C2)C1 ; N[C@H]1C[C@@H](N2CCC3=C(CNC3)C2)CO[C@@H]1c1cc(F)ccc1F]

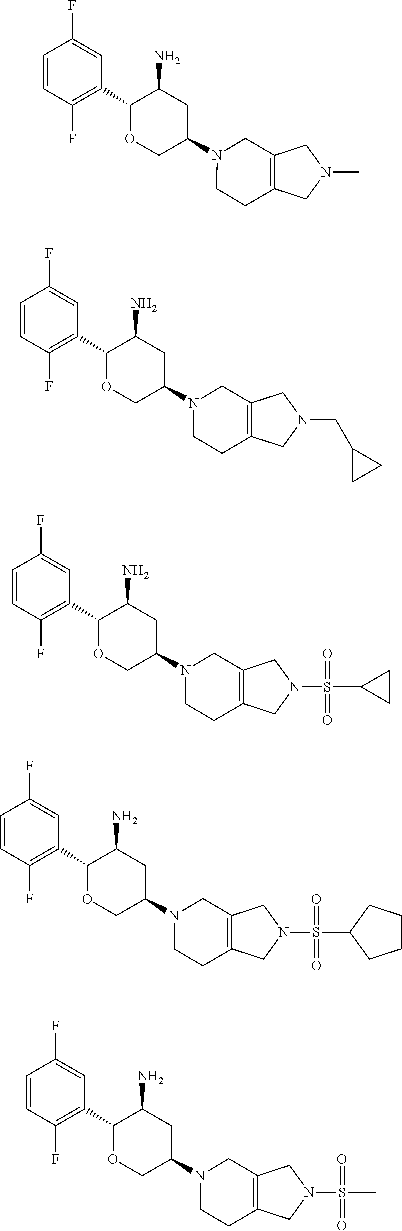 [SMILES: CN1CC2=C(C1)CN([C@H]1CO[C@H](c3cc(F)ccc3F)[C@@H](N)C1)CC2 ; N[C@H]1C[C@@H](N2CCC3=C(CN(CC4CC4)C3)C2)CO[C@@H]1c1cc(F)ccc1F ; N[C@H]1C[C@@H](N2CCC3=C(C2)CN(S(=O)(=O)C2CC2)C3)CO[C@@H]1c1cc(F)ccc1F ; N[C@H]1C[C@@H](N2CCC3=C(C2)CN(S(=O)(=O)C2CCCC2)C3)CO[C@@H]1c1cc(F)ccc1F ; CS(=O)(=O)N1CC2=C(CN([C@H]3CO[C@H](c4cc(F)ccc4F)[C@@H](N)C3)CC2)C1]

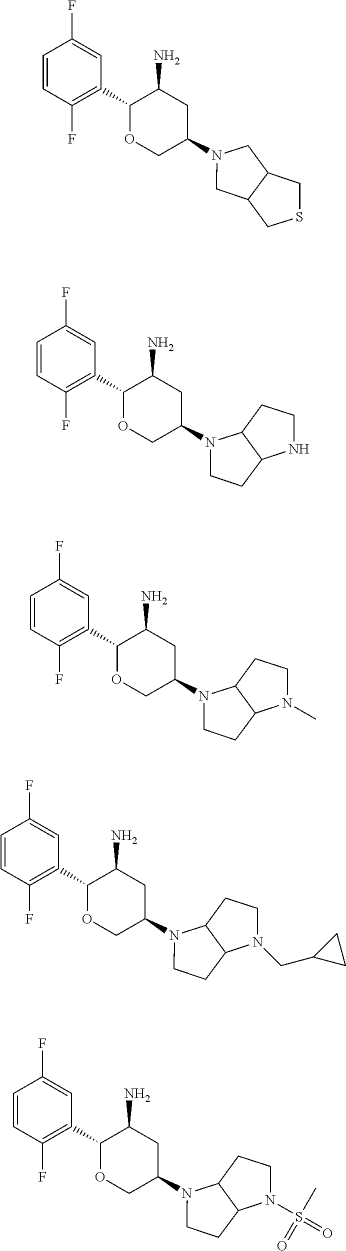 [SMILES: N[C@H]1C[C@@H](N2CC3CSCC3C2)CO[C@@H]1c1cc(F)ccc1F ; N[C@H]1C[C@@H](N2CCC3NCCC32)CO[C@@H]1c1cc(F)ccc1F ; CN1CCC2C1CCN2[C@H]1CO[C@H](c2cc(F)ccc2F)[C@@H](N)C1 ; N[C@H]1C[C@@H](N2CCC3C2CCN3CC2CC2)CO[C@@H]1c1cc(F)ccc1F ; CS(=O)(=O)N1CCC2C1CCN2[C@H]1CO[C@H](c2cc(F)ccc2F)[C@@H](N)C1]

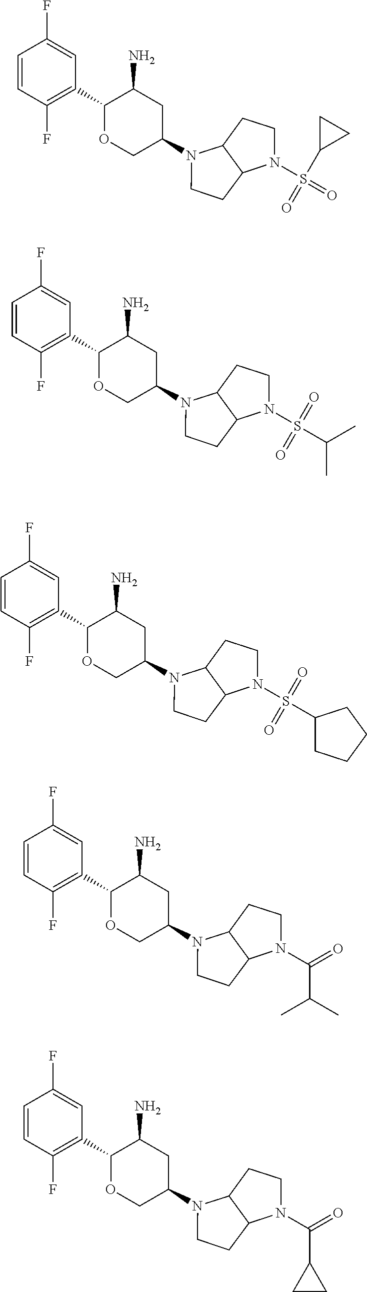 [SMILES: N[C@H]1C[C@@H](N2CCC3C2CCN3S(=O)(=O)C2CC2)CO[C@@H]1c1cc(F)ccc1F ; CC(C)S(=O)(=O)N1CCC2C1CCN2[C@H]1CO[C@H](c2cc(F)ccc2F)[C@@H](N)C1 ; N[C@H]1C[C@@H](N2CCC3C2CCN3S(=O)(=O)C2CCCC2)CO[C@@H]1c1cc(F)ccc1F ; CC(C)C(=O)N1CCC2C1CCN2[C@H]1CO[C@H](c2cc(F)ccc2F)[C@@H](N)C1 ; N[C@H]1C[C@@H](N2CCC3C2CCN3C(=O)C2CC2)CO[C@@H]1c1cc(F)ccc1F]

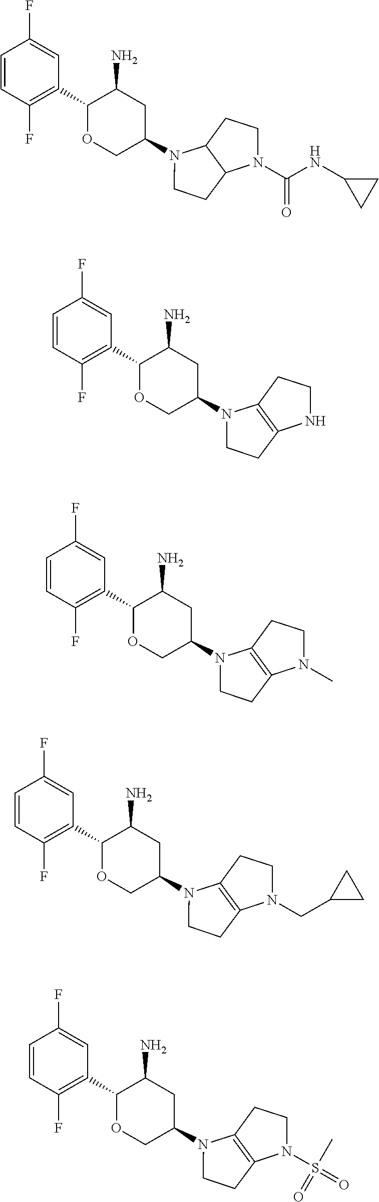 [SMILES: N[C@H]1C[C@@H](N2CCC3C2CCN3C(=O)NC2CC2)CO[C@@H]1c1cc(F)ccc1F ; N[C@H]1C[C@@H](N2CCC3=C2CCN3)CO[C@@H]1c1cc(F)ccc1F ; CN1CCC2=C1CCN2[C@H]1CO[C@H](c2cc(F)ccc2F)[C@@H](N)C1 ; N[C@H]1C[C@@H](N2CCC3=C2CCN3CC2CC2)CO[C@@H]1c1cc(F)ccc1F ; CS(=O)(=O)N1CCC2=C1CCN2[C@H]1CO[C@H](c2cc(F)ccc2F)[C@@H](N)C1]

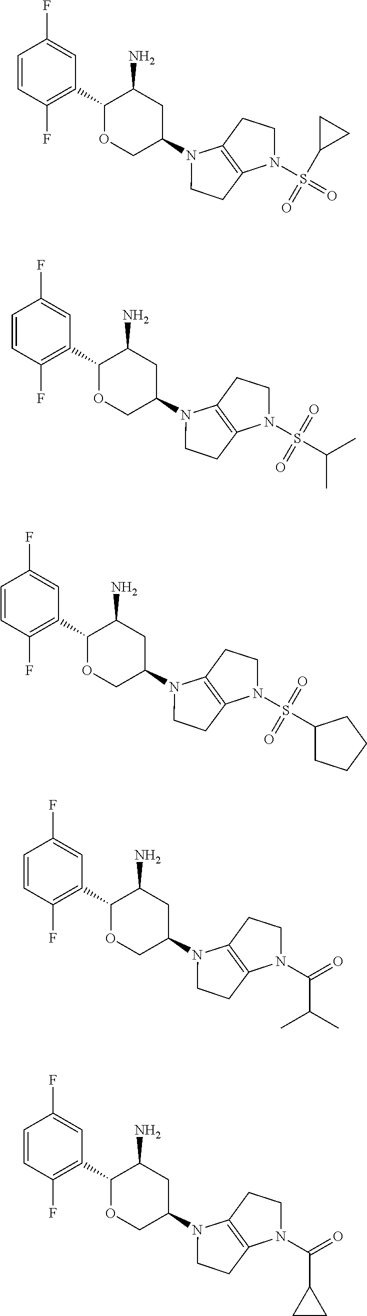 [SMILES: N[C@H]1C[C@@H](N2CCC3=C2CCN3S(=O)(=O)C2CC2)CO[C@@H]1c1cc(F)ccc1F ; CC(C)S(=O)(=O)N1CCC2=C1CCN2[C@H]1CO[C@H](c2cc(F)ccc2F)[C@@H](N)C1 ; N[C@H]1C[C@@H](N2CCC3=C2CCN3S(=O)(=O)C2CCCC2)CO[C@@H]1c1cc(F)ccc1F ; CC(C)C(=O)N1CCC2=C1CCN2[C@H]1CO[C@H](c2cc(F)ccc2F)[C@@H](N)C1 ; N[C@H]1C[C@@H](N2CCC3=C2CCN3C(=O)C2CC2)CO[C@@H]1c1cc(F)ccc1F]

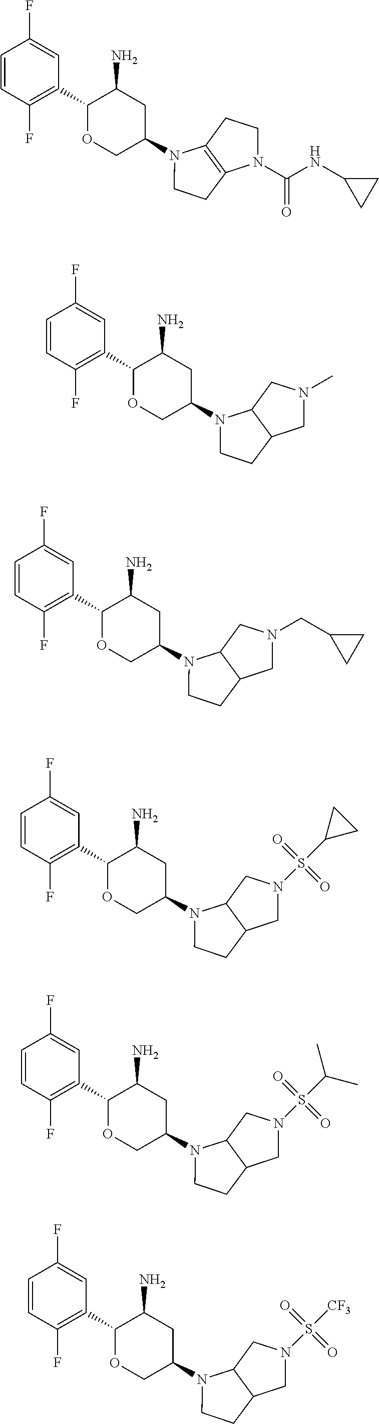 [SMILES: N[C@H]1C[C@@H](N2CCC3=C2CCN3C(=O)NC2CC2)CO[C@@H]1c1cc(F)ccc1F ; CN1CC2CCN([C@H]3CO[C@H](c4cc(F)ccc4F)[C@@H](N)C3)C2C1 ; N[C@H]1C[C@@H](N2CCC3CN(CC4CC4)CC32)CO[C@@H]1c1cc(F)ccc1F ; N[C@H]1C[C@@H](N2CCC3CN(S(=O)(=O)C4CC4)CC32)CO[C@@H]1c1cc(F)ccc1F ; CC(C)S(=O)(=O)N1CC2CCN([C@H]3CO[C@H](c4cc(F)ccc4F)[C@@H](N)C3)C2C1 ; N[C@H]1C[C@@H](N2CCC3CN(S(=O)(=O)C(F)(F)F)CC32)CO[C@@H]1c1cc(F)ccc1F]

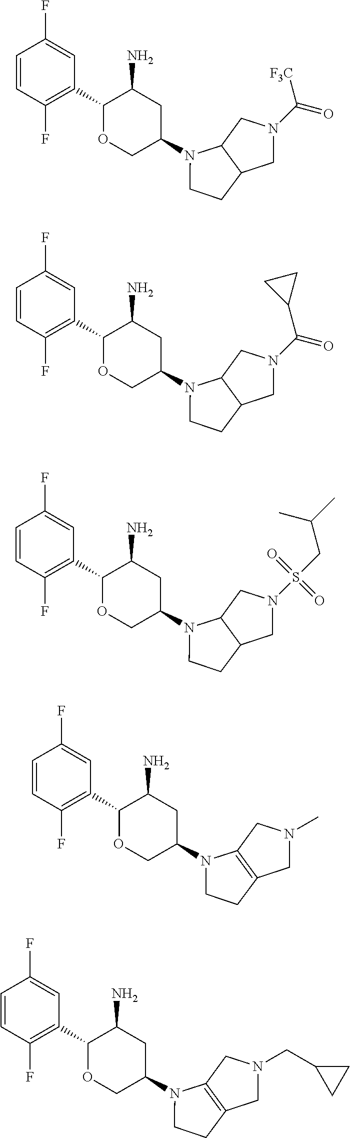 [SMILES: N[C@H]1C[C@@H](N2CCC3CN(C(=O)C(F)(F)F)CC32)CO[C@@H]1c1cc(F)ccc1F ; N[C@H]1C[C@@H](N2CCC3CN(C(=O)C4CC4)CC32)CO[C@@H]1c1cc(F)ccc1F ; CC(C)CS(=O)(=O)N1CC2CCN([C@H]3CO[C@H](c4cc(F)ccc4F)[C@@H](N)C3)C2C1 ; CN1CC2=C(C1)N([C@H]1CO[C@H](c3cc(F)ccc3F)[C@@H](N)C1)CC2 ; N[C@H]1C[C@@H](N2CCC3=C2CN(CC2CC2)C3)CO[C@@H]1c1cc(F)ccc1F]

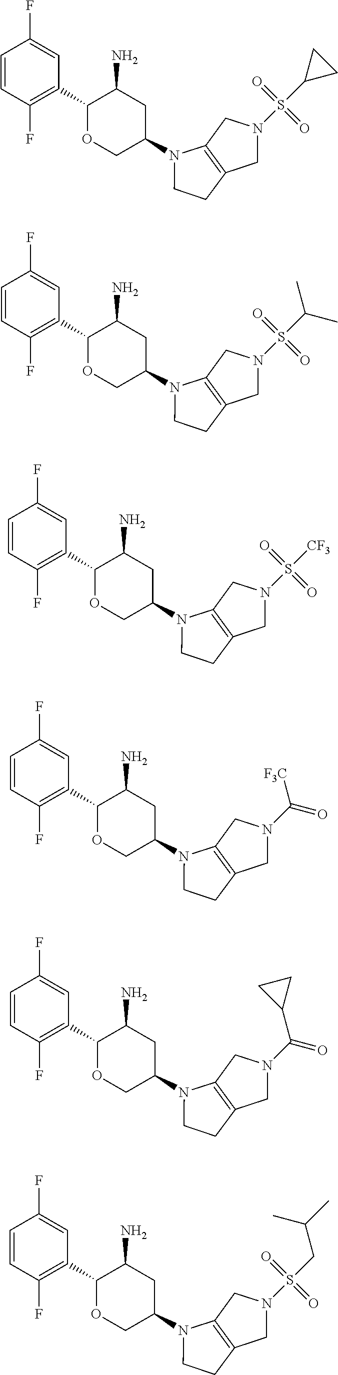 [SMILES: N[C@H]1C[C@@H](N2CCC3=C2CN(S(=O)(=O)C2CC2)C3)CO[C@@H]1c1cc(F)ccc1F ; CC(C)S(=O)(=O)N1CC2=C(C1)N([C@H]1CO[C@H](c3cc(F)ccc3F)[C@@H](N)C1)CC2 ; N[C@H]1C[C@@H](N2CCC3=C2CN(S(=O)(=O)C(F)(F)F)C3)CO[C@@H]1c1cc(F)ccc1F ; N[C@H]1C[C@@H](N2CCC3=C2CN(C(=O)C(F)(F)F)C3)CO[C@@H]1c1cc(F)ccc1F ; N[C@H]1C[C@@H](N2CCC3=C2CN(C(=O)C2CC2)C3)CO[C@@H]1c1cc(F)ccc1F ; CC(C)CS(=O)(=O)N1CC2=C(C1)N([C@H]1CO[C@H](c3cc(F)ccc3F)[C@@H](N)C1)CC2]

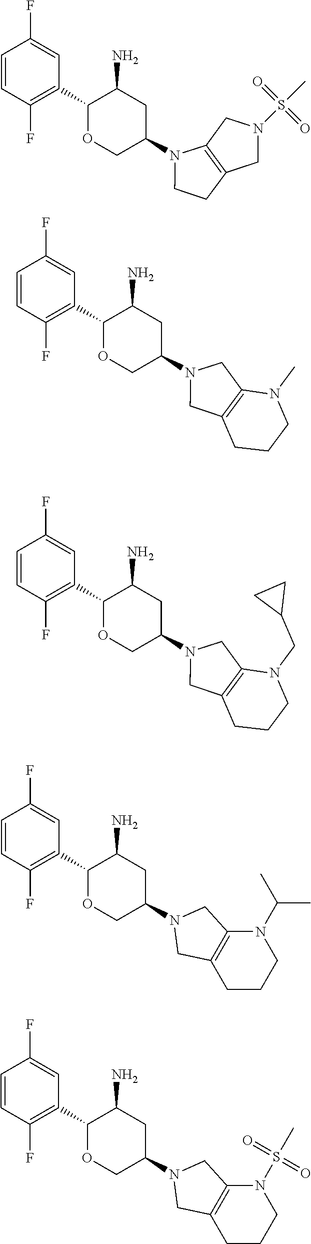 [SMILES: CS(=O)(=O)N1CC2=C(C1)N([C@H]1CO[C@H](c3cc(F)ccc3F)[C@@H](N)C1)CC2 ; CN1CCCC2=C1CN([C@H]1CO[C@H](c3cc(F)ccc3F)[C@@H](N)C1)C2 ; N[C@H]1C[C@@H](N2CC3=C(C2)N(CC2CC2)CCC3)CO[C@@H]1c1cc(F)ccc1F ; CC(C)N1CCCC2=C1CN([C@H]1CO[C@H](c3cc(F)ccc3F)[C@@H](N)C1)C2 ; CS(=O)(=O)N1CCCC2=C1CN([C@H]1CO[C@H](c3cc(F)ccc3F)[C@@H](N)C1)C2]

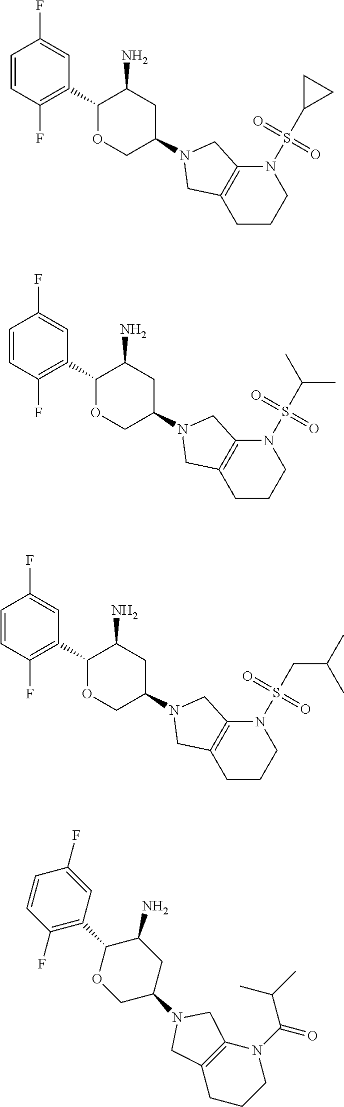 [SMILES: N[C@H]1C[C@@H](N2CC3=C(C2)N(S(=O)(=O)C2CC2)CCC3)CO[C@@H]1c1cc(F)ccc1F ; CC(C)S(=O)(=O)N1CCCC2=C1CN([C@H]1CO[C@H](c3cc(F)ccc3F)[C@@H](N)C1)C2 ; CC(C)CS(=O)(=O)N1CCCC2=C1CN([C@H]1CO[C@H](c3cc(F)ccc3F)[C@@H](N)C1)C2 ; CC(C)C(=O)N1CCCC2=C1CN([C@H]1CO[C@H](c3cc(F)ccc3F)[C@@H](N)C1)C2]

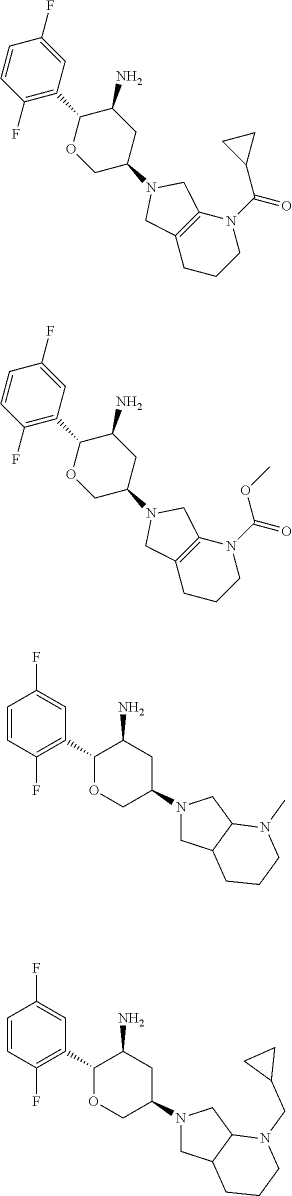 [SMILES: N[C@H]1C[C@@H](N2CC3=C(C2)N(C(=O)C2CC2)CCC3)CO[C@@H]1c1cc(F)ccc1F ; COC(=O)N1CCCC2=C1CN([C@H]1CO[C@H](c3cc(F)ccc3F)[C@@H](N)C1)C2 ; CN1CCCC2CN([C@H]3CO[C@H](c4cc(F)ccc4F)[C@@H](N)C3)CC21 ; N[C@H]1C[C@@H](N2CC3CCCN(CC4CC4)C3C2)CO[C@@H]1c1cc(F)ccc1F]

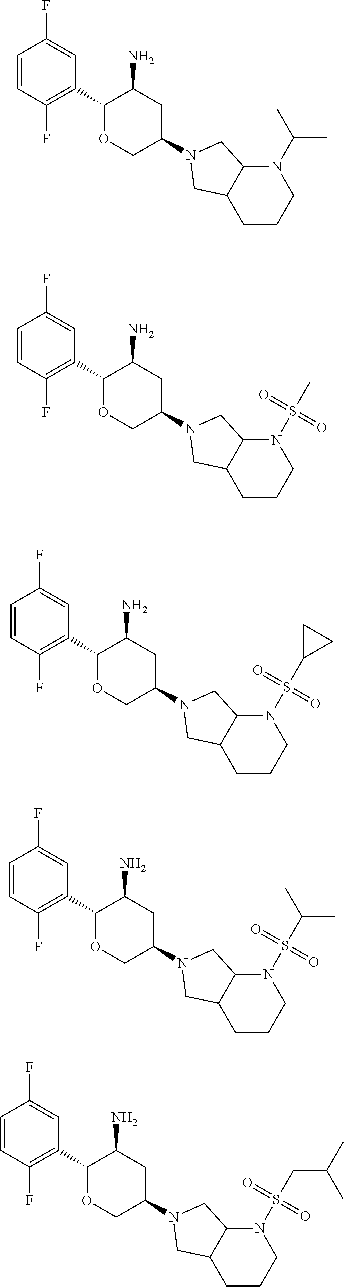 [SMILES: CC(C)N1CCCC2CN([C@H]3CO[C@H](c4cc(F)ccc4F)[C@@H](N)C3)CC21 ; CS(=O)(=O)N1CCCC2CN([C@H]3CO[C@H](c4cc(F)ccc4F)[C@@H](N)C3)CC21 ; N[C@H]1C[C@@H](N2CC3CCCN(S(=O)(=O)C4CC4)C3C2)CO[C@@H]1c1cc(F)ccc1F ; CC(C)S(=O)(=O)N1CCCC2CN([C@H]3CO[C@H](c4cc(F)ccc4F)[C@@H](N)C3)CC21 ; CC(C)CS(=O)(=O)N1CCCC2CN([C@H]3CO[C@H](c4cc(F)ccc4F)[C@@H](N)C3)CC21]

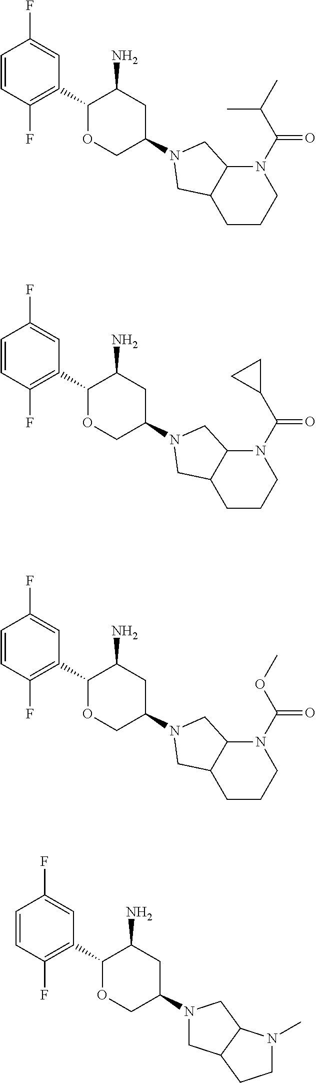 [SMILES: CC(C)C(=O)N1CCCC2CN([C@H]3CO[C@H](c4cc(F)ccc4F)[C@@H](N)C3)CC21 ; N[C@H]1C[C@@H](N2CC3CCCN(C(=O)C4CC4)C3C2)CO[C@@H]1c1cc(F)ccc1F ; COC(=O)N1CCCC2CN([C@H]3CO[C@H](c4cc(F)ccc4F)[C@@H](N)C3)CC21 ; CN1CCC2CN([C@H]3CO[C@H](c4cc(F)ccc4F)[C@@H](N)C3)CC21]

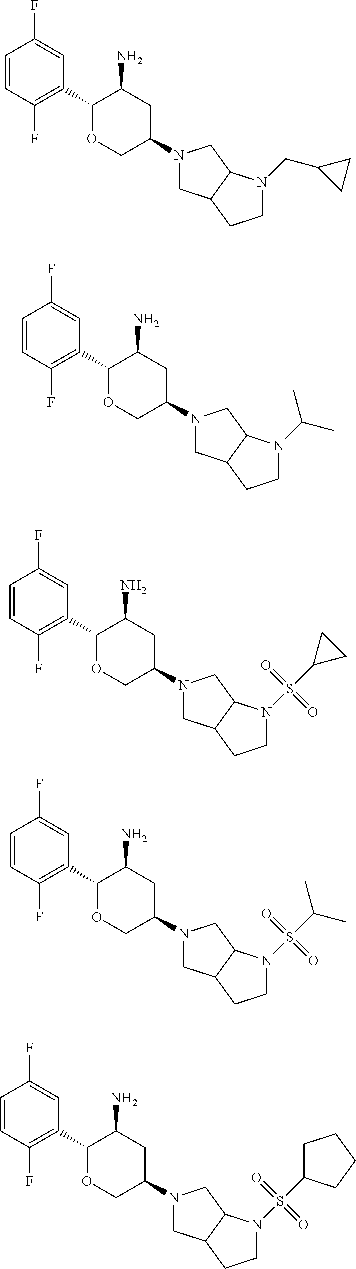 [SMILES: N[C@H]1C[C@@H](N2CC3CCN(CC4CC4)C3C2)CO[C@@H]1c1cc(F)ccc1F ; CC(C)N1CCC2CN([C@H]3CO[C@H](c4cc(F)ccc4F)[C@@H](N)C3)CC21 ; N[C@H]1C[C@@H](N2CC3CCN(S(=O)(=O)C4CC4)C3C2)CO[C@@H]1c1cc(F)ccc1F ; CC(C)S(=O)(=O)N1CCC2CN([C@H]3CO[C@H](c4cc(F)ccc4F)[C@@H](N)C3)CC21 ; N[C@H]1C[C@@H](N2CC3CCN(S(=O)(=O)C4CCCC4)C3C2)CO[C@@H]1c1cc(F)ccc1F]

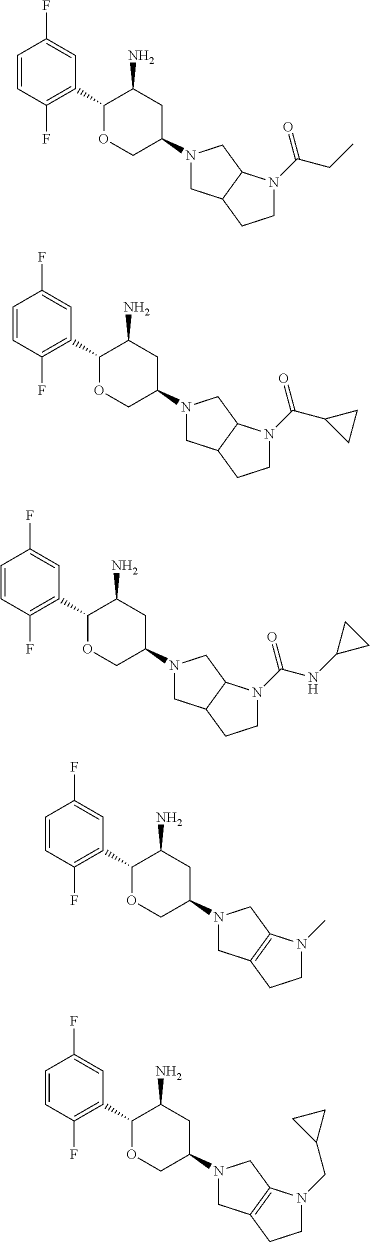 [SMILES: CCC(=O)N1CCC2CN([C@H]3CO[C@H](c4cc(F)ccc4F)[C@@H](N)C3)CC21 ; N[C@H]1C[C@@H](N2CC3CCN(C(=O)C4CC4)C3C2)CO[C@@H]1c1cc(F)ccc1F ; N[C@H]1C[C@@H](N2CC3CCN(C(=O)NC4CC4)C3C2)CO[C@@H]1c1cc(F)ccc1F ; CN1CCC2=C1CN([C@H]1CO[C@H](c3cc(F)ccc3F)[C@@H](N)C1)C2 ; N[C@H]1C[C@@H](N2CC3=C(C2)N(CC2CC2)CC3)CO[C@@H]1c1cc(F)ccc1F]

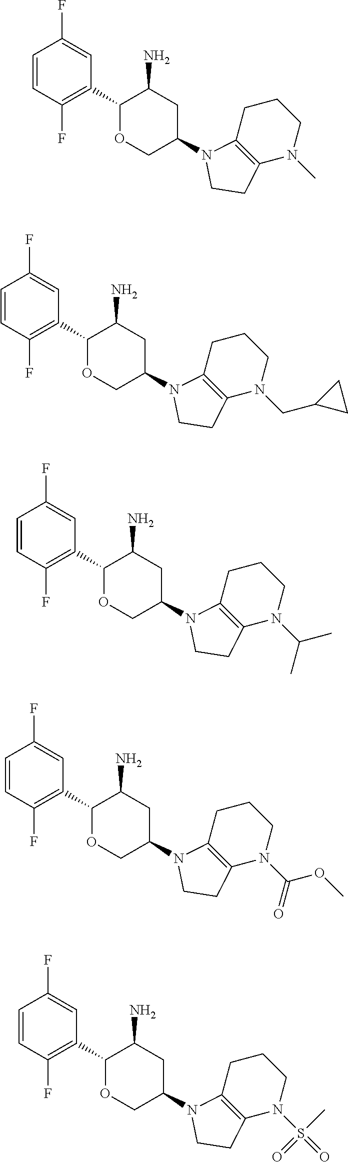 [SMILES: CN1CCCC2=C1CCN2[C@H]1CO[C@H](c2cc(F)ccc2F)[C@@H](N)C1 ; N[C@H]1C[C@@H](N2CCC3=C2CCCN3CC2CC2)CO[C@@H]1c1cc(F)ccc1F ; CC(C)N1CCCC2=C1CCN2[C@H]1CO[C@H](c2cc(F)ccc2F)[C@@H](N)C1 ; COC(=O)N1CCCC2=C1CCN2[C@H]1CO[C@H](c2cc(F)ccc2F)[C@@H](N)C1 ; CS(=O)(=O)N1CCCC2=C1CCN2[C@H]1CO[C@H](c2cc(F)ccc2F)[C@@H](N)C1]

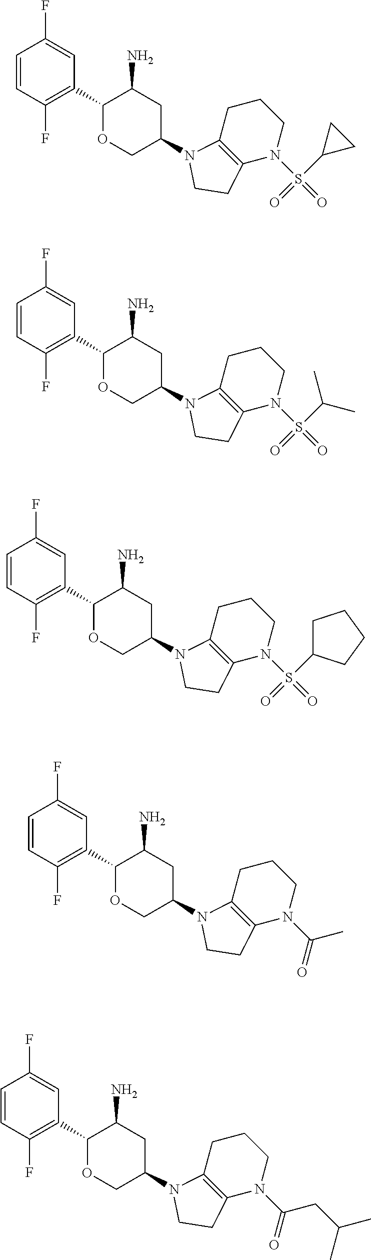 [SMILES: N[C@H]1C[C@@H](N2CCC3=C2CCCN3S(=O)(=O)C2CC2)CO[C@@H]1c1cc(F)ccc1F ; CC(C)S(=O)(=O)N1CCCC2=C1CCN2[C@H]1CO[C@H](c2cc(F)ccc2F)[C@@H](N)C1 ; N[C@H]1C[C@@H](N2CCC3=C2CCCN3S(=O)(=O)C2CCCC2)CO[C@@H]1c1cc(F)ccc1F ; CC(=O)N1CCCC2=C1CCN2[C@H]1CO[C@H](c2cc(F)ccc2F)[C@@H](N)C1 ; CC(C)CC(=O)N1CCCC2=C1CCN2[C@H]1CO[C@H](c2cc(F)ccc2F)[C@@H](N)C1]

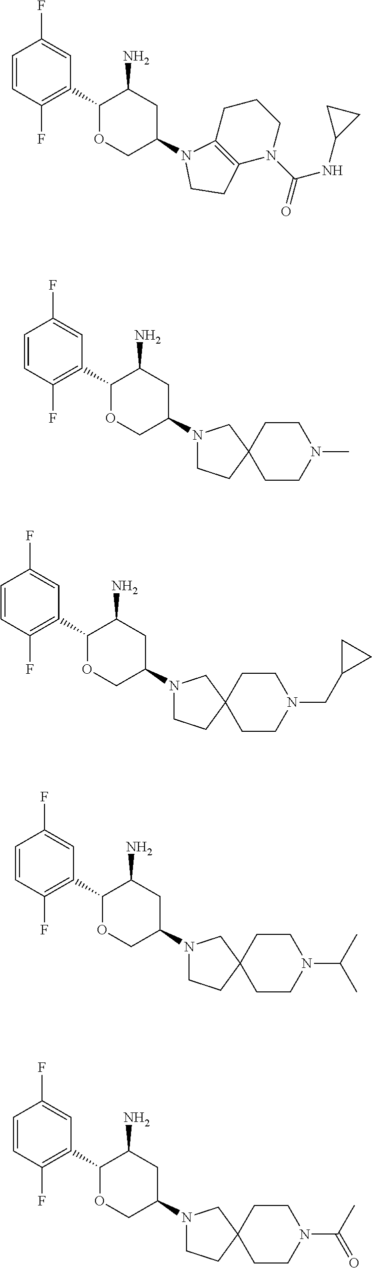 [SMILES: N[C@H]1C[C@@H](N2CCC3=C2CCCN3C(=O)NC2CC2)CO[C@@H]1c1cc(F)ccc1F ; CN1CCC2(CC1)CCN([C@H]1CO[C@H](c3cc(F)ccc3F)[C@@H](N)C1)C2 ; N[C@H]1C[C@@H](N2CCC3(CCN(CC4CC4)CC3)C2)CO[C@@H]1c1cc(F)ccc1F ; CC(C)N1CCC2(CC1)CCN([C@H]1CO[C@H](c3cc(F)ccc3F)[C@@H](N)C1)C2 ; CC(=O)N1CCC2(CC1)CCN([C@H]1CO[C@H](c3cc(F)ccc3F)[C@@H](N)C1)C2]

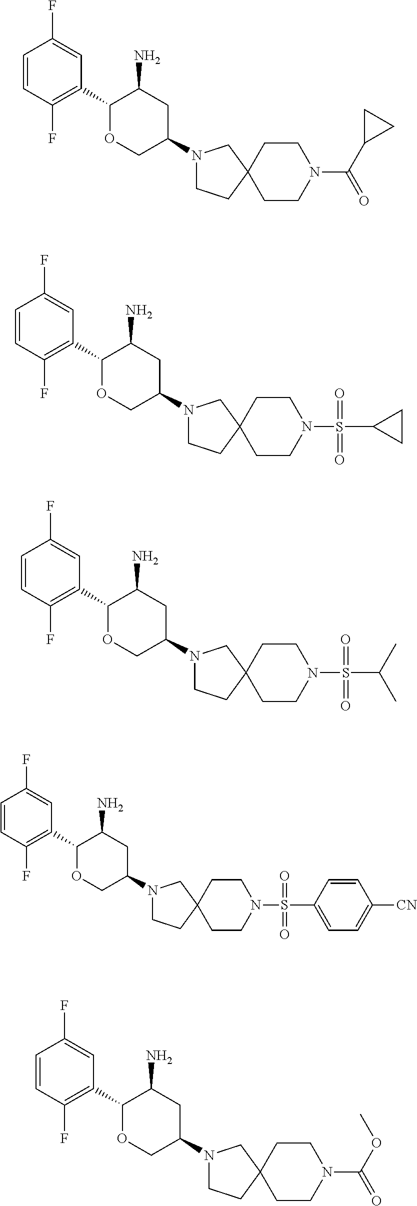 [SMILES: N[C@H]1C[C@@H](N2CCC3(CCN(C(=O)C4CC4)CC3)C2)CO[C@@H]1c1cc(F)ccc1F ; N[C@H]1C[C@@H](N2CCC3(CCN(S(=O)(=O)C4CC4)CC3)C2)CO[C@@H]1c1cc(F)ccc1F ; CC(C)S(=O)(=O)N1CCC2(CCN([C@H]3CO[C@H](c4cc(F)ccc4F)[C@@H](N)C3)C2)CC1 ; N#Cc1ccc(S(=O)(=O)N2CCC3(CCN([C@H]4CO[C@H](c5cc(F)ccc5F)[C@@H](N)C4)C3)CC2)cc1 ; COC(=O)N1CCC2(CC1)CCN([C@H]1CO[C@H](c3cc(F)ccc3F)[C@@H](N)C1)C2]

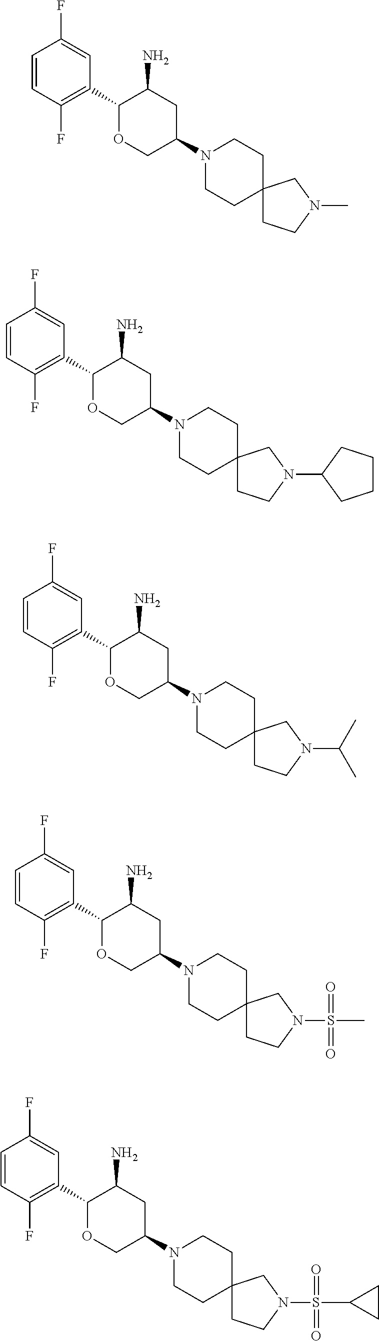 [SMILES: CN1CCC2(CCN([C@H]3CO[C@H](c4cc(F)ccc4F)[C@@H](N)C3)CC2)C1 ; N[C@H]1C[C@@H](N2CCC3(CCN(C4CCCC4)C3)CC2)CO[C@@H]1c1cc(F)ccc1F ; CC(C)N1CCC2(CCN([C@H]3CO[C@H](c4cc(F)ccc4F)[C@@H](N)C3)CC2)C1 ; CS(=O)(=O)N1CCC2(CCN([C@H]3CO[C@H](c4cc(F)ccc4F)[C@@H](N)C3)CC2)C1 ; N[C@H]1C[C@@H](N2CCC3(CC2)CCN(S(=O)(=O)C2CC2)C3)CO[C@@H]1c1cc(F)ccc1F]

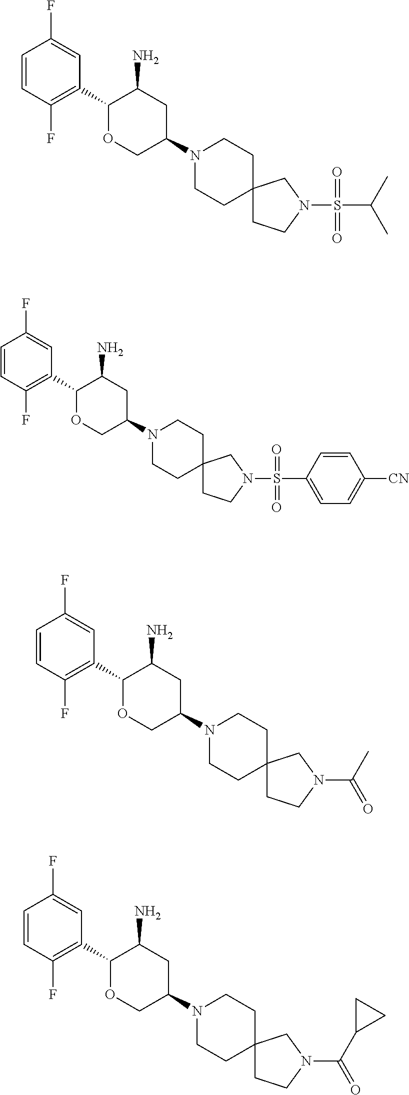 [SMILES: CC(C)S(=O)(=O)N1CCC2(CCN([C@H]3CO[C@H](c4cc(F)ccc4F)[C@@H](N)C3)CC2)C1 ; N#Cc1ccc(S(=O)(=O)N2CCC3(CCN([C@H]4CO[C@H](c5cc(F)ccc5F)[C@@H](N)C4)CC3)C2)cc1 ; CC(=O)N1CCC2(CCN([C@H]3CO[C@H](c4cc(F)ccc4F)[C@@H](N)C3)CC2)C1 ; N[C@H]1C[C@@H](N2CCC3(CCN(C(=O)C4CC4)C3)CC2)CO[C@@H]1c1cc(F)ccc1F]

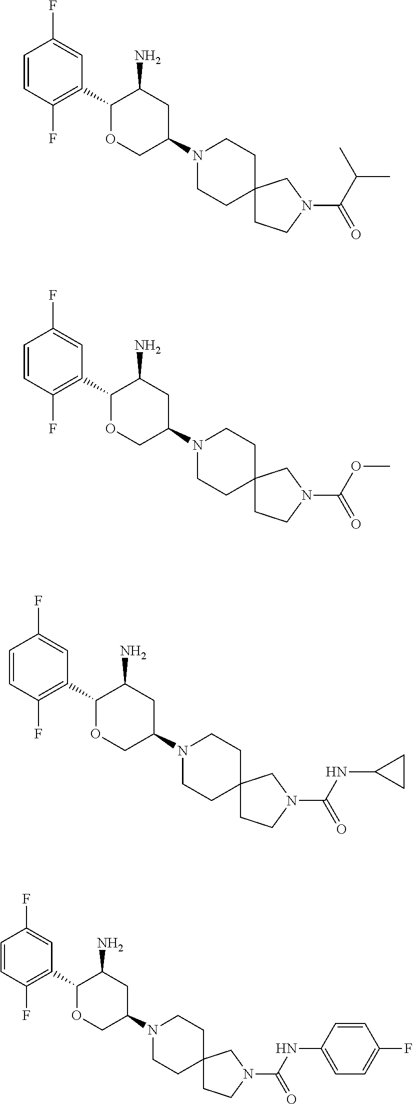 [SMILES: CC(C)C(=O)N1CCC2(CCN([C@H]3CO[C@H](c4cc(F)ccc4F)[C@@H](N)C3)CC2)C1 ; COC(=O)N1CCC2(CCN([C@H]3CO[C@H](c4cc(F)ccc4F)[C@@H](N)C3)CC2)C1 ; N[C@H]1C[C@@H](N2CCC3(CCN(C(=O)NC4CC4)C3)CC2)CO[C@@H]1c1cc(F)ccc1F ; N[C@H]1C[C@@H](N2CCC3(CCN(C(=O)Nc4ccc(F)cc4)C3)CC2)CO[C@@H]1c1cc(F)ccc1F]

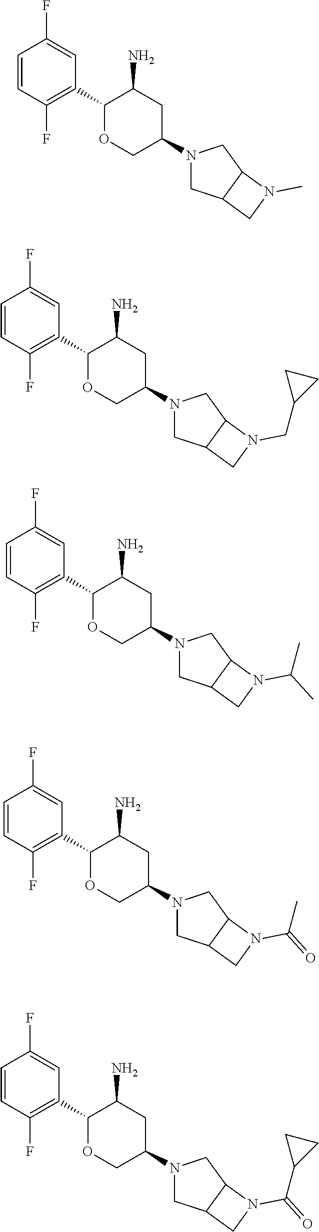 [SMILES: CN1CC2CN([C@H]3CO[C@H](c4cc(F)ccc4F)[C@@H](N)C3)CC21 ; N[C@H]1C[C@@H](N2CC3CN(CC4CC4)C3C2)CO[C@@H]1c1cc(F)ccc1F ; CC(C)N1CC2CN([C@H]3CO[C@H](c4cc(F)ccc4F)[C@@H](N)C3)CC21 ; CC(=O)N1CC2CN([C@H]3CO[C@H](c4cc(F)ccc4F)[C@@H](N)C3)CC21 ; N[C@H]1C[C@@H](N2CC3CN(C(=O)C4CC4)C3C2)CO[C@@H]1c1cc(F)ccc1F]

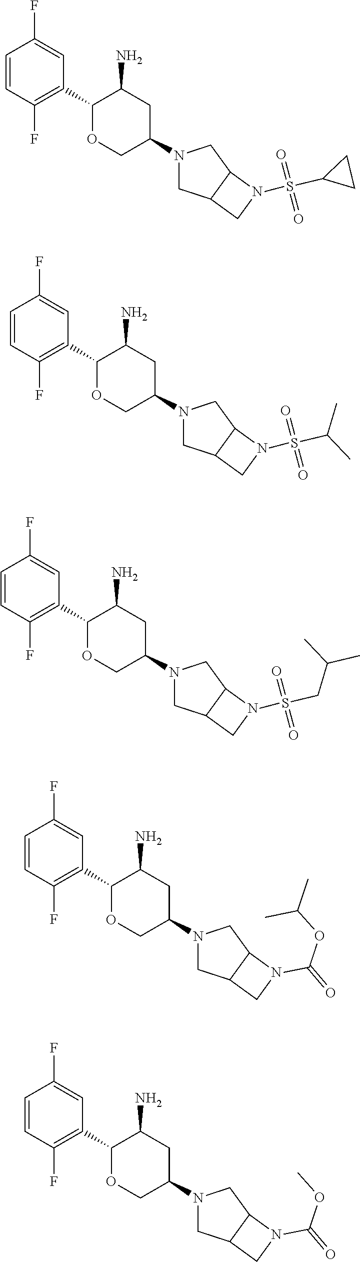 [SMILES: N[C@H]1C[C@@H](N2CC3CN(S(=O)(=O)C4CC4)C3C2)CO[C@@H]1c1cc(F)ccc1F ; CC(C)S(=O)(=O)N1CC2CN([C@H]3CO[C@H](c4cc(F)ccc4F)[C@@H](N)C3)CC21 ; CC(C)CS(=O)(=O)N1CC2CN([C@H]3CO[C@H](c4cc(F)ccc4F)[C@@H](N)C3)CC21 ; CC(C)OC(=O)N1CC2CN([C@H]3CO[C@H](c4cc(F)ccc4F)[C@@H](N)C3)CC21 ; COC(=O)N1CC2CN([C@H]3CO[C@H](c4cc(F)ccc4F)[C@@H](N)C3)CC21]

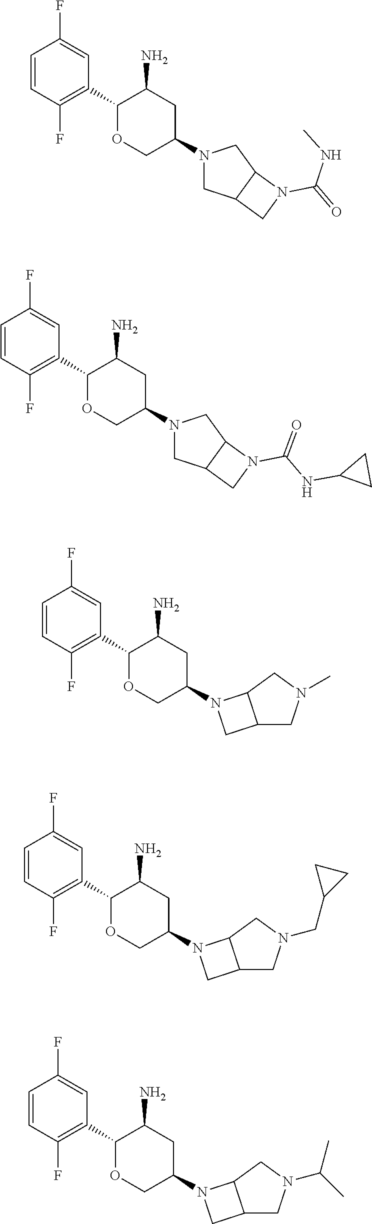 [SMILES: CNC(=O)N1CC2CN([C@H]3CO[C@H](c4cc(F)ccc4F)[C@@H](N)C3)CC21 ; N[C@H]1C[C@@H](N2CC3CN(C(=O)NC4CC4)C3C2)CO[C@@H]1c1cc(F)ccc1F ; CN1CC2CN([C@H]3CO[C@H](c4cc(F)ccc4F)[C@@H](N)C3)C2C1 ; N[C@H]1C[C@@H](N2CC3CN(CC4CC4)CC32)CO[C@@H]1c1cc(F)ccc1F ; CC(C)N1CC2CN([C@H]3CO[C@H](c4cc(F)ccc4F)[C@@H](N)C3)C2C1]

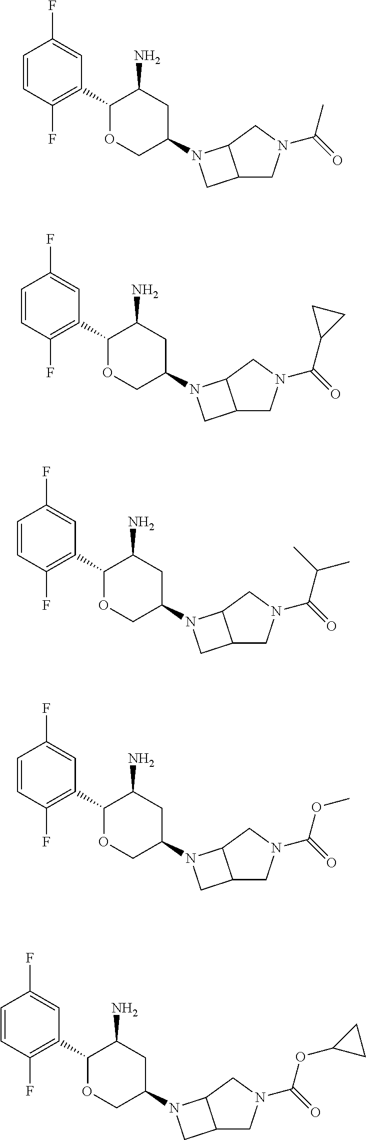 [SMILES: CC(=O)N1CC2CN([C@H]3CO[C@H](c4cc(F)ccc4F)[C@@H](N)C3)C2C1 ; N[C@H]1C[C@@H](N2CC3CN(C(=O)C4CC4)CC32)CO[C@@H]1c1cc(F)ccc1F ; CC(C)C(=O)N1CC2CN([C@H]3CO[C@H](c4cc(F)ccc4F)[C@@H](N)C3)C2C1 ; COC(=O)N1CC2CN([C@H]3CO[C@H](c4cc(F)ccc4F)[C@@H](N)C3)C2C1 ; N[C@H]1C[C@@H](N2CC3CN(C(=O)OC4CC4)CC32)CO[C@@H]1c1cc(F)ccc1F]

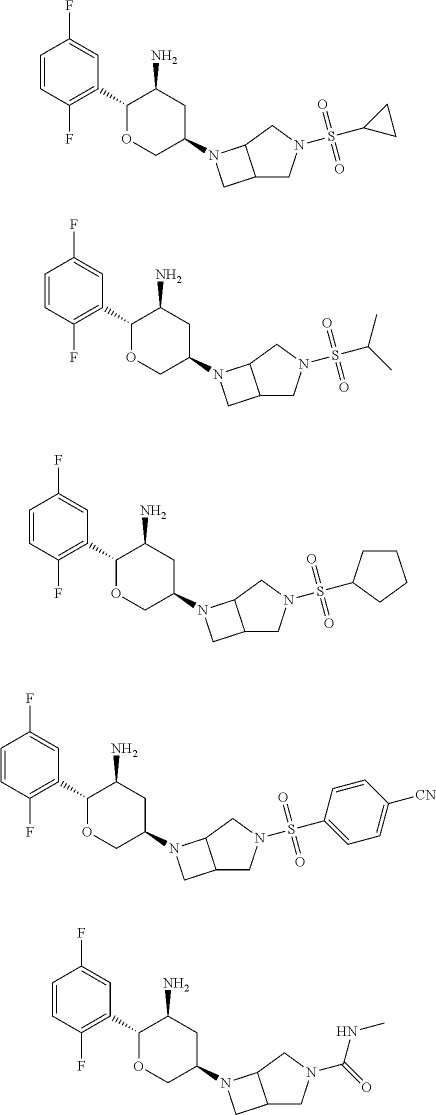 [SMILES: N[C@H]1C[C@@H](N2CC3CN(S(=O)(=O)C4CC4)CC32)CO[C@@H]1c1cc(F)ccc1F ; CC(C)S(=O)(=O)N1CC2CN([C@H]3CO[C@H](c4cc(F)ccc4F)[C@@H](N)C3)C2C1 ; N[C@H]1C[C@@H](N2CC3CN(S(=O)(=O)C4CCCC4)CC32)CO[C@@H]1c1cc(F)ccc1F ; N#Cc1ccc(S(=O)(=O)N2CC3CN([C@H]4CO[C@H](c5cc(F)ccc5F)[C@@H](N)C4)C3C2)cc1 ; CNC(=O)N1CC2CN([C@H]3CO[C@H](c4cc(F)ccc4F)[C@@H](N)C3)C2C1]

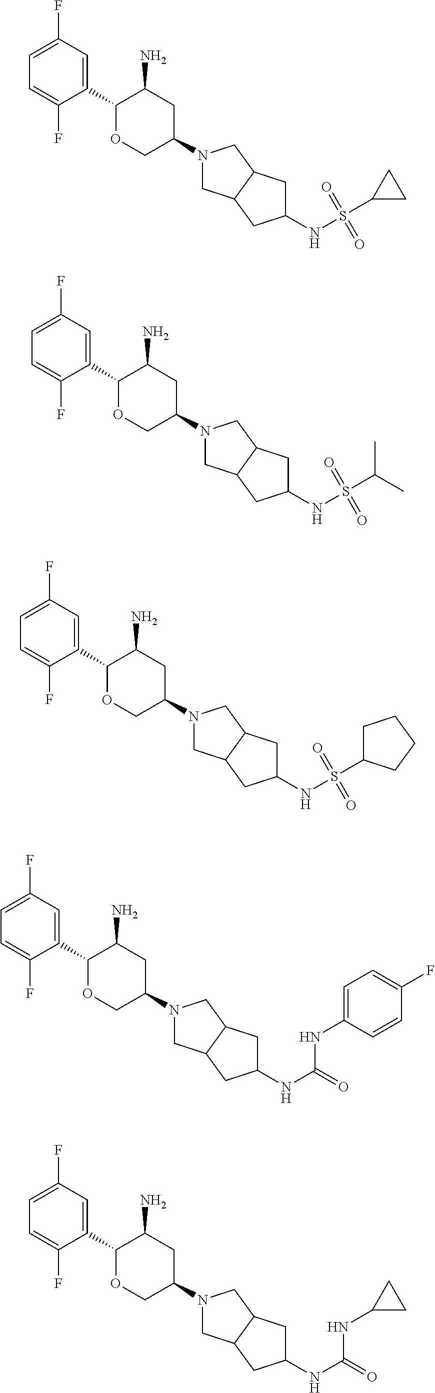 [SMILES: N[C@H]1C[C@@H](N2CC3CC(NS(=O)(=O)C4CC4)CC3C2)CO[C@@H]1c1cc(F)ccc1F ; CC(C)S(=O)(=O)NC1CC2CN([C@H]3CO[C@H](c4cc(F)ccc4F)[C@@H](N)C3)CC2C1 ; N[C@H]1C[C@@H](N2CC3CC(NS(=O)(=O)C4CCCC4)CC3C2)CO[C@@H]1c1cc(F)ccc1F ; N[C@H]1C[C@@H](N2CC3CC(NC(=O)Nc4ccc(F)cc4)CC3C2)CO[C@@H]1c1cc(F)ccc1F ; N[C@H]1C[C@@H](N2CC3CC(NC(=O)NC4CC4)CC3C2)CO[C@@H]1c1cc(F)ccc1F]

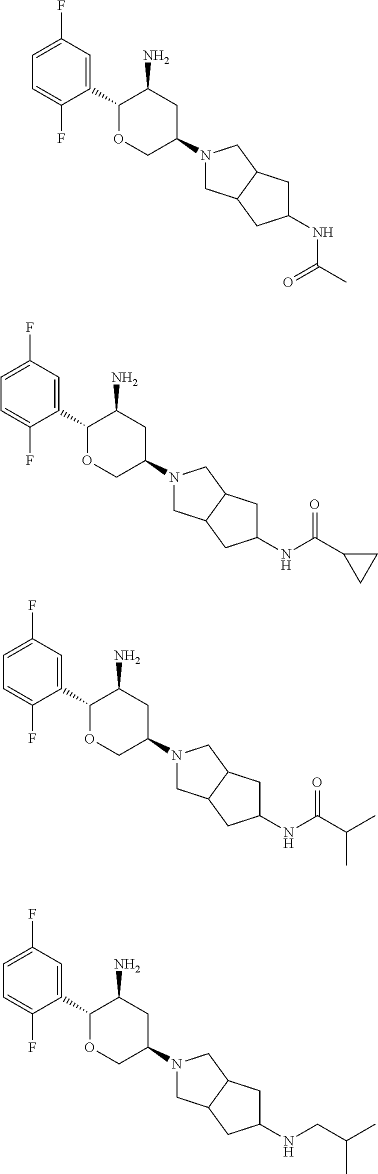 [SMILES: CC(=O)NC1CC2CN([C@H]3CO[C@H](c4cc(F)ccc4F)[C@@H](N)C3)CC2C1 ; N[C@H]1C[C@@H](N2CC3CC(NC(=O)C4CC4)CC3C2)CO[C@@H]1c1cc(F)ccc1F ; CC(C)C(=O)NC1CC2CN([C@H]3CO[C@H](c4cc(F)ccc4F)[C@@H](N)C3)CC2C1 ; CC(C)CNC1CC2CN([C@H]3CO[C@H](c4cc(F)ccc4F)[C@@H](N)C3)CC2C1]

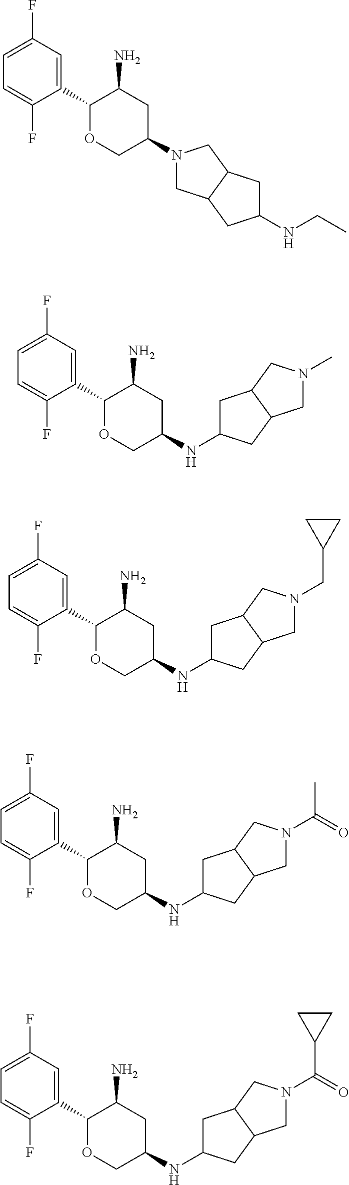 [SMILES: CCNC1CC2CN([C@H]3CO[C@H](c4cc(F)ccc4F)[C@@H](N)C3)CC2C1 ; CN1CC2CC(N[C@H]3CO[C@H](c4cc(F)ccc4F)[C@@H](N)C3)CC2C1 ; N[C@H]1C[C@@H](NC2CC3CN(CC4CC4)CC3C2)CO[C@@H]1c1cc(F)ccc1F ; CC(=O)N1CC2CC(N[C@H]3CO[C@H](c4cc(F)ccc4F)[C@@H](N)C3)CC2C1 ; N[C@H]1C[C@@H](NC2CC3CN(C(=O)C4CC4)CC3C2)CO[C@@H]1c1cc(F)ccc1F]

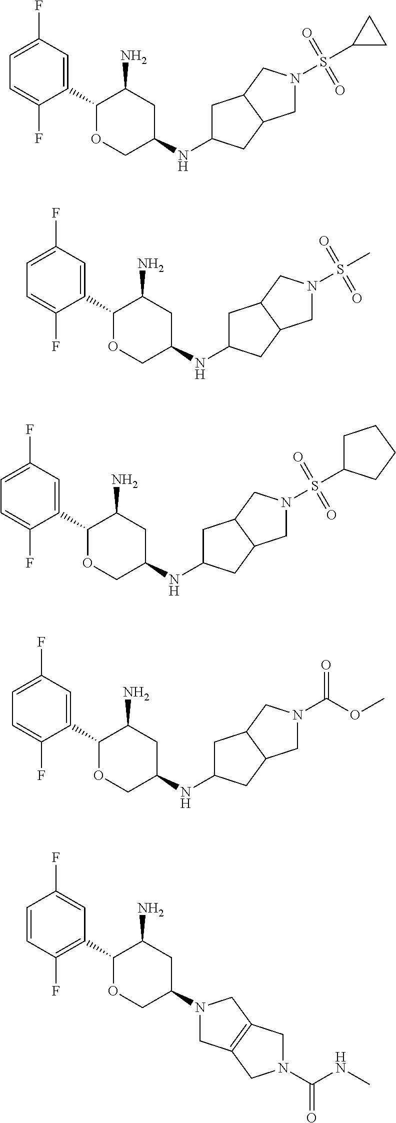 [SMILES: N[C@H]1C[C@@H](NC2CC3CN(S(=O)(=O)C4CC4)CC3C2)CO[C@@H]1c1cc(F)ccc1F ; CS(=O)(=O)N1CC2CC(N[C@H]3CO[C@H](c4cc(F)ccc4F)[C@@H](N)C3)CC2C1 ; N[C@H]1C[C@@H](NC2CC3CN(S(=O)(=O)C4CCCC4)CC3C2)CO[C@@H]1c1cc(F)ccc1F ; COC(=O)N1CC2CC(N[C@H]3CO[C@H](c4cc(F)ccc4F)[C@@H](N)C3)CC2C1 ; CNC(=O)N1CC2=C(C1)CN([C@H]1CO[C@H](c3cc(F)ccc3F)[C@@H](N)C1)C2]

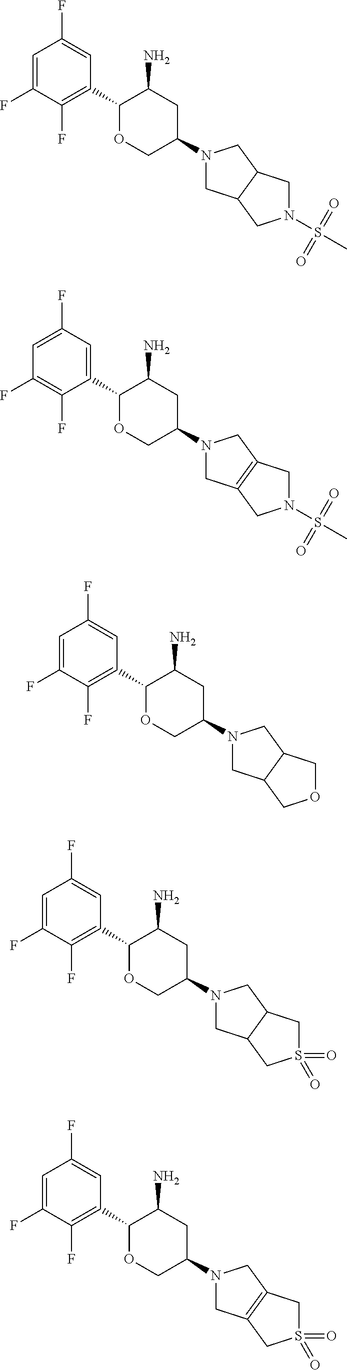 [SMILES: CS(=O)(=O)N1CC2CN([C@H]3CO[C@H](c4cc(F)cc(F)c4F)[C@@H](N)C3)CC2C1 ; CS(=O)(=O)N1CC2=C(CN([C@H]3CO[C@H](c4cc(F)cc(F)c4F)[C@@H](N)C3)C2)C1 ; N[C@H]1C[C@@H](N2CC3COCC3C2)CO[C@@H]1c1cc(F)cc(F)c1F ; N[C@H]1C[C@@H](N2CC3CS(=O)(=O)CC3C2)CO[C@@H]1c1cc(F)cc(F)c1F ; N[C@H]1C[C@@H](N2CC3=C(C2)CS(=O)(=O)C3)CO[C@@H]1c1cc(F)cc(F)c1F]

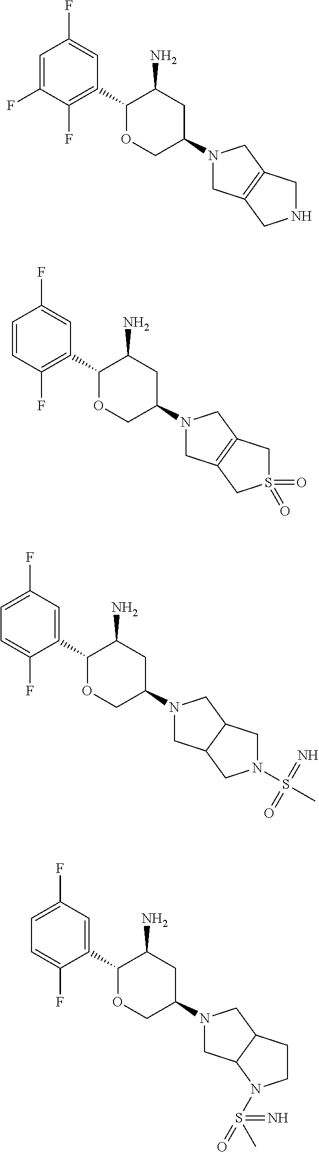 [SMILES: N[C@H]1C[C@@H](N2CC3=C(CNC3)C2)CO[C@@H]1c1cc(F)cc(F)c1F ; N[C@H]1C[C@@H](N2CC3=C(C2)CS(=O)(=O)C3)CO[C@@H]1c1cc(F)ccc1F ; CS(=N)(=O)N1CC2CN([C@H]3CO[C@H](c4cc(F)ccc4F)[C@@H](N)C3)CC2C1 ; CS(=N)(=O)N1CCC2CN([C@H]3CO[C@H](c4cc(F)ccc4F)[C@@H](N)C3)CC21]

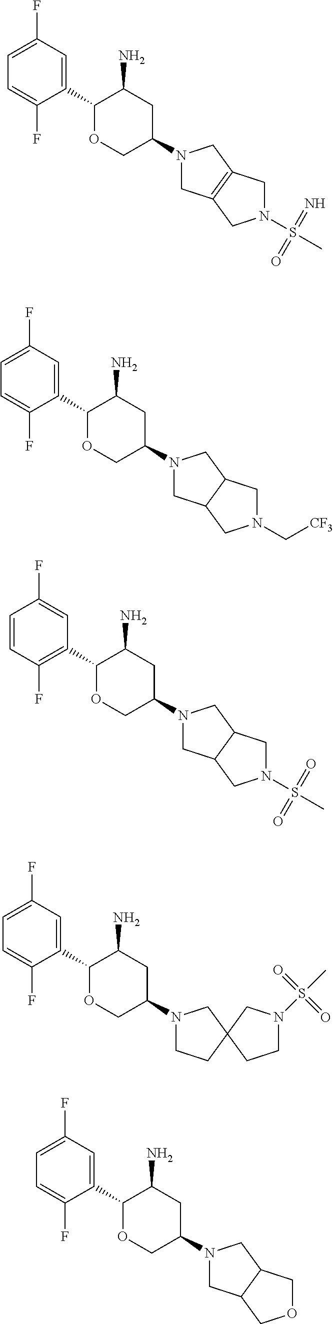 [SMILES: CS(=N)(=O)N1CC2=C(CN([C@H]3CO[C@H](c4cc(F)ccc4F)[C@@H](N)C3)C2)C1 ; N[C@H]1C[C@@H](N2CC3CN(CC(F)(F)F)CC3C2)CO[C@@H]1c1cc(F)ccc1F ; CS(=O)(=O)N1CC2CN([C@H]3CO[C@H](c4cc(F)ccc4F)[C@@H](N)C3)CC2C1 ; CS(=O)(=O)N1CCC2(CCN([C@H]3CO[C@H](c4cc(F)ccc4F)[C@@H](N)C3)C2)C1 ; N[C@H]1C[C@@H](N2CC3COCC3C2)CO[C@@H]1c1cc(F)ccc1F]

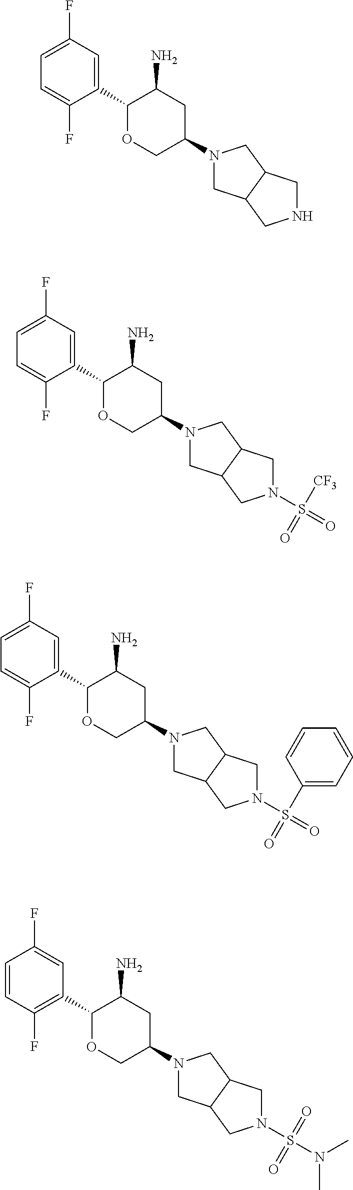 [SMILES: N[C@H]1C[C@@H](N2CC3CNCC3C2)CO[C@@H]1c1cc(F)ccc1F ; N[C@H]1C[C@@H](N2CC3CN(S(=O)(=O)C(F)(F)F)CC3C2)CO[C@@H]1c1cc(F)ccc1F ; N[C@H]1C[C@@H](N2CC3CN(S(=O)(=O)c4ccccc4)CC3C2)CO[C@@H]1c1cc(F)ccc1F ; CN(C)S(=O)(=O)N1CC2CN([C@H]3CO[C@H](c4cc(F)ccc4F)[C@@H](N)C3)CC2C1]

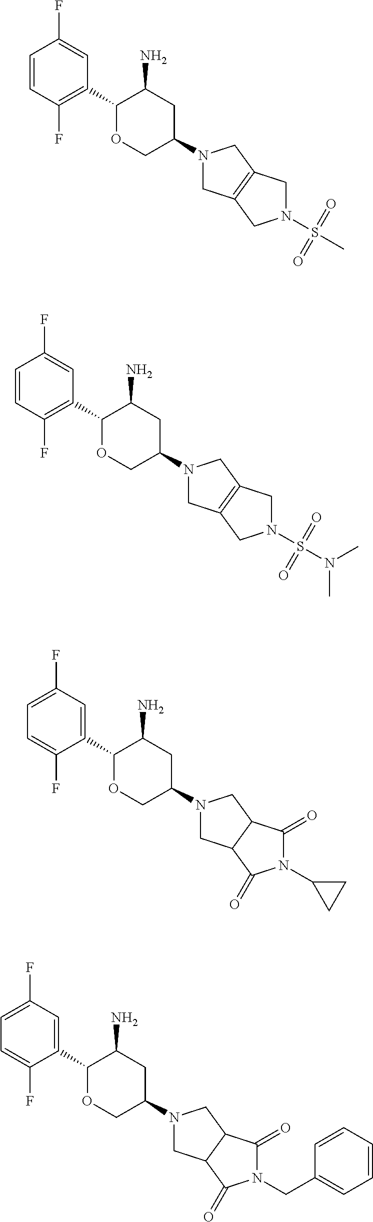 [SMILES: CS(=O)(=O)N1CC2=C(CN([C@H]3CO[C@H](c4cc(F)ccc4F)[C@@H](N)C3)C2)C1 ; CN(C)S(=O)(=O)N1CC2=C(CN([C@H]3CO[C@H](c4cc(F)ccc4F)[C@@H](N)C3)C2)C1 ; N[C@H]1C[C@@H](N2CC3C(=O)N(C4CC4)C(=O)C3C2)CO[C@@H]1c1cc(F)ccc1F ; N[C@H]1C[C@@H](N2CC3C(=O)N(Cc4ccccc4)C(=O)C3C2)CO[C@@H]1c1cc(F)ccc1F]

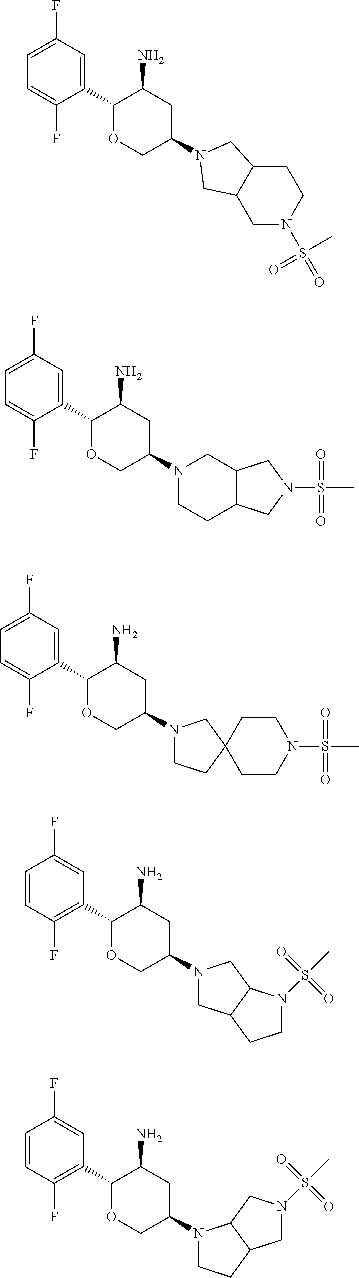 [SMILES: CS(=O)(=O)N1CCC2CN([C@H]3CO[C@H](c4cc(F)ccc4F)[C@@H](N)C3)CC2C1 ; CS(=O)(=O)N1CC2CCN([C@H]3CO[C@H](c4cc(F)ccc4F)[C@@H](N)C3)CC2C1 ; CS(=O)(=O)N1CCC2(CCN([C@H]3CO[C@H](c4cc(F)ccc4F)[C@@H](N)C3)C2)CC1 ; CS(=O)(=O)N1CCC2CN([C@H]3CO[C@H](c4cc(F)ccc4F)[C@@H](N)C3)CC21 ; CS(=O)(=O)N1CC2CCN([C@H]3CO[C@H](c4cc(F)ccc4F)[C@@H](N)C3)C2C1]

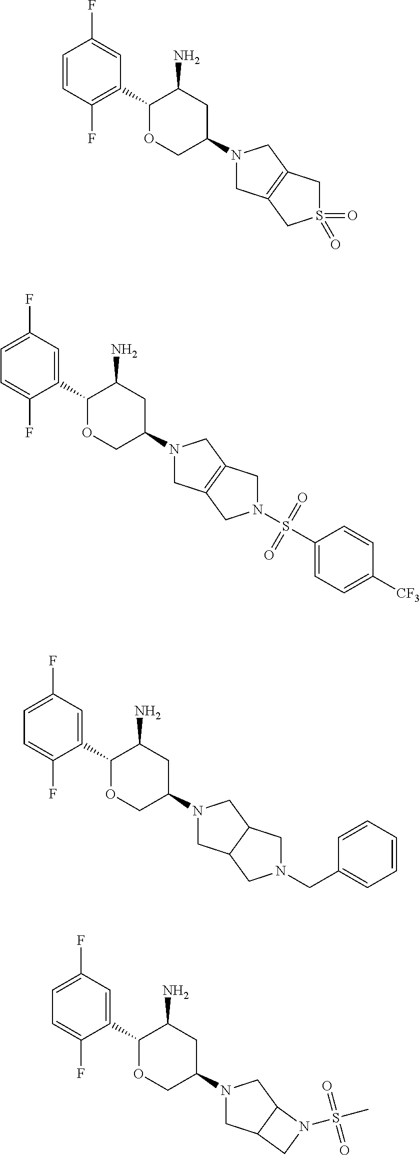 [SMILES: N[C@H]1C[C@@H](N2CC3=C(C2)CS(=O)(=O)C3)CO[C@@H]1c1cc(F)ccc1F ; N[C@H]1C[C@@H](N2CC3=C(C2)CN(S(=O)(=O)c2ccc(C(F)(F)F)cc2)C3)CO[C@@H]1c1cc(F)ccc1F ; N[C@H]1C[C@@H](N2CC3CN(Cc4ccccc4)CC3C2)CO[C@@H]1c1cc(F)ccc1F ; CS(=O)(=O)N1CC2CN([C@H]3CO[C@H](c4cc(F)ccc4F)[C@@H](N)C3)CC21]

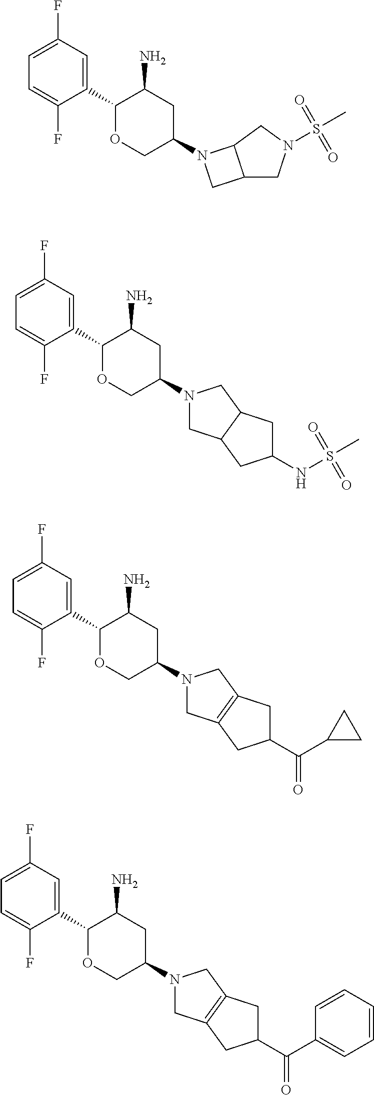 [SMILES: CS(=O)(=O)N1CC2CN([C@H]3CO[C@H](c4cc(F)ccc4F)[C@@H](N)C3)C2C1 ; CS(=O)(=O)NC1CC2CN([C@H]3CO[C@H](c4cc(F)ccc4F)[C@@H](N)C3)CC2C1 ; N[C@H]1C[C@@H](N2CC3=C(CC(C(=O)C4CC4)C3)C2)CO[C@@H]1c1cc(F)ccc1F ; N[C@H]1C[C@@H](N2CC3=C(CC(C(=O)c4ccccc4)C3)C2)CO[C@@H]1c1cc(F)ccc1F]

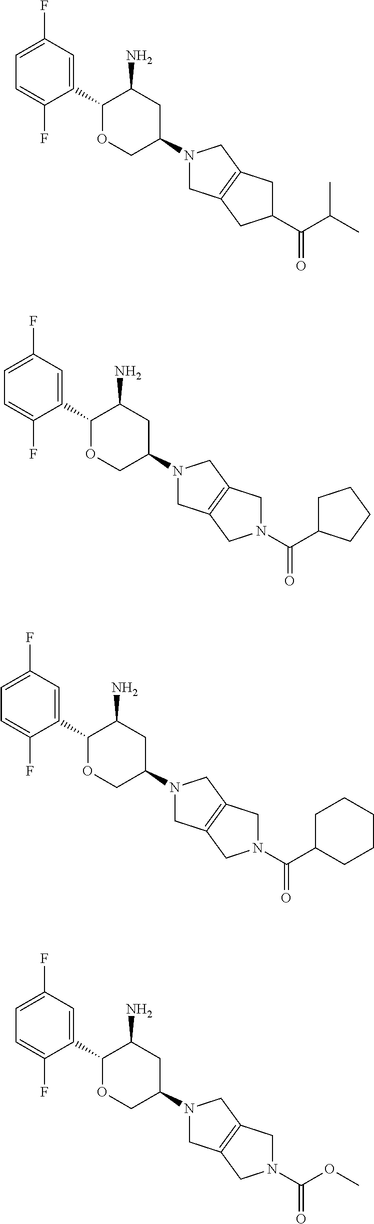 [SMILES: CC(C)C(=O)C1CC2=C(C1)CN([C@H]1CO[C@H](c3cc(F)ccc3F)[C@@H](N)C1)C2 ; N[C@H]1C[C@@H](N2CC3=C(CN(C(=O)C4CCCC4)C3)C2)CO[C@@H]1c1cc(F)ccc1F ; N[C@H]1C[C@@H](N2CC3=C(CN(C(=O)C4CCCCC4)C3)C2)CO[C@@H]1c1cc(F)ccc1F ; COC(=O)N1CC2=C(C1)CN([C@H]1CO[C@H](c3cc(F)ccc3F)[C@@H](N)C1)C2]

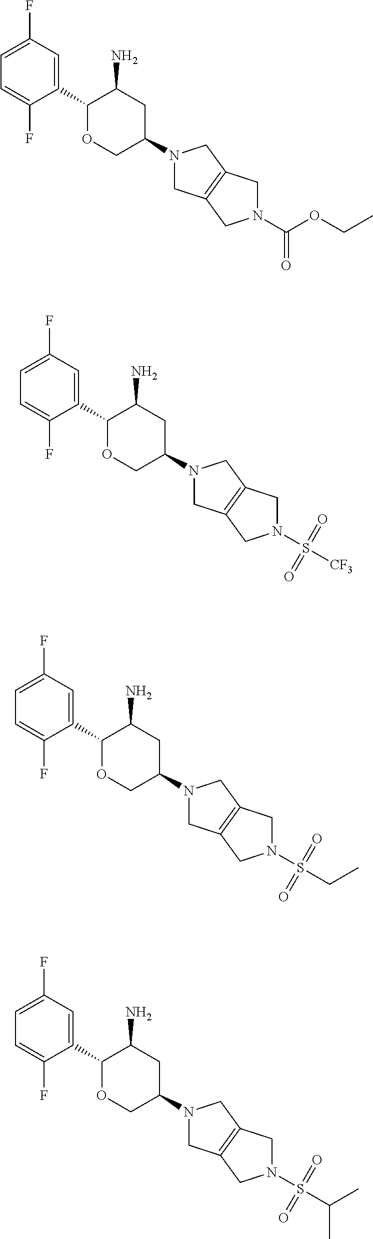 [SMILES: CCOC(=O)N1CC2=C(C1)CN([C@H]1CO[C@H](c3cc(F)ccc3F)[C@@H](N)C1)C2 ; N[C@H]1C[C@@H](N2CC3=C(C2)CN(S(=O)(=O)C(F)(F)F)C3)CO[C@@H]1c1cc(F)ccc1F ; CCS(=O)(=O)N1CC2=C(CN([C@H]3CO[C@H](c4cc(F)ccc4F)[C@@H](N)C3)C2)C1 ; CC(C)S(=O)(=O)N1CC2=C(CN([C@H]3CO[C@H](c4cc(F)ccc4F)[C@@H](N)C3)C2)C1]

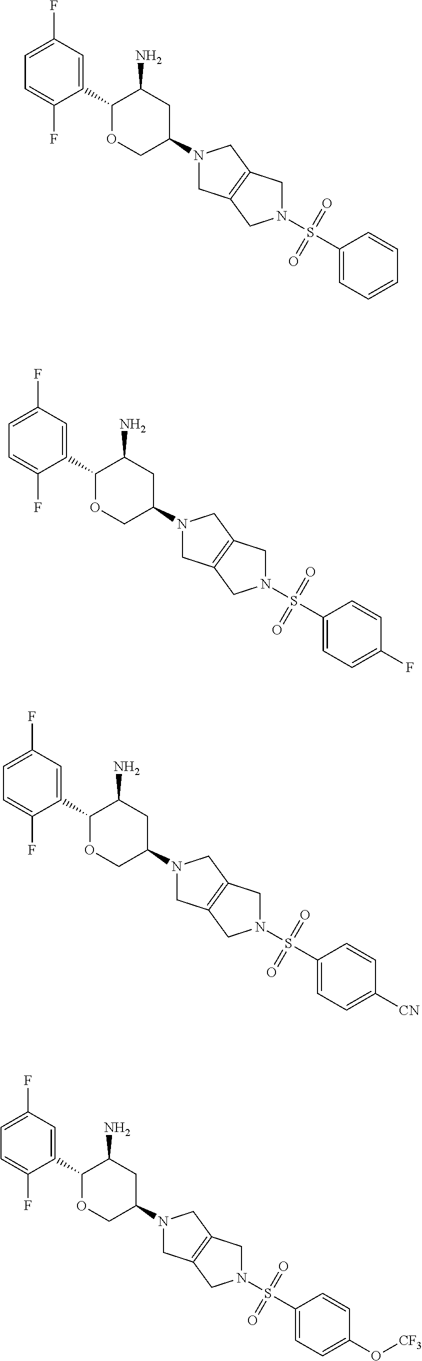 [SMILES: N[C@H]1C[C@@H](N2CC3=C(C2)CN(S(=O)(=O)c2ccccc2)C3)CO[C@@H]1c1cc(F)ccc1F ; N[C@H]1C[C@@H](N2CC3=C(C2)CN(S(=O)(=O)c2ccc(F)cc2)C3)CO[C@@H]1c1cc(F)ccc1F ; N#Cc1ccc(S(=O)(=O)N2CC3=C(CN([C@H]4CO[C@H](c5cc(F)ccc5F)[C@@H](N)C4)C3)C2)cc1 ; N[C@H]1C[C@@H](N2CC3=C(C2)CN(S(=O)(=O)c2ccc(OC(F)(F)F)cc2)C3)CO[C@@H]1c1cc(F)ccc1F]

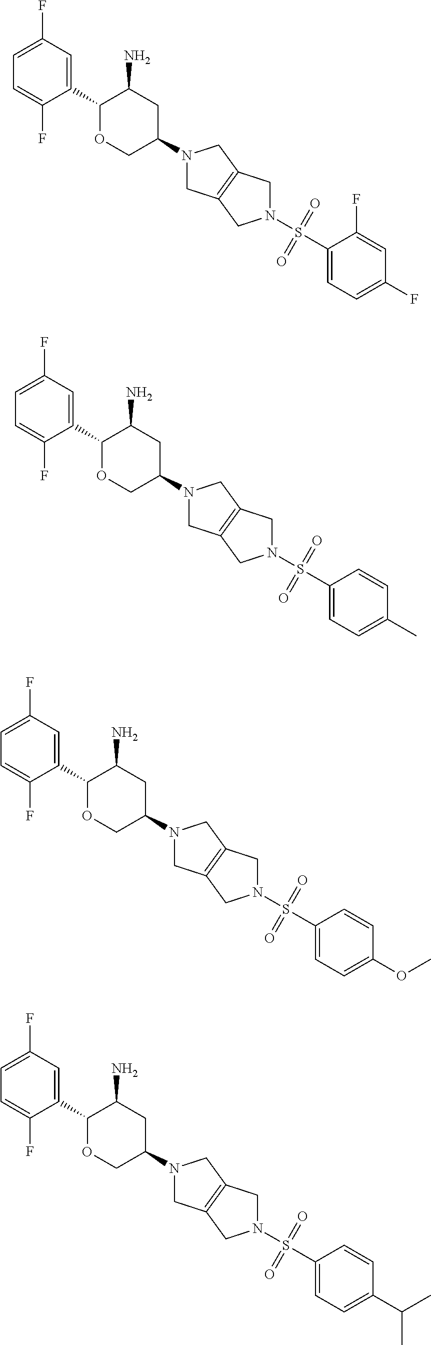 [SMILES: N[C@H]1C[C@@H](N2CC3=C(C2)CN(S(=O)(=O)c2ccc(F)cc2F)C3)CO[C@@H]1c1cc(F)ccc1F ; Cc1ccc(S(=O)(=O)N2CC3=C(CN([C@H]4CO[C@H](c5cc(F)ccc5F)[C@@H](N)C4)C3)C2)cc1 ; COc1ccc(S(=O)(=O)N2CC3=C(CN([C@H]4CO[C@H](c5cc(F)ccc5F)[C@@H](N)C4)C3)C2)cc1 ; CC(C)c1ccc(S(=O)(=O)N2CC3=C(CN([C@H]4CO[C@H](c5cc(F)ccc5F)[C@@H](N)C4)C3)C2)cc1]

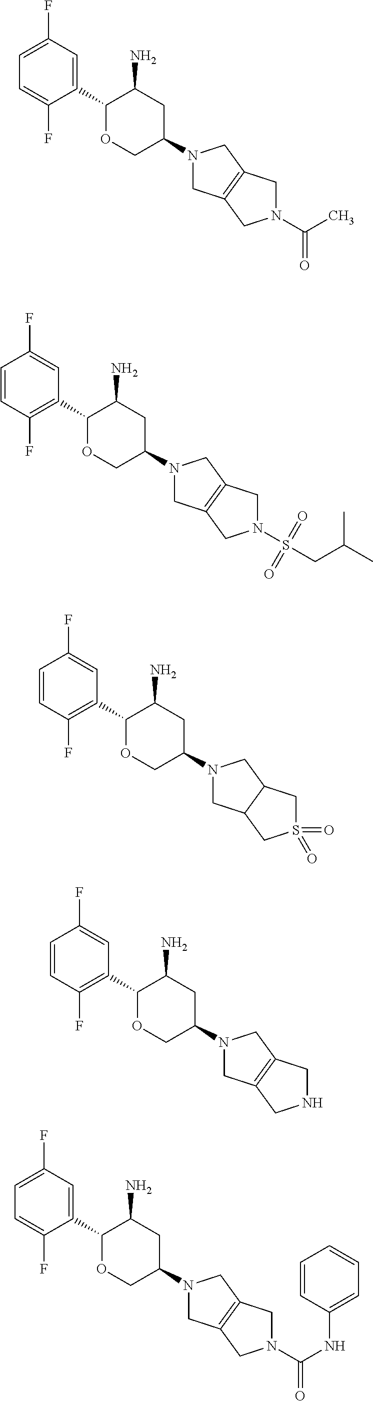 [SMILES: CC(=O)N1CC2=C(C1)CN([C@H]1CO[C@H](c3cc(F)ccc3F)[C@@H](N)C1)C2 ; CC(C)CS(=O)(=O)N1CC2=C(CN([C@H]3CO[C@H](c4cc(F)ccc4F)[C@@H](N)C3)C2)C1 ; N[C@H]1C[C@@H](N2CC3CS(=O)(=O)CC3C2)CO[C@@H]1c1cc(F)ccc1F ; N[C@H]1C[C@@H](N2CC3=C(CNC3)C2)CO[C@@H]1c1cc(F)ccc1F ; N[C@H]1C[C@@H](N2CC3=C(CN(C(=O)Nc4ccccc4)C3)C2)CO[C@@H]1c1cc(F)ccc1F]

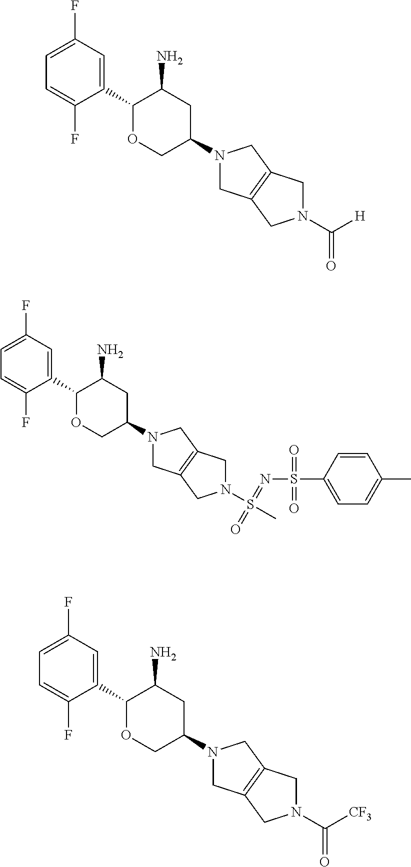 [SMILES: N[C@H]1C[C@@H](N2CC3=C(CN(C=O)C3)C2)CO[C@@H]1c1cc(F)ccc1F ; Cc1ccc(S(=O)(=O)N=S(C)(=O)N2CC3=C(CN([C@H]4CO[C@H](c5cc(F)ccc5F)[C@@H](N)C4)C3)C2)cc1 ; N[C@H]1C[C@@H](N2CC3=C(CN(C(=O)C(F)(F)F)C3)C2)CO[C@@H]1c1cc(F)ccc1F]

7. The compound as claimed in preferably selected from the group comprising of:

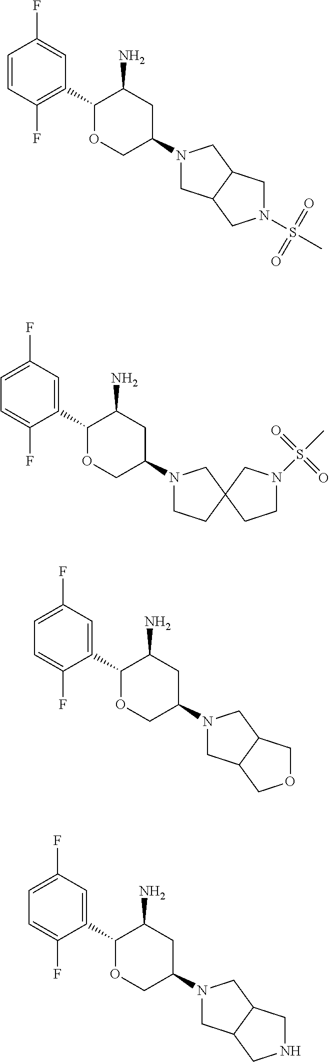 [SMILES: CS(=O)(=O)N1CC2CN([C@H]3CO[C@H](c4cc(F)ccc4F)[C@@H](N)C3)CC2C1 ; CS(=O)(=O)N1CCC2(CCN([C@H]3CO[C@H](c4cc(F)ccc4F)[C@@H](N)C3)C2)C1 ; N[C@H]1C[C@@H](N2CC3COCC3C2)CO[C@@H]1c1cc(F)ccc1F ; N[C@H]1C[C@@H](N2CC3CNCC3C2)CO[C@@H]1c1cc(F)ccc1F]

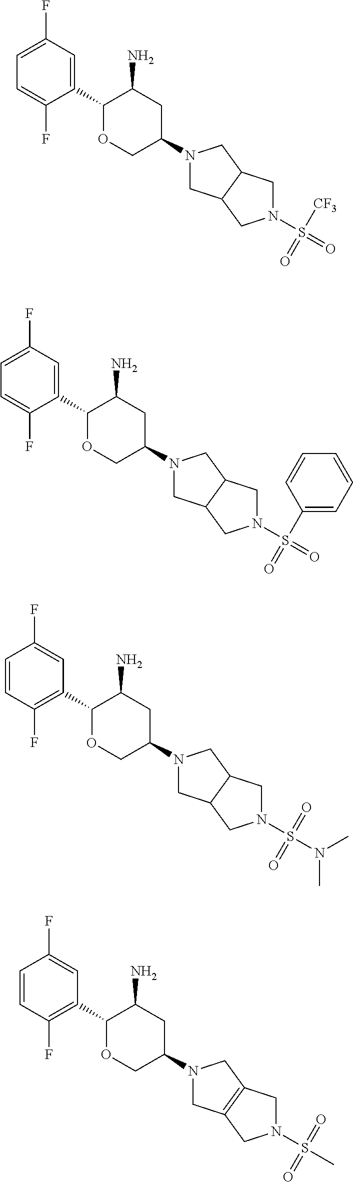 [SMILES: N[C@H]1C[C@@H](N2CC3CN(S(=O)(=O)C(F)(F)F)CC3C2)CO[C@@H]1c1cc(F)ccc1F ; N[C@H]1C[C@@H](N2CC3CN(S(=O)(=O)c4ccccc4)CC3C2)CO[C@@H]1c1cc(F)ccc1F ; CN(C)S(=O)(=O)N1CC2CN([C@H]3CO[C@H](c4cc(F)ccc4F)[C@@H](N)C3)CC2C1 ; CS(=O)(=O)N1CC2=C(CN([C@H]3CO[C@H](c4cc(F)ccc4F)[C@@H](N)C3)C2)C1]

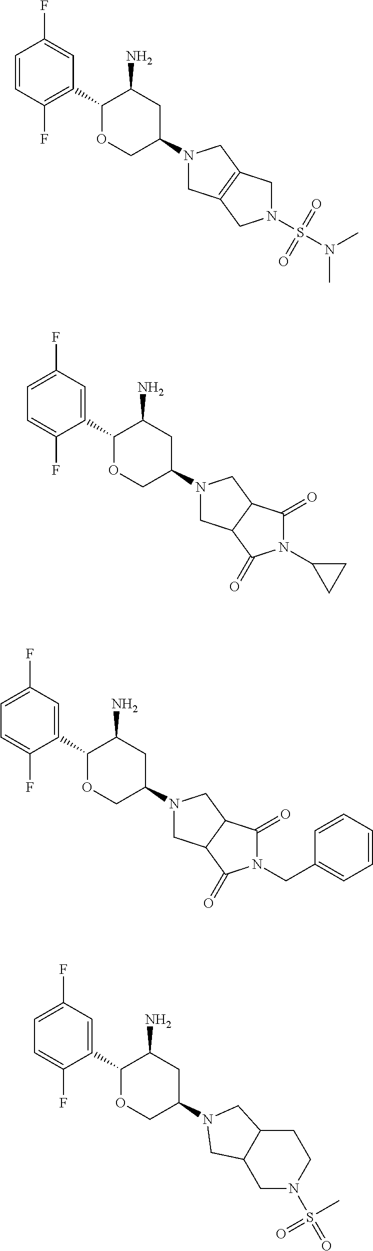 [SMILES: CN(C)S(=O)(=O)N1CC2=C(CN([C@H]3CO[C@H](c4cc(F)ccc4F)[C@@H](N)C3)C2)C1 ; N[C@H]1C[C@@H](N2CC3C(=O)N(C4CC4)C(=O)C3C2)CO[C@@H]1c1cc(F)ccc1F ; N[C@H]1C[C@@H](N2CC3C(=O)N(Cc4ccccc4)C(=O)C3C2)CO[C@@H]1c1cc(F)ccc1F ; CS(=O)(=O)N1CCC2CN([C@H]3CO[C@H](c4cc(F)ccc4F)[C@@H](N)C3)CC2C1]

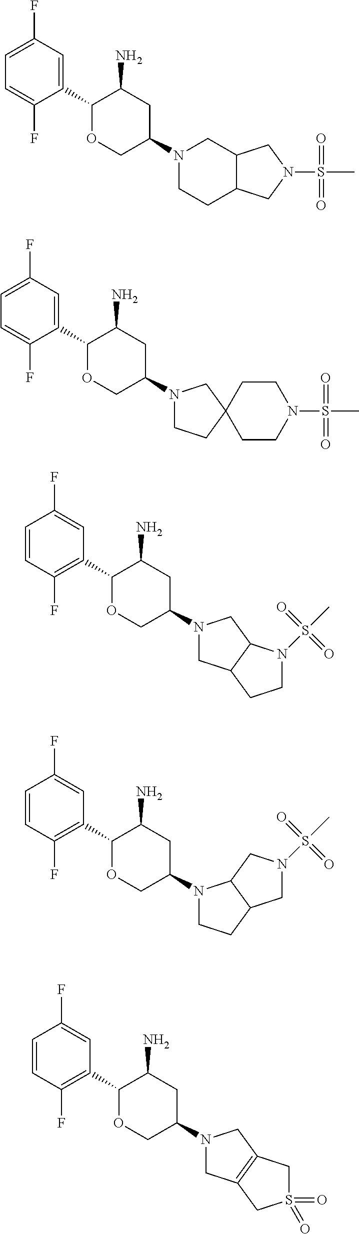 [SMILES: CS(=O)(=O)N1CC2CCN([C@H]3CO[C@H](c4cc(F)ccc4F)[C@@H](N)C3)CC2C1 ; CS(=O)(=O)N1CCC2(CCN([C@H]3CO[C@H](c4cc(F)ccc4F)[C@@H](N)C3)C2)CC1 ; CS(=O)(=O)N1CCC2CN([C@H]3CO[C@H](c4cc(F)ccc4F)[C@@H](N)C3)CC21 ; CS(=O)(=O)N1CC2CCN([C@H]3CO[C@H](c4cc(F)ccc4F)[C@@H](N)C3)C2C1 ; N[C@H]1C[C@@H](N2CC3=C(C2)CS(=O)(=O)C3)CO[C@@H]1c1cc(F)ccc1F]

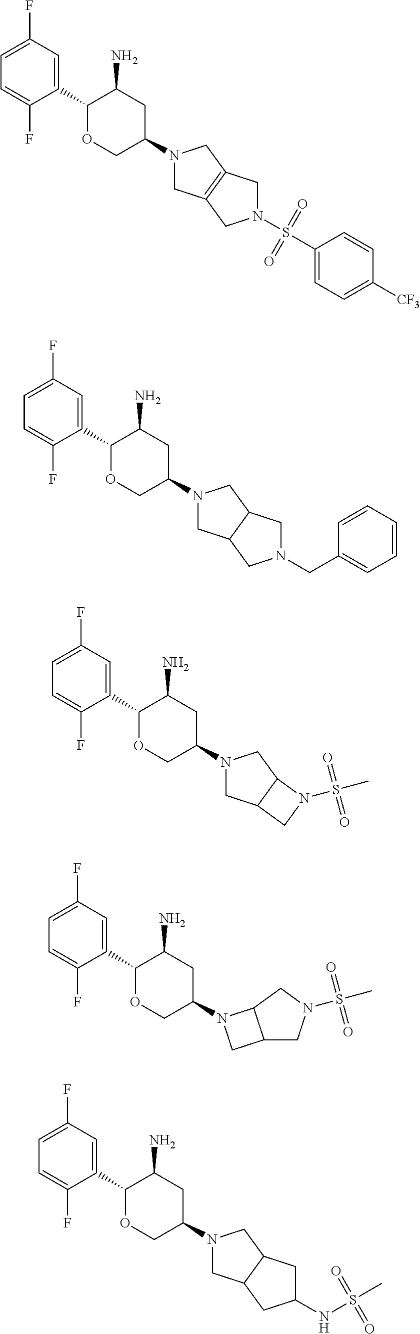 [SMILES: N[C@H]1C[C@@H](N2CC3=C(C2)CN(S(=O)(=O)c2ccc(C(F)(F)F)cc2)C3)CO[C@@H]1c1cc(F)ccc1F ; N[C@H]1C[C@@H](N2CC3CN(Cc4ccccc4)CC3C2)CO[C@@H]1c1cc(F)ccc1F ; CS(=O)(=O)N1CC2CN([C@H]3CO[C@H](c4cc(F)ccc4F)[C@@H](N)C3)CC21 ; CS(=O)(=O)N1CC2CN([C@H]3CO[C@H](c4cc(F)ccc4F)[C@@H](N)C3)C2C1 ; CS(=O)(=O)NC1CC2CN([C@H]3CO[C@H](c4cc(F)ccc4F)[C@@H](N)C3)CC2C1]

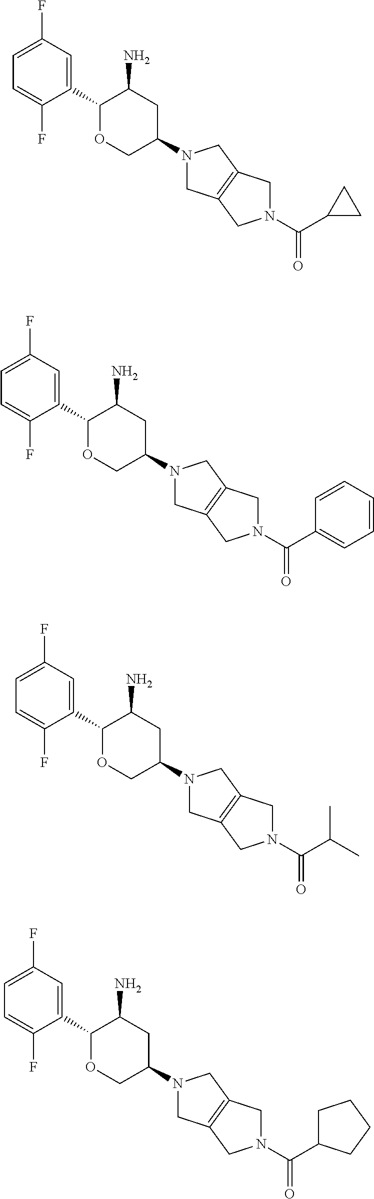 [SMILES: N[C@H]1C[C@@H](N2CC3=C(CN(C(=O)C4CC4)C3)C2)CO[C@@H]1c1cc(F)ccc1F ; N[C@H]1C[C@@H](N2CC3=C(CN(C(=O)c4ccccc4)C3)C2)CO[C@@H]1c1cc(F)ccc1F ; CC(C)C(=O)N1CC2=C(C1)CN([C@H]1CO[C@H](c3cc(F)ccc3F)[C@@H](N)C1)C2 ; N[C@H]1C[C@@H](N2CC3=C(CN(C(=O)C4CCCC4)C3)C2)CO[C@@H]1c1cc(F)ccc1F]

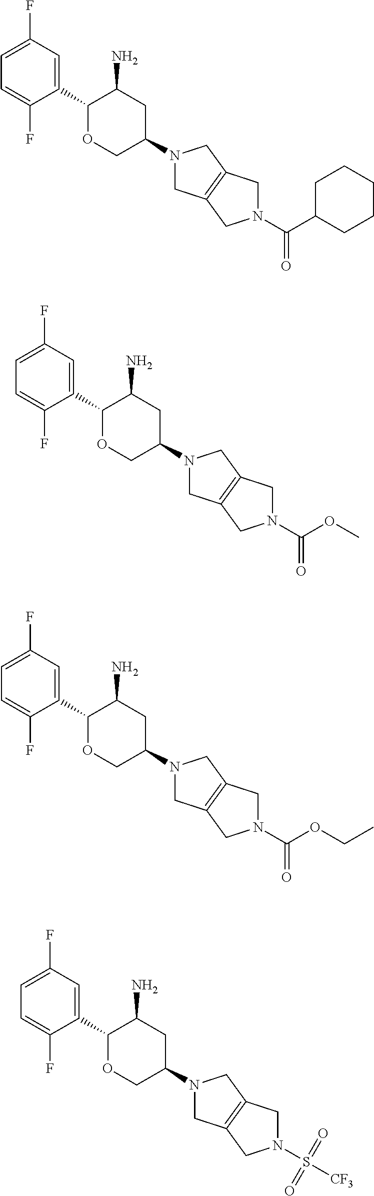 [SMILES: N[C@H]1C[C@@H](N2CC3=C(CN(C(=O)C4CCCCC4)C3)C2)CO[C@@H]1c1cc(F)ccc1F ; COC(=O)N1CC2=C(C1)CN([C@H]1CO[C@H](c3cc(F)ccc3F)[C@@H](N)C1)C2 ; CCOC(=O)N1CC2=C(C1)CN([C@H]1CO[C@H](c3cc(F)ccc3F)[C@@H](N)C1)C2 ; N[C@H]1C[C@@H](N2CC3=C(C2)CN(S(=O)(=O)C(F)(F)F)C3)CO[C@@H]1c1cc(F)ccc1F]

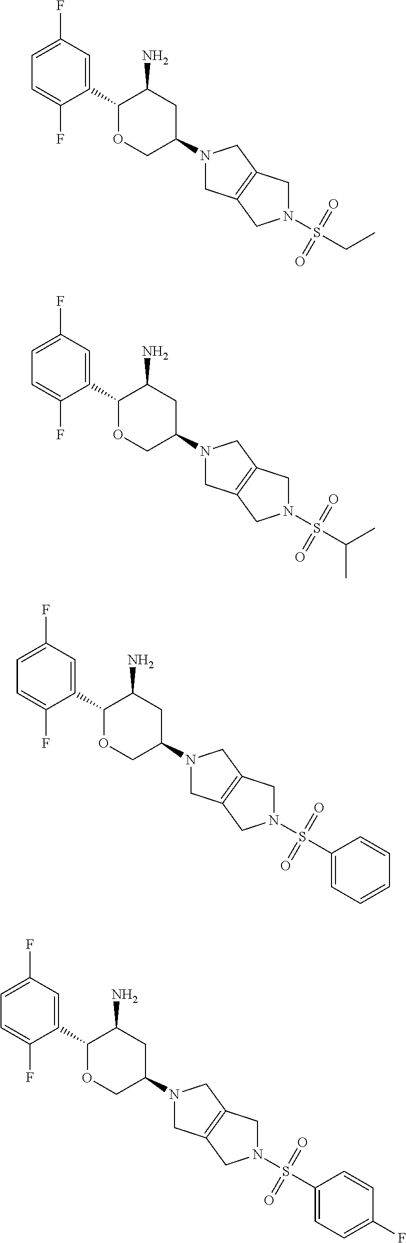 [SMILES: CCS(=O)(=O)N1CC2=C(CN([C@H]3CO[C@H](c4cc(F)ccc4F)[C@@H](N)C3)C2)C1 ; CC(C)S(=O)(=O)N1CC2=C(CN([C@H]3CO[C@H](c4cc(F)ccc4F)[C@@H](N)C3)C2)C1 ; N[C@H]1C[C@@H](N2CC3=C(C2)CN(S(=O)(=O)c2ccccc2)C3)CO[C@@H]1c1cc(F)ccc1F ; N[C@H]1C[C@@H](N2CC3=C(C2)CN(S(=O)(=O)c2ccc(F)cc2)C3)CO[C@@H]1c1cc(F)ccc1F]

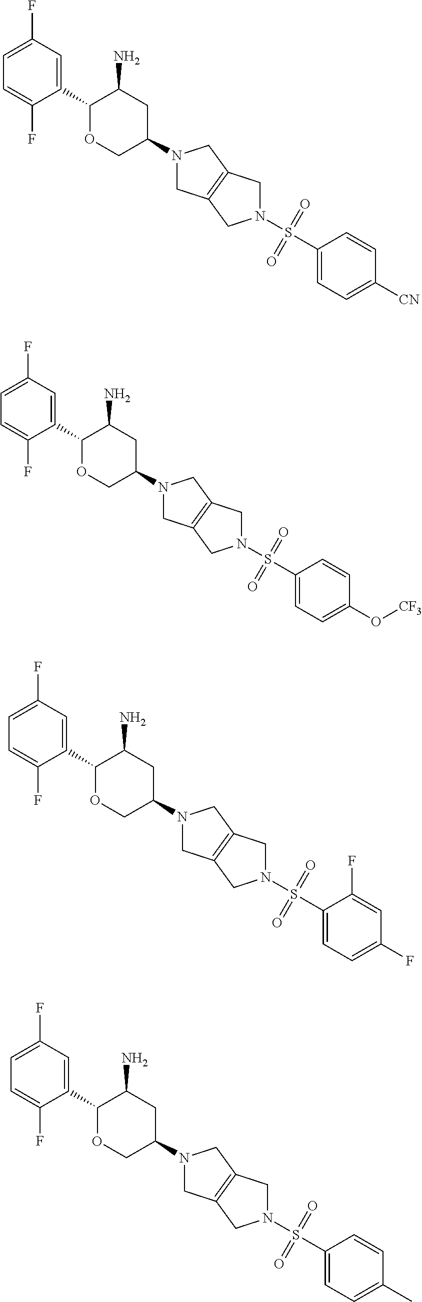 [SMILES: N#Cc1ccc(S(=O)(=O)N2CC3=C(CN([C@H]4CO[C@H](c5cc(F)ccc5F)[C@@H](N)C4)C3)C2)cc1 ; N[C@H]1C[C@@H](N2CC3=C(C2)CN(S(=O)(=O)c2ccc(OC(F)(F)F)cc2)C3)CO[C@@H]1c1cc(F)ccc1F ; N[C@H]1C[C@@H](N2CC3=C(C2)CN(S(=O)(=O)c2ccc(F)cc2F)C3)CO[C@@H]1c1cc(F)ccc1F ; Cc1ccc(S(=O)(=O)N2CC3=C(CN([C@H]4CO[C@H](c5cc(F)ccc5F)[C@@H](N)C4)C3)C2)cc1]

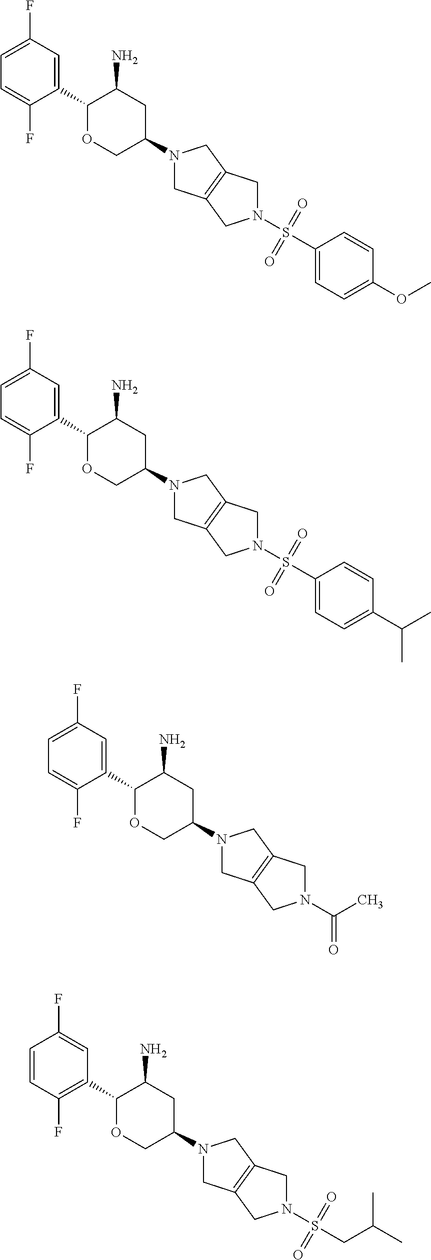 [SMILES: COc1ccc(S(=O)(=O)N2CC3=C(CN([C@H]4CO[C@H](c5cc(F)ccc5F)[C@@H](N)C4)C3)C2)cc1 ; CC(C)c1ccc(S(=O)(=O)N2CC3=C(CN([C@H]4CO[C@H](c5cc(F)ccc5F)[C@@H](N)C4)C3)C2)cc1 ; CC(=O)N1CC2=C(C1)CN([C@H]1CO[C@H](c3cc(F)ccc3F)[C@@H](N)C1)C2 ; CC(C)CS(=O)(=O)N1CC2=C(CN([C@H]3CO[C@H](c4cc(F)ccc4F)[C@@H](N)C3)C2)C1]

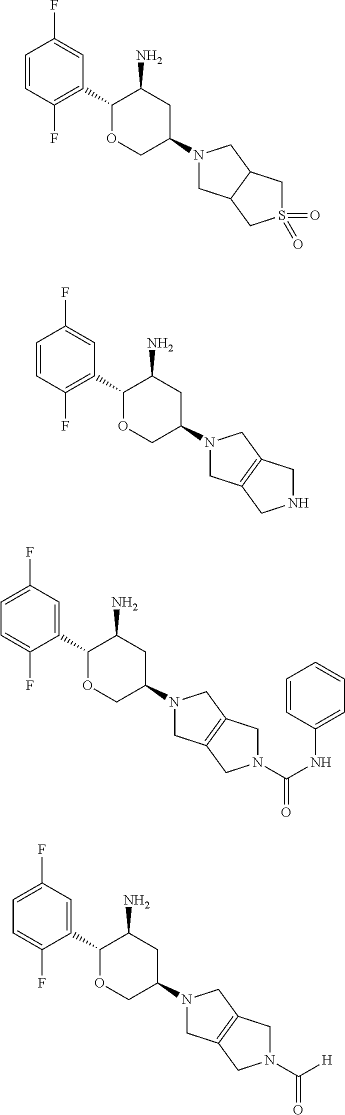 [SMILES: N[C@H]1C[C@@H](N2CC3CS(=O)(=O)CC3C2)CO[C@@H]1c1cc(F)ccc1F ; N[C@H]1C[C@@H](N2CC3=C(CNC3)C2)CO[C@@H]1c1cc(F)ccc1F ; N[C@H]1C[C@@H](N2CC3=C(CN(C(=O)Nc4ccccc4)C3)C2)CO[C@@H]1c1cc(F)ccc1F ; N[C@H]1C[C@@H](N2CC3=C(CN(C=O)C3)C2)CO[C@@H]1c1cc(F)ccc1F]

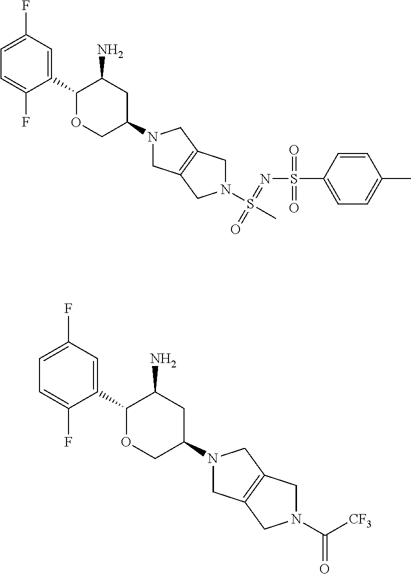 [SMILES: Cc1ccc(S(=O)(=O)N=S(C)(=O)N2CC3=C(CN([C@H]4CO[C@H](c5cc(F)ccc5F)[C@@H](N)C4)C3)C2)cc1 ; N[C@H]1C[C@@H](N2CC3=C(CN(C(=O)C(F)(F)F)C3)C2)CO[C@@H]1c1cc(F)ccc1F]

8. A pharmaceutical composition comprising a therapeutically effective amount of a compound of Formula (I) as claimed in claim 1 and optionally one or more pharmaceutically acceptable carriers, diluents or excipients.

9. The pharmaceutical composition which is useful for reducing blood glucose levels for treating type II diabetes.

10. A method of treating type II diabetes comprising administering to a patient in need thereof an effective amount of a compound of Formula (I) according to claim 1 or its suitable pharmaceutical composition.

11. Use of a compound of Formula (I) or its pharmaceutical composition according to claim 1 for the manufacture of a medicament for increasing insulin secretion for treating type II diabetes.

12. A medicine for the treatment of type II diabetes which comprises administering a therapeutically effective amount of compound of Formula (I) or its pharmaceutical composition as defined in claim 1 to a patient or subject in need thereof.

13. A pharmaceutical composition comprising the compound of the present invention in combination with one or more suitable pharmaceutically active agents selected from insulin, insulin derivatives and mimetics, insulin secretagogues, insulin sensitizers, biguanide agents, alpha-glucosidase inhibitors, insulinotropic sulfonylurea receptor ligands, meglitinides, GLP-1, GLP-1 analogs, DPP-IV inhibitors, GPR-119 activators, sodium-dependent glucose co-transporter (SGLT2) inhibitors, PPAR modulators, non-glitazone type PPAR.delta agonist, HMG-CoA reductase inhibitors, cholesterol-lowering drugs, rennin inhibitors, anti-thrombotic and anti-platelet agents and anti-obesity agents or their suitable pharmaceutically acceptable salts.

14. Use of the compound of formula (I) and a suitable pharmaceutically acceptable agent selected from insulin, insulin derivatives and mimetics, insulin secretagogues, insulin sensitizers, biguanide agents, alpha-glucosidase inhibitors, insulinotropic sulfonylurea receptor ligands, meglitinides, GLP-1, GLP-1 analogs, DPP-IV inhibitors, GPR-119 activators, sodium-dependent glucose co-transporter (SGLT2) inhibitors, PPAR modulators, non-glitazone type PPAR.delta agonist, HMG-CoA reductase inhibitors, cholesterol-lowering drugs, rennin inhibitors, anti-thrombotic and anti-platelet agents and anti-obesity agents or their pharmaceutically acceptable salts for the treatment of diabetes and its associated disorders.
